(19) United States
(12) Patent Application Publication
(10) Pub. No.: US 2026/0226133 A1
(43) Pub. Date: Aug. 6, 2026

(54) METHODS AND COMPOSITIONS FOR GENE TRANSDUCTION AND TO CONTROL THE ACTIVITY OF SYNTHETIC AND IMMUNE RECEPTORS

(71) Applicant: Angeles Therapeutics, Inc., Toluca Lake, CA (US)

(72) Inventor: Preet M Chaudhary, Toluca Lake, CA (US)

(21) Appl. No.: 19/145,170

(22) PCT Filed: Jan. 5, 2024
(86) PCT No.: PCT/US2024/010592

§ 371 Date: Jul. 1, 2025

Related U.S. Application Data
(60) Provisional application No. 63/478,612, filed on Jan. 5, 2023.

(51) Int. Cl. C07K 14/705; C07K 16/00; C07K 19/00; C12N 15/62

(52) U.S. Cl. CPC C07K 14/7051; C07K 16/00; C07K 19/00; C12N 15/62; C07K 2317/24; C07K 2317/52; C07K 2319/03

(57) ABSTRACT

The application provides novel viral envelope glycoproteins for pseudotyping viral vectors, novel designs for synthetic antigen receptors (SARs), novel signaling chains for construction of SARs, novel antigen binding domains, and novel methods for production of SAR-expressing cells. These novel methods and compositions have broad use in cellular therapy.

### CROSS REFERENCE TO RELATED APPLICATIONS

The application claims priority to U.S. Provisional Application No. 63/478,612, filed Jan. 5, 2023, the disclosures of which are incorporated entirely herein by reference.

### TECHNICAL FIELD

The present disclosure relates to the field of biotechnology, and more specifically, to single-chain and multi-chain synthetic antigen receptors.

### INCORPORATION BY REFERENCE OF SEQUENCE LISTING

The instant application includes a Sequence Listing electronically submitted herewith in XLM file format and is incorporated entirely herein by reference. The XLM file was created on Jan. 5, 2024, and named “NKSAR-13.xlm” and is 99.899 megabytes in size.

### BACKGROUND

Chimeric Antigen Receptors (CARs) are synthetic receptors, which can redirect T cells to selectively kill tumor cells. To overcome some of the design limitation of conventional 2^{nd} generation CARs, several alternative designs, collectively termed next generation CARs, have been described, including Ab-TCR (WO 2017/070608 A1 incorporated entirely herein by reference), TCR receptor fusion proteins or TFP (WO 2016/187349 A1 incorporated entirely herein by reference), Synthetic Immune Receptors (SIRs) (WO 2018/102795 A1, incorporated entirely herein by reference), Tri-functional T cell antigen coupler (Tri-TAC) (WO 2015/117229 A1, incorporated entirely herein by reference) and Synthetic antigen receptors (SARs), including universal TCR-SAR (or uTCR-SAR) (PCT/US22/17177, incorporated entirely herein by reference). The STAR (WO2020029774, incorporated entirely herein by reference) and HLA-independent TCR (HIT) (WO201915745 A1, incorporated entirely herein by reference) are similar in design to the SIR platform. These alternative CAR designs, in general, lack a co-stimulatory domain. The present disclosure describes novel Synthetic Antigen Receptors (SAR), novel antigen binding domains, novel viral envelopes, and in vivo approaches for manufacturing SARs.

### SUMMARY

The disclosure provides uni-specific, bispecific, multi-specific and universal Synthetic Antigen Receptor (SAR) designs. The term SAR refers to any non-native antigen binding receptor that is expressed on the surface of a cell (e.g., immune cell). In an embodiment, a SAR comprises a single polypeptide chain. In an embodiment, a SAR comprises more than one polypeptide chains. In an embodiment, the SAR comprises two polypeptide chains. The disclosure also provides novel accessory modules comprising co-receptors (e.g., CD8a, CD8b, and CD4) that can be co-expressed with the SARs (e.g., uTCR-SAR, HC-SAR, zSIR, zCD16-SAR, etc.) of the disclosure. Examples of co-stimulatory co-receptors are provided in (SEQ ID NO (DNA): 644-646 and SEQ ID NO (PRT): 9024-9026).

The disclosure provides a method of producing a cell that expresses any one or more of the accessory modules with any one or more of the SARs of the disclosure.

The disclosure provides novel design of uni-specific, bispecific, and multi-specific SARs (e.g., SIR, Ab-TCR, etc.) comprising one or more hybrid TCR constant chains or functional variants thereof, including variants from non-human species (e.g., mouse, cat, dog, monkey, etc.). Example hybrid TCR constant chains are provided in SEQ ID NOs (DNA): 529-544, 21350-21814, 22794-23019, 23021-23070, 23072-23231, 23901-27920, and SEQ ID NOs (PRT): 8909-8924, 22112-22576, 23420-23677, 27936-31955. The disclosure also provides that SIR can be constructed using TCR constant chains that encode for polypeptides with at least 70%, 80%, 90%, 95%, 98%, 99% or 100% amino acid identity to the hybrid TCR constant chains that are provided in SEQ ID NO (PRT): 8909-8924, 22112-22576, 23420-23677, 27936-31955. The disclosure also provides hybrid TCR constant chains that encode for polypeptides with N-terminal deletions of between 1 to 100 amino acids of the TCR constant chains with SEQ ID NO (PRT): 8909-8924, 22112-22576, 23420-23677, 27936-31955. The disclosure also provides that SIR can be constructed using TCR constant chains that are functional variants and functional fragments (including deletion mutants) of the hybrid TCR constant chains that are provided in SEQ ID NO (PRT): 8909-8924, 22112-22576, 23420-23677, 27936-31955, including homologs from non-human species. In an embodiment, the hybrid TCR chains are codon optimized, optionally human codon optimized. In some embodiments, the hybrid TCR chains comprise cysteine mutations at specific residue that result in the formation of an additional (second) disulfide bond between the complementary TCR chains. In some embodiments, the hybrid TCR chains comprise mutations in which the human amino acid residues are replaced with the corresponding amino acids from a non-human species, such as mouse TCR chains. In some embodiment, the SAR comprises a hybrid T-cell receptor α (Cα) chain constant region and a hybrid T-cell receptor beta (Cβ) chain constant region, where the Cα region comprises mutations at positions-amino acids 10C, 15C, 21F, 32I, 45C, 48C, 61R, 72T, 91S, 92D, 93V, 94P, 95R, 95S, 116L, 119V, 120L and any combination thereof corresponding to a reference Ca chain represented by SEQ ID NO: 8833, and the Cβ region comprises mutations at positions amino acids 15C, 17C, 18K or R, 22A, 23R, 39P, 54D, 57C, 59C, 77C, 79G, 131G, 131S, 133I, 136A, 139H and any combination thereof corresponding to reference Cβ chains represented by SEQ ID NO: 8847 and 8848. In an embodiment, both the chains of a double chain SAR (e.g., a SIR, Ab-TCR, HIT, STAR, zSIR, zSAR, zCD16 SAR, etc.) or a one and a half chain SAR (e.g., a SIR, Ab-TCR, HIT or STAR, etc.) comprise hybrid TCR chains. In an embodiment, one of the two chains of a double chain SAR or a one and a half chain (OHC) SAR comprises a hybrid TCR constant chain and the second chain of the double chain SAR or a one and a half chain (OHC) SAR comprises a non-hybrid TCR constant chain. In an embodiment, at least one chain of a double chain SAR or a OHC comprises a hybrid TCR constant chain. In an embodiment, at least one chain of a double chain SAR or a OHC comprises a hybrid TCR constant chain that carries additional modifications, wherein the modifications are optionally selected from deletions, substitutions and/or mutations of one or more amino acids. Examples of double chain and one and half chain SARs (e.g., SIR) with hybrid TCR constant chains are provided in Tables 16-20 of provisional application. Human genome comprises two highly homologous TCRβ constant chains: Cβ1 and Cβ2. Although, most of the Cβ chains sequences (e.g., SAR, hybrid TCR chain and fragments thereof) in the current application are based on the sequence of the Cβ2 chain, one with the ordinary skills in the art will recognize that these sequences can be replaced by the corresponding sequences based on Cβ1 chain.

The 2^{nd} generation CAR-T cells in current clinical use are associated with several toxicities, such as cytokine release syndrome (CRS), immune effector cell associated neurological complications (ICAN), non-ICAN neurological complications and secondary cancers. A number of these complications are linked to tonic signaling via the CAR construct, which results in antigen independent CAR-T cell proliferation. In some embodiment, the disclosure provides that a SAR with hybrid TCR chains shows less tonic signaling and antigen independent proliferation as compared to a 2^{nd} generation CAR. The CAR-T cells have been also shown to recognize low level antigens expressed on normal healthy tissues. This results in on-target off-tumor toxicity. To overcome this problem affinity turned CAR-T constructs have been described which use antigen binding domains (e.g., scFvs, vHH) with lower affinity. However, affinity tuning of an antigen binding domain is an expensive and time-consuming process. It can also lead to loss of affinity or acquisition of new and unexpected binding properties for the antigen binding domain. To overcome this limitation, the disclosure provides a simple method of generating a diverse panel of SAR with varying affinity by using different signaling chains. In one embodiment, the disclosure provides that the hybrid and/or mutant TCR chains of the disclosure can be used to quickly generate a diverse panel of SAR (e.g., a SIR, Ab-TCR, HIT, STAR, zSIR, zSAR, zCD16 SAR, etc.) constructs of varying affinities that comprise the same antigen binding domain. Therefore, the current disclosure provides a simple method for generation of a diverse panel of affinity-tuned and affinity-enhanced SAR constructs with different affinities that are based on the same antigen binding domain (e.g., vL, vH, scFv, Fv, vHH, etc.). Thus, the method of the disclosure overcomes the limitations and risk associated with mutagenesis of antigen binding domain to generate diverse panel of affinity tuned and/or affinity enhanced SAR and CAR constructs. The diversity of the SAR pool is further increased by the use of different junctions in the hybrid chains and by the use of different linkers that can be present between the different domains of a SAR (e.g., antigen binding domain and TCR constant domain). The diversity of T cells expressing the pool can be further increased by use of different accessory modules and therapeutic controls described in the disclosure.

This diverse pool of SARs can be used to provide a diverse immune response against disease causing or disease associated cells expressing the said antigen. Alternatively, the diverse pool of SARs can be optionally DNA barcoded using techniques known the art and subsequently used to select a single or a subgroup of SIRs with optimal biological and clinical characteristics. These characteristics may include but are not limited to, performance in the in vitro biological assays (e.g., cytotoxicity, cytokine secretion, binding affinity, cell surface expression, off-target effects, T cell proliferation, expression of exhaustion markers and terminal differentiation etc.), performance in the in vivo assays (e.g., survival, tumor reduction, T cell persistence, T cell expansion etc.) and clinical experience (e.g., disease remission, relapse rate, toxicities, etc.). The SARs of the disclosure can be used singly or in combination with other SIRs, CARs, cTCRs, zSIR, zCD16 SAR and other natural and synthetic immune receptors known in the art to generate a diverse pool of immune effector cells for the prevention and treatment of various disease conditions caused by or associated with cells expressing their target antigens.

In any of the embodiments described herein, an effector cell expressing a SAR of one type shows diverse properties as compared to an effector cell expressing a SIR of different type (such as an effector cell presenting on its surface a SIR comprising the antigen binding domain of the first SIR but with different TCR chains, e.g., a SIR comprising an scFv, a vL and/or a vH fragment comprising the antigen binding domains of the first SIR but with different TCR chains) when compared under similar conditions. As SIRs are modular in design, additional SIR types can be generated by one skilled in the art by replacing one module with another. Exemplary properties in which SIRs of different type may show diversity when expressed in an immune effector cell include, but are not limited to, binding affinity, cell-surface expression, cytotoxicity, cytokine production, cellular proliferation, terminal differentiation, exhaustion and in vivo biological activity. In an exemplary embodiments, an effector cell expressing a SAR1 (SEQ ID NO: 40140) containing a hu-mROO5-1 based CD19-targeting domain has a higher binding to CD19-ECD-GGSG-NLuc-AcV5 fusion protein after 60 minutes incubation at 4° C. as compared to a corresponding effector cells expressing SAR2 (SEQ ID NO: 40146) or SAR3 (SEQ ID NO: 40147) targeting CD19 when examined under similar conditions and when both SAR types are targeted to the TRAC (TCR alpha constant chain) genomic locus to rule out any variance in expression due to random sites of integration of different SIR constructs. In some embodiments, the target antigen-binding of an effector cell expressing a SIR of one type (e.g. SAR1) after 60 minutes incubation at 4° C. is at least 5, 10, 20, 30, 40, 50% or 100% more than the target antigen-binding of an effector cell expressing a SAR of a different type (e.g., SAR2) containing the same binding domain when examined under similar conditions and when both SAR types are targeted to the TRAC (TCR alpha constant chain) genomic locus. In some embodiments, the target antigen-binding of effector cells expressing SAR of different types (e.g., SAR1, SAR2, SAR3 and so on) containing the same binding domain after 60 minutes incubation at 4° C. varies by more than 5-fold, 10-fold, 20 fold, 50 fold or 100 fold when examined under similar conditions and when both SIR types are targeted to the TRAC (TCR alpha constant chain) genomic locus. Techniques to target a genomic insert to a specific genomic locus are known in the art. In some embodiments, the standard deviation in the target antigen-binding of effector cells expressing SIR of different types (e.g. SIR1, SIR2, SIR3 and so on) containing the same binding domain after 60 minutes incubation at 4° C. is more than 2-fold, 5-fold, 10-fold, 20 fold, 50 fold or 100 fold as compared to the standard deviation in the target antigen-binding of independently isolated populations of effector cells expressing a corresponding cTCR when examined under similar conditions and when the different SIR types and the cTCR are targeted to the TRAC locus. In other embodiments of the disclosure, the standard deviation in the cytotoxicity of effector cells expressing SIR of different types (e.g. SAR1, SAR2, SAR3 and so on) containing the same binding domain after 4 hours incubation at 37° C. with the target cells is more than 2-fold, 5-fold, 10-fold, 20 fold, 50 fold or 100 fold as compared to the standard deviation in the cytotoxicity of independently isolated populations of effector cells expressing a corresponding cTCR when each of the SIR types and the cTCR are inserted at the TRAC locus. Standard deviation is square root of variance and can be measured by methods known in the art. In some embodiments, the SIR-expressing effector cell is a SIR T cell. In some embodiments, the SIR-expressing effector cell is a SIR-expressing Jurkat T cell.

The disclosure also provides that a SAR with one or more hybrid TCR chains is less likely to pair with the endogenous TCR chains as compared to a cTCR (chimeric T cell receptor) or a SIR. In some embodiment, the disclosure provides that a SAR with one or more hybrid TCR chains shows higher expression and functional activity (e.g., cytokine production, cytotoxicity etc.) as compared to a SAR with wild-type TCR constant chains (i.e., a cTCR) when expressed in immune cells (e.g., T, NK cells). In some embodiment, the disclosure provides that a SAR with one or more hybrid TCR chains shows lower expression and functional activity (e.g., cytokine production, cytotoxicity etc.) as compared to a second-generation CAR construct when expressed in immune cells (e.g., T, NK cells). In some embodiment, the disclosure provides that a SAR with one or more hybrid TCR chains shows lower expression, binding affinity and functional activity (e.g., cytokine production, cytotoxicity etc.) as compared to a SAR with non-hybrid TCR chains (e.g., SIR, STAR, HIT etc.) or a second-generation CAR when expressed in immune cells (e.g., T, NK cells). In some embodiment, the disclosure provides that immune cells expressing a SAR with one or more hybrid TCR chains show less cytokine production and produce less side effects when administered to a subject. In some embodiment, immune cells expressing a SAR with one or more hybrid TCR chains show less on-target off-tumor toxicity when administered to a subject. In an embodiment, immune cells expressing a SAR with one or more hybrid TCR chains are safer when administered to a subject. In an embodiment, immune cells expressing SAR with one or more hybrid TCR chains show effective anti-tumor activity when administered to a subject. In an embodiment, immune cells expressing a SAR with one or more hybrid TCR chains show superior anti-tumor activity against solid tumors as compared to a conventional second-generation CAR.

The disclosure also provides SAR with one or more hybrid TCR chains that are expressed from an endogenous TCR gene locus. The disclosure also provides SAR with one or more hybrid TCR chains that are targeted to the gene locus of an endogenous T cell gene (e.g., TCRα, TCRβ, TCRγ, TCRδ or CD3z). Example targeting constructs for targeting SARs with hybrid TCR constant chains to the TRAC locus are also provided in SEQ ID NO: 8396-8404. Additional targeting constructs can be constructed by replacing one or more modules (e.g., vL, vH, vHH, FHVH, TCR chains etc.) of the constructs shown in SEQ ID 0: 8396-8404 with different modules described herein.

The disclosure provides novel SIR, Ab-TCR and MHC-SAR/HLA-SAR designs, including those with hybrid chains, comprising an activation domain attached to one or both TCR constant chains. In an embodiment the activation domain is derived from cytosolic domain of CD3z or FcRy or a functional fragment or variant thereof. In an embodiment, the activation domain is operationally linked to the C-terminus of the one or both TCR constant chains comprising a SIR or an Ab-TCR. In an embodiment, the SIR comprises a co-stimulatory domain that is operationally linked to one or both TCR chains. In an embodiment, the co-stimulatory is operationally linked to the C-terminus region of the one or both TCR constant chains. In an embodiment, the co-stimulatory is operationally linked to the C-terminus region of the one or both activation domain that are attached to one or both TCR constant chains. In an embodiment, the co-stimulatory domain is derived from cytosolic domain of CD28, 4-1BB, OX40, CD40, CD27, CD2 etc. Example TCR chains with activation domains are presented in SEQ ID NO (DNA): 23901-25240 and SEQ ID NO (PRT): 27936-29275. Example TCR chains with activation domains and costimulatory domains are presented in SEQ ID NO (DNA):25241-27920 and SEQ ID NO (PRT):29276-31955. Example SIR with TCR chains comprising activation and/or co-stimulatory domains are provided in SEQ ID NO (DNA): 27922-27927 and SEQ ID NO (PRT):31957-31962. In an embodiment, the SIR, Ab-TCR and MHC-SAR with activation and/or costimulatory domains in the TCR constant chains show improved (e.g., at least 5% improvement) cytokine production, cytotoxicity and proliferation as compared to SIR, Ab-TCR and MHC-SAR lacking the activation and/or costimulatory domains in the TCR chains.

The double chain SAR constructs comprising antigen binding domain attached via optional linkers to one or two CD3z polypeptide chains have been described (see WO 2016/187349 A1, WO 2018/102795 A1, PCT/US22/17177, incorporated herein by reference). The disclosure provides SAR (e.g., uTCR-SAR, zSIR, zCD16-SAR etc.) comprising CD3z chains with deletion of residue Q101 (dQ101) in the cytosolic domain or functional variants thereof or a homolog from a non-human species (e.g., mouse, dog, monkey etc.). Example CD3z chains with deletion of residue Q101 (CD3zECDTMCP-dQ101) are provided in SEQ ID NO (DNA): 943-966 and SEQ ID NO (PRT): 9323-9346. The disclosure also provides that SAR (e.g., uTCR, zSIR, zCD16SAR etc.) can be constructed using CD3z chains with at least 70%, 75%, 80%, 85%, 90%, 95%, 98%, 99% amino acid homology to the CD3z chains that are provided in SEQ ID NO (PRT): 9323-9346, 40592-40605. In an embodiment, both chains of a double chain SAR comprise CD3z chains with deletion of Q101 residue. In an embodiment, a double chain SAR comprises at least one chain comprising a CD3z chain with deletion of amino acid residue Q101.

In an embodiment, a double chain SAR comprises one CD3z chains with deletion of Q101 residue and a second chain comprising a transmembrane domain or a membrane anchoring domain. In an embodiment, the second chain may also comprise a hinge domain and optionally a cytosolic domain. In an embodiment, a double chain SAR comprises one CD3z chains with deletion of Q101 residue and a second chain comprising a CD16 hinge and transmembrane domains. Such a SAR is designated a z16SIR or zCD16SAR or zCD16SIR. In an embodiment, the second chain of a zCD16SAR may further comprise a CD16 cytosolic domain. In an embodiment, the second chain of a zCD16SAR may comprise a costimulatory domain. In an embodiment, the second chain of a zCD16SAR may comprise a costimulatory domain but lacks a CD16 cytosolic domain or may comprise a partial CD16 cytosolic domain. In an embodiment, the costimulatory domain is derived from 4-1BB, CD28, OX40, 2B4, CD8a, CD8b or CD4. In an embodiment, the second chain of a zCD16SAR may comprise a signaling domain. In an embodiment, the signaling domain may be derived from a kinase. In an embodiment, the signaling domain is derived from Lck, mutant Lck, LAT, ZAP-70, SLP-76 or a mutant or variant thereof.

In an embodiment, a double chain SAR comprises one CD3z chain with deletion of Q101 residue and a second chain comprising a FcRy hinge and transmembrane domains. Such a SAR is designated a zFcRy-SAR. In an embodiment, the second chain may further comprise a cytosolic domain. In an embodiment, the second chain of a zCD16SAR may comprise a costimulatory domain. In an embodiment, the cytosolic domain of the first chain and/or the second may comprise one or more ITAM. In an embodiment, the cytosolic domain of the first chain and/or the second may comprise one or more co-stimulatory domains. In an embodiment, the cytosolic domain of the first chain and/or the second may comprise one or more co-receptor domains. Example coreceptor cytosolic domains comprise cytosolic domains CD8a, CD8b or CD4. In an embodiment, the SAR comprising a dQ101 mutation in the CD3z chain shows enhanced activity (e.g., NFAT activation, cytokine production, or cytotoxicity etc.) as compared to a SAR comprising a CD3z chain that lacks the dQ101 mutation. The disclosure also provides that SAR (e.g., uTCR, zSIR etc.) can be constructed using CD3z chains that are functional variants of the hybrid CD3z chains that are provided in SEQ ID NO (PRT): 9323-9346), including homologs from non-human species. Example SAR with CD3z chains with deletion of residue Q101 are provided in SEQ ID NO (DNA): 3171-3243 and SEQ ID (PRT): 11551-11623.

The present disclosure also features novel SAR (e.g., “MIC-SARs”) comprising a portion of an MHC molecule (e.g., class I, class II, non-classical MHC) and a portion of a non-TCR signaling receptor or a non-TCR signaling chain. Example such non-TCR signaling receptors and non-TCR signaling chains include CD3z, CD3z-dQ101, FcRγ, DAP10, DAP12, CD16A, CD16B, NKp30, NKp44 and NKp46 etc. and functional variants thereof, including homologs from non-human species. In some embodiments, the MHC-SAR comprises a portion of an antigen peptide. In an embodiment, the peptide comprises a disulfide trap to enhance the stability and expression of the MICH-SAR. The present invention also features cells, such as T cells or NK cells, macrophages, B cells, dendritic cells, granulocytes etc., expressing said MHC-SARs (cells expressing an MHC-SAR are herein referred to as “redirected cells”). The MHC-SARs are adapted to recognize and bind to appropriate (specific) TCRs. Redirected cells (e.g., redirected T cells, NK cells or macrophage etc.) expressing a MHC-SAR would mimic antigen presenting cells (APCs), the cells that normally express MHC molecules. In some cases, binding of a TCR of a target T cell to the MIC-SAR of the redirected cell may then result in destruction of the target T cell, thus, in this case, the redirected cells may function as “anti-T cell” T, NK cells or macrophages. The present invention is not limited to redirected cells functioning to destroy a target. For example, in some embodiments, the redirected cell is adapted to help reprogram a target cell, e.g., the redirected cell may deliver instructions to the target cell. In an embodiment, the M-HC-SAR can be used to eliminate auto-reactive T cells for the prevention and/or treatment of autoimmune disorders (e.g., multiple sclerosis, diabetes mellitus etc.). Example MHC-SAR are provided in SEQ ID NO (DNA): 23780-23794 and SEQ ID NO (PRT): 23877-23891. These constructs comprise a RQ13 peptide attached to the N-terminus of HLA-DRB-B1 (or D1) domain. These MHC-SAR react to T cell expressing the F24 CAR (SEQ ID NO (DNA):23236 and SEQ ID NO (PRT): 23682) that recognizes RQ13 peptide/HLA-DR complex. As MHC-SAR are modular in design, different MHC SAR can be constructed by replacing the signaling chains of the above MHC-SAR with the different signaling chains described in this disclosure. For example, the [hTCRb-S57C] and [hTCRa-T48C] chains of the MHC-SAR CD8SP-Sph-R13Q-Kpn-G4S-RI-1-HLA-DRB-B1-[hTCRb-S57C]-F-P2A-SP-HILA-DRA-A1-[hTCRa-T48C] represented by SEQ ID NO: 23887 can be substituted by different TCRβ and TCRα chains or TCRγ and TCRδ chains, including hybrid TCRβ, TCRα, TCRγ and TCRδ chains described in this disclosure. In addition, the [hTCRb-S57C] and [hTCRa-T48C] chains can be replaced by TCR chains comprising activation domain and/or costimulatory domains. Similarly, the RQ13 peptide RFYKTLRAEQ (SEQ ID NO: 23892) can be replaced by a different peptide (e.g., MOG peptide, NY-ESO-1 or (Glia-gamma1 peptide) to target TCRs recognizing these peptide antigens in complex with the 1-HLA molecules. The peptide may further comprise disulfide traps to enhance the stability of the peptide/MHC complex. Finally, the HLA-DRB-D1 and HLA-DRA-A1 modules can be replaced by different HLA modules, such as HLA-DQB1-D1 (SEQ ID NO: 23775) and HLA-DQA1-D1 (SEQ ID NO: 23771) or HLA-DPB1-D1 (SEQ ID NO: 23755) and HLA-DPA1-D1 (SEQ ID NO: 23752). For example, an MHC-SAR in which Glia-gamma1 peptide (SEQ ID NO: 20377) replaces the RQ13 peptide and the HLA-DQB1-D1 (SEQ ID NO: 23775) and HLA-DQA1-D1 (SEQ ID NO: 23771) replace the [hTCRb-S57C] and [hTCRa-T48C] modules can be used to target immunoreactive T cells targeting gluten-derived (Glia-gamma1/HLA-DQ8.5 complex for the prevention and treatment of celiac disease.

The disclosure also provides novel accessory modules comprising co-stimulatory co-receptors (e.g., CD8a, CD8b and CD4) that can be co-expressed with the SARs (e.g., uTCR-SAR, zSIR, MHC-SAR, hybrid chain SAR, SIR, HC-SAR, Ab-TCR etc.) of the disclosure. Example co-stimulatory co-receptors are provided in SEQ ID NO (DNA): 644-646 and SEQ ID NO (PRT): 9024-9026).

The present invention also features engineered cells expressing both a SAR (e.g., MHC-SAR, uTCR-SAR, hybrid chain SAR, SIR, zSIR etc.) and a surrogate coreceptor (SCR). The engineered cells co-expressing a SAR (e.g., MHC-SAW, uTCR-SAR, SIR, hybrid chain SAR, SIR, zSIR etc.) and an SCR can show enhanced effects (e.g., increased IL-2 expression) as compared to engineered cells expressing a SAR without co-expression of an SCR.

The disclosure provides novel SAR comprising signaling chains comprising CD4, CD8b and CD8a cytosolic domains or functional variants thereof. The example signaling chains with CD4, CD8b and CD8a cytosolic domains are provided in (SEQ ID NO (DNA): 555-558 and SEQ ID NO (PRT): 8925-8934. The disclosure also provides that SAR (e.g., uTCR, zSIR etc.) can be constructed using signaling chains with at least 70%, 75%, 80%, 85%, 90%, 95%, 98%, 99% amino acid homology or identity to the signaling chains that are provided in SEQ ID NO (PRT): 8925-8934. The disclosure also provides that SAR (e.g., uTCR, zSIR etc.) can be constructed using signaling chains that are functional variants of the signaling chains that are provided in SEQ ID NO (PRT): 8925-8934, including homologs from non-human species. An example SAR with a signaling chain comprising a CD4 cytosolic domain is represented by SEQ ID NO (DNA): 1029. An example SAR with a signaling chain comprising a CD8a cystolic domain is represented by SEQ ID NO (DNA): 1028. An example SAR with a signaling chain comprising a CD8a cytosolic domain and a second signaling chain comprising CD8b cytosolic domain is represented by SEQ ID NO (DNA): 1046. An example SAR with a signaling chain comprising a CD4 cytosolic domain and a second signaling chain comprising CD8a cytosolic domain is represented by SEQ ID NO (DNA): 1047.

The disclosure provides novel SAR comprising signaling chains comprising co-stimulatory domains or functional variants thereof. The example signaling chains with 41BB, CD28, CD30 cytosolic domains are provided in (SEQ ID NO (DNA): 545-554 and SEQ ID NO (PRT): 8935-8938. The disclosure also provides SAR comprising signaling chains with at least 70%, (e.g., 70%, 75%, 80%, 90%, 95%, 98%, 99% or 100%) amino acid identity to signaling chains provided in SEQ ID NO (PRT): 8935-8938 and functional variants thereof.

The disclosure provides novel linkers, including novel Ig Linkers, that can be used to generate SARs (e.g., SIR) of the disclosure. In an embodiment, a novel Ig linker comprises deleted or mutated immunoglobulin like linker domains derived from antibodies and TCR constant chains. In an embodiment, a novel Ig linker comprises a deletion or point mutant of any of linkers described in SEQ ID (PRT): 8961-8994 or a functional variant thereof. In an embodiment, a novel Ig linker comprises a linker with at least 70% (e.g., 70%, 75%, 80%, 90%, 95%, 98%, 99% or 100%) amino acid identity to any one of linkers described in SEQ ID (PRT): 8961-8994. In an embodiment, a novel Ig linker of the disclosure comprises a linker with N-terminal deletion of between 1-100 amino acids of any of the linkers described in SEQ ID (PRT): 8961-8994 or a functional variant thereof. Example TCR constant chains, including hybrid TCR constant chains, comprising N-terminal deletion of immunoglobulin like linker domains are presented in SEQ ID NO (PRT): 18259-18914. The disclosure also describes that SAR can be generated using functional variants or mutants of the above TCR constant chains and/or linkers, including homologs from non-human species.

In an embodiment, the linker comprises the Ig-like constant domain of a TCR chain and further comprises a TCR connecting peptide. Example long Ig-like linkers are provided in SEQ ID NO: 22827-22829, 22833-22835, 22839-22840, 22843-22844, respectively and also include functional variants and homologs which encodes for polypeptide with at least 75% sequence identity to a polypeptide encoded by any of the above sequences. In an embodiment, the long Ig-like linker comprises N-terminal or C-terminal deletion mutants of in SEQ ID NO: 22827-22829, 22833-22835, 22839-22840, 22843-22844, respectively, in which between 1-40 (e.g., 1, 5, 10, 15, 20, 25, 30, 40) N-terminal or C-terminal amino acid residues are deleted.

The disclosure provides novel double chain SAR (e.g., SIR) and novel one and half chain SAR (e.g., SIR) comprising one or more non-TCR antigen binding domains in which the immunoglobulin like linker domain (e.g., SEQ ID NO (DNA): 597-614 and SEQ ID NO (PRT):8977-8994) of the one or both TCR constant chains of a double chain SAR or of a one and half chain SAR are replaced by Ig-like linker domains derived from a different TCR chain or a mutant or a functional variant thereof. The disclosure provides novel double chain SAR (e.g., SIR) and novel one and half chain SAR (e.g., SIR) comprising one or more non-TCR antigen binding domains in which the immunoglobulin like linker domain (e.g., SEQ ID NO (DNA): 597-614 and SEQ ID NO (PRT):8977-8994) and connecting peptide/hinge domain (e.g., SEQ ID NO (DNA): 615-622 and SEQ ID NO (PRT): 8995-9002) of the one or both TCR constant chains of a double chain SAR (e.g., SIR) or of a one and half chain SAR (e.g., SIR) are replaced by Ig-like linker domains and connecting peptide (or hinge domains) derived from a different TCR chain or a mutant or a functional variant thereof. The example connecting peptides/Hinge domains of TCRα are represented by SEQ ID NO: 8995-8996), TCRβ (SEQ ID NO: 8997-8998), TCRγ (SEQ ID NO: 8999-9000) and TCRδ (SEQ ID NO: 9001-9002) and are provided in Table 7 of provisional patent application. The disclosure also provides SAR comprising hybrid TCR constant chains comprising connecting peptides with at least 70% amino acid homology to connecting peptides provided in SEQ ID NO (PRT): 8909-8924, and functional variants and mutants thereof, including homologs from non-human species.

The disclosure provides novel double chain SAR (e.g., SIR) and novel one and half chain SAR (e.g., SIR) comprising one or more non-TCR antigen binding domains in which the immunoglobulin like linker domain (e.g., SEQ ID NO (DNA): 597-614 and SEQ ID NO (PRT):8977-8994 of the one or both TCR constant chains of a double chain SAR or of a one and half chain SAR are replaced by linker domains that are not derived from TCR chains or a mutant or a functional variant thereof.

The disclosure also provides novel one and half chain SAR comprising one or more non-TCR antigen binding domains in which the immunoglobulin like linker domain (e.g., SEQ ID NO (DNA): 597-614 and SEQ ID NO (PRT):8977-8994 of the one or both TCR constant chains of a double chain SAR or of a one and half chain SAR are replaced by Ig linker domains derived from a different antibodies/immunoglobulins or a mutant or a functional variant thereof. Example Ig linker domains derived from a different antibodies/immunoglobulins are provided in Table 7 of the provisional application (SEQ ID NO (DNA):581-596 and SEQ ID NO (PRT): 8961-8976). The disclosure also provides SAR comprising hybrid TCR constant chains comprising Ig linkers with at least 70% (e.g., 70%, 75%, 80%, 90%, 95%, 98%, 99% or 100%) amino acid identity to Ig linkers provided in SEQ ID NO (PRT): 8909-8924, and functional variants and mutants thereof, including homologs from non-human species.

The example immunoglobulin like linker domains of TCRα constant chains are provided in SEQ ID NO (PRT):8977-78, 8986-88 and include functional variants, mutants, and homologs thereof, including homologs from non-human species. In an embodiment, immunoglobulin like linker domains of TCRα constant chains comprise a polypeptide with at least 70% (e.g., 70%, 75%, 80%, 85%, 90%, 95%, 98%, 99% of 100%) amino acid sequence identity to any of the sequences represented by SEQ ID NO (PRT):8977-78 and 8986-88 or a functional variant, mutant or homolog thereof. In an embodiment, immunoglobulin like linker domains of TCRα constant chains comprise a polypeptide with deletion of up to 60 (e.g., 1, 2, 5, 10, 15, 20, 25, 30, 35, 40, 45, 50, 55, 60 etc.) N-terminal amino acids of any of the sequences represented by SEQ ID NO (PRT):8977-78 and 8986-88 or a functional variant, mutant or homolog thereof.

The example immunoglobulin like linker domains of TCRβ constant chains are provided in SEQ ID NO (PRT):8979-80, 8985, 8989-90 and include functional variants, mutants, and homologs thereof, including homologs from non-human species. In an embodiment, immunoglobulin like linker domains of TCRβ constant chains comprise a polypeptide with at least 70% (e.g., 70%, 75%, 80%, 85%, 90%, 95%, 98%, 99% of 100%) amino acid sequence identity to any of the sequences represented by SEQ ID NO (PRT): 8979-80, 8985, 8989-90 and functional variants, mutants, and homologs thereof. In an embodiment, immunoglobulin like linker domains of TCRα constant chains comprise a polypeptide with deletion of up to 60 (e.g., 1, 2, 5, 10, 15, 20, 25, 30, 35, 40, 45, 50, 55, 60 etc.) N-terminal amino acids of any of the sequences represented by SEQ ID NO (PRT): 8979-80, 8985, 8989-90 or a functional variant, mutant or homolog thereof.

The example immunoglobulin like linker domains of TCRγ constant chains are provided in SEQ ID NO (PRT):8981-82 and 8991-92 and include functional variants, mutants, and homologs thereof, including homologs from non-human species. In an embodiment, immunoglobulin like linker domains of TCRγ constant chains comprise a polypeptide with at least 70% (e.g., 70%, 75%, 80%, 85%, 90%, 95%, 98%, 99% of 100%) amino acid sequence identity to any of the sequences represented by SEQ ID NO (PRT): 8981-82 and 8991-92 or a functional variant, mutant or homolog thereof. In an embodiment, immunoglobulin like linker domain of TCRγ constant chains comprises a polypeptide with deletion of up to 60 (e.g., 1, 2, 5, 10, 15, 20, 25, 30, 35, 40, 45, 50, 55, 60 etc.) N-terminal amino acids of any of the sequences represented by SEQ ID NO (PRT): 8981-82 and 8991-92 or a functional variant, mutant or homolog thereof.

The example immunoglobulin like linker domains of TCRδ constant chains are provided in SEQ ID NO (PRT):8983-84 and 8993-94 and include functional variants, mutants, and homologs thereof, including homologs from non-human species. In an embodiment, immunoglobulin like linker domains of TCRδ constant chains comprise a polypeptide with at least 70% (e.g., 70%, 75%, 80%, 85%, 90%, 95%, 98%, 99% of 100%) amino acid sequence identity to any of the sequences represented by SEQ ID NO (PRT): 8983-84 and 8993-94 or a functional variant, mutant or homolog thereof. In an embodiment, immunoglobulin like linker domain of TCRδ constant chains comprises a polypeptide with deletion of up to 60 (e.g., 1, 2, 5, 10, 15, 20, 25, 30, 35, 40, 45, 50, 55, 60 etc.) N-terminal amino acids of any of the sequences represented by SEQ ID NO (PRT): 8983-84 and 8993-94 or a functional variant, mutant or homolog thereof.

In example embodiments, the disclosure provides a novel double chain (DC) SAR and novel one and half chain (OHC) SAR comprising one or more non-TCR antigen binding domains that are operationally linked via optional linkers to one or more hybrid TCR constant chains. In an embodiment, the hybrid TCR constant chains comprises a chain in which immunoglobulin like linker domain of a TCR constant chain is replaced by the immunoglobulin like linker domain of a different TCR constant chain or a homolog or variant thereof with at least 70% (e.g., 70%, 75%, 80%, 85%, 90%, 95%, 98%, 99% of 100%) amino acid sequence identity. In an embodiment, a hybrid TCRα constant chain comprises a chain in which the immunoglobulin like linker domain of TCRα constant chain is replaced by the immunoglobulin like linker domain of TCRβ, TCRγ, TCRδ or pre-TCRα constant chain or a homolog or variant thereof with at least 70% (e.g., 70%, 75%, 80%, 85%, 90%, 95%, 98%, 99% of 100%) amino acid sequence identity. In an embodiment, a hybrid TCRβ constant chain comprises a chain in which the immunoglobulin like linker domain of TCRβ constant chain is replaced by the immunoglobulin like linker domain of TCRα, TCRγ, TCRδ or pre-TCRα constant chain or a homolog or variant thereof with at least 70% (e.g., 70%, 75%, 80%, 85%, 90%, 95%, 98%, 99% of 100%) amino acid sequence identity. In an embodiment, a hybrid TCRγ constant chain comprises a chain in which the immunoglobulin like linker domain of TCRγ constant chain is replaced by the immunoglobulin like linker domain of TCRα, TCRβ, TCRδ or pre-TCRα constant chain or a homolog or variant thereof with at least 70% (e.g., 70%, 75%, 80%, 85%, 90%, 95%, 98%, 99% of 100%) amino acid sequence identity. In an embodiment, a hybrid TCRδ constant chain comprises a chain in which the immunoglobulin like linker domain of TCRδ constant chain is replaced by the immunoglobulin like linker domain of TCRα, TCRβ, TCRγ or pre-TCRα constant chain or a homolog or variant thereof with at least 70% (e.g., 70%, 75%, 80%, 85%, 90%, 95%, 98%, 99% of 100%) amino acid sequence identity. In an embodiment, a hybrid pre-TCRα constant chain comprises a chain in which the immunoglobulin like linker domain of pre-TCRα, constant chain is replaced by the immunoglobulin like linker domain of TCRα, TCRβ, TCRγ, TCRδ constant chain or a homolog or variant thereof with at least 70% (e.g., 70%, 75%, 80%, 85%, 90%, 95%, 98%, 99% of 100%) amino acid sequence identity.

In example embodiments, the disclosure provides a novel double chain (DC) SAR and novel one and half chain (OHC) SAR comprising one or more non-TCR antigen binding domains that are operationally linked via optional linkers to hybrid TCR chains in which immunoglobulin like linker domain of TCRα constant chain is replaced by the immunoglobulin like linker domain of TCRβ constant chain or a mutant or variant thereof and the immunoglobulin like linker domain of TCRβ constant chain is replaced by the immunoglobulin like linker domain of TCRα constant chain or a mutant or variant thereof.

In example embodiments, the disclosure provides a novel double chain (DC) SAR and novel one and half chain (OHC) SAR comprising one or more non-TCR antigen binding domains that are operationally linked via optional linkers to hybrid TCR chains in which the immunoglobulin like linker domain of TCRγ constant chain is replaced by the immunoglobulin like linker domain of TCRδ constant chain or a mutant or variant thereof and the immunoglobulin like linker domain of TCRδ constant chain is replaced by the immunoglobulin like linker domain of TCRγ constant chain or a mutant or variant thereof.

In example embodiments, the disclosure provides a novel double chain (DC) SAR and novel one and half chain (OHC) SAR comprising one or more non-TCR antigen binding domains that are operationally linked via optional linkers to hybrid TCR chains in which the immunoglobulin like linker domain of TCRα constant chain is replaced by the immunoglobulin like linker domain of TCRγ constant chain or a mutant or variant thereof and the immunoglobulin like linker domain of TCRβ constant chain is replaced by the immunoglobulin like linker domain of TCRδ constant chain or a mutant or variant thereof.

In example embodiments, the disclosure provides a novel double chain (DC) SAR and novel one and half chain (OHC) SAR comprising one or more non-TCR antigen binding domains that are operationally linked via optional linkers to hybrid TCR chains in which the immunoglobulin like linker domain of TCRα constant chain is replaced by the immunoglobulin like linker domain of TCRδ constant chain or a mutant or variant thereof and the immunoglobulin like linker domain of TCRβ constant chain is replaced by the immunoglobulin like linker domain of TCRγ constant chain or a mutant or variant thereof.

In example embodiments, the disclosure provides a novel double chain (DC) SAR and novel one and half chain (OHC) SAR comprising one or more non-TCR antigen binding domains that are operationally linked via optional linkers to hybrid TCR chains in which the immunoglobulin like linker domain of TCRγ constant chain is replaced by the immunoglobulin like linker domain of TCRα constant chain or a mutant or variant thereof and the immunoglobulin like linker domain of TCRδ constant chain is replaced by the immunoglobulin like linker domain of TCRβ constant chain or a mutant or variant thereof.

In example embodiments, the disclosure provides a novel double chain (DC) SAR and novel one and half chain (OHC) SAR comprising one or more non-TCR antigen binding domains that are operationally linked via optional linkers to hybrid TCR chains in which the immunoglobulin like linker domain of TCRγ constant chain is replaced by the immunoglobulin like linker domain of TCRβ constant chain or a mutant or variant thereof and the immunoglobulin like linker domain of TCRδ constant chain is replaced by the immunoglobulin like linker domain of TCRα constant chain or a mutant or variant thereof.

In example embodiments, the disclosure provides a novel double chain (DC) SAR and novel one and half chain (OHC) SAR comprising one or more non-TCR antigen binding domains that are operationally linked via optional linkers to hybrid TCR chains in which the immunoglobulin like linker domain (e.g., SEQ ID NO (DNA): 597-6714 and SEQ ID NO (PRT):8977-8994) of the one or both TCR constant chains of a double chain SAR or of a one and half chain SAR are replaced by Ig linker domains derived from an immunoglobulin (Ig) light chain or an immunoglobulin (Ig) heavy chain or a mutant or variant thereof.

An example immunoglobulin linker domain derived from immunoglobulin light chain (IgCL) is provided in SEQ ID NO (PRT):8961 and include functional variants, mutants and homologs thereof, including homologs from non-human species. In an embodiment, immunoglobulin linker domain of immunoglobulin light chain (IgCL) comprises a polypeptide with at least 70% (e.g., 70%, 75%, 80%, 85%, 90%, 95%, 98%, or 995%) amino acid sequence homology to a sequence represented by SEQ ID NO (PRT): 8961 or a functional variant, mutant or homolog thereof. In an embodiment, immunoglobulin linker domain of immunoglobulin light chain (IgCL) chain comprises a polypeptide with deletion of up to 80 (e.g., 1, 2, 5, 10, 15, 20, 25, 30, 35, 40, 45, 50, 55, 60, 65, 70, 75, 80 etc.) N-terminal amino acids of any of the sequences represented by SEQ ID NO (PRT): 8961 or a functional variant, mutant or homolog thereof.

Example immunoglobulin linker domains derived from immunoglobulin heavy chains (e.g., IgG1-CH1, IgG2-OC-CH1, IgG4-CHI1 etc.) are provided in SEQ ID NO s(PRT):8962-8976 and include functional variants, mutants, and homologs thereof, including homologs from non-human species. In an embodiment, immunoglobulin linker domain of immunoglobulin heavy chain comprises a polypeptide with at least 70% (e.g., 70%, 75%, 80%, 85%, 90%, 95%, 98%, or 995%) amino acid sequence homology to a sequence represented by SEQ ID NO (PRT): 8962-8976 or a functional variant, mutant or homolog thereof. In an embodiment, immunoglobulin linker domain of immunoglobulin heavy chain comprises a polypeptide with deletion of up to 80 (e.g., 1, 2, 5, 10, 15, 20, 25, 30, 35, 40, 45, 50, 55, 60, 65, 70, 75 etc.) N-terminal amino acids of any of the sequences represented by SEQ ID NO (PRT): 8962-8976 or a functional variant, mutant or homolog thereof.

In example embodiments, the disclosure provides a novel one and half chain (OHC) SAR comprising one or more non-TCR antigen binding domains that are operationally linked via optional linkers to hybrid TCR chains in which the immunoglobulin like linker domain of TCRα or TCRγ constant chain is replaced by the immunoglobulin linker domain of immunoglobulin light chain (e.g., SEQ ID NO (PRT): 8961) or a mutant or variant thereof and the immunoglobulin like linker domain of TCRβ or TCRδ constant chain is replaced by the immunoglobulin linker domain of immunoglobulin heavy chain (e.g., SEQ ID NOs (PRT):8962-8976) or a mutant or variant thereof.

In example embodiments, the disclosure provides a novel one and half chain (OHC) SAR comprising one or more non-TCR antigen binding domains that are operationally linked via optional linkers to hybrid TCR chains in which the immunoglobulin like linker domain of TCRβ or TCRδ constant chain is replaced by the immunoglobulin linker domain of immunoglobulin light chain (e.g., SEQ ID NO (PRT): 8961) or a mutant or variant thereof and the immunoglobulin like linker domain of TCRα or TCRγ constant chain is replaced by the immunoglobulin linker domain of immunoglobulin heavy chain (e.g., SEQ ID NOs (PRT):8962-8976) or a mutant or variant thereof.

In example embodiments, the disclosure provides a novel one and half chain (OHC) SAR comprising one or more non-TCR antigen binding domains that are operationally linked via optional linkers to hybrid TCR chains in which the immunoglobulin like linker domain of TCRγ constant chain is replaced by the immunoglobulin linker domain of immunoglobulin light chain (e.g., SEQ ID NO (PRT): 8961) or a mutant or variant thereof and the immunoglobulin like linker domain of TCRδ constant chain is replaced by the immunoglobulin linker domain of immunoglobulin heavy chain (e.g., SEQ ID NOs (PRT):8962-8976) or a mutant or variant thereof.

The disclosure provides novel one and half chain SAR in which the immunoglobulin like linker domain (e.g., SEQ ID NO (DNA): 597-614 and SEQ ID NO (PRT):8977-8994) of the one or both TCR constant chains of a one and half chain SIR are replaced by Ig linker domains derived from an antibody (e.g., IgCL, IgG1-CH1 domain) or a mutant or variant thereof. The Ig linker domain derived from an antibody are provided in SEQ ID NO (DNA): 581-596 and SEQ ID NO (PRT): 8961-8975). Example such SAR constructs are provided in SEQ ID NO (DNA):18023-18258 and SEQ ID NO (PRT):18915-19740.

The disclosure also provides novel hybrid chain TCRs comprising two chains in which the TCR variable domains (e.g., Va and Vb or Vg and Vd) are linked via Ig linkers (e.g., IgCL and IgG1-CH1 or IgCL and IgG4-CH1 etc.) to TCR modules comprising the transmembrane domains of TCR chains. In an embodiment, the TCR modules further comprises the connecting peptide and cytosolic domain of TCR chains. Example such hybrid chain TCR targeting NY-ESO-1 peptide/HILA-A2 complex are presented in SEQ ID NO(DNA): 19760 and 19761 and SEQ ID NO (PRT): 20380 and 20381, respectively. In the construct NY-ESO-1-IG4-Vb-[IgCL-TCRb-wt-opt2-6MD]-F-P2A-NY-ESO-1-IG4-Va-[IgG1-CH1-TCRa-wt-op2-6MD](SEQ ID NO: 19760), the Vb domain of a TCR targeting NY-ESO-1 TCR is attached via IgCL linker to a TCRb-wt-opt2-6MD module comprising the connecting peptide, transmembrane domain and cytosolic domain of human TCRβ chain, while the Va domain of the TCR is attached via a IgG1-CH1 linker to a TCRa-wt-op2-6MD module comprising the connecting peptide, transmembrane domain and cytosolic domain of human TCRα chain. The hybrid chain TCR construct with SEQ ID NO (DNA): 19761 and SEQ ID NO (PRT): 20381 is similar except that the TCRb-wt-opt2-6MD module is replaced by a module IgCL-TCRg-6MD comprising the connecting peptide, transmembrane domain and cytosolic domain of human TCRγ chain while the TCRa-wt-op2-6MD is replaced by a module TCRδ-6MD comprising the connecting peptide, transmembrane domain and cytosolic domain of human TCRδ chain. The two TCR chains of the hybrid chain TCR are less likely to pair with the endogenous TCR chains and therefore T cells expressing such hybrid chain TCR are less likely to cause autoimmunity or gvhd. In some embodiments, the IgCL and IgG1-CH1 linkers are replaced by other Ig linkers (e.g., IgCL and IgG4-CH1) described in this application to generate hybrid chain TCR with unique structural and functional properties. Similarly, the TCR modules can be replaced with other TCR modules described in this application to generate unique hybrid chain TCRs. Finally, the variable domains can be replaced with other variable domains described in this application to generate hybrid chain TCR targeting different peptide/HLA complexes. In some embodiments, one or more autonomous antigen binding domains (AABD) (e.g., vHH, FHVH, DARPIN, AFFIBODY, CENTYRIN, svd-TCR etc.) are attached to the N-terminus or near the N-terminus of the TCR variable domains (e.g., Va, Vb, Vg, Vd) of the hybrid chain TCRs to generate bispecific and multi-specific hybrid chain TCRs that are capable of targeting more than one antigen and/or antigen peptide/MHC complex.

In some embodiment, immune responsive cells (e.g., T, NK, NKT etc.) or hematopoietic stem cells for expression of SAR are obtained from the circulating blood of an individual by apheresis. In one aspect, the cells are collected from a subject in whom the T, NK or stem cells have been mobilized by administration of an agent. In some embodiments, the immune cells are collected from a donor who has been administered a CXCR4 antagonist (e.g., Plerixafor, BL-8040, BPRCX714, BPRCX807), a cytokine (e.g., G-CSF, GM-CSF or sargramostim, Neulasta or Pegfilgastrim, IL2, IL15), a beta2 agonist (e.g., epinephrine), a tyrosine kinase inhibitor (e.g., dasatinib), chemotherapy drug(s) (e.g., cyclophosphamide, doxorubicin etc.) either singly or in combination, prior to the collection of immune cells. In some embodiments, the donor is an autologous donor while in other embodiments, the donor is an allogeneic donor.

The disclosure also provides an expansion free method for generating immune responsive cells expressing a SAR (e.g., SIR, zSIR, Ab-TCR, uTCR-SAR, CD16-SAR etc.) of the disclosure. In an embodiment, the SAR is a double chain SAR. In an embodiment, the SAR is a one and a half chain SAR. In an embodiment, the SAR lacks a cytosolic signaling domain. In an embodiment, the SAR lacks a cytosolic activation domain comprising an ITAM. In an embodiment, the SAR lacks a costimulatory domain. In an embodiment, the SAR does not comprise a CD28 or 41BB costimulatory domain. In an embodiment, the SAR provides physiological T cell receptor or NK receptor signaling. In an embodiment, the SAR comprises a T cell receptor module. In an embodiment, the SAR is capable of recruiting a signaling adaptor. In an embodiment, the signaling adaptor is selected from the group of CD3z, FcRy, DAP10, DAP12. In an embodiment, the SAR is not a second-generation chimeric antigen receptor. In an embodiment, the SAR does not comprise a CD3z or FcRy activation domain. In an embodiment, the SAR comprises a TCR signaling chain selected from TCRα, TCRβ, TCRγ, TCRδ, preTCRα, CD16, NKp30, NKp44, NKp46 or a functional fragment, variant or homolog thereof. In an embodiment, the SAR is selected from the group of a double chain SIR, a one and a half chain SIR, a double chain Ab-TCR, a one and a half chain Ab-TCR, a double chain SIR with hybrid TCR chains; a one and a half chain SIR with hybrid TCR chains; a zSIR, a uTCR-SAR, a TFP, CD16-SAR, a FceRγ-SIR and a vFLIP-CAR.

In an embodiment, one or more steps of the expansion free method for manufacturing of the cell therapy product are carried out in a closed system. In an embodiment, one or more steps of the expansion free method for manufacturing of the cell therapy product are carried out in an automated manner. In an embodiment one or more steps of the expansion free method for manufacturing of the cell therapy product are carried out using a Prodigy (Miltenyi), Cocoon (Lonza), or a cell shuttle (Cellares). In an embodiment, one or more steps of the expansion free method for manufacturing of the cell therapy product are carried out using a gas permeable device. In an embodiment, one or more steps of the expansion free method for manufacturing of the cell therapy product are carried out in a G-Rex device (Wilson-Wolf manufacturing). In an embodiment, one or more steps of the expansion free method for manufacturing of the cell therapy product are carried out using a Wave bioreactor.

In an embodiment, the expansion free method for generation of SAR expressing SAR involves the steps of a) obtaining a population of immune effector cells (e.g., PBMC) from a subject, wherein optionally the subject has been administered one or more mobilizing agents (e.g., CXCR4 antagonist, e.g., Plerixafor, BL-8040, BPRCX714, BPRCX807 etc.), a cytokine (e.g., G-CSF, GM-CSF or sargramostim, Neulasta or Pegfilgastrim, IL2, IL15); b) an optional step to isolate a subpopulation of immune effector cells (e.g., T cell, CD4, CD8, Treg, αβT cell, γδ T cell, NK, NKT cell, Pgp-expressing stem like T or NK cells etc.); c) an optional step to eliminate the expression of one or more genes (e.g., β2M, TRAC, TRBC etc.) in the immune effector cells; d) an optional step to activate the immune effector cells with one or more cytokines, wherein optionally the cytokines are selected from the group of IL2, IL7, IL15 or a combination thereof; e) an optional step to activate the immune effector cells with an agent that activate the TCR, wherein optionally the agent is an antibody against CD3 or CD3 antibody coated beads; f) an optional step to add a co-stimulatory agent during the activation step of (e), wherein optionally the costimulatory agent activates the CD28 receptor or 4-1BB receptor, wherein optionally the agent is an antibody or antibody coated beads targeting CD28 or 4-1BB; g) introducing a SAR expression construct into the immune effector cells; wherein optionally the SAR expression construct is introduced by contacting immune effector cells for sufficient time with a viral vector encoding the SAR, wherein optionally the viral vector is a lentiviral vector or a γ retroviral vector; wherein optionally the contact is carried out in the presence of an agent that increases the gene transduction with the viral vector, wherein optionally the agent is selected from the group of polybrene, protamine, retronectin, vectofusin or a combination thereof; h) an optional step to remove the activating agent or activating agent coated beads (i.e., debeading); i) an optional wash step; j) an optional step to concentrate the cell therapy product; k) an optional step to cryopreserve the cell therapy product; 1) optional steps for safety and potency testing.

In an embodiment, the SAR expression construct (e.g., RNA, viral vector) is introduced into the immune effector cells or stem cells that can give rise to immune effector cells in less than 12 h (e.g., 10 h, 8 h, 6 h, 4 h, 2 h, 1 h, 30 min, 20 min, 10 min, 5 min, 1 min etc.) of removal of the cells from a subject. In an embodiment, the SAR expression construct (e.g., RNA, viral vector) is introduced into the immune effector cells or stem cells that can give rise to immune effector cells during the step of collection (e.g., apheresis). In an embodiment, the SAR expression construct (e.g., RNA, viral vector) is introduced into the immune effector cells or stem cells that can give rise to immune effector cells in the same container (e.g., bag) in which the apheresis cell product is collected from the subject. In an embodiment, the SAR expression construct (e.g., RNA, viral vector) is introduced into the immune effector cells or stem cells that can give rise to immune effector cells in the same location (e.g., collection facility, room) in which the apheresis cell product is collected from the subject. In an embodiment, the SAR expression construct (e.g., RNA, viral vector) is introduced into the immune effector cells or stem cells that can give rise to immune effector cells using a unit that is operationally attached to the apheresis machine. In an embodiment, the SAR expression construct (e.g., RNA, viral vector) is introduced into the immune effector cells or stem cells that can give rise to immune effector cells using a closed unit that is operationally attached to the apheresis machine.

In an embodiment, the cells expressing the SAR expression construct are administered to the subject in less than 36 h (e.g., less than 36 h, 24 h, 20 h, 16 h, 12 h, 10 h, 8 h, 6 h, 4 h, 2 h, 1 h, 30 min, 20 min, 10 min, 5 min, 1 min etc.) after their collection from the subject (i.e., vein to vein time). In an embodiment, the cells expressing the SAR expression construct are administered to the subject from the same container (e.g., bag) in which the cells are collected at the time of apheresis. In an embodiment, the cells expressing the SAR expression construct are administered to the subject at the same location (e.g., collection facility, collection room) in which the cells are collected from the subject at the time of apheresis. In an embodiment, the cells expressing the SAR expression construct are administered to the subject using a unit that is operationally attached to the apheresis machine or is part of the apheresis machine or is integrated with the apheresis machine. In an embodiment, the cells expressing the SAR expression construct are administered to the subject using a closed unit that is operationally attached to the apheresis machine.

In an embodiment, the steps of collection (i.e., apheresis) of the immune or stem cells, introduction of the SAR into immune or stem cells and administration of the SAR-expressing immune or stem cells to the subject are conducted in less than 36 h (e.g., less than 36 h, 24 h, 20 h, 16 h, 12 h, 10 h, 8 h, 6 h, 4 h, 2 h, 1 h, 30 min, 20 min, 10 min, 5 min, 1 min etc.). In an embodiment, the steps of collection (i.e., apheresis) of the immune or stem cells, introduction of the SAR into immune or stem cells and administration of the SAR-expressing immune or stem cells to the subject are conducted in a single visit. In an embodiment, the steps of collection (i.e., apheresis) of the immune or stem cells, introduction of the SAR into immune or stem cells and administration of the SAR-expressing immune or stem cells to the subject are conducted in a single location (e.g., collection facility, collection room). In an embodiment, the steps of collection (i.e., apheresis) of the immune or stem cells, introduction of the SAR into immune or stem cells and administration of the SAR-expressing immune or stem cells to the subject are conducted using a single machine. In an embodiment, the steps of collection (i.e., apheresis) of the immune or stem cells, introduction of the SAR into immune or stem cells and administration of the SAR-expressing immune or stem cells to the subject are conducted using machines (units) that are operationally linked to each other. In an embodiment, the steps of collection (i.e., apheresis) of the immune or stem cells, introduction of the SAR into immune or stem cells and administration of the SAR-expressing immune or stem cells to the subject are conducted using single machine or machines (units) that share a single power (e.g., electric source) source. In an embodiment, the steps of collection (i.e., apheresis) of the immune or stem cells, introduction of the SAR into immune or stem cells and administration of the SAR-expressing immune or stem cells to the subject are conducted using single machine or machines (units) that are controlled by a single computer or software. In an embodiment, the steps of collection (i.e., apheresis) of the immune or stem cells, introduction of the SAR into immune or stem cells and administration of the SAR-expressing immune or stem cells to the subject are conducted using single closed system. In an embodiment, the SAR is introduced into the immune cells or stem cells that can give rise to immune cells via any methods of gene transduction known in the art, including but not limited to, viral vector (e.g., lentiviral, γ retroviral), virus like particles, lipid nano-particles, electroporation, lipofection or by causing transient perturbations in cell membranes.

In an embodiment, the expansion free cell method involves incubating a population of immune cells (e.g., T cells, e.g., freshly isolated T cells, e.g., freshly isolated quiescent T cells) in a medium that does not comprise serum, or comprises no more than 0.1, 0.2, 0.3, 0.4, 0.5, 0.6, 0.7, 0.8, 0.9, 1, 1.2, 14, 1.6, 1.8, or 2% serum, e.g., for at least about 1-10 hours, e.g., for at least about 2, 3, 4, 5, 6, 7, 8, 9, or 10 hours, e.g., for at least about 2 to 6 hours; and (ii) transducing the population of immune cells with a nucleic acid molecule, e.g., a nucleic acid molecule on a lentiviral vector, encoding the SAR, in a medium comprising serum (e.g., at least about 4, 5, or 6% serum) and optionally deoxynucleosides (e.g., at least about 40 μM-1.5 mM deoxynucleosides, e.g., at least about 40 μM, 45 μM, 50 μM, 55 μM, 60 μM, 70 μM, 80 μM, 90 μM, 1 mM, 1.1 mM, 1.2 mM, 1.3 mM, 1.4 mM, or 1.5 mM deoxynucleosides), e.g., for about 14-30 hours, e.g., for about 14, 16, 18, 20, 22, 24, 26, or 28 hours, optionally wherein step (ii) is performed at a cell concentration of at least about 0.7×10⁷, 0.8×10⁷, 0.9×10⁷, 1×10⁷, 2×10⁷, 4×10⁷, 6×10⁷, 8×10⁷, or 1×10⁸ cells/mL, e.g. step (ii) is performed at a cell concentration of about 1×10⁷ cells/mi. In an embodiment, optionally the population of immune cells is not contacted in vitro with an agent that stimulates a CD3/TCR complex and/or an agent that stimulates a costimulatory molecule, e.g., anti-CD3 antibody and/or anti-CD28 antibody. In an embodiment, the expansion free method comprises (iii) harvesting the population of immune cells for storage (e.g., reformulating the population of immune cells in cryopreservation media) or administration, wherein: (a) step (iii) is performed no later than 48 hours, e.g., no later than 14, 16, 18, 20, 22, 24, 26, 28, 30, or 32 hours after the beginning of step (i), (b) the population of immune cells from step (iii) is not expanded, or is expanded by no more than 10, 20, 30, 40, or 50%, e.g., no more than 10%, compared with the population of immune cells at the beginning of step (i), (c) the percentage of naïve cells, e.g., naïve T cells, in the population of immune cells from step (iii) is not reduced, or is reduced by no more than 10, 20, or 30%, compared with the percentage of naïve cells, e.g., naïve T cells, in the population of immune cells at the beginning of step (i), and/or (d) the percentage of differentiated cells, e.g., differentiated T cells, e.g., terminally differentiated T cells, e.g., CCR7^{low} T cells, in the population of immune cells from step (iii) is not increased, or is increased by no more than 10, 20, or 30%, compared with the percentage of differentiated cells, e.g., differentiated T cells, e.g., terminally differentiated T cells, e.g., CCR7^{low} T cells, in the population of immune cells at the beginning of step (i). In an embodiment, wherein step (i) comprises incubating the population of immune cells (e.g., T cells, e.g., freshly isolated T cells, e.g., freshly isolated quiescent T cells) in a medium that does not comprise serum for about 2-6 hours.

In an embodiment, the cell therapy product is expanded for 1-10 days after introduction of the SAR expression construct.

In an embodiment, the viral vector (e.g., lentiviral or γ retroviral) encoding the SAR is pseudotyped with a modified baboon envelope glycoprotein, a modified HERV-W1 envelope glycoprotein, a VSVG envelope glycoprotein. In an embodiment, the viral vector (e.g., lentiviral or γ retroviral) encoding the SAR is pseudotyped with a modified baboon envelope glycoprotein described herein. In an embodiment, the modified baboon envelope glycoprotein comprises a sequence with SEQ ID NO (DNA):70-96, 98, 115-122, 264-273 or SEQ ID NO(PRT):8450-8476, 8478, 8495-8502, 8644-8655, 50005-50010 or an encoded envelope glycoprotein with at least 70%, 75%, 80%, 85%, 90%, 95%, 98% sequence identity to the SEQ ID NO(PRT):8450-8476, 8478, 8495-8502, 8644-8655, 50005-50010 in the extracellular domain. In an embodiment, the modified baboon envelope glycoprotein encodes for a protein comprising a sequence with at least 70%, 75%, 80%, 85%, 90%, 95%, 98% sequence identity to the SEQ ID NO(PRT):8450-8476, 8478, 8495-8502, 8644-8655, 50005-50010 in the transmembrane domain. In an embodiment, the modified baboon envelope glycoprotein encodes for a protein that binds to hASCT1 or hASCT2.

In an embodiment, the viral vector (e.g., lentiviral or γ retroviral) encoding the SAR is pseudotyped with a modified HERV-W1 envelope glycoprotein described herein. In an embodiment the modified HERV-W1 envelope glycoprotein is encoded by a recombinant polynucleotide that encodes for an exogenous signal peptide sequence. In an embodiment, the viral vector (e.g., lentiviral or γ retroviral) encoding the SAR is pseudotyped with a modified HERV-W1 envelope glycoprotein with SEQ ID NO (DNA): 149-151, 159-164 or SEQ ID NO(PRT): 8529-8530, 8539-8544, 8581 or an encoded envelope glycoprotein with at least 70%, 75%, 80%, 85%, 90%, 95%, 98% sequence identity to the SEQ ID NO(PRT): 8529-8530, 8539-8544, 8581 in the extracellular domain. In an embodiment, the modified HERV-W1 envelope glycoprotein encodes for a protein comprising a sequence with at least 70%, 75%, 80%, 85%, 90%, 95%, 98% sequence homology to the SEQ ID NO(PRT): 8529-8530, 8539-8544, 8581 in the transmembrane domain. In an embodiment, the modified HERV-W1 envelope glycoprotein encodes for a protein that binds to hASCT1 or hASCT2.

In an embodiment, the immune effector cells are transduced with SAR encoding viral vectors that are pseudotyped with a combination of two or more different envelope glycoproteins (e.g., baboon plus VSVG or HERV-W1 or VSVG etc.). In an embodiment, the SAR expression construct is introduced into immune effector cells using a non-viral delivery method. In an embodiment, the non-viral method comprises a virus like particle (VLP) or a lipid nanoparticle (LNP). In an embodiment the SAR expression construct is introduced into immune effector cells using electroporation, or by using lipofection or by causing transient perturbation in cell membrane. In an embodiment, the SAR is expressed from an endogenous gene locus. In an embodiment, the SAR expression cassette is inserted at the genomic locus of an endogenous gene. In an embodiment, the SAR expression cassette is inserted at the genomic locus of an endogenous gene. In an embodiment, the insertion of SAR at the endogenous gene locus results in disrupted expression of the endogenous gene. In an embodiment, SAR is expressed under the promoter and regulator elements of an endogenous gene. In an embodiment, the endogenous gene locus is selected from TRAC. TRBC, TRDC, TRGC, CD3z, CD16, FceRy1 gene locus.

In an embodiment, the SAR encoding cell therapy product generated using the expansion-free method is superior to a product manufactured using a method that involves expansion of the cell therapy product in one or more of the following: a) manufactured in a shorter period of time (e.g., between 1-12 days); b) results in cost-saving (e.g., between 5-95% cost reduction as compared to expansion method); c) results in fewer manufacturing failure (e.g., 5-95% fewer manufacturing failures vs the expansion method; d) shows superior in vitro properties (e.g., at least 5% improvement in one or more of the following parameters as compared to expansion method: cell killing, cytokine production, lack of exhaustion markers, maintains CD4:CD8 ratio; lack of terminal differentiation, maintains stem like/naïve/memory phenotype; e) shows superior in vivo activity (e.g., at least 5% improvement in one or more of the following parameters as compared to expansion method: long term persistence, lack of exhaustion, anti-tumor activity etc.); f) at least 5% superior disease control (e.g., tumor control) when administered to a subject in need thereof; g) shows superior safety (e.g., at least 5% reduced cytokine release syndrome or neurotoxicity as compared to a product manufactured using expansion method etc.); h) requires administration of less cell dose (e.g., at least 5% reduction in administered cell dose as compared to a cell therapy product manufactured using expansion method.

The disclosure provides single chain and double chain next generation SAR designs that provide physiological signaling.

The disclosure provides next generation single chain and double chain SAR comprising one or more heterologous antigen binding domains (e.g., vL, vH, scFv, vHH, FHVH, non-immunoglobulin antigen binding scaffold, epitope etc.) that are operationally linked to the N-terminus or near the N-terminus of the extracellular domains of CD16 chains that comprise deleted and/or mutated CD16 cytoplasmic domains. Example CD16 chains with deleted and mutated cytoplasmic domains that can be used in the construction of SAR are provided in SEQ ID NO (PRT): 8945-8948. In an embodiment, the SAR can be constructed using CD16 chains with at least 70% amino acid sequence homology to SEQ ID NO (PRT): 8945-8948 or functional variants thereof. Example SARs with deleted and mutated cytoplasmic domains are presented in SEQ ID NO (DNA): 1111-2234 and SEQ ID NO (PRT): 9491-10614. In an embodiment, the SAR can be constructed in which one or more antigen binding domains are attached to N-terminus or near the N-terminus of entire or partial extracellular domain of CD16 chains represented by SEQ ID NO (PRT): 8945-8948 or functional variants thereof.

The disclosure provides next generation single chain and double chain SAR comprising one or more heterologous antigen binding domains that are operationally linked to the N-terminus or near the N-terminus of the extracellular domains of CD16 chains (e.g., FCGR1A, FCGR1B, FCGR1C) that comprise deleted and/or mutated CD64 cytoplasmic domains. Example CD64 chains with deleted and mutated cytoplasmic domains that can be used in the construction of SAR are provided in SEQ ID NO (PRT): 20383. In an embodiment, the SAR can be constructed using CD64 chains with at least 70% amino acid sequence homology to SEQ ID NO (PRT): 20382-83 or functional variants thereof. In an embodiment, the SAR can be constructed in which one or more antigen binding domains are attached to N-terminus or near the N-terminus of entire or partial extracellular domain of CD64 chains represented by SEQ ID NO (PRT): 20382-83 or functional variants thereof.

The disclosure provides novel antigen binding domains (e.g., scFv, vL, vH, vHH etc.) targeting different antigens. The novel vL fragments are represented by SEQ ID NO (DNA): 339-354, 19766-19776, and 32006-32068, the complementary vH fragments are represented by 363-378, 19785-19795, and 32069-32131 (Tables 3 and 5). The disclosure also provides novel vHH domains, including humanized vHH, targeting different antigens. These vHH domains are represented by SEQ ID NO: 412-426, 32195-32213. These novel antigen binding domains (e.g., scFv, vL, vH, vHH etc.) can be used in the construction of SAR (e.g., SIR, zSIR, Ab-TCR, CAR, etc.), antibodies, bispecific antibodies, and antibody drug conjugates etc. The disclosure also provides novel antigen binding domains with at least 75% (e.g., 75%, 80%, 85%, 90%, 95%, 99%) amino acid sequence identity in the framework region to the novel antigen binding domains (e.g., scFv, vL, vH, vHH etc.) provided in Tables 3 and 5. The disclosure also provides novel antigen binding domains with up to 2 amino acid differences (i.e., 1, 2) in each of the CDR of the novel antigen binding domains (e.g., scFv, vL, vH, vHH etc.) provided in Tables 3 and 5. The light chain complementary determining regions 1-3 (LC-CDR1-3) for these novel vL domains are as set forth in any of SEQ ID Nos: 20989-21015, 41591-41861; 21024-21050, 41862-42132; and 21059-21085, 42133-42403. The heavy chain complementary determining regions 1-3 (HC-CDR1-3) are as set forth in any of SEQ ID Nos: 21094-21120 and 42404-42674; 21129-21155, 42675-42945; and 21164-21190, 42946-43216. The CDR1-3 for the novel vHH domains are provided in SEQ ID NO: 43304-43318. Example SAR comprising these novel antigen binding domains are provided in SEQ ID NO (DNA): 3303-3929, 19823-21960, 33860-40139 and 40173-40427.

The disclosure provides novel TCR variable domains (Table 4) that can be used in the construction of SAR (e.g., uTCR-SAR) of the disclosure. Example uTCR-SAR comprising these TCR variable domains are provided in Table 12 of the provisional patent application. In an embodiment, the TCR variable domains can bind to the peptide/MHC complex independent of the co-receptors. In an embodiment, the TCR variable domains can bind to the peptide/MHC complex independent of CD8a, CD8b or CD4. In an embodiment, the TCR variable domains can bind to the peptide/MHC complex with higher affinity than a naturally occurring TCR. In an embodiment, the TCR variable regions comprise exogenous disulfide bonds. In an embodiment, the disclosure provides a SAR (e.g., uTCR-SAR) that can bind to the peptide/MHC complex independent of the co-receptors. In an embodiment, the SAR (e.g., uTCR-SAR) can bind to the peptide/MHC complex independent of CD8a, CD8b or CD4. In an embodiment, the SAR (e.g., uTCR-SAR) comprise TCR variable regions comprising exogenous disulfide bonds. In an embodiment, the SAR (e.g., uTCR-SAR) comprise TCR constant regions comprising exogenous disulfide bonds. In an embodiment, the SAR (e.g., uTCR-SAR) comprise mutations that result in cysteine residues in the TCR variable domains that result in the formation of exogenous disulfide bonds. In an embodiment, the exogenous disulfide bonds are intra-chain disulfide bonds. In an embodiment, the SAR (e.g., uTCR-SAR) comprise TCR constant regions comprising exogenous disulfide bonds. In an embodiment, the exogenous disulfide bonds are inter-chains disulfide bonds between the two TCR constant chains.

The disclosure provides γδ T cell expressing a double chain SAR or a one and a half chain SAR (e.g., double chain SIR or one and a half chain SIR) comprising non-TCR antigen binding domains (e.g., vL, vH, scFv, vHH etc.) that are operationally linked to TCRα and TCRβ constant chains. In an embodiment, the SAR (SIR) comprise TCRα/β constant chains that have wild-type nucleic acid sequence. In an embodiment, the SAR (SIR) comprise TCRα/β constant chains that have wild-type amino acid sequence. In an embodiment, the TCRα and TCRβ constant chains comprise mutations (e.g., T48C and S57C) that result in the formation of a second disulfide bond. In an embodiment, the TCRα and TCRβ constant chains comprise mutations that result in their better pairing with each other and reduced pairing with the endogenous TCRα and/or TCRβ constant chains. In an embodiment, the TCRα and TCRβ constant chains of the SAR (e.g., SIR) comprise murine amino acid residues that result in better expression. In an embodiment, the TCRα and TCRβ constant chains of the SAR are murine in origin. In an embodiment, the TCRα and TCRβ constant chains of the SAR have deletions (e.g., N-terminal deletions of 1-60 amino acids

The disclosure provides γδ T cell expressing a double chain SAR or a one and a half chain SAR (e.g., double chain SIR or one and a half chain SIR) comprising non-TCR antigen binding domains (e.g., vL, vH, scFv, vHH etc.) that are operationally linked to hybrid TCR constant chains. (e.g., hybrid TCRα and TCRβ constant chains or hybrid TCRγ and TCRδ chains). In an embodiment, the SAR (SIR) comprise hybrid TCRα/β or hybrid TCRγ/δ constant chains that have wild-type nucleic acid sequences of the TCRα/β or TCRγ/δ constant chains. In an embodiment, the SAR (SIR) comprise hybrid TCRα/β constant chains or hybrid TCRγ/δ constant chains that have wild-type amino acid sequence. In an embodiment, the hybrid TCRα and TCRβ constant chains comprise mutations (e.g., T48C and S57C) that result in the formation of a second disulfide bond. In an embodiment, the hybrid TCRα and TCRβ constant chains comprise mutations that result in their better pairing with each other and reduced pairing with the endogenous TCRα and/or TCRβ constant chains. In an embodiment, the hybrid TCRα and TCRβ constant chains of the SAR (e.g., SIR) comprise murine amino acid residues that result in better expression. In an embodiment, the hybrid TCRα, β, γ or δ constant chains of the SAR are murine in origin. In an embodiment, the hybrid TCRα, β, γ or δ constant chains of the SAR are derived from a species other than human (i.e., mouse, cat, dog, monkey etc.). In an embodiment, the hybrid TCRα, β, γ or δ constant chains of the SAR have deletions (e.g., N-terminal deletions of 1-60 amino acids).

The disclosure provides γδ T cell expressing a double chain SAR or a one and a half chain SAR (e.g., double chain SIR or one and a half chain SIR) comprising non-TCR antigen binding domains (e.g., vL, vH, scFv, vHH etc.) that are operationally linked to TCRγ and TCRδ constant chains. In an embodiment, the SAR (SIR) comprises TCRγ/δ constant chains that have wild-type nucleic acid sequence. In an embodiment, the SAR (e.g., SIR) comprises TCRγ/δ constant chains that have wild-type amino acid sequence. In an embodiment, the TCRγ and TCRδ constant chains of the SAR have deletions (e.g., N-terminal deletions of 1-60 amino acids). In an embodiment, the T cells lacks or have reduced expression of endogenous TCRγ or/and TCRδ chains.

The disclosure provides γδ T cell expressing a double chain SAR or a one and a half chain SAR (e.g., double chain SIR or one and a half chain SIR) comprising non-TCR antigen binding domains (e.g., vL, vH, scFv, vHH etc.) that are operationally linked to pre-TCRα and TCRβ constant chains. In an embodiment, the SAR (SIR) comprise pre-TCRα and TCRβ constant chains that have wild-type nucleic acid sequence. In an embodiment, the SAR (SIR) comprise pre-TCRα and TCRβ constant chains that have wild-type amino acid sequence. In an embodiment, the pre-TCRα and TCRβ constant chains of the SAR have deletions (e.g., N-terminal deletions of 1-60 amino acids). In an embodiment, the T cells lacks or have reduced expression of endogenous TCR pre-TCRα or/and TCRβ chains.

In an embodiment, the γδ T cells expressing the SAR (e.g., SIR or NK-SAR) are derived from embryonic stem cells. In an embodiment, the γδ T cells expressing the SAR (e.g., SIR or NK-SAR) are derived from induced pluripotent embryonic stem cells (iPSC). In an embodiment, the γδ T cells expressing the SAR (e.g., SIR or NK-SAR) are derived from cord blood. In an embodiment, the γδ T cells expressing the SAR (e.g., SIR or NK-SAR) are derived from a donor. In an embodiment, the donor is an autologous donor. In an embodiment, the donor is an allogeneic donor.

The disclosure provides up T cell expressing a double chain SAR or a one and a half chain SAR (e.g., double chain SIR or one and a half chain SIR) comprising non-TCR antigen binding domains (e.g., vL, vH, scFv, vHH etc.) that are operationally linked to TCRγ and TCRδ constant chains. In an embodiment, the SAR (SIR) comprise TCRγ/δ constant chains that have wild-type nucleic acid sequence. In an embodiment, the SAR (SIR) comprise TCRγ/δ constant chains that have wild-type amino acid sequence. In an embodiment, the TCRγ and TCRδ constant chains of the SAR have deletions (e.g., N-terminal deletions of 1-60 amino acids). In an embodiment, the SAR (SIR) comprise hybrid TCRα/β or TCRγ/δ constant chains. In an embodiment, the up T cells lacks or have reduced expression of endogenous TCR α or/and TCRβ chains.

In an embodiment, the up T cells expressing the SAR (e.g., SIR or NK-SAR) are derived from embryonic stem cells. In an embodiment, the up T cells expressing the SAR (e.g., SIR or NK-SAR) are derived from induced pluripotent embryonic stem cells (iPSC). In an embodiment, the up T cells expressing the SAR (e.g., SIR or NK-SAR) are derived from cord blood. In an embodiment, the as T cells expressing the SAR (e.g., SIR or NK-SAR) are derived from a donor. In an embodiment, the donor is an autologous donor. In an embodiment, the donor is an allogeneic donor.

The disclosure also provides embryonic stem cells (e.g., iPSC) expressing one or more SARs (e.g., SIR, NK-SAR, uTCR-SAR) of the disclosure. The disclosure also provides as and γδ T cells derived from embryonic stem cells (e.g., iPSC) expressing one or more SARs (e.g., SIR, NK-SAR, uTCR-SAR) of the disclosure. In an embodiment, the embryonic stem cells (e.g., iPSC) lack or have reduced expression of one or more endogenous TCR chains. In an embodiment, the embryonic stem cells (e.g., iPSC) lack or have reduced expression of 02M and/or HLA molecules.

The disclosure also provides NK cells, NKT cells, umbilical cord derived NK cells, umbilical cord derived T cells, umbilical cord derived stem cells that express any of the SAR (e.g., SIR, HC-SAR, zSIR, zCD16-SIR, uTCR-SAR etc.) described in this disclosure.

In an embodiment, the SAR expressing cells (e.g., iPSC, NK, T, NKT, CD34+ cells etc.) lack or have reduced expression of TNFα, IL1a, IL1β and/or IFNγ. In an embodiment, the SAR expressing cells (e.g., iPSC, NK, T, NKT, CD34+ cells etc.) lack or have genetic disruption of TNFα, IL1a, IL1β and/or IFNγ genes. In an embodiment, the SAR expressing cells (e.g., iPSC, NK, T, NKT, CD34+ cells etc.) overexpress CD47 and/or an Fc receptor (e.g., CD64, CD16). In an embodiment, the SAR expressing cells (e.g., iPSC, NK, T, NKT, CD34+ cells etc.) express a CD16- or a CD64-based SAR (i.e., a SAR comprising an exogenous antigen binding domain attached to a CD64 or CD16 chain comprising the extracellular domain, hinge domain, transmembrane domain, and optionally cytosolic domain of CD64 or CD16. Example CD16-SAR are provided in Tables 9 and 10 of the provisional patent application. Example CD64 based SAR have been described in WO2022178367, which is incorporated by reference herein. Alternatively, CD64 based SAR can be generated by replacing the CD16 signaling chain in SARs described in Tables 9 and 10 of the provisional patent application with a CD64 signaling chain (SEQ ID NO (DNA): 900-901 or SEQ ID NO (PRT): 9280-81.

The disclosure also provides regulatory T cells (Tregs) expressing one or more SARs (e.g., SIR, NK-SAR, uTCR-SAR) of the disclosure. In an embodiment, the Tregs overexpress Fox3P. The disclosure provides that Tregs expressing the SARs of the disclosure can be used to modulate immune response. In an embodiment, the Tregs expressing the SARs of the disclosure (e.g., a SAR targeting HLA molecule) can be used to enhance transplant tolerance in a subject given an HLA-mismatched graft. In an embodiment, the Tregs expressing the SARs of the disclosure can be used to modulate immune response in a subject with autoimmune disorder (e.g., inflammatory bowel disease or multiple sclerosis etc.).

The disclosure provides novel viral envelope proteins for pseudotyping of lentiviral vectors. Example viral envelope proteins include modified baboon envelope (mBaEV) and modified HERV-W1 envelope proteins. These viral envelope proteins can be used for transduction of hard to infect cells such as NK cell and CD34+ve stem cells. In an embodiment, the viral envelope proteins can be used for in vivo delivery of nucleic acids without eliciting significant immune response. The disclosure provides that combination of two different pseudotyped viruses can be used to enhance gene transduction into cells. The disclosure also provides novel envelope glycoproteins (e.g., SEQ ID NO: 8539 and 8450-8476) that can be used to transduce cells without prior activation or pre-stimulation. Example novel viral envelope glycoproteins are provided in SEQ ID NO(DNA): 70-96, 98, 115-122, 145, 159 and SEQ ID NO (PRT): 8539 and 8450-8476, 8525, 8539.

The disclosure provides cell lines stably expressing reporter genes (e.g., marine luciferase and heat-stable beetle luciferases etc.) that are made replication incompetent by treatment but can be used for measurement of cytotoxicity using Matador assay or Matador-Glo cytotoxicity assay. In an embodiment, the cell lines are made replication competent by treatment with mitomycin-C. In an embodiment, the cell lines are made replication competent by treatment with irradiation. In an embodiment, the cell lines are made replication competent by treatment with ionizing irradiation.

A SAR of the disclosure can be expressed in any mammalian cell and be functionally active. In an embodiment, the mammalian cells is a T cell, NK cell, macrophage, granulocyte etc. In an embodiment, the cell is an umbilical cord derived cell. In an embodiment, the cell is an umbilical cord derived T cell, NK cell, NKT cell or stem cell. In some embodiments of any of the mammalian cells described herein, the mammalian cell is selected from the group of a CD8+ T cell, a CD4⁺ T cell, a memory T cell, naïve T cell, T stem cell, a Treg cell, natural killer T (NKT) cell, iNKT (innate natural killer cell), NK cell, g-NK cell, memory like NK cells, cytokine induced killer cell (CIK), iPSC-derived NK cell, α/β T cell, γ/δ T cell, iPSC-derived T cell, B cell, a macrophage/monocyte, iPSC. In some embodiments of any of the mammalian cells described herein, the mammalian cell is selected from the group consisting of an iPSC (induced pluripotent stem cell), an embryonic stem cell and a hematopoietic stem cell that can give rise to an immune effector cell (e.g., a T cell, NK cell or NKT cell). In some embodiments, the mammalian cell is an immortalized cell line, such as NK92, NK92MI, YTS or a derivative thereof. In some embodiments of any of the mammalian cells described herein, the mammalian cell is a mammalian cell obtained from a subject. In some embodiments of any of the mammalian cells described herein, the subject is diagnosed or identified as having a cancer or an autoimmune disease (e.g., lupus, multiple sclerosis etc.). In some embodiments of any of the mammalian cells described herein, the subject is human. In some embodiment, the cell is autologous. In some embodiment, the cell is allogeneic. Example diseases that can be targeted by the SAR targeting an antigen of the invention are provided in PCT/US19/035096, which is incorporated by reference herein.

Also, provided herein are SAR (e.g., uTCR-SAR, HC-SAR, zCD16-SAR etc.) that can be functionally expressed in cells other than T cells including, but not limited to, NK cells, monocytes, macrophages, dendritic cells, granulocytes, stem cell, embryonic stem cell and/or iPSC. In an embodiment, the cell expressing the SAR (e.g., uTCR-SAR, HC-SAR, zCD16-SAR etc.) is engineered to express a co-receptor. In an embodiment, the co-receptor is CD8a, CD8b or CD4. In an embodiment, the cell overexpresses a chimeric CD8 molecule. In an embodiment, the cell overexpresses a chimeric CD8a/CD8b molecule. In an embodiment, the cell overexpresses a chimeric CD8/CD4 molecule. In an embodiment, the cell expressing the SAR (e.g., uTCR-SAR, HC-SAR, zCD16-SAR etc.) is engineered to express IL12 or IL12 fusion protein (e.g., IL12f or membrane-anchored IL12f). In an embodiment, the cell lacks expression of β2M, MHC-class I and class II and HLA-E. In an embodiment, the cell overexpresses CD47 and a Fc receptor (e.g., CD64). In an embodiment, the cell has a mutation in calreticulin gene. In an embodiment, the cell has genetic disruption or reduced expression of TNFα, IL1α, IL1β, IL6, IFNα, IFNβ, and/or IFNγ genes. In an embodiment, the cell expresses IL2, 117 or IL15, optionally in a membrane bound form.

Also provided herein are polypeptides encoding any of the SAR described herein.

Also provided herein are pharmaceutical compositions that include any of the mammalian cells described herein and a pharmaceutically acceptable carrier. Also provided herein are kits that include any of the pharmaceutical compositions described herein.

Also provided herein are pharmaceutical compositions that include any of the nucleic acids described herein that encode any of the single chain, double chain and multi-chain SARs and/or accessory modules described herein, or any of the sets of nucleic acids described herein that together encode any of the single chain, double chain and multi chain SARs and/or accessory modules described herein, and a pharmaceutically acceptable carrier. Also provided herein are kits that include any of the pharmaceutical compositions described herein.

In some embodiments, there is provided a method of killing a target cell presenting one or more target antigens, comprising contacting the target cell with an effector cell expressing a SAR according to any of the SARs (such as isolated SARs) described above, wherein the SAR specifically binds to one or more target antigens.

In some embodiments, according to any of the methods of killing a target cell described above, the contacting is in vivo. In some embodiments, the contacting is in vitro.

In some embodiment, there are provided methods for detection, isolation, purification, expansion, enrichment, and elimination of cells expressing any of the SAR described herein.

Also provided herein are methods of generating a cell expressing a single chain, double chain and multi-chain SAR and/or accessory modules that include introducing into a mammalian cell any of the nucleic acids described herein that encode any of the SARs and accessory modules described herein, or any of the sets of nucleic acids described herein that encode any of the multi-chain SARs described herein.

Also provided herein are methods of treating or preventing a disease (e.g., cancer, infection, allergy, autoimmune disorder etc.) in a subject that include administering a therapeutically effective amount of any of the mammalian cell described herein to the subject. The disclosure also provides a method comprising administering a SAR molecule, a cell expressing a SAR molecule or a cell comprising a nucleic acid encoding a SAR molecule to a subject. In one embodiment, the subject has a disorder described herein, e.g., the subject has cancer, infectious disease, allergic disease, degenerative disease, or autoimmune disease, which expresses a target antigen described herein. In yet one embodiment, the subject has increased risk of a disorder described herein, e.g., the subject has increased risk of cancer, infectious disease, allergic disease, degenerative disease, or autoimmune disease (e.g., lupus, multiple sclerosis, diabetes mellitus, inflammatory bowel disease etc.), which expresses a target antigen described herein.

In some embodiments of any of the SARs described herein, the heterologous antigen-binding domain is selected from the group of: an antibody, an antibody fragment (vL, vH, Fab etc.) a scFv, a (scFv)₂, a VHH domain, FHVH (a fully human vH domain), a single domain antibody, a non-immunoglobulin antigen binding scaffold (e.g., Centyrin, affibody, ZIP domain, an adaptor etc.), a VNAR domain, a ligand, a TCR, variable domain (Va, Vb, Vg, Vd) of a TCR and a receptor.

In some embodiments of any of the SARs described herein, the heterologous antigen-binding region binds specifically to a single antigen. In some embodiments of any of the single-chain SARs described herein, the single antigen is a tumor antigen. In some embodiments of any of the SARs described herein, the tumor antigen is selected from an antigen listed in Table B.

Also provided herein are mammalian cells that include any of the vectors described herein.

Also provided herein are methods of generating a SAR-expressing cell, the method comprising introducing into a mammalian cell any of the nucleic acids described herein or any of the vectors described herein.

In another or further embodiment of any of the foregoing, the immune cell or stem cell of any comprises a plurality of SAR polypeptides. In an embodiment, the plurality of SAR polypeptides are based on different SAR architectures (e.g., backbones), such as SIR, CAR, zSIR, uTCR-SAR, TFP etc. In another or further embodiment of any of the foregoing, at least one SIR polypeptide of the plurality of SAR (e.g., a SIR) polypeptides targets a different antigen than at least one other SAR polypeptide. In some embodiments, one of the SAR targets an antigen expressed on blood cells (e.g., CD19, CD20, CD22, BCMA etc.) and a second SAR targets an antigen expressed preferentially on solid tumor cells (e.g., PSMA, Her2, MSLN etc.). In another or further embodiment of any of the foregoing, at least one SAR polypeptide of the plurality of SAR polypeptides target the same antigen. In another or further embodiment of any of the foregoing, at least one SAR polypeptide of the plurality of SAR polypeptides comprises a different binding affinity for the antigen than at least one other SAR polypeptide. In another or further embodiment of any of the foregoing, the immune cell further comprises at least one SAR encoding a SIR or zSIR or Ab-TCR and a 2^{nd} SAR encoding a chimeric antigen receptor (CAR) polypeptide. In another or further embodiment of any of the foregoing, the antigen binding domain of the first SAR polypeptide targets a different antigen than the antigen binding domain of the CAR polypeptide. In another or further embodiment of any of the foregoing, the CAR polypeptide comprises an intracellular signaling domain comprising a costimulatory signaling domain but does not comprise a primary signaling domain or comprises an intracellular signaling domain comprising a primary signaling domain but does not comprise a costimulatory signaling domain. In another or further embodiment of any of the foregoing, the CAR polypeptide comprises a costimulatory signaling domain comprising a functional signaling domain of a protein selected from the group consisting of 4-1BB, CD28, CD27 or OX-40, or the CAR molecule comprises a primary signaling domain comprising a functional signaling domain of CD3 zeta. In another or further embodiment of any of the foregoing, the CAR polypeptide is an inhibitory CAR polypeptide, wherein the inhibitory CAR polypeptide comprises an antigen binding domain, a transmembrane domain, and an intracellular domain of an inhibitory molecule, wherein the inhibitory molecule is selected from the group consisting of: PD1, PD-L1, CTLA4, TIM3, LAG3, VISTA, BTLA, TIGIT, LAIR1, CD160, 2B4, TGFR beta, CEACAM-1, CEACAM-3, and CEACAM-5. In another or further embodiment of any of the foregoing, the CAR polypeptide further comprises an intracellular signaling domain comprising a primary signaling domain and/or an intracellular signaling domain, wherein the intracellular signaling domain comprises a primary signaling domain comprising the functional domain of CD3 zeta and a costimulatory signaling domain comprising the functional domain of 4-1BB or CD28 or both.

Also provided herein are methods of treating a cancer or an autoimmune disease in a subject that include administering a therapeutically effective amount of any of the mammalian cells described herein to the subject. Some embodiments of any of the methods described herein further include, prior to the administering step, obtaining an initial cell from the subject; and introducing any of the nucleic acids described herein or any of the vectors described herein into the initial cell, to yield the mammalian cell that is administered to the subject. Some embodiments of any of the methods described herein further include, between the introducing step and the administering step, a step of culturing the cell that is administered to the subject in a liquid culture medium. In some embodiment, the SAR-T expressing cells are generated in vivo. In some embodiments of any of the methods described herein, the subject is human.

In some embodiments, the cells are exposed to an agent that improve the efficacy and/or safety of the cells. In an embodiment, the agent is selected from a tyrosine kinase inhibitor, e.g., Src kinase inhibitor, e.g., Lck inhibitor, e.g., Dasatinib, Ponatinib, a JAK/STAT inhibitor (e.g., Ruxolitinib), an mTOR inhibitor or a bispecific or a multi-specific T- or NK-cell activating antibody (e.g., BiTE, BiKE, TriKE etc.), or a CSF1R antibody. In some embodiments, the cells are exposed to the agent in vitro. In some embodiments, the cells are exposed to the agent in vivo. In some embodiments, the subject is administered the agent prior to, concurrent with or after the administration of the cells expressing the SAR. In some embodiments, the subject is administered the agent prior to, concurrent with or after the administration of a vector expressing a SAR. In some embodiments, the subject receives one dose of the agents, whereas in other embodiments, the subject receives multiple doses of the agent.

In some embodiments of any of the multi-chain SARs described herein, the SAR lacks an ITAM but recruits a signaling protein comprising a primary stimulating domain containing an ITAM. In some embodiments, the SARs recruits a signaling protein selected from the group of CD3z, FcRγ, DAP10 and DAP12.

In some embodiments, a method of making a T cell that expresses a hybrid chain Synthetic Antigen Receptor (HC-SAR or HC-SAR) or a hybrid-SAR is provided. The method can comprise contacting a T cell with a first nucleic acid that encodes a first non-TCR antigen binding domain (e.g., vL, vH, vHH, FHVH, scFv, non-immunoglobulin antigen binding scaffold, adaptor, epitope, receptor, or ligand etc.) that is operationally linked via an optional linker to a first hybrid TCR constant chain in which the first hybrid TCR chain comprises a first TCR chain transmembrane domain and a second TCR chain constant domain but does not comprise a first TCR chain constant domain. The method can comprise contacting the T cell with a second nucleic acid that encodes a second non-TCR antigen binding domain (e.g., vL, vH, vHH, FHVH, scFv, non-immunoglobulin antigen binding scaffold, adaptor, epitope, receptor or ligand etc.) that is operationally linked via an optional linker to a second hybrid TCR chain, in which the second hybrid TCR chain comprises a second chain transmembrane domain, and a first TCR chain constant domain, but does not comprise a second TCR chain constant domain. In an embodiment, the non-TCR antigen binding domain operationally linked to the first hybrid TCR constant chain is vL fragment of an antibody and the non-TCR antigen binding domain operationally linked to the second hybrid TCR constant chain is the complementary vH fragment of that antibody. In an embodiment, the vL and vH fragments can form a Fv like antigen-binding module that specifically binds to a target antigen.

In some embodiments, a method of making a T cell that expresses a hybrid chain Synthetic Antigen Receptor (HC-SAR) or a hybrid-SAR is provided. The method can comprise contacting a T cell with a first nucleic acid that encodes a first hybrid TCR constant chain in which the first hybrid TCR chain comprises a first TCR chain transmembrane domain, a variable domain of light chain of an antibody (vL) and a second TCR chain constant domain but does not comprise a first TCR chain constant domain. The method can comprise contacting the T cell with a second nucleic acid that encodes a second hybrid TCR chain, in which the second hybrid TCR chain comprises a second chain transmembrane domain, a variable domain of heavy chain of an antibody (vH) and a first TCR chain constant domain but does not comprise a second TCR chain constant domain. The first chain variable domain can comprise a vL variable domain and the first chain constant domain can comprise an alpha or a gamma chain constant domain and the first chain transmembrane domain can comprise an alpha or a gamma chain transmembrane domain and the second chain variable domain can comprise a vH variable domain and the second chain constant domain can comprise a beta or a delta chain constant domain and the second chain transmembrane domain can comprises a beta or a delta chain transmembrane domain; or the first chain variable domain can comprise a vH variable domain and the first chain constant domain can comprise an alpha or a delta chain constant domain and the first chain transmembrane domain can comprise an alpha or a delta chain transmembrane domain and the second chain variable domain can comprise vL variable domain and the second chain constant domain can comprise a beta or a gamma chain constant domain and the second chain transmembrane domain can comprise a beta or a gamma chain transmembrane domain. As such, the method can comprise configuring the T cell to express a HC-SAR comprising the first hybrid chain and the second hybrid chain. In some embodiments, the first hybrid chain further comprises a second chain connecting peptide and the second hybrid chain can further comprises a first chain connecting peptide, in which the first chain connecting peptide comprises an alpha or gamma chain connecting peptide and the second chain connecting peptide comprises a beta or delta chain connecting peptide, or in which the first chain connecting peptide comprises an alpha or delta chain connecting peptide and the second chain connecting peptide comprises a beta or gamma chain connecting peptide. In some embodiments, the first chain variable domain comprises a vL variable domain and the first chain constant domain comprises an alpha chain constant domain and the first chain transmembrane domain comprises a beta chain transmembrane domain and the second chain variable domain comprises a vH variable domain and the second chain constant domain can comprise a beta chain constant domain and the second chain transmembrane domain comprises an alpha chain transmembrane domain. In some embodiments, the first chain variable domain comprises a vL variable domain and the first chain constant domain comprises a gamma chain constant domain and the first chain transmembrane domain comprises a gamma chain transmembrane domain and the second chain variable domain comprises a vH domain and the second chain constant domain comprises a delta chain constant domain and the second chain transmembrane domain comprises a delta chain transmembrane domain. In some embodiments, the first chain variable domain comprises a vL variable domain and the first chain constant domain comprises an alpha chain constant domain and the first chain transmembrane domain comprises a gamma chain transmembrane domain and the second chain variable domain comprises a vH variable domain and the second chain constant domain comprises a beta chain constant domain and the second chain transmembrane domain comprises a delta chain transmembrane domain. In some embodiments, the first chain variable domain comprises an vL domain and the first chain constant domain comprises an alpha chain constant domain and the first chain transmembrane domain comprises a delta chain transmembrane domain and the second chain variable domain comprises a vH variable domain and the second chain constant domain comprises a beta chain constant domain and the second chain transmembrane domain comprises a gamma chain transmembrane domain. It is to be understood that the vL and vH domains in the above constructs can be substituted so that first chain may comprise vH domain and the second chain may comprise the complementary vL domain. Furthermore, one or more autonomous antigen binding domains (AABD) such as vHH, FHVH, DARPIN, Centyrin, adaptor, receptor, ligand etc. may be attached to the N-terminus or near the N-terminus of the vL and/or vH domains of double chain hybrid SIR. Several example uni-specific, bispecific, and universal SIRs targeting different antigens are provided in Tables 16-18 of the provisional patent application.

In some embodiments, a method of making a T cell that expresses a hybrid chain Synthetic Antigen Receptor or a hybrid-SAR is provided. The method can comprise contacting a T cell with a first nucleic acid that encodes a non-TCR antigen binding domain (e.g., vL, vH, vHH, FHVH, scFv, non-immunoglobulin antigen binding scaffold, adaptor, epitope, receptor, or ligand etc.) that is operationally linked via an optional linker to a first Hybrid TCR constant chain in which the first hybrid TCR chain comprises a first TCR chain transmembrane domain that is a hybrid of two TCR chains (e.g., TCRα and TCRδ or TCRβ and TCRγ) and a second TCR chain constant domain. The method can comprise contacting the T cell with a second nucleic acid that encodes a second hybrid TCR chain, in which the second hybrid TCR chain transmembrane domain is a hybrid of two TCR chains (e.g., TCRα and TCRδ or TCRβ and TCRγ).

The method can comprise contacting a T cell with a first nucleic acid that encodes a non-TCR antigen binding domain (e.g., vL, vH, vHH, FHVH, scFv, non-immunoglobulin antigen binding scaffold, adaptor, epitope, receptor, or ligand, etc.) that is operationally linked via an optional linker to a first Hybrid TCR constant chain in which the first hybrid TCR chain comprises a first TCR chain transmembrane domain that is a hybrid of two TCR chains (e.g., TCRα and TCRδ or TCRβ and TCRγ) and a second TCR chain constant domain. The method can comprise contacting the T cell with a second nucleic acid that encodes a second hybrid TCR chain, in which the second hybrid TCR chain transmembrane domain is a hybrid of two TCR chains (e.g., TCRα and TCRδ or TCRβ and TCRγ).

In some embodiments, a method of making an immune cell (e.g., a T cell) that expresses a hybrid chain Synthetic Antigen Receptor (HC-SAR) or a hybrid-SAR is provided. The method can comprise contacting a T cell with a first nucleic acid that encodes a non-TCR antigen binding domain (e.g., vL, vH, vHH, FHVH, scFv, non-immunoglobulin antigen binding scaffold, adaptor, epitope, receptor, or ligand etc.) that is operationally linked via an optional linker to a first hybrid TCR constant chain in which the first hybrid TCR chain comprises a first TCR chain transmembrane domain and a second TCR chain Ig-like domain but does not comprise a first TCR chain Ig-like domain. The method can comprise contacting the T cell with a second nucleic acid that encodes a second hybrid (or hybrid) TCR chain, in which the second hybrid TCR chain comprises a second chain transmembrane domain, a non-TCR antigen binding domain (e.g., vL, vH, vHH, FHVH, scFv, non-immunoglobulin antigen binding scaffold, adaptor, epitope, receptor or ligand etc.) and a first TCR chain Ig-like domain, but does not comprise a second TCR chain constant domain. In an embodiment, the vL of the first hybrid TCR chain and vH of the second hybrid TCR chain interact to form a fragment variable (Fv) that can specifically bind to a target antigen when expressed on the surface of an immune cell (e.g., T cell). In an embodiment, an immune cell expressing the hybrid SAR can initiate a signal transduction pathway when exposed to a target antigen expressing cell. In an embodiment, an immune cell expressing the hybrid SAR can initiate cell activation, differentiation, proliferation, cytokine secretion and/or cytotoxicity when exposed to a target antigen expressing cell.

In an embodiment, the SAR with the hybrid TCR chains show higher cell surface expression when expressed on an immune cell (e.g., T cell) as compared to a cTCR comprising wild-type nucleic acid and amino acid sequences of TCR chains but comprising the identical antigen binding domains. In an embodiment, the SAR with the hybrid TCR chains show at least 5% (e.g., 5%, 10%, 15%, 20%, 25%, 30%, 35%, 40%, 45%, 50%, 60%, 70%, 80%, 90%, 100% etc.) higher cell surface expression when expressed on an immune cell (e.g., T cell) as compared to a cTCR comprising wild-type nucleic acid and amino acid sequences of TCR chains but comprising the identical antigen binding domains. Expression of hybrid chain SAR and cTCR is measured using techniques known in the art, such as Protein L staining and/or Topanga Assay.

In an embodiment, the SAR with the hybrid TCR chains show reduced chain pairing with endogenous TCR chains when expressed on an immune cell (e.g., T cell) as compared to a cTCR or a SIR comprising the identical antigen binding domains. In an embodiment, the SAR with the hybrid TCR chains show at least 5% (e.g., 5%, 10%, 15%, 20%, 25%, 30%, 35%, 40%, 45%, 50%, 60%, 70%, 80%, 90%, 100% etc.) lower chain pairing with endogenous TCR chains when expressed on an immune cell (e.g., T cell) as compared to a cTCR and/or SIR comprising the identical antigen binding domains.

In an embodiment, the SAR with the hybrid TCR chains show higher cell activation, differentiation, proliferation, cytokine secretion and/or cytotoxicity when expressed on an immune cell (e.g., T cell) as compared to a cTCR comprising wild-type nucleic acid and amino acid sequences of TCR chains but comprising the identical antigen binding domains. In an embodiment, the SAR with the hybrid TCR chains show at least 5% (e.g., 5%, 10%, 15%, 20%, 25%, 30%, 35%, 40%, 45%, 50%, 60%, 70%, 80%, 90%, 100% etc.) higher cell activation, differentiation, proliferation, cytokine secretion and/or cytotoxicity when expressed on an immune cell (e.g., T cell) as compared to a cTCR comprising wild-type nucleic acid and amino acid sequences of TCR chains but comprising the identical antigen binding domains. Cell activation, differentiation, proliferation, cytokine secretion and cytotoxicity are measured using techniques known in the art, such as Flow cytometry, ELISA and Matador cytotoxicity assay.

In some embodiments, a method of making a T cell that expresses a hybrid chain Synthetic Antigen Receptor (HC-SAR) or a hybrid-SAR is provided. The method can comprise contacting a T cell with a first nucleic acid that encodes a first hybrid (or Hybrid) TCR constant chain in which the first hybrid TCR chain comprises a first TCR chain transmembrane domain, a variable domain of light chain of an antibody (vL) and a second TCR chain Ig-like domain but does not comprise a first TCR chain Ig-like domain. The method can comprise contacting the T cell with a second nucleic acid that encodes a second hybrid (or hybrid) TCR chain, in which the second hybrid TCR chain comprises a second chain transmembrane domain, a variable domain of heavy chain of an antibody (vH) and a first TCR Ig-like domain but does not comprise a second TCR chain Ig-like domain. The first chain variable domain can comprise a vL variable domain and the first chain Ig-like domain can comprise an alpha or a gamma chain Ig-like domain and the first chain transmembrane domain can comprise an alpha or a gamma chain transmembrane domain and the second chain variable domain can comprise a vH variable domain and the second chain Ig-like domain can comprise a beta or a delta chain Ig-like domain and the second chain transmembrane domain can comprises a beta or a delta chain transmembrane domain; or the first chain variable domain can comprise a vH variable domain and the first chain Ig-like domain can comprise an alpha or a delta chain constant domain and the first chain transmembrane domain can comprise an alpha or a delta chain transmembrane domain and the second chain variable domain can comprise vL variable domain and the second chain Ig-like domain can comprise a beta or a gamma chain Ig-like domain and the second chain transmembrane domain can comprise a beta or a gamma chain transmembrane domain. As such, the method can comprise configuring the T cell to express a HC-SAR comprising the first hybrid chain and the second hybrid chain. In some embodiments, the first hybrid chain further comprises a second chain connecting peptide and the second hybrid chain can further comprises a first chain connecting peptide, in which the first chain connecting peptide comprises an alpha or gamma chain connecting peptide and the second chain connecting peptide comprises a beta or delta chain connecting peptide, or in which the first chain connecting peptide comprises an alpha or delta chain connecting peptide and the second chain connecting peptide comprises a beta or gamma chain connecting peptide. In some embodiments, the first chain variable domain comprises a vL variable domain and the first chain constant domain comprises an alpha chain constant domain and the first chain transmembrane domain comprises a beta chain transmembrane domain and the second chain variable domain comprises a vH variable domain and the second chain constant domain can comprise a beta chain constant domain and the second chain transmembrane domain comprises an alpha chain transmembrane domain. In some embodiments, the first chain variable domain comprises a vL variable domain and the first chain Ig-like domain comprises a gamma chain Ig-like domain and the first chain transmembrane domain comprises a gamma chain transmembrane domain and the second chain variable domain comprises a vH domain and the second chain Ig-like domain comprises a delta chain Ig-like domain and the second chain transmembrane domain comprises a delta chain transmembrane domain. In some embodiments, the first chain variable domain comprises a vL variable domain and the first chain Ig-like domain comprises an alpha chain Ig-like domain and the first chain transmembrane domain comprises a gamma chain transmembrane domain and the second chain variable domain comprises a vH variable domain and the second chain Ig-like domain comprises a beta chain Ig-like domain and the second chain transmembrane domain comprises a delta chain transmembrane domain. In some embodiments, the first chain variable domain comprises an vL domain and the first chain Ig-like domain comprises an alpha chain Ig-like domain and the first chain transmembrane domain comprises a delta chain transmembrane domain and the second chain variable domain comprises a vH variable domain and the second chain Ig-like domain comprises a beta chain Ig-like domain and the second chain transmembrane domain comprises a gamma chain transmembrane domain. It is to be understood that the vL and vH domains in the above constructs can be substituted so that first chain may comprise vH domain and the second chain may comprise the complementary vL domain. Furthermore, one or more autonomous antigen binding domains (AABD) such as vHH, FHVH, DARPIN, Centyrin, adaptor, receptor, ligand etc. may be attached to the N-terminus or near the N-terminus of the vL and/or vH domains of double chain hybrid SIR.

It is also understood that a multichain SIR with hybrid (or hybrid) TCR chains may comprise an antigen binding domain (e.g., scFV, vHH, FHVH, Darpin, non-immunoglobulin antigen binding domain, receptor, ligand, auto-antigen etc.) attached to only one of the hybrid (or hybrid) TCR chains, which is the co-expressed with the complementary hybrid (or hybrid) TCR chain that lacks an antigen binding domain. Such a hybrid-chain SIR is called a One and half chain hybrid SIR. Furthermore, one or more autonomous antigen binding domains (AABD) such as vHH, FHVH, DARPIN, Centyrin, adaptor, receptor, ligand may be attached to the N-terminus or near the N-terminus of the antigen binding domain of the hybrid (or hybrid chain) SIR. Several example uni-specific, bispecific and universal one and half chain hybrid SIR targeting different antigens are provided in Tables 19 and 20 of the provisional patent application.

In some embodiments, an expression vector is provided. The expression vector can comprise a first nucleic acid that encodes a non-TCR antigen binding domain (e.g., vL, vH, vHH, FHVH, scFv, non-immunoglobulin antigen binding scaffold, adaptor, epitope, receptor, or ligand, etc.) that is operationally linked via an optional linker to a first hybrid (or hybrid) TCR chain in which the first hybrid (or Hybrid) TCR chain comprises a first TCR chain transmembrane domain and a second TCR chain constant domain but does not comprise a first TCR chain constant domain. The method can comprise contacting the T cell with a second nucleic acid that encodes a second hybrid (or hybrid) TCR chain, in which the second hybrid TCR chain comprises a second chain transmembrane domain, a non-TCR antigen binding domain (e.g., vL, vH, vHH, FHVH, scFv, non-immunoglobulin antigen binding scaffold, adaptor, epitope, receptor or ligand etc.) and a first TCR chain constant domain, but does not comprise a second TCR chain constant domain. In an embodiment, the vL of the first hybrid TCR chain and vH of the second hybrid TCR chain interact when expressed on the surface of an immune cell (e.g., T cell) to form a fragment variable (Fv), which can bind specifically to a target antigen.

In some embodiments, an expression vector is provided. The expression vector can comprise a first nucleic acid that encodes a non-TCR antigen binding domain (e.g., vL, vH, vHH, FHVH, scFv, non-immunoglobulin antigen binding scaffold, adaptor, epitope, receptor, or ligand, etc.) that is operationally linked via an optional linker to a first hybrid (or hybrid) TCR chain in which the first hybrid (or hybrid) TCR chain comprises a first TCR chain transmembrane domain and a second TCR chain Ig-like domain but does not comprise a first TCR chain Ig-like domain. The method can comprise contacting the T cell with a second nucleic acid that encodes a second hybrid (or hybrid) TCR chain, in which the second hybrid TCR chain comprises a second chain transmembrane domain, a non-TCR antigen binding domain and a first TCR chain Ig-like domain but does not comprise a second TCR chain Ig-like domain. In an embodiment, the vL of the first hybrid TCR chain and vH of the second hybrid TCR chain interact when expressed on the surface of an immune cell (e.g., T cell) to form a fragment variable (Fv), which can bind specifically to a target antigen.

The expression vector can comprise a first nucleic acid that encode a first hybrid (or Hybrid) TCR constant chain in which the first hybrid (or Hybrid) TCR chain comprises a first TCR chain transmembrane domain, a variable domain of light chain of an antibody (vL) and a second TCR chain Ig-like domain but does not comprise a first TCR chain Ig-like domain. The expression vector can comprise a second nucleic acid that encodes a second hybrid (or hybrid) TCR chain, in which the second hybrid TCR chain comprises a second chain transmembrane domain, a variable domain of heavy chain of an antibody (vH) and a first TCR Ig-like domain but does not comprise a second TCR chain Ig-like domain. The first chain variable domain can comprise a vL variable domain and the first chain Ig-like domain can comprise an alpha or a gamma chain Ig-like domain and the first chain transmembrane domain can comprise an alpha or a gamma chain transmembrane domain and the second chain variable domain can comprise a vH variable domain and the second chain Ig-like domain can comprise a beta or a delta chain Ig-like domain and the second chain transmembrane domain can comprises a beta or a delta chain transmembrane domain; or the first chain variable domain can comprise a vH variable domain and the first chain Ig-like domain can comprise an alpha or a delta chain constant domain and the first chain transmembrane domain can comprise an alpha or a delta chain transmembrane domain and the second chain variable domain can comprise vL variable domain and the second chain Ig-like domain can comprise a beta or a gamma chain Ig-like domain and the second chain transmembrane domain can comprise a beta or a gamma chain transmembrane domain. As such, the expression vector can comprise a hybrid chain SAR comprising the first hybrid chain and the second hybrid chain. In some embodiments, the first hybrid chain further comprises a second chain connecting peptide and the second hybrid chain can further comprises a first chain connecting peptide, in which the first chain connecting peptide comprises an alpha or gamma chain connecting peptide and the second chain connecting peptide comprises a beta or delta chain connecting peptide, or in which the first chain connecting peptide comprises an alpha or delta chain connecting peptide and the second chain connecting peptide comprises a beta or gamma chain connecting peptide. In some embodiments, the first chain variable domain comprises a vL variable domain and the first chain constant domain comprises an alpha chain constant domain and the first chain transmembrane domain comprises a beta chain transmembrane domain and the second chain variable domain comprises a vH variable domain and the second chain constant domain can comprise a beta chain constant domain and the second chain transmembrane domain comprises an alpha chain transmembrane domain. In some embodiments, the first chain variable domain comprises a vL variable domain and the first chain Ig-like domain comprises a gamma chain Ig-like domain and the first chain transmembrane domain comprises a gamma chain transmembrane domain and the second chain variable domain comprises a vH domain and the second chain Ig-like domain comprises a delta chain Ig-like domain and the second chain transmembrane domain comprises a delta chain transmembrane domain. In some embodiments, the first chain variable domain comprises a vL variable domain and the first chain Ig-like domain comprises an alpha chain Ig-like domain and the first chain transmembrane domain comprises a gamma chain transmembrane domain and the second chain variable domain comprises a vH variable domain and the second chain Ig-like domain comprises a beta chain Ig-like domain and the second chain transmembrane domain comprises a delta chain transmembrane domain. In some embodiments, the first chain variable domain comprises an vL domain and the first chain Ig-like domain comprises an alpha chain Ig-like domain and the first chain transmembrane domain comprises a delta chain transmembrane domain and the second chain variable domain comprises a vH variable domain and the second chain Ig-like domain comprises a beta chain Ig-like domain and the second chain transmembrane domain comprises a gamma chain transmembrane domain. It is to be understood that the vL and vH domains in the above constructs can be substituted so that first chain may comprise vH domain and the second chain may comprise the complementary vL domain. Furthermore, the expression vector may comprise one or more autonomous antigen binding domains (AABD) may be attached to the N-terminus or near the N-terminus of the vL and/or vH domains of double chain hybrid SIR.

The expression vector can comprise a first nucleic acid that encodes a first hybrid chain comprising a first chain transmembrane domain, a vL domain and a second chain constant domain. The expression vector can comprise a second nucleic acid that encodes a second hybrid chain comprising a second chain transmembrane domain, a vH domain and a first chain constant domain. The first chain variable domain can comprise vL domain and the first chain constant domain can comprise an alpha or a gamma chain constant domain and the first chain transmembrane domain can comprise an alpha or a gamma chain transmembrane domain and the second chain variable domain can comprise a vH domain and the second chain constant domain can comprise a beta or a delta chain constant domain and the second chain transmembrane domain can comprise a beta or a delta chain transmembrane domain; or the first chain variable domain can comprise vL domain and the first chain constant domain can comprise an alpha or a delta chain constant domain and the first chain transmembrane domain can comprise an alpha or a delta chain transmembrane domain and the second chain variable domain can comprise a vH and the second chain constant domain can comprise a beta or a gamma chain constant domain and the second chain transmembrane domain can comprise a beta or a gamma chain transmembrane domain. In some embodiments, the first hybrid chain further comprises a second chain connecting peptide but not a first chain connecting peptide, and the second hybrid chain further comprises a first chain connecting peptide but not a second chain connecting peptide, in which the first chain connecting peptide comprises an alpha or gamma chain connecting peptide and the second chain connecting peptide comprises a beta or delta chain connecting peptide; or the first chain connecting peptide comprises an alpha or delta chain connecting peptide and the second chain connecting peptide comprises a beta or gamma chain connecting peptide.

In some embodiments, the first nucleic acid and the second nucleic acid are part of the same expression vector and the expression vector further comprises a 2A peptide-encoding sequence flanked by the first nucleic acid and the second nucleic acid in which the first nucleic acid and the second nucleic acid are driven by a single promoter. In some embodiments, the first nucleic acid and the second nucleic acid are part of separate expression vectors. Optionally, the first nucleic acid and the second nucleic acid can be driven by their own separate promoters. Optionally, the first nucleic acid and the second nucleic acid can be part of a single expression vector and driven by their own separate promoters. In some embodiments, the expression vector comprises a lentiviral vector, retroviral vector, adenoviral vector, or adeno-associated viral vector. In some embodiment, the first and/or the second and/or the third nucleic acid comprising the SAR or the accessory module/therapeutic control is driven by the activation of a Synthetic Notch (SynNotch) receptor or a variant thereof. SynNotch receptors are described in WO2022140159A1, which is incorporated in its entirety by reference.

In some embodiments, a genetically engineered cell is provided. In an embodiment, the cell is a T cell. In an embodiment, the cell is an NK cell, NKT cell, iNKT cell, G-NK cell, macrophage, monocyte, granulocyte, embryonic stem cell, iPSC, or a hematopoietic stem cell. The genetically engineered T cell can comprise a first nucleic acid that encodes a first hybrid chain comprising a first chain transmembrane domain, a vL domain and a second chain constant domain. The genetically engineered T cell can comprise a second nucleic acid that encodes a second hybrid chain comprising a second chain transmembrane domain, a vH domain and a first chain constant domain. The first chain variable domain can comprise a vL variable domain and the first chain constant domain can comprise an alpha or a gamma chain constant domain and the first chain transmembrane domain can comprise an alpha or a gamma chain transmembrane domain and the second chain variable domain can comprise a vH chain variable domain and the second chain constant domain can comprise a beta or a delta chain constant domain and the second chain transmembrane domain can comprise a beta or a delta chain transmembrane domain; or the first chain variable domain can comprise an vH chain variable domain and the first chain constant domain can comprise an alpha or a delta chain constant domain and the first chain transmembrane domain can comprise an alpha or a delta chain transmembrane domain and the second chain variable domain can comprise a vL domain and the second chain constant domain can comprise a beta or a gamma chain constant domain and the second chain transmembrane domain can comprise a beta or a gamma chain transmembrane domain. As such, the T cell can be configured to express a HC-SAR comprising the first hybrid chain and the second hybrid chain. In some embodiments, the first hybrid chain further comprises a second chain connecting peptide and the second hybrid chain further comprises a first chain connecting peptide, in which the first chain connecting peptide comprises an alpha or gamma chain connecting peptide and the second chain connecting peptide comprises a beta or delta chain connecting peptide; or in which the first chain connecting peptide comprises an alpha or delta chain connecting peptide and the second chain connecting peptide comprises a beta or gamma chain connecting peptide. In some embodiments, the genetically engineered T cell is configured to express the first hybrid chain as a first polypeptide and the second hybrid chain as a second polypeptide, in which the first hybrid chain and second hybrid chain are separate molecules. In some embodiments, expression of an endogenous TCR is repressed or eliminated in the genetically engineered T cell.

In some embodiments, a method of inducing an immune response in a subject is provided. The method can comprise configuring an isolated T cell to express a first hybrid chain that comprises a first chain transmembrane domain, a vL domain, and a second chain constant domain, but does not comprise a first chain variable domain and does not comprise a first chain constant domain. The method can comprise configuring the isolated T cell to express a second hybrid chain that comprises a second chain transmembrane domain, a vH domain, and a first chain constant domain, but does not comprise a second chain constant domain. The first chain variable domain can comprise vL domain and the first chain constant domain can comprise an alpha or a gamma chain constant domain and the first chain transmembrane domain can comprise an alpha or a gamma chain transmembrane domain and the second chain variable domain can comprise a vH domain and the second chain constant domain can comprise a beta or a delta chain constant domain and the second chain transmembrane domain can comprise a beta or a delta chain transmembrane domain; or the first chain variable domain can comprise a vH domain and the first chain constant domain can comprise an alpha or a delta chain constant domain and the first chain transmembrane domain can comprise an alpha or a delta chain transmembrane domain and the second chain variable domain can comprise a vL domain and the second chain constant domain can comprise a beta or a gamma chain constant domain and the second chain transmembrane domain can comprise a beta or a gamma chain transmembrane domain. As such, the genetically engineered T cell configured to express a hybrid Synthetic Immune receptor (HC-SAR) comprising the first hybrid chain and the second hybrid chain can be administered to the subject. In some embodiments, the first hybrid chain further comprises a second chain connecting peptide and the second hybrid chain can further comprise a first chain connecting peptide, in which the first chain connecting peptide comprises an alpha or gamma chain connecting peptide and the second chain connecting peptide comprises a beta or delta chain connecting peptide; or in which the first chain connecting peptide comprises an alpha or delta chain connecting peptide and the second chain connecting peptide comprises a beta or gamma chain connecting peptide. In some embodiments, the isolated T cell is autologous to the subject. In some embodiments, the isolated T cell is allogeneic to the subject. In some embodiments, the T cell comprises a CD4 T cell. In some embodiments, the T cell comprises a CD8 T cell. In some embodiments, the T cell comprises a regulatory T cell (Treg). In some embodiments, the T cell is co-administered with a second genetically engineered T cell population. In some embodiments, the T cell is administered in a single dose. In some embodiments, the T cell is administered in in multiple doses. In some embodiments, the subject has at least one of a tumor, a cancer, an infectious disease, an autoimmune disease and is in need of treatment therefor. In some embodiments, the subject has diminished or ineffective or exhausted T cells and is in need of treatment therefor. In some embodiments, the T cell is induced to express a plurality of HC-SAR against an array of antigens. In some embodiments, the T cell can be administered to the subject via at least one of intramuscular injection, intravaginal injection, intravenous injection, intraperitoneal injection, subcutaneous injection, epicutaneous administration, intradermal administration, or nasal administration. In some embodiments, the administered T cell is further monitored over time. In some embodiments, the method can be repeated as desired.

### BRIEF DESCRIPTION OF THE DRAWINGS

FIG. 1 shows bioluminescence imaging of NSG mice xenografted with JEKO-1 cells and administered either control T cells or T cells expressing the indicated CD79b SAR constructs.

FIG. 2 shows bioluminescence imaging of NSG mice xenografted with LNCaP cells and administered either control T cells or T cells expressing the indicated STEAP2 SAR constructs.

FIG. 2 shows bioluminescence imaging of NSG mice xenografted with NALM6 cells and administered either control NK cells or T cells expressing the indicated CD19 SAR constructs.

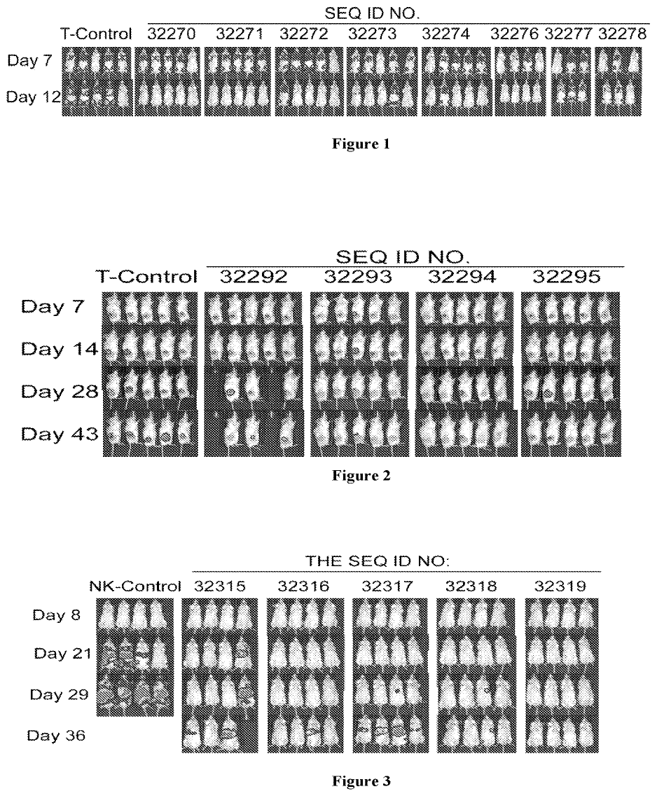

### DETAILED DESCRIPTION

The invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the disclosure are generally performed according to conventional methods well-known in the art and as described in various general and more specific references that are cited and discussed throughout the present specification unless otherwise indicated. See, e.g., Sambrook et al., Molecular Cloning: A Laboratory Manual (4th ed., Cold Spring Harbor Laboratory Press, Cold Spring Harbor, N.Y. (2013). The nomenclatures used in connection with, and the laboratory procedures and techniques of, immunology, molecular biology, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “autonomous antigen binding domain” or “AABD” as used herein refers to an antigen binding domain that can bind to an antigen autonomously, i.e., in the absence of another antigen binding domain. An example AABD is a single vH domain or an autonomous vH domain (aVH), typically a single human vH domain (SVH) that can bind an antigen in the absence of a vL domain. Another example AABD is a fully human vH domain (FHVH). Another example AABD is a single vL domain or an autonomous vL domain, typically a single human vL domain (SVL) that can bind an antigen in the absence of a vH domain. AABD also refers to other antigen binding domains that can bind an antigen autonomously. In an embodiment, the AABD is a non-scFv antigen binding domain. An example non-scFV based autonomous antigen binding domain includes but is not limited to a vHH domain, a humanized vHH domain, a single variable domain-TCR (svd-TCR), and non-immunoglobulin antigen binding scaffold such as a DARPIN, an affibody, a ZIP domain (e.g., RZIP, EZIP, E4, R4 etc.), an affilin, an adnectin, an affitin, an obody, a repebody, a fynomer, an alphabody, an avimer, an atrimer, a centyrin, a pronectin, an anticalin, a kunitz domain, an Armadillo repeat protein or a fragment thereof. Additional examples of non-scFV based autonomous antigen binding domains include the ligand binding domain of a receptor (e.g., CD16-V158A, NKG2D) or a fragment thereof, the receptor binding domain of a ligand (e.g., APRIL, Thrombopoietin etc.) or a fragment thereof, an adaptor (e.g., RZIP, EZIP, E4, K4, NKG2D-YA, NKG2D-AF etc.) or a fragment thereof, an adatptor binding protein (e.g. ULBP2R, ULBP2-S3 etc.) or a fragment thereof, an epitope or a tag (e.g., Streptag, FLAG tag etc.), an autoantigen or a fragment thereof and the like.

The disclosure described the use of AABD, such as human VH (or vH) domains, such as multiple human VH domains, as building blocks to make uni-specific, bispecific, and multi-specific SARs.

The term “about” when referring to a measurable value such as an amount, a temporal duration, and the like, is meant to encompass variations of 20% or in some instances ±10%, or in some instances+5%, or in some instances+1%, or in some instances ±0.1% from the specified value, as such variations are appropriate to perform the disclosed methods or describe the compositions herein. Moreover, any value or range (e.g., less than 20 or similar terminology) explicitly includes any integer between such values or up to the value. Thus, for example, “one to five mutations” explicitly includes 1, 2, 3, 4 and/or 5 mutations.

The term “Ab-TCR” or “AbTCR” refers to a next generation CAR platform as described in WO 2017/070608 A1 which is incorporated herein by reference.

The term “accessory module” refers to any one or more of PDL1, PDL2, CD80, CD86, crmA, p35, K13-opt, MC159, MyD88-L265P, TCL-1a, 41BBL, CD40L, vFLIP-K13, MC159, cFLIP-L/MRITα, IgSP-[hTRAC-opt2], IgSP-[hTRBC-opt2], a multi-purpose switch (e.g., IL2-tBCMA, IL15-tBCMA, IL2-RQR, IL15-RQR etc.), NKG2C, CD94, DAP10, DAP12, CD3ε, CD3γ, CD3δ, CD3ζ, FcRy, and combination thereof that is expressed in an immune cell (e.g., NK cell or T cell, e.g., SAR-NK cell, SAR-T cell or TCR-T cell) to decrease, regulate or modify the activity of the immune cell. In an embodiment, an accessory module is a therapeutic control (e.g., icapase 9). The nucleic and amino acids SEQ ID NOs of several example accessory modules and therapeutic controls are provided in Table 7 of the provisional patent application (e.g., SEQ ID NO: 9038-9047, 9284-9308, 9348-9349). In some embodiments, the accessory module is co-expressed with an immune receptor such as a SAR or a TCR to increase, decrease, regulate, or modify the expression or activity of a SAR or a TCR or a SAR-expressing or a TCR-expressing cell.

The term “antibody,” as used herein, refers to a protein, or polypeptide sequence derived from an immunoglobulin molecule which specifically binds with an antigen. Antibodies can be monoclonal or polyclonal, multiple or single chain, or intact immunoglobulins, and may be derived from natural sources or from recombinant sources. The antibody may be ‘humanized,’ ‘chimeric,’ fully human or non-human. An antibody may have a single domain (e.g., a single vH domain).

The term “antibody fragment” refers to at least one portion of an antibody that retains the ability to specifically interact with (e.g., by binding, steric hindrance, stabilizing/destabilizing, spatial distribution) an epitope of an antigen

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

“Anticancer agent” refers to agents that inhibit aberrant cellular division and growth, inhibit migration of neoplastic cells, inhibit invasiveness, or prevent cancer growth and metastasis.

The term “anticancer effect” or “anti-tumor effect” refers to a biological effect which can be manifested by various means, including but not limited to, a decrease in tumor volume. An “anticancer effect” can also be manifested by the ability of the SARs to prevent the occurrence of cancer in the first place.

The term “antigen” or “Ag” refers to a molecule that provokes an immune response. This immune response may involve either antibody production, or the activation of specific immunologically competent cells, or both. Non-limiting examples of antigen or antigens that can be specifically bound by any of the antigen-binding domains are described in Table B.

An “antigen binding domain” or “antigen binding module” or “antigen binding segment” or “antigen specific domain” (ASD) refers to a polypeptide or peptide that due to its primary, secondary, or tertiary sequence, post-translational modifications and/or charge binds to an antigen with a high degree of specificity. In example embodiments, the target antigens and SEQ ID Nos of various antigen binding domains are set forth herein in Tables 3-7. In example embodiments, the target antigen and SEQ ID NOs of vL, vH, scFVs, and their CDR regions are set forth herein in Tables 6A-C of patent application PCT/US18/53247 and in Tables 3-4 of patent application PCT/US19/035096, which are incorporated in their entirety by reference herein.

The term “autoantigen” refers to an endogenous antigen that stimulates production of an autoimmune response, such as production of autoantibodies. Examples of autoantigens include, but are not limited to, desmoglein 1, desmoglein 3, and fragments thereof.

“Avidity” refers to the strength of interaction between an agent and its target.

As used herein, the term “backbone” or “architecture” refers to the configuration of the different components (e.g., antigen binding domains, hinge domains, transmembrane domains, signaling domains) that comprise different SAR and any accessory module which is generally optional.

Table 1: Conventional CAR architectures. First generation conventional CARs (Conventional CAR I) have an intracellular signaling (ISD) domain (e.g., CD3z) and no costimulatory domain. The TCR fusion proteins (TFP) are another example of conventional CAR 1. Second generation conventional CARs (Conventional CAR 2 or CAR II) have one costimulatory domain (e.g., 41BB or CD28) and an intracellular signaling (ISD) domain (e.g., CD3z). Third generation conventional CARs (Conventional CAR 3 or CAR III) have two costimulatory domains (e.g., 41BB and CD28) and an intracellular signaling (ISD) domain (e.g., CD3z). Ab-TCRs are duel chain receptors incorporating a vL-linker-TCR domain (TCRD and a vH-linker-TCR domain (TCRD) and have been described in PCT/US2016/058305. cTCRs (chimeric T cell receptors) are single chain, one-and-half, or double chain receptors consisting of antigen binding domain derived from a vL and vH fragment that are fused to one or more TCR constant chain (TCR-C) and result in activation of T cell signaling. The TCR constant chains of cTCRs are encoded by wild-type nucleic acid sequences and corresponding wild-type amino acid sequences. Different configurations of cTCR are described in PCT/US2017/064379 or WO 2018/102795 A1. Synthetic immune receptors are next generation CARs and are described in PCT/US2017/064379 or WO 2018/102795 A1. SIRs are single chain, one-and-half, or double chain receptors. In one embodiment, the antigen binding domain of SIR are derived from a vL and vH fragment that are fused to one or more TCR constant chain (TCR-C) and result in activation of T cell signaling. In some embodiments, the TCR constant chains of SIR are encoded by human codon-optimized nucleic acid sequences and comprise one or more mutations that enhance their expression and chain-pairing. zSIRs are double chain receptors comprising antigen binding domains (e.g., vL, vH etc.) that are operationally linked to two CD3z chains or fragments thereof with optional linkers and are described in PCT/US2019/035096.

TABLE 1
Example CONVENTIONAL CAR Architectures

| | | | | | | | |
|---|---|---|---|---|---|---|---|
| 1 | CAR 1 or CAR I | ASD | HR | TMD | ISD | | |
| | (including TFP) | | | | | | |
| 2 | CAR 2 (CAR II) | ASD | HR | TMD | CSD | ISD | |
| 3 | CAR 3 (CAR III) | ASD | HR | TMD | CSD-I | CSD-II | ISD |
| 4 | Ab-TCR | vL-CL | TCRD(1) | 2A | vH-CH1 | TCRD (II) | |
| 5 | Double Chain | vL | TCR-C(1) | 2A | vH | TCR-C (II) | |
| | cTCR/SIR-1 | | | | | | |
| 6 | Double Chain zSIR | vL- | CD3z | 2A | vH-linker | CD3z | |
| | | linker | | | | | |
| 6 | One & Half Chain | | TCR-C(1) | 2A | ASD | TCR-C (II) | |
| | cTCR/SIR-3 | | | | | | |

TABLES 2A to 2J provide example architectures of uni-specific, bispecific, and multi-specific SARs of this disclosure. The abbreviations used are: SP (signal peptide); AADB (autonomous antigen binding domain); L (optional linker); LL (Long linker), (AABD-L)n (n copies of AABD with optional linker where n=0, 1, 2, 3, 4 or more), AABD1-4 (different AABD targeting one or more antigens), V1 (vL, vH, Va, Vb, Vg or Vd chains), Ig (Ig linker), TCR-Ig (Ig linker domain derived from TCR chains), ConP (connecting peptide), TM (transmembrane domain), CP (cytosolic domain), IC (intracellular domain), Ca (Constant chain of TCRα), Cb (constant chain of TCRβ), Cg (constant chain of TCRγ), Cd (constant chain of TCRδ), scFv (single chain fragment variable), scTFv (single chain fragment comprising two variable fragments of a TCR, e.g., Va and Vb), dCa/dCb/dCg/dCd (N-terminally deleted constant chain of TCRα, β, γ or δ lacking their Ig linker domain), TCR-ConP (connecting peptide of TCRα, β, γ or δ constant chain), Ca-ConP (connecting peptide of TCRα constant chain), IgCL (Ig linker from immunoglobulin light chain), IgCH1 (Ig-liker from immunoglobulin heavy chain), CD3εγδ ECD (extracellular domain of CD3ε, γ or δ chains), CSD (costimulatory domain), 4-1BB or BB (costimulatory domain of 4-1BB), CD28 or 28 (costimulatory domain of CD28), CD3z or zd or z (activation domain of CD3z). NKp30-lg (Immunoglobulin like domain of Nkp30), NKp44-Ig (Immunoglobulin like domain of Nkp44), NKp46-Ig1-Ig2 (Immunoglobulin like domain 1 and 2 of Nkp46), CD16-D1 (Domain 1 of CD16), CD16-D2 (Domain 2 of CD16), scTCR (Single chain TCR), Extracellular domain (ECD), activation domain (AD). Va, Vb, Vg, Vd (variable domains of TCRα, β, γ and δ), FCRG (FcRγ); Hinge domain (Hn).

TABLE 2A
EXAMPLE TCR-SAR WITH BACKBONE OF zSAR

| | | | | | | | | | |
|---|---|---|---|---|---|---|---|---|---|
| 1 | SP | L | (AABD-L)n | Va | L | Ig | ECD | TM | CP |
| | SP | L | (AABD-L)n | Vb | L | Ig | ECD | TM | CP |
| 2 | SP | L | (AABD-L)n | Va | L | Ig | Hinge | TM | CP |
| | SP | L | (AABD-L)n | Vb | L | Ig | Hinge | TM | |
| 3 | SP | L | (AABD-L)n | Va | L | Ig | CD3z-ECD | CD3z-TM | CD3z-CP |
| | SP | L | (AABD-L)n | Vb | L | lg | CD16-ECD | CD16-TM | |
| 4 | SP | L | (AABD-L)n | Va | L | TCR-Ig | CD3z-ECD | CD3z-TM | CD3z-CP |
| | SP | L | (AABD-L)n | Vb | L | TCR-Ig | CD16-HN | CD16-TM | 41BB or CD28 |
| 5 | SP | L | (AABD-L)n | Va | L | TCRa-Ig | CD3z-ECD | CD3z-TM | CD3z-CP |
| | SP | L | (AABD-L)n | Vb | L | TCRb-Ig | CD16-HN | CD16-TM | CD8a or CD8b |
| | | | | | | | | | or CD4 |
| 6 | SP | L | (AABD-L)n | Va | L | TCRa-Ig | CD3z-ECD | CD3z-TM | 41BB-CD3zCP |
| | SP | L | (AABD-L)n | Vb | L | TCRb-Ig | CD16-HN | CD16-TM | Lck or LAT or |
| | | | | | | | | | SLP-76 |
| 7 | SP | L | (AABD-L)n | Va | L | TCRa-Ig | CD3z-ECD | CD3z-TM | 41BB-CD3zCP |
| | SP | L | (AABD-L)n | Vb | L | TCRb-Ig | CD3z-ECD | CD3z-TM | Lck or LAT or |
| | | | | | | | | | SLP-76 |
| 7 | SP | L | (AABD-L)n | Va | L | Ig linker | FcRy-ECD | FcRy-TM | 41BB-FcRyCP |
| | SP | L | (AABD-L)n | Vb | L | Ig linker | CD3z-ECD | CD3z-TM | Lck or LAT or |
| | | | | | | | | | SLP-76 |
| 7 | SP | L | (AABD-L)n | Va | L | Ig linker | FcRy-ECD | FcRy-TM | 41BB-FcRyCP |
| | SP | L | (AABD-L)n | Vb | L | Ig linker | CD16-HN | CD16-TM | Lck or LAT or |
| | | | | | | | | | SLP-76 |

The Va and Vb in the constructs in Table 2A can be replaced with vL and vH fragments derived from antibodies. The order of Va, Vb, vL and vH can be switched. Similarly, one of more CD3z fragments can be replaced by corresponding FcRy fragments. The Ig linker domains can be derived from immunoglobulins or TCR constant chains. The one or both CD3z cytosolic domains can have the dQ101 mutation.

TABLE 2C
EXAMPLE SAR

| | | | | | | | | | |
|---|---|---|---|---|---|---|---|---|---|
| SP | L | (AABD-L)n | (scFv-L)n | L | CD16-D1 | CD16-D2 | CD16-Hn | CD16-TM | 4-1BB |
| SP | L | (AABD-L)n | (scFv-L)n | L | CD16-D1 | CD16-D2 | CD16-HN | CD16-TM | |

TABLE 2D
EXAMPLE TCR-SAR WITH BACKBONE OF zSAR

| 1 | SP | L | (AABD-L)n | vL | L | Ig- | CD3z- | CD3z- | CD3z-CP | |
|---|---|---|---|---|---|---|---|---|---|---|
| | | | | | | linker | ECD | TM | | |
| | SP | L | (AABD-L)n | vH | L | Ig- | CD16- | CD16- | | |
| | | | | | | linker | HN | TM | | |
| 2 | SP | L | (AABD-L)n | VL | L | IgCL | CD3z- | CD3z- | CD3z-CP | |
| | | | | | | | ECD | TM | | |
| | SP | L | (AABD-L)n | VH | L | IgG1- | CD16- | CD16- | 4-1BB or CD28 or CD8a, | |
| | | | | | | CH1 | HN | TM | or CD8b or CD4 cytosolic | |
| | | | | | | | | | domain | |
| 3 | SP | L | (AABD-L)n | VL | L | IgCL | CD3z- | CD3z- | 4-1BB | CD3z-CP |
| | | | | | | | ECD | TM | | |
| | SP | L | (AABD-L)n | VH | L | IgG1- | CD16- | CD16- | 4-1BB | CD3z-CP |
| | | | | | | CH1 | HN | TM | | |

The vL and vH in the constructs in Table 2D can be replaced with Va, Vb, Vg, or Vd fragments. The order of Va, Vb, Vg, Vd, vL and vH can be switched so that they are attached to different chains. Similarly, one of more CD3z fragments can be replaced by corresponding FcRy fragments. The Ig linker domains can be derived from immunoglobulins or TCR constant chains. The one or both CD3z cytosolic domains can have the dQ101 mutation.

TABLE 2E
EXAMPLE zSAR and zCD16-SAR

| | | | | | | | | | | |
|---|---|---|---|---|---|---|---|---|---|---|
| 1 | SP | L | (AABD-L)n | vL | L | Ig (Ig linker) | ECD | TM | CSD | CP (AD) |
| | SP | L | (AABD-L)n | vH | L | Ig (Ig linker) | ECD | TM | CSD | CP (AD) |
| 2 | SP | L | (AABD-L)n | vL | L | Ig (Ig linker) | Hinge | TM | CSD | CP (AD) |
| | SP | L | (AABD-L)n | vH | L | Ig (Ig linker) | Hinge | TM | CSD | CP (AD) |
| 3 | SP | L | (AABD-L)n | vL | L | Ig (Ig linker) | CD3z-ECD | CD3z-TM | 41BB | CD3z-CP |
| | SP | L | (AABD-L)n | vH | L | Ig (Ig linker) | CD16-HN | CD16-TM | 41BB | CD16-CP |
| 4 | SP | L | (AABD-L)n | vL | L | IgCL | CD3z-ECD | CD3z-TM | 41BB | CD3z-CP |
| | SP | L | (AABD-L)n | vH | L | IgG1-CH1 | CD3z-ECD | CD3z-TM | CD28 | CD3z-CP |
| 5 | SP | L | (AABD-L)n | vL | L | IgCL | CD3z-ECD | CD3z-TM | | CD3z-CP |
| | SP | L | (AABD-L)n | vH | L | IgG1-CH1 | CD3z-ECD | CD3z-TM | | Lck, LAT or |
| 5 | SP | L | (AABD-L)n | vL | L | IgCL | CD3z-ECD | CD3z-TM | | LAT |
| | SP | L | (AABD-L)n | vH | L | IgG1-CH1 | CD3z-ECD | CD3z-TM | | SLP-76 |

The vL and vH in the constructs in Table 2E can be replaced with Va, Vb, Vg, or Vd fragments. The order of Va, Vb, Vg, Vd, vL and vH can be switched so that they are attached to different chains. Similarly, one of more CD3z fragments can be replaced by corresponding FcRy fragments. The Ig linker domains can be derived from immunoglobulins or TCR constant chains. The one or both CD3z cytosolic domains can have the dQ101 mutation.

TABLE 2F
EXAMPLE SAR (SIR) with HYBRID TCR CHAINS

| | | | | | Ig-like | | | |
|---|---|---|---|---|---|---|---|---|
| | | | | | linker | | | |
| | | | | | (constant | Connecting | | CP |
| | | | | | domain) | Peptide | TM | (IC) |
| 1 | SP | L | vL | L | alpha | alpha | alpha | Alpha |
| | SP | L | vH | L | beta | beta | beta | Beta |
| 2 | SP | L | vL | L | alpha | beta | beta | Beta |
| | SP | L | vH | L | beta | alpha | alpha | Alpha |
| 3 | SP | L | vL | L | alpha | alpha | beta | Beta |
| | SP | L | vH | L | beta | beta | alpha | Alpha |
| 4 | SP | L | vL | L | gamma | delta | delta | Delta |
| | SP | L | vH | L | delta | gamma | gamma | Gamma |
| 5 | SP | L | vL | L | gamma | gamma | delta | Delta |
| | SP | L | vH | L | delta | delta | gamma | Gamma |
| 6 | SP | L | vL | L | gamma | delta | delta | Delta |
| | SP | L | vH | L | delta | gamma | gamma | Gamma |
| 7 | SP | L | vL | L | alpha | gamma | gamma | Gamma |
| | SP | L | vH | L | gamma | alpha | alpha | Alpha |
| 8 | SP | L | vL | L | alpha | alpha | gamma | Gamma |
| | SP | L | vH | L | gamma | gamma | alpha | Alpha |
| 9 | SP | L | vL | L | alpha | delta | delta | Delta |
| | SP | L | vH | L | delta | alpha | alpha | Alpha |
| 10 | SP | L | vL | L | Beta | gamma | gamma | Gamma |
| | SP | L | vH | L | gamma | Beta | Beta | Beta |
| 11 | SP | L | vL | L | Beta | Beta | gamma | Gamma |
| | SP | L | vH | L | gamma | gamma | Beta | Beta |
| 12 | SP | L | vL | L | Beta | delta | delta | Delta |
| | SP | L | vH | L | delta | Beta | Beta | Beta |
| 13 | SP | L | vL | L | Beta | Beta | delta | Delta |
| | SP | L | vH | L | delta | delta | Beta | Beta |
| 14 | SP | L | vL | L | Beta | Beta | delta | Delta |
| | SP | L | vH | L | delta | delta | Beta | Beta |
| 15 | SP | L | vL | L | alpha | gamma | gamma | Alpha |
| | SP | L | vH | L | Beta | delta | delta | Beta |
| 16 | SP | L | vL | L | alpha | gamma | gamma | Alpha |
| | SP | L | vH | L | Beta | delta | delta | Beta |
| 17 | SP | L | vL | L | alpha | delta | delta | Alpha |
| | SP | L | vH | L | Beta | gamma | gamma | Beta |
| 18 | SP | L | vL | L | gamma | alpha | alpha | Gamma |
| | SP | L | vH | L | delta | Beta | Beta | Delta |
| 19 | SP | L | vL | L | gamma | Beta | Beta | Gamma |
| | SP | L | vH | L | delta | alpha | alpha | Delta |
| 20 | SP | L | vL | L | alpha | alpha | alpha | Beta |
| | SP | L | vH | L | beta | beta | beta | Alpha |
| 21 | SP | L | vL | L | gamma | gamma | gamma | Delta |
| | SP | L | vH | L | delta | delta | delta | Gamma |
| 22 | SP | L | vL | L | alpha | alpha | alpha | Gamma |
| | SP | I | vH | L | gamma | gamma | gamma | Alpha |
| 23 | SP | L | vL | L | alpha | alpha | alpha | Delta |
| | SP | L | vH | L | delta | delta | delta | Alpha |
| 24 | SP | L | vL | L | beta | beta | beta | Gamma |
| | SP | L | vH | L | gamma | gamma | gamma | Beta |
| 25 | SP | L | vL | L | beta | beta | beta | Delta |
| | SP | L | vH | L | delta | delta | delta | Beta |
| 26 | SP | L | vL | L | alpha | alpha | alpha | Gamma |
| | SP | L | vH | L | beta | beta | beta | Delta |
| 27 | SP | L | vL | L | gamma | gamma | gamma | Alpha |
| | SP | L | vH | L | delta | delta | delta | Beta |
| 28 | SP | L | vL | L | beta | beta | gamma | gamma |
| | SP | L | vH | L | delta | delta | delta | delta |
| 29 | SP | L | vL | L | beta | beta | gamma | gamma |
| | SP | L | vH | L | alpha | alpha | alpha | alpha |
| 30 | SP | L | vL | L | beta | beta | gamma | gamma |
| | SP | L | vH | L | alpha | alpha | delta | delta |
| 31 | SP | L | vL | L | beta | gamma | gamma | gamma |
| | SP | L | vH | L | alpha | delta | delta | delta |
| 32 | SP | L | vL | L | gamma | gamma | beta | beta |
| | SP | L | vH | L | delta | delta | alpha | alpha |
| 33 | SP | L | vL | L | Ig-linker | beta | gamma | gamma |
| | SP | L | vH | L | Ig-linker | delta | delta | delta |
| 34 | SP | L | vL | L | Ig-linker | beta | gamma | gamma |
| | SP | L | vH | L | Ig-linker | delta | delta | delta |
| 35 | SP | L | vL | L | Ig-linker | beta | gamma | gamma |
| | SP | L | vH | L | Ig-linker | alpha | alpha | alpha |
| 36 | SP | L | vL | L | IgCL | beta | gamma | gamma |
| | SP | L | vH | L | IgG1-CH1 | delta | delta | delta |
| 37 | SP | L | vL | L | IgCL | beta | gamma | gamma |
| | SP | L | vH | L | IgG1-CH1 | alpha | alpha | alpha |
| 38 | SP | L | vL | L | IgCL | beta | gamma | gamma |
| | SP | L | vH | L | IgG1-CH | delta | delta | delta |
| 39 | SP | L | vL | L | IgCL | beta | gamma | gamma |
| | SP | L | vH | L | IgG1-CH | alpha | alpha | alpha |
| 40 | SP | L | vL | L | IgCL | beta | gamma | gamma |
| | SP | L | vH | L | IgG1-CH | alpha | delta | delta |
| 41 | SP | L | vL | L | IgCL | gamma | gamma | gamma |
| | SP | L | vH | L | IgG1-CH1 | delta | delta | delta |
| 42 | SP | L | vL | L | IgCL | gamma | beta | beta |
| | SP | L | vH | L | IgG1-CH | delta | alpha | alpha |
| 43 | SP | L | vL | L | IgCL | gamma | beta | gamma |
| | SP | L | vH | L | IgG1-CH | delta | alpha | alpha |
| 44 | SP | L | vH | L | IgG1-CH | gamma | beta | gamma |
| | SP | L | vL | L | IgCL | delta | alpha | alpha |
| 45 | SP | L | vL | L | beta | beta | gamma | gamma |
| | SP | L | vH | L | alpha | alpha | alpha | alpha |

TABLE 2G
EXAMPLE SAR (SIR) with HYBRID TCR CHAINS

| | | | | | Ig-like | | | |
|---|---|---|---|---|---|---|---|---|
| | | | | | linker | Connecting | | |
| | | | | | (constant | Peptide | | |
| | | | | | domain) | (ConnP) | TM | CP (IC) |
| 1 | SP | L | vL | L | Ig-Linker | alpha | alpha | alpha |
| | SP | L | vH | L | Ig-Linker | beta | gamma | gamma |
| 2 | SP | L | vL | L | Ig-Linker | beta | beta | beta |
| | SP | L | vH | L | Ig-Linker | alpha | delta | delta |
| 3 | SP | L | vL | L | IgCL | alpha | beta | beta |
| | SP | L | vH | L | IgG-CH1 | beta | alpha | alpha |
| 4 | SP | L | vL | L | IgCL | delta | delta | delta |
| | SP | L | vH | L | IgG-CH1 | beta | gamma | gamma |
| 5 | SP | L | vL | L | IgCL | gamma | delta | delta |
| | SP | L | vH | L | IgG-CH1 | delta | gamma | gamma |
| 6 | SP | L | vL | L | IgCL | delta | delta | delta |
| | SP | L | vH | L | delta | gamma | gamma | gamma |
| 7 | SP | L | vL | L | IgCL | gamma | gamma | gamma |
| | SP | L | vH | L | gamma | alpha | alpha | alpha |
| 8 | SP | L | vL | L | IgCL | alpha | gamma | gamma |
| | SP | L | vH | L | gamma | gamma | alpha | alpha |
| 9 | SP | L | vL | L | IgCL | delta | delta | delta |
| | SP | L | vH | L | delta | alpha | alpha | alpha |
| 10 | SP | L | vL | L | IgG-CH1 | gamma | gamma | gamma |
| | SP | L | vH | L | gamma | Beta | Beta | Beta |
| 11 | SP | L | vL | L | Beta | Beta | gamma | gamma |
| | SP | L | vH | L | IgG1-CH1 | gamma | Beta | Beta |
| 12 | SP | L | vL | L | IgCL | delta | delta | delta |
| | SP | L | vH | L | IgG-CH1 | Beta | Beta | Beta |
| 13 | SP | L | vL | L | IgCL | Beta | delta | delta |
| | SP | L | vH | L | IgG4-CH1 | delta | Beta | Beta |
| 14 | SP | L | vL | L | IgCL | Beta | delta | delta |
| | SP | I | vH | L | IgG-CH1 | delta | Beta | Beta |
| 15 | SP | L | vL | L | IgCL | gamma | gamma | alpha |
| | SP | L | vH | L | IgG1-CH1 | delta | delta | Beta |
| 16 | SP | L | vL | L | IgCL | gamma | gamma | alpha |
| | SP | L | vH | L | IgG-CH1 | delta | delta | Beta |
| 17 | SP | L | vL | L | IgCL | delta | delta | alpha |
| | SP | L | vH | L | IgG-CH1 | gamma | gamma | Beta |
| 18 | SP | L | vL | L | IgCL | alpha | alpha | gamma |
| | SP | L | vH | L | IgG-CH1 | Beta | Beta | delta |
| 19 | SP | L | vL | L | IgCL | Beta | Beta | gamma |
| | SP | L | vH | L | IgG4-CH1 | alpha | alpha | delta |
| 20 | SP | L | vL | L | IgCL | alpha | alpha | beta |
| | SP | L | vH | L | IgG-CH1 | beta | beta | alpha |
| 21 | SP | L | vL | L | IgCL | gamma | gamma | delta |
| | SP | L | vH | L | IgG-CH1 | delta | delta | gamma |
| 22 | SP | L | vL | L | IgCL | alpha | alpha | gamma |
| | SP | L | vH | L | IgG-CH1 | gamma | gamma | alpha |
| 23 | SP | L | vL | L | IgCL | alpha | alpha | delta |
| | SP | L | vH | L | IgG-CH1 | delta | delta | alpha |
| 24 | SP | L | vL | L | IgCL | beta | beta | gamma |
| | SP | L | vH | L | IgG-CH1 | gamma | gamma | beta |
| 25 | SP | L | vL | L | IgCL | beta | beta | delta |
| | SP | L | vH | L | IgG-CH1 | delta | delta | beta |
| 26 | SP | L | vL | L | IgCL | alpha | alpha | gamma |
| | SP | L | vH | L | IgG-CH1 | beta | beta | delta |
| 27 | SP | L | vL | L | IgCL | gamma | gamma | alpha |
| | SP | L | vH | L | IgG-CH1 | delta | delta | beta |
| 28 | SP | L | vL | L | IgCL | beta | gamma | gamma |
| | SP | L | vH | L | IgG-CH1 | delta | delta | delta |
| 29 | SP | L | vL | L | IgCL | beta | gamma | gamma |
| | SP | L | vH | L | IgG-CH1 | alpha | alpha | alpha |
| 30 | SP | L | vL | L | IgCL | beta | gamma | gamma |
| | SP | L | vH | L | IgG-CH1 | alpha | delta | delta |
| 31 | SP | L | vL | L | IgCL | gamma | gamma | gamma |
| | SP | L | vH | L | IgG-CH1 | delta | delta | delta |
| 32 | SP | L | vL | L | IgCL | gamma | beta | beta |
| | SP | L | vH | L | IgG-CH1 | delta | alpha | alpha |
| 33 | SP | L | vL | L | Ig-linker | beta | gamma | gamma |
| | SP | L | vH | L | Ig-linker | delta | delta | delta |
| 34 | SP | L | vL | L | Ig-linker | beta | gamma | gamma |
| | SP | L | vH | L | Ig-linker | delta | delta | delta |
| 35 | SP | L | vL | L | Ig-linker | beta | gamma | gamma |
| | SP | L | vH | L | Ig-linker | alpha | alpha | alpha |
| 36 | SP | L | vL | L | IgCL | beta | gamma | gamma |
| | SP | L | vH | L | IgG1-CH1 | delta | delta | delta |
| 37 | SP | L | vL | L | IgCL | beta | gamma | gamma |
| | SP | L | vH | L | IgG1-CH1 | alpha | alpha | alpha |
| 38 | SP | L | vL | L | IgCL | beta | gamma | gamma |
| | SP | L | vH | L | IgG1-CH | delta | delta | delta |
| 39 | SP | L | vL | L | IgCL | beta | gamma | gamma |
| | SP | L | vH | L | IgG1-CH | alpha | alpha | alpha |
| 40 | SP | L | vL | L | IgCL | beta | gamma | gamma |
| | SP | L | vH | L | IgG1-CH | alpha | delta | delta |
| 41 | SP | L | vL | L | IgCL | gamma | gamma | gamma |
| | SP | L | vH | L | IgG1-CH1 | delta | delta | delta |
| 42 | SP | L | vL | L | IgCL | gamma | beta | beta |
| | SP | L | vH | L | IgG1-CH1 | delta | alpha | alpha |
| 43 | SP | L | vL | L | IgCL | gamma | beta | gamma |
| | SP | L | vH | L | IgG4-CH1 | delta | alpha | alpha |
| 44 | SP | L | vH | L | IgG1-CH | gamma | beta | gamma |
| | SP | L | vL | L | IgCL | delta | alpha | alpha |
| 45 | SP | L | vL | L | IgG4-CH | beta | gamma | gamma |
| | SP | L | vH | L | IgCL | alpha | alpha | alpha |
| 46 | SP | L | vL | L | IgCL | gamma | beta | beta |
| | SP | L | vH | L | IgG4-CH | alpha | alpha | alpha |
| 47 | SP | L | vL | L | IgCL | beta | gamma | gamma |
| | SP | L | vH | L | IgG1-CH | delta | delta | delta |
| 48 | SP | L | vL | L | IgCL | alpha | delta | delta |
| | SP | L | vH | L | IgG4-CH | beta | beta | beta |
| 49 | SP | L | vL | L | IgCL | alpha | delta | delta |
| | SP | L | vH | L | IgG1-CH | gamma | gamma | gamma |
| 50 | SP | L | vL | L | IgCL | delta | alpha | alpha |
| | SP | L | vH | L | IgG1-CH | beta | beta | beta |
| 51 | SP | L | vL | L | IgCL | delta | alpha | alpha |
| | SP | L | vH | L | IgG1-CH | gamma | gamma | gamma |

The vL and vH fragments in the above can be replaced by Vα, Vβ or Vγ and Vδ fragments. One or both Ig linkers (IgCL and IgG1-CH1) can be replaced by TCR constant domains.

TABLE 2H
EXAMPLE SAR (SIR) with HYBRID TCR CHAINS

| | | | | | Ig-like | | | |
|---|---|---|---|---|---|---|---|---|
| | | | | | linker | Connecting | | |
| | | | | | (constant | Peptide | | CP |
| | | | | | domain) | (ConnP) | TM | (IC) |
| | SP | L | scFv | L | Hybrid TCR constant chain | | | |
| | SP | L | | L | Hybrid TCR constant chain | | | |
| 1 | SP | L | scFv | L | alpha | alpha | alpha | Alpha |
| | SP | L | | L | beta | beta | beta | Beta |
| 2 | SP | L | scFv | L | alpha | beta | beta | Beta |
| | SP | L | | L | beta | alpha | alpha | Alpha |
| 3 | SP | L | | L | alpha | alpha | beta | Beta |
| | SP | L | scFv | L | beta | beta | alpha | Alpha |
| 4 | SP | L | scFv | L | gamma | delta | delta | Delta |
| | SP | L | | L | delta | gamma | gamma | gamma |
| 5 | SP | L | scFv | L | gamma | gamma | delta | Delta |
| | SP | L | | L | delta | delta | gamma | gamma |
| 6 | SP | L | scFv | L | gamma | delta | delta | Delta |
| | SP | L | | L | delta | gamma | gamma | gamma |
| 7 | SP | L | scFv | L | alpha | gamma | gamma | gamma |
| | SP | L | | L | gamma | alpha | alpha | Alpha |
| 8 | SP | L | | L | alpha | alpha | gamma | gamma |
| | SP | L | scFv | L | gamma | gamma | alpha | alpha |
| 9 | SP | L | scFv | L | alpha | delta | delta | delta |
| | SP | L | | L | delta | alpha | alpha | alpha |
| 10 | SP | L | scFv | L | Beta | gamma | gamma | gamma |
| | SP | L | | L | gamma | Beta | Beta | Beta |
| 11 | SP | L | scFv | L | Beta | Beta | gamma | gamma |
| | SP | L | | L | gamma | gamma | Beta | Beta |
| 12 | SP | L | scFv | L | Beta | delta | delta | delta |
| | SP | L | | L | delta | Beta | Beta | Beta |
| 13 | SP | L | scFv | L | Beta | Beta | delta | delta |
| | SP | L | | L | delta | delta | Beta | Beta |
| 14 | SP | L | scFv | L | Beta | Beta | delta | delta |
| | SP | L | | L | delta | delta | Beta | Beta |
| 15 | SP | L | scFv | L | alpha | gamma | gamma | alpha |
| | SP | L | | L | Beta | delta | delta | Beta |
| 16 | SP | L | scFv | L | alpha | gamma | gamma | alpha |
| | SP | L | | L | Beta | delta | delta | Beta |
| 17 | SP | L | scFv | L | alpha | delta | delta | alpha |
| | SP | L | | L | Beta | gamma | gamma | Beta |
| 18 | SP | L | | L | gamma | alpha | alpha | gamma |
| | SP | L | scFv | L | delta | Beta | Beta | delta |
| 19 | SP | L | scFv | L | gamma | Beta | Beta | gamma |
| | SP | L | | L | delta | alpha | alpha | delta |
| 20 | SP | L | scFv | L | alpha | alpha | alpha | beta |
| | SP | L | | L | beta | beta | beta | alpha |
| 21 | SP | L | scFv | L | gamma | gamma | gamma | delta |
| | SP | L | | L | delta | delta | delta | gamma |
| 23 | SP | L | scFv | L | alpha | alpha | alpha | delta |
| | SP | L | | L | delta | delta | delta | alpha |
| 24 | SP | L | | L | beta | beta | beta | gamma |
| | SP | L | scFv | L | gamma | gamma | gamma | beta |
| 25 | SP | L | scFv | L | beta | beta | beta | delta |
| | SP | L | | L | delta | delta | delta | beta |
| 26 | SP | L | scFv | L | alpha | alpha | alpha | gamma |
| | SP | L | | L | beta | beta | beta | delta |
| 27 | SP | L | scFv | L | gamma | gamma | gamma | alpha |
| | SP | L | | L | delta | delta | delta | beta |
| 28 | SP | L | scFv | L | gamma | gamma | gamma | alpha |
| | SP | L | scFv | L | delta | delta | delta | beta |
| 29 | SP | L | | L | alpha | beta | beta | beta |
| | SP | L | vHH | L | beta | alpha | alpha | alpha |
| 30 | SP | L | FHVH | L | alpha | alpha | beta | beta |
| | SP | L | | L | beta | beta | alpha | alpha |
| 31 | SP | L | vHH1 | L | gamma | delta | delta | delta |
| | SP | L | vHH2 | L | delta | gamma | gamma | gamma |
| 32 | SP | L | scFv | L | gamma | gamma | delta | delta |
| | SP | L | FHVH | L | delta | delta | gamma | gamma |
| 33 | SP | L | FHVH | L | gamma | delta | delta | delta |
| | SP | L | DAR | L | delta | gamma | gamma | gamma |
| 34 | SP | L | CENT | L | alpha | gamma | gamma | gamma |
| | SP | L | vHH | L | gamma | alpha | alpha | alpha |
| 35 | SP | L | vHH | L | alpha | alpha | gamma | gamma |
| | SP | L | scFv | L | gamma | gamma | alpha | alpha |
| 45 | SP | L | scFv | L | IgCL | alpha | beta | beta |
| | SP | L | scFv | L | IgG1-CH1 | beta | alpha | Alpha |
| 46 | SP | L | scFv | L | IgCL | delta | delta | Delta |
| | SP | L | scFv | L | IgG4-CHI1 | gamma | gamma | Gamma |
| 47 | SP | L | DARP | L | IgCL | gamma | delta | Delta |
| | SP | L | scFv | L | IgG1-CHI1 | delta | gamma | Gamma |
| 48 | SP | L | CENT | L | IgCL | delta | delta | Delta |
| | SP | L | scFv | L | IgG1-CHI1 | gamma | gamma | Gamma |
| 49 | SP | L | CENT | L | IgCL | gamma | beta | gamma |
| | SP | L | scFv | L | IgG1-CH | delta | alpha | alpha |
| 50 | SP | L | DARP | L | IgG1-CH | gamma | beta | gamma |
| | SP | L | CENT | L | IgCL | delta | alpha | alpha |
| 51 | SP | L | vHH1 | L | beta | beta | gamma | gamma |
| | SP | L | vHH2 | L | alpha | alpha | alpha | alpha |
| 52 | SP | L | AABD | L | IgCL | gamma | beta | gamma |
| | SP | L | AABD | L | IgG1-CH | delta | alpha | alpha |
| 53 | SP | L | AABD | L | IgG1-CH | gamma | beta | gamma |
| | SP | L | AABD | L | IgCL | delta | alpha | alpha |
| 54 | SP | L | AABD | L | beta | beta | gamma | gamma |
| | SP | L | AABD | L | alpha | alpha | alpha | alpha |

TABLE 21
EXAMPLE SAR with HYBRID TCR CHAINS

| | | | | | | Ig-like | | | |
|---|---|---|---|---|---|---|---|---|---|
| | | | | | | linker | Connecting | | |
| | | | | Variable | | (Constant | Peptide | | CP |
| | | | | Domain | | Domain) | (ConnP) | TM | (IC) |
| 1 | SP | L | (AABD-L)n | vL | L | alpha | alpha | Alpha | alpha |
| | SP | L | (AABD-L)n | vH | L | beta | beta | Beta | beta |
| 2 | SP | L | (AABD-L)n | vL | L | alpha | beta | Beta | beta |
| | SP | L | (AABD-L)n | vH | L | beta | alpha | Alpha | alpha |
| 3 | SP | L | (AABD-L)n | vL | L | alpha | alpha | Beta | beta |
| | SP | L | (AABD-L)n | vH | L | beta | beta | Alpha | alpha |
| 4 | SP | L | (AABD-L)n | vL | L | gamma | delta | Delta | delta |
| | SP | L | (AABD-L)n | vH | L | delta | gamma | gamma | gamma |
| 5 | SP | L | (AABD-L)n | vL | L | gamma | gamma | Delta | delta |
| | SP | L | (AABD-L)n | vH | L | delta | delta | gamma | gamma |
| 6 | SP | L | (AABD-L)n | vL | L | gamma | delta | Delta | delta |
| | SP | L | (AABD-L)n | vH | L | delta | gamma | gamma | gamma |
| 7 | SP | L | (AABD-L)n | vL | L | alpha | gamma | gamma | gamma |
| | SP | L | (AABD-L)n | vH | L | gamma | alpha | Alpha | alpha |
| 8 | SP | L | (AABD-L)n | vL | L | alpha | alpha | gamma | gamma |
| | SP | L | (AABD-L)n | vH | L | gamma | gamma | Alpha | alpha |
| 9 | SP | L | (AABD-L)n | vL | L | alpha | delta | Delta | delta |
| | SP | L | (AABD-L)n | vH | L | delta | alpha | Alpha | alpha |
| 10 | SP | L | (AABD-L)n | vL | L | Beta | gamma | gamma | gamma |
| | SP | L | (AABD-L)n | vH | L | gamma | Beta | Beta | Beta |
| 11 | SP | L | (AABD-L)n | vL | L | Beta | Beta | gamma | gamma |
| | SP | L | (AABD-L)n | vH | L | gamma | gamma | Beta | Beta |
| 12 | SP | L | (AABD-L)n | vL | L | Beta | delta | Delta | delta |
| | SP | L | (AABD-L)n | vH | L | delta | Beta | Beta | Beta |
| 13 | SP | L | (AABD-L)n | vL | L | Beta | Beta | Delta | delta |
| | SP | L | (AABD-L)n | vH | L | delta | delta | Beta | Beta |
| 14 | SP | L | (AABD-L)n | vL | L | Beta | Beta | Delta | delta |
| | SP | L | (AABD-L)n | vH | L | delta | delta | Beta | Beta |
| 15 | SP | L | (AABD-L)n | vL | L | alpha | gamma | gamma | alpha |
| | SP | L | (AABD-L)n | vH | L | Beta | delta | Delta | Beta |
| 16 | SP | L | (AABD-L)n | vL | L | alpha | gamma | gamma | alpha |
| | SP | L | (AABD-L)n | vH | L | Beta | delta | Delta | Beta |
| 17 | SP | L | (AABD-L)n | vL | L | alpha | delta | Delta | alpha |
| | SP | L | (AABD-L)n | vH | L | Beta | gamma | gamma | Beta |
| 18 | SP | L | (AABD-L)n | vL | L | gamma | alpha | Alpha | gamma |
| | SP | L | (AABD-L)n | vH | L | delta | Beta | Beta | delta |
| 19 | SP | L | (AABD-L)n | vL | L | gamma | Beta | Beta | gamma |
| | SP | L | (AABD-L)n | vH | L | delta | alpha | Alpha | delta |
| 20 | SP | L | (AABD-L)n | vL | L | alpha | alpha | Alpha | beta |
| | SP | L | (AABD-L)n | vH | L | beta | beta | Beta | alpha |
| 21 | SP | L | (AABD-L)n | vL | L | gamma | gamma | gamma | delta |
| | SP | L | (AABD-L)n | vH | L | delta | delta | Delta | gamma |
| 22 | SP | L | (AABD-L)n | vL | L | alpha | alpha | Alpha | gamma |
| | SP | L | (AABD-L)n | vH | L | gamma | gamma | gamma | alpha |
| 23 | SP | L | (AABD-L)n | vL | L | alpha | alpha | Alpha | delta |
| | SP | L | (AABD-L)n | vH | L | delta | delta | Delta | alpha |
| 24 | SP | L | (AABD-L)n | vL | L | beta | beta | Beta | gamma |
| | SP | L | (AABD-L)n | vH | L | gamma | gamma | gamma | beta |
| 25 | SP | L | (AABD-L)n | vL | L | beta | beta | Beta | delta |
| | SP | L | (AABD-L)n | vH | L | delta | delta | Delta | beta |
| 26 | SP | L | (AABD-L)n | vL | L | alpha | alpha | Alpha | gamma |
| | SP | L | (AABD-L)n | vH | L | beta | beta | beta | delta |
| 27 | SP | L | (AABD-L)n | vL | L | gamma | gamma | gamma | alpha |
| | SP | L | (AABD-L)n | vH | L | delta | delta | delta | beta |
| 28 | SP | L | (AABD-L)n | scFV | L | alpha | alpha | alpha | alpha |
| | SP | L | | | L | beta | beta | beta | beta |
| 29 | SP | L | | | L | alpha | beta | beta | beta |
| | SP | L | (AABD-L)n | scFV | L | beta | alpha | alpha | alpha |
| 30 | SP | L | (AABD-L)n | scFV | L | alpha | alpha | beta | beta |
| | SP | L | | | L | beta | beta | alpha | alpha |
| 31 | SP | L | | | L | gamma | delta | delta | delta |
| | SP | L | (AABD-L)n | scFV | L | delta | gamma | gamma | gamma |
| 32 | SP | L | (AABD-L)n | scFV | L | gamma | gamma | delta | delta |
| | SP | L | | | L | delta | delta | gamma | gamma |
| 33 | SP | L | | | L | gamma | delta | delta | delta |
| | SP | L | (AABD-L)n | scFV | L | delta | gamma | gamma | gamma |
| 34 | SP | L | (AABD-L)n | scFV | L | alpha | gamma | gamma | gamma |
| | SP | L | | | L | gamma | alpha | alpha | alpha |
| 35 | SP | L | | | L | alpha | alpha | gamma | gamma |
| | SP | L | (AABD-L)n | scFV | L | gamma | gamma | alpha | alpha |
| 36 | SP | L | (AABD-L)n | scFV | L | alpha | delta | delta | delta |
| | SP | L | | | L | delta | alpha | alpha | alpha |
| 37 | SP | L | | | L | Beta | gamma | gamma | gamma |
| | SP | L | (AABD-L)n | scFV | L | gamma | Beta | Beta | Beta |
| 38 | SP | L | (AABD-L)n | vHH | L | alpha | alpha | alpha | alpha |
| | SP | L | | | L | beta | beta | beta | beta |
| 39 | SP | L | | | L | alpha | beta | beta | beta |
| | SP | L | (AABD-L)n | vHH | L | beta | alpha | alpha | alpha |
| 43 | SP | L | (AABD-L)n | vHH | L | gamma | delta | delta | delta |
| | SP | L | (AABD-L)n | vHH | L | delta | gamma | gamma | gamma |
| 44 | SP | L | (AABD-L)n | FHVH | L | alpha | gamma | gamma | gamma |
| | SP | L | (AABD-L)n | vHH | L | gamma | alpha | alpha | alpha |
| 45 | SP | L | (AABD-L)n | vHH | L | alpha | alpha | gamma | gamma |
| | SP | L | (AABD-L)n | scFV | L | gamma | gamma | alpha | alpha |
| 46 | SP | L | (AABD-L)n | scFV | L | alpha | delta | delta | delta |
| | SP | L | (AABD-L)n | vHH | L | delta | alpha | alpha | alpha |
| 47 | SP | L | (AABD-L)n | FHVH | L | Beta | gamma | gamma | gamma |
| | SP | L | (AABD-L)n | scFV | L | gamma | Beta | Beta | Beta |
| 48 | SP | L | (AABD-L)n | vL | L | IgCL | gamma | beta | gamma |
| | SP | L | (AABD-L)n | vH | L | IgG1-CH | delta | alpha | alpha |
| 49 | SP | L | (AABD-L)n | vL | L | IgG1-CH | gamma | beta | gamma |
| | SP | L | (AABD-L)n | vH | L | IgCL | delta | alpha | alpha |
| 50 | SP | L | (AABD-L)n | vL | L | beta | beta | gamma | gamma |
| | SP | L | (AABD-L)n | vH | L | alpha | alpha | alpha | alpha |
| 51 | SP | L | (AABD-L)n | vH | L | IgCL | gamma | beta | gamma |
| | SP | L | (AABD-L)n | vL | L | IgG1-CH | delta | alpha | alpha |
| 52 | SP | L | (AABD-L)n | vH | L | IgG1-CH | gamma | beta | gamma |
| | SP | L | (AABD-L)n | vL | L | IgCL | delta | alpha | alpha |
| 53 | SP | L | (AABD-L)n | vL | L | beta | beta | gamma | gamma |
| | SP | L | (AABD-L)n | vH | L | alpha | alpha | alpha | alpha |

TABLE 2J
Example TCR with hybrid chains comprising
antibody constant domains

| | | | | | Ig-like | | | |
|---|---|---|---|---|---|---|---|---|
| | | | | | linker | Connecting | | |
| | | | | | (constant | Peptide | | CP |
| | | | | | domain) | (ConnP) | TM | (IC) |
| 1 | SP | L | Va | L | Ig-Linker | alpha | alpha | alpha |
| | SP | L | Vb | L | Ig-Linker | beta | beta | beta |
| 2 | SP | L | Vg | L | Ig-Linker | alpha | alpha | alpha |
| | SP | L | Vd | L | Ig-Linker | beta | beta | beta |
| 3 | SP | L | Va | L | IgCL | alpha | beta | beta |
| | SP | L | Vb | L | IgG-CH1 | beta | alpha | alpha |
| 4 | SP | L | Va | L | IgCL | delta | delta | delta |
| | SP | L | Vb | L | IgG-CH1 | beta | gamma | gamma |
| 5 | SP | L | Va | L | IgCL | gamma | delta | delta |
| | SP | L | Vb | L | IgG-CH1 | delta | gamma | gamma |
| 6 | SP | L | Va | L | IgCL | delta | delta | delta |
| | SP | L | Vb | L | delta | gamma | gamma | gamma |
| 7 | SP | L | Va | L | IgCL | gamma | gamma | gamma |
| | SP | L | Vb | L | gamma | alpha | alpha | alpha |
| 8 | SP | L | Va | L | IgCL | alpha | gamma | gamma |
| | SP | L | Vb | L | gamma | gamma | alpha | alpha |
| 9 | SP | L | Va | L | IgCL | delta | delta | delta |
| | SP | L | Vb | L | delta | alpha | alpha | alpha |
| 10 | SP | L | Va | L | IgG-CH1 | gamma | gamma | gamma |
| | SP | L | Vb | L | gamma | Beta | Beta | Beta |

“Binds the same epitope as” means the ability of an antibody, scFv, or other antigen binding domain to bind to a target antigen and having the same epitope as an exemplified antibody, scFv, or other antigen binding domain.

It is to be inferred without explicit recitation and unless otherwise intended, that when the disclosure relates to a polypeptide, protein, polynucleotide, antibody, SAR, or fragment thereof, an equivalent or a biologically equivalent of such is intended within the scope of this disclosure. As used herein, the term “biological equivalent thereof” or “variant” or “functional variant” is intended to be synonymous with “equivalent thereof” when referring to a reference protein or a fragment thereof, antibody or a fragment thereof, receptor or a fragment thereof, ligand or a fragment thereof, non-immunoglobulin antigen binding domain or fragment thereof, SAR or a fragment thereof, SIR or a fragment thereof, CAR or a fragment thereof. A “biological equivalent thereof” or “variant” or “functional variant” polypeptide or nucleic acid intends those having minimal homology while still maintaining desired structure or functionality. Unless specifically recited herein, it is contemplated that any of the above also includes equivalents thereof, including alternatively spliced isoforms and equivalents from other animal species. For example, a variant intends at least about 70% homology or identity, or at least 80% homology or identity and alternatively, or at least about 85%, or alternatively at least about 90%, or alternatively at least about 95%, or alternatively at least 98% percent homology or identity and exhibits substantially equivalent biological activity to the reference protein, polypeptide, antibody, or fragment thereof, or nucleic acid. Alternatively, when referring to polynucleotides, a variant thereof is a polynucleotide that hybridizes under stringent conditions to the reference polynucleotide or its complement. Alternatively, when referring to polypeptides or proteins, a variant thereof is an expressed polypeptide or protein from a polynucleotide that hybridizes under stringent conditions to the polynucleotide or its complement that encodes the reference polypeptide or protein.

It will be recognized that proteins can have identity or homology to one another and retain similar or identical functions. In an embodiment, a polypeptide “variant” or “functional variant” as used herein, is a polypeptide that differs from the recited polypeptide in conservative substitutions and/or modifications, such that therapeutic, antigenic and/or immunogenic properties of the polypeptide are retained. Polypeptide variants typically exhibit at least about 70%, more typically at least about 90% and most typically at least about 95% homology to the identified polypeptides. For polypeptides with immunoreactive properties, variants can, alternatively, be identified by modifying the amino acid sequence of one of the above polypeptides and evaluating the immunoreactivity of the modified polypeptide. Such modified sequences can be prepared and tested using, for example, the representative procedures described herein. The disclosure includes functional variants of SAR, SAR components and SAR fragments (e.g., extracellular, hinge, transmembrane and cytosolic regions of CD16, TCRα, TCRβ, TCRγ, TCRδ, and CD3z etc.) that have at least 70%, 80%, 85%, 90%, 95%, 97%, 98%, 98.5%, 99% or 99.9% identity to any of the amino acid sequences described herein while retaining the biological activity. The disclosure also includes antigen binding domains, extracellular domains, hinge domains, transmembrane domains, cytosolic domains, costimulatory domains, accessory modules that have at least 70%, 80%, 85%, 90%, 95%, 97%, 98%, 98.5%, 99% or 99.9% identity to any of the sequences described herein while retaining the biological activity. Variants and functional variants include homologs from other species (e.g., mouse, dog, cat, monkey etc.) and alternative spliced isoforms.

Each of the embodiments and aspects of the invention includes sequence variants and fragments of the proteins, fusion proteins and the polynucleotides encoding the proteins and fusion proteins wherein the length of each peptide region or domain comprising a fusion protein individually varies on the amino terminus, carboxy terminus, or both ends, by up to 10 amino acids based on the native sequence of the polypeptide from which the peptide region or domain is obtained. In certain aspects, sequence variants and fragments have at least 70% % (e.g., 70%, 75%, 80%, 85%, 90%, 95%, 98%, 99% etc.) of the activity of the specific fusion protein upon which they are based.

In one aspect of this embodiment, the sequence variant or fragment is a sequence variant or fragment wherein the length of at least one peptide region or domain comprising the fusion protein individually varies on the amino terminus, carboxy terminus, or both ends, by up to 10 amino acids based on the native sequence of the polypeptide from which the peptide region or domain is obtained.

Each of the embodiments and aspects of the invention also includes sequence variants and fragments of the protein, polypeptide and fusion proteins, and polynucleotides encoding the same, having at least 70% (e.g., 70%, 75%, 80%, 85%, 90%, 95%, 98%, 99% etc.) sequence identity with a specific protein, polypeptide or fusion protein defined herein, over the entire length of that specific fusion protein. In certain aspects, these sequence variants will have at least 70% % (e.g., 70%, 75%, 80%, 85%, 90%, 95%, 98%, 99% etc.) of the activity of the protein, polypeptide, and specific fusion protein upon which they are based.

In an embodiment, the invention includes sequence variants and fragments of an antibody, antibody fragment (e.g., Fab, vL, vH, vHH, scFv, FHVH etc.), TCR, and TCR variable domains (e.g. Vα, Vβ, Vγ, Vδ etc.), and polynucleotides encoding the same, having at least 70% (e.g., 70%, 75%, 80%, 85%, 90%, 95%, 98%, 99% etc.) sequence identity with a specific antibody, antibody fragment, TCR, and TCR variable domains defined herein, over their entire length excluding the complementary determining regions (CDR).

As used herein, the term “CD3 complex” refers to a cell surface molecule assembly comprising numerous proteins for transmembrane signaling of TCR activation.

As used herein, the term “CDR” or “complementarity determining region” is intended to mean the non-contiguous antigen combining sites found within the variable region of both heavy and light chain polypeptides. These particular regions have been described by Kabat et al., J. Bio. Chem. 252:6609-6616 (1977); Kabat et al., U.S. Dept. of Health and Human Services, “Sequences of proteins of immunological interest” (1991); Chothia et al., J. Mol. Bio. 196:901-917 (1987); and MacCallum et al., J. Mol. Bio. 25 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. As used herein, the different CDRs of an antibody could be also defined by a combination of the different definitions. For example, vHCDR1 could be defined based on Kabat and VHCDR2 could be defined based on Chothia. The amino acid residues which encompass the CDRs as defined by each of the above cited references are as follows:

| | CDR DEFINITIONS | | |
|---|---|---|---|
| | Kabat | Chothia | MacCallum |
| VHCDR1 | 31-35 | 26-32 | 30-35 |
| VHCDR2 | 50-65 | 53-55 | 47-58 |
| VHCDR3 | 95-102 | 96-10 | 193-101 |
| VLCDR1 | 24-34 | 26-32 | 30-36 |
| VLCDR2 | 50-56 | 50-52 | 46-55 |
| VLCDR3 | 89-97 | 91-96 | 89-96 |

### (Residue Numbers Correspond to the Identified Reference).

The SEQ IDs of the CDRs of the example vL and vH segments that can make up antigen binding domains of SAR, bispecific antibodies and other immunotherapeutics of the current disclosure are provided in SEQ ID NO: 13204-14121 and SEQ ID NO: 14122-15039, respectively (Tables 6A, B) of PCT/US2018/053247, in Tables 5-6 of PCT/US2017/064379 and in Table 39 of PCT/US2021/022641, which are incorporated herein by reference. The SEQ IDs of the example vL and vH segments that can make up antigen binding domains of SAR, antibodies and other immunotherapeutics are also provided in Table 3 of the current disclosure. The light chain CDR1, CDR2 and CDR3 of the vL fragments and scFvs provided in the current disclosure are provided in SEQ ID NO: 20989-21015, 41591-41861; 21024-21050, 41862-42132; and 21059-21085, 42133-42403, respectively. The heavy chain CDR1, CDR2 and CDR3 of the vH fragments and scFvs provided in the current disclosure (e.g., Table 3) are provided in SEQ ID NO: 21094-21120 and 42404-42674; 21129-21155, 42675-42945; and 21164-21190, 42946-43216, respectively. The CDR1-3 of select novel binders are provided in Table 8.

In an embodiment, the disclosure provides an antibody, an antibody fragment, or a SAR comprising (1) heavy chain variable domain (vH) comprising the heavy chain CDR1-3 sequences and a complementary light chain variable domain (vL) comprising the light chain CDR1-3, which binds to the same epitope as a monoclonal antibody comprising the variable domains (vL and vH) of sequences depicted in Table 3 or which binds to the same epitope as an scFv whose sequence is depicted in Table 3; or (2) the heavy chain variable domain depicted in Table 3 and the light chain variable domain depicted in Table 3; or (3) an scFv depicted in Table 3; or (4) a heavy chain variable domain having at least 85% amino acid sequence identity to the amino acid sequence depicted in Table 3 and a light chain variable domain having at least 85% amino acid sequence identity to the amino acid sequence depicted in Table 3; or (5)) a heavy chain variable domain having at least 75% amino acid sequence identity in the framework regions to the amino acid sequence depicted in Table 3 and a light chain variable domain having at least 75% amino acid sequence identity in the framework regions to the amino acid sequence depicted in Table 3. In an embodiment, the antibody or the antibody fragment is a bispecific antibody, a multi-specific antibody, an Fv, an scFv, a Fab. In an embodiment, the SAR is a CAR or a next generation CAR (e.g., SIR, Ab-TCR, zSIR, uTCR-SAR, CD16 SAR, zCD16SAR etc.).

In an embodiment, the disclosure provides a single domain antibody or antibody fragment, or a SAR comprising (1) a vHH domain with sequence depicted in Table 5; or (2) a vHH domain having a sequence with at least 85% amino acid sequence identity to the amino acid sequence depicted in Table 5; or (3) vHH domain having a sequence with at least 75% amino acid sequence identity in the framework regions of amino acid sequence depicted in Table 5 and which contain the CDR1-3 of the sequence depicted in Table 5. In an embodiment, the single domain antibody or the antibody fragment is a bispecific antibody or a multi-specific antibody. In an embodiment, the SAR is a CAR or a next generation CAR (e.g., SIR, Ab-TCR, zSIR, uTCR-SAR, CD16 SAR, zCD16 SAR etc.).

“Cancer” and “cancerous” refer to or describe the physiological condition in mammals that is typically characterized by unregulated cell growth.

“Cell therapy” or “Cell-based therapy” or “Immune cell therapy” or Immune effector cell therapy” refers to a therapy that involves the use of cells for the prevention or treatment of a disease.

“Chimeric antigen receptors” (CARs) are artificial (non-naturally occurring) immune cell (e.g., T cell) receptors contemplated for use as a therapy for cancer, using a technique called adoptive cell transfer. In various embodiments, CARs are recombinant polypeptides comprising an antigen-specific domain (ASD), a hinge region (HR), a transmembrane domain (TMD), an optional co-stimulatory domain (CSD) and an intracellular signaling domain (ISD).

“Codon optimization” or “controlling for species codon bias” refers to the preferred codon usage of a particular host cell. In an embodiment, the invention describes proteins, polypeptides, and fragments thereof that are human codon-optimized.

“co-express” refers to expression of two or more polynucleotides or genes.

A “conservative substitution” or “conservative sequence modifications” refers to amino acid modifications that do not significantly affect or alter the binding characteristics or function of the encoded protein.

A “costimulatory intracellular signaling domain” or “Co-stimulatory domain” or “CSD” as used herein refers to the portion of a SAR which enhances the proliferation, survival and/or development of T cells. Each co-stimulatory domain comprises the costimulatory domain of any one or more of, for example, members of the TNFR superfamily, CD28, (4-1BB), CD134, BAFF-R, HVEM, CD27, CD2, CD5, Fas, CD30, CD40, or combinations thereof.

The term a “costimulatory molecule” or a “costimulatory receptor” refers to a cognate binding partner on an immune cell (that specifically binds with a costimulatory ligand, thereby mediating a costimulatory response by the immune cell such as, but not limited to, proliferation, activation, or cytokine secretion.

The term “cTCR” or “chimeric T cell receptor” refers to a wild-type TCR nucleic acid coding sequence and the corresponding wild-type TCR protein linked to an antigen binding domain that is not derived from a TCR. cTCR have been described in (Gross, Waks, & Eshhar, 1989). cTCRs are used in some embodiments and as reference controls.

The term “cytosolic” or “cytoplasmic” refers to an agent, e.g., a protein that is situated in the cytoplasm of a cell in its mature form.

The term “degenerative disorders” refers to a disease that is the result of a continuous process based on degenerative cell changes, affecting tissues or organs.

“Derived from” as that term is used herein, indicates a relationship between a first and a second molecule. It generally refers to structural similarity between the first molecule and a second molecule and does not connotate or include a process or source limitation on a first molecule that is derived from a second molecule.

“Dimerization molecule,” as that term is used herein refers to a molecule that promotes the association of a first switch domain with a second switch domain.

“Disease targeted by genetically modified cells” encompasses the targeting of any cell involved in any manner in any disease by the genetically modified cells of the disclosure.

As used herein a “diverse set of non-naturally occurring immune receptors” or “diverse set of SARs” refers to a plurality of non-naturally occurring immune receptors or SARS targeting an antigen. In embodiment, diverse set of SARs have the same binding domain linked to a diverse set of signaling chains or “backbones.” In an embodiment, the diverse set of SARs may possess diverse range of binding affinities to a target antigen. In an embodiment, the diverse set of SARs may exhibit varied expression levels.

As used herein, an “epitope” is defined to be the portion of an antigen capable of eliciting an immune response, or the portion of an antigen that binds to an antibody or antibody fragment. Epitopes can be a protein sequence or subsequence.

As used herein, the term “engager” refers to a molecule, e.g., a fusion polypeptide, which is capable of forming a link between an immune cell (e.g., a T cell, a NK cell, a NKT cell, a B cell, a macrophage, a neutrophil) and a tumor cell that results in activation of the immune cell.

The term “expression vector” refers to a vector comprising a recombinant polynucleotide comprising expression control sequences operatively linked to a nucleotide sequence to be expressed.

A “functional portion” (“biologically active portion”) of a protein refers to a portion of a protein that retains one or more functions of full length or mature protein.

The term “FcRγ” or “FCER1G” or “FCRG” or “FcRγ” as used herein refers to gene represented by Gene ID: 2207.

The term “functional portion” when used in reference to a SAR refers to any part or fragment of the SAR, which part or fragment retains the biological activity of the SAR of which it is a part (the parent SAR). Functional portions encompass, for example, those parts of a SAR that retain the ability to recognize target cells, or detect, treat, or prevent a disease, to a similar extent, the same extent, or to a higher extent, as the parent SAR. In reference to the parent SAR, the functional portion can comprise, for instance, about 10%, 25%, 30%, 50%, 68%, 80%, 90%, 95%, or more, of the parent SAR.

The term “flexible polypeptide linker” as used herein refers to a peptide linker that consists of amino acids such as glycine and/or serine residues used alone or in combination, to link polypeptide chains together (e.g., variable heavy and variable light chain regions together). In one embodiment, the flexible polypeptide linker is a Gly/Ser linker.

“Genetically modified cells,” “redirected cells,” “genetically engineered cells” or “modified cells” as used herein refer to cells that express a SAR of the disclosure.

An “HLA-independent TCR” or an “MHC-independent TCR” as defined herein is a TCR that can recognize an antigen independent of MHC restriction.

An “HLA-independent TCR variable domain” as defined herein is the variable domain of a TCR that can bind to an antigen in an HLA-independent manner.

As used herein, “HLA-restricted” or “MHC-restricted” refers to antigen recognition requiring both MHC molecule and its peptide. Unlike antigen recognition that is “not HLA-restricted” or “HLA-independent” or “not MHC-restricted.”

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). As another example, a heterologous gene includes a gene expressed in a previous or future cell lineage or differentiation state of a cell. Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

The term “heterologous” when used in context of protein domains refer to domains that is not in its natural environment. For example, a heterologous protein domain is not part of a single naturally occurring polypeptide or protein. Stated in another way, two domains are heterologous if they are derived from two different polypeptides or proteins found in nature. For example, TCRα constant domain and TCRβ transmembrane domains are heterologous to each other as they are derived from two different polypeptide or proteins.

The two domains are considered “interspecies heterologous domains” if they are derived from homologs of the same protein but are derived from two different species and if there is <80% amino acid sequence identity between the two domains. For example, human TCRα constant domain and mouse TCRα transmembrane domains are heterologous to each other if the human TCRα constant domain is less than 80% identical to mouse TCRα constant domain at the amino acid level or if the human TCRα transmembrane domain is less than 80% identical to mouse TCRα transmembrane at the amino acid level. For the purpose of this disclosure, two domains are not considered interspecies heterologous domains if <20% of amino acid residues of a protein domain belonging to one species are replaced by the corresponding residues found in a different species. For example, human TCRα transmembrane domain and a variant of human TCR constant domain are not considered interspecies heterologous if <20% amino acid residues of the human TCRα constant domain are replaced by the corresponding residues found in the mouse TCRα constant domain.

“Hinge region” (HR) as used herein refers to the hydrophilic region which is between the antigen binding domain and the transmembrane domain of a SAR. Several example hinge regions are provided in Table 29 of the provisional patent application.

“Hybrid TCR Chain” or “Hybrid Chain” as the term is used herein, refers to a chain that comprises at least one domain selected from the group of TCR constant domain (CD or C), TCR connecting peptide (ConnP), TCR transmembrane (TM) domain and TCR intracytoplasmic domain (CP or IC) that is heterologous. In an example embodiment, a hybrid TCR chain refers to a TCR chain in which the TM domain is derived from one TCR chain and at least one of the domains selected from CD, ConnP, and IC is derived from a different TCR chain. For example, if TM is derived from human TCRα, then at least one of the domains selected from CD, ConnP, and IC is derived from TCRβ1/β2, TCRγ, TCRδ or pre-TCRα. A hybrid chain may also comprise of domains derived from two different species. Thus, a hybrid chain may comprise of human TCRα constant domain and a non-human TCRα transmembrane domain. A hybrid TCR chain may have 1, 2, 3 or more domains that are heterologous.

“Hybrid Chain SIR” or (HC-SIR) or a Hybrid Chain SAR (HC-SAR) as the term is used herein, refers to a heterodimeric synthetic immune receptor (SIR) or synthetic antigen receptor (SAR) in which at least one TCR constant chain is a hybrid chain. A hybrid chain SIR or a hybrid chain SAR may have both TCR chains that are hybrid.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, monocytes/macrophages, and myeloid-derived phagocytes.

“Immune effector function” or “immune effector response,” “effector function” refers to the specialized function of a differentiated cell. Effector function of a T-cell or NK-cells, for example, may be cytolytic activity or helper activity including the secretion of cytokines. For example, an immune effector function or response refers a property of a T or NK cell that promotes killing or the inhibition of growth or proliferation, of a target cell.

An “intracellular signaling domain,” (ISD) or “activation domain” as the term is used herein, refers to an intracellular signaling portion of a molecule.

The term “isolated” as used herein refers to molecules or biologicals or cellular materials being substantially free from other materials.

A “long linker” or “long linker domain” is a linker that is between 25 to 500 amino acids in length. In an embodiment, a long linker is about 25-500 amino acids and any number in between in length. In an embodiment, a long linker is between 25 and 125 amino acids in length. In an embodiment, a long linker is between 50 and 150 amino acids in length.

In an embodiment, the linker encodes for or comprises of an immunoglobulin (Ig) domain or an Ig-like domain or a fragment thereof. The terms “Ig domain”, “Ig linker domain,” “Ig-like domains” or “Ig-like linker domains” are used interchangeably in this disclosure. Example Ig linker domains are IgCL (SEQ ID NO:8961) and IgG1-CH1 (SEQ ID NO:8962). Additional example Ig linkers are presented in SEQ ID NO (PRT): 8962-8976.

In some embodiments, the peptide linkers are derived from TCR subunit constant regions. In an embodiment, the linker comprises the Ig-like constant domain of a TCR chain and further comprises a TCR connecting peptide. Example long Ig-like linkers are provided in SEQ ID NO: 22827-22829, 22833-22835, 22839-22840, 22843-22844, respectively and also include functional variants and homologs which encodes for polypeptide with at least 75% sequence identity to a polypeptide encoded by any of the above sequences. In an embodiment, the long Ig-like linker comprises N-terminal or C-terminal deletion mutants of in SEQ ID NO: 22827-22829, 22833-22835, 22839-22840, 22843-22844, respectively, in which between 1-40 (e.g., 1, 5, 10, 15, 20, 25, 30, 40) N-terminal or C-terminal amino acid residues are deleted.

As used herein, the term “linker” (also “linker domain” or “linker region”) refers to an oligo or a polypeptide (or an oligo encoding the polypeptide) that joins together two or more domains or regions of a SAR polynucleotide or polypeptide, respectively, disclosed herein. The linker can be anywhere from 1 to 500 amino acids in length or 3 to 1500 nucleotide in length. In some embodiments the “linker” is cleavable or non-cleavable. Linker modules also refer to TCR and Antibody linkers presented in Table 7 of the provisional patent application.

The term “lentivirus” refers to a genus of the Retroviridae family. The term “lentiviral vector” refers to a vector derived from at least a portion of a lentivirus genome, including especially a self-inactivating lentiviral vector.

A “multipurpose switch” or “multipurpose gene” encodes for a protein that provide suicide, survival and marker functions. In an embodiment, all the above functions are provided by a single polypeptide chain. Example multipurpose switches include IL2-tBCMA, IL15-tBCMA, IL2-RQR8, and IL2-tHer2 etc.

The term “multi-chain synthetic antigen receptor” “multi-chain SAR” means a synthetic antigen receptor comprising two or more polypeptide chains. A multi-chain SAR can be a double chain SAR. A double chain SAR comprises two membrane associated domain (e.g., transmembrane or membrane anchoring domains).

“Native” or “Naturally occurring” or “endogenous” as used herein refers to a gene, protein, nucleic acid (e.g., DNA, RNA etc.) or fragment thereof that is native to a cell or is naturally expressed in a cell. Thus, a native or endogenous TCRα chain polypeptide of a T cell consists of a variable domain (Va) joined to a TCRα constant chain.

“Native receptor” or “Naturally occurring receptor” or “endogenous receptor” or “native receptor” as used herein refers to any receptor that occurs in nature and comprises an antigen binding or a ligand binding domain. The term includes functional variants, isoforms, and homologs from other mammalian species. A native receptor can be “native signaling receptor” or a “naturally occurring signaling receptor” if it is capable of transmitting a cell signal upon binding to its target. A naturally occurring receptor or native receptor is native to a cell or is naturally expressed in a cell. Examples of naturally occurring signaling receptors or native receptors include, but are not limited to, CD16A, CD16B, NKp30, NKp44, NKp46, KIR2DS4, NKG2D etc. For the purpose of this disclosure, the CD3 signaling chains (CD3ε, CD3γ, CD3δ and CD3ζ) are not included within the definition of a “naturally occurring receptor” and are instead classified as a signaling adaptor.

As used herein, the term “non-TCR naturally occurring receptor” or “non-TCR naturally occurring signaling receptor” or “non-TCR receptor” or ‘non-TCR signaling receptor” refers to a receptor that is not a T cell receptor (TCR). A non-TCR receptor can be expressed in cells other than a T cell. A non-TCR receptor can be expressed in cells that lack the expression of CD3ζ, CD3ε, CD3δ and/or CD3γ chains.

As used herein, the term “non-T cell receptor module” or “non-TCR module” or “non-TCR signaling module” or “NTCRM” refers to a module that lacks sequences comprised of the T cell receptor transmembrane domains and may further lack all or a portion of T cell receptor connecting peptides and/or intracellular domains. An NTCRM lacks sequences comprised of the transmembrane domains of TCRα, TCRβ, TCRγ, TCRδ or pre-TCRα. An NTCRM may further lack all or a portion of the connecting peptides and/or intracellular domains of TCRα, TCRβ, TCRγ, TCRδ or pre-TCRα. An example non-TCR module (NTCRM) comprises of two CD3z transmembrane domains. Another example NTCRM comprises of a CD3z transmembrane domain and a CD16 transmembrane domain.

As used herein, the term “non-CD3 adaptor module” or “non-CD3 adaptor” or “non-TCR/CD3 adaptor” or “non-TCR/CD3 signaling adaptor” or “NCAM” refers to a signaling adaptor that is not a component of the T cell receptor/CD3 receptor complex. In an embodiment, a “non-TCR/CD3 adaptor” does not comprise the transmembrane and/or cytosolic regions of CD3ε, CD3ζ, CD3γ or CD3δ chains or variants thereof.

The term “near the N-terminus” as used herein means within the N-terminal 30 amino acids. For example, the term “an AABD operably linked to the N-terminus or near the N-terminus of a vL and/or vH domain”, mean an AABD that is operably linked at the N-terminus of a vL or a vH fragment or operably linked to the N-terminal 2, 3, 4, 5, 6, 7, 8, 9, 10, 11, 12, 13, 14, 15, 16, 17, 18, 19, 25 or 30 amino acid comprising the vL or the vH domain. Similarly, the term “an AABD operably linked to the N-terminus or near the N-terminus of a Va and/or Vb domain”, mean an AABD that is operably linked at the N-terminus of a Va or a Vb fragment or operably linked to the N-terminal 2, 3, 4, 5, 6, 7, 8, 9, 10, 11, 12, 13, 14, 15, 16, 17, 18, 19, 20, 21, 22, 23, 24, 25 or 30 amino acid comprising the Va or the Vb domain. An AABD of the disclosure may be operably linked to or near the N-terminus of another domain either directly or via an intervening linker sequence.

As used herein, “Natural Killer Cell Receptor” or “NK receptor” refers to a cell surface receptor that is expressed in natural killer (NK) cells.

As used herein, “Natural Killer Cells” (“NK cells”) refer to a type of cytotoxic lymphocyte of the immune system.

As used herein a “non-naturally occurring agent” or “non-native” or “exogenous” refers to an agent that is not naturally expressed in a cell. Stated another way, the non-naturally occurring agent is “engineered” to be expressed in a cell. A non-naturally occurring agent may be a cloned version of a naturally occurring agent. Example non-naturally occurring agents include SARs (e.g., CAR, SIRs, Ab-TCRs, TFPs, recombinant TCR). A non-naturally occurring agent may be expressed into a cell using techniques of gene transfer known in the art, such as lentiviral or retroviral mediated gene transfer.

As used herein a “non-naturally occurring immune receptor” or “exogenous immune receptor” “non-naturally occurring receptor” refers to an immune receptor that is not naturally expressed in an immune cell. Stated another way, the non-naturally occurring immune receptor is “engineered” to be expressed in an immune cell. An example non-naturally occurring immune receptors is a SAR (e.g., 2^{nd} generation CAR, SIR, cTCR, STAR, zSIR, Ab-TCRs, TFPs and recombinant TCR).

As used herein a “non-naturally occurring TCR antigen binding domain” or “exogenous TCR antigen binding domain” refers to a binding domain operably linked to a TCR constant region that is chimeric and non-naturally occurring with respect to a TCR present in nature. Stated another way, the non-naturally occurring TCR antigen binding domain is “engineered” using recombinant molecular biology techniques to be operably linked to a TCR and moreover, that the antigen binding domain is obtain or derived from a molecule that is distinct from a TCR found in nature. An antigen binding domain that is distinct from a TCR in nature includes antibody, antibody fragments, vH and vL fragments, scFv, humanized antibody fragments, chimeric antibody fragments, adaptors, non-immunoglobulin antigen binding scaffold, receptor, ligands, and the like.

As used herein a “non-naturally occurring antigen binding domain” or “non-naturally occurring extracellular antigen binding domain” or “heterologous antigen binding domain” refers to an antigen binding domain that is not part of a naturally occurring receptor. Example heterologous antigen binding domains include antibodies, antibody fragments (e.g., vL, vH, scFv, Fab, F(ab)2 etc.), single domain antibodies (e.g., sVH, FHVH, vHH etc.), non-immunoglobulin antigen binding domains, single variable domain-TCR (svd-TCR), recombinant TCRs, HLA-independent TCR, scTCR, epitopes, adaptors, ligands, and receptors.

The term “non-TCR antigen binding domain” refers to an antigen binding domain that is not a TCR antigen binding domain. A non-TCR antigen binding domain is structurally distinct from the variable domains (i.e., Vα, Vβ, Vγ and Vδ) found in a TCR. A non-TCR antigen binding domain does not include the variable domains (i.e., Vα, Vβ, Vγ and Vδ) present in a TCR in nature. A non-TCR antigen binding domain also does not include variable domains (i.e., Vα, Vβ, Vγ and Vδ) TCR generated using recombinant molecular biology techniques. Example non-TCR antigen binding domains include antibody, antibody fragments (e.g., vL, vH, scFv, Fab, F(ab)2 etc.), single domain antibodies (e.g., sVH, FHVH, vHH etc.), chimeric antibody fragments, adaptors, non-immunoglobulin antigen binding scaffold (e.g., DARPIN, Centyrins, D domains etc.), adaptors, extracellular Fc binding domains of receptors (e.g., CD16, CD64 etc.), ligands, cytokines and the like.

The term “operably linked” or “functionally linked” or “operationally linked” refers to functional linkage or association between a first component and a second component such that each component can be functional.

“Percent identity” in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more typically over a region that is 100 to 500 or 1000 or more nucleotides (or 20, 50, 200 or more amino acids) in length.

Two examples of algorithms that can be used for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

Non-limiting examples of target antigens are listed in Table B. A SAR of the disclosure may bind one or more (e.g., 2, 3, 4, 5 or more) target antigens listed in Table B either directly or via SAR adaptors described herein.

TABLE B
TABLE B: Example Antigens Targeted by Antibodies, antibody fragments (e.g., scFv),
AABD (e.g., FHVH, vHH, DARPIN, Centryin, D domains, Adaptors etc.) and SARS

CD19; CD5; CD123; CD22; CD30; CD171; CS-1 (CRACC, SLAMF7, CD319, and 19A24);
CD45, C-type lectin-like molecule-1 (CLL-1 or CLECL1); CD33; epidermal growth factor
receptor variant III (EGFRviii); ganglioside G2 (GD2); ganglioside GD3; TNF receptor family
member B cell maturation (BCMA); Tn antigen ((Tn Ag) or (GalNAcα-Ser/Thr)); prostate-
specific membrane antigen (PSMA); Receptor tyrosine kinase-like orphan receptor 1 (ROR1);
FmsLike Tyrosine Kinase 3 (FLT3); Tumor-associated glycoprotein 72 (TAG72); CD38;
CD44v6; a glycosylated CD43 epitope expressed on acute leukemia or lymphoma but not on
hematopoietic progenitors; a glycosylated CD43 epitope expressed on non-hematopoietic
cancers; Carcinoembryonic antigen (CEA); Epithelial cell adhesion molecule (EPCAM); B7H3
(CD276); KIT (CD117); Interleukin-13 receptor subunit alpha-2 (IL-13Ra2 or CD213A2);
Mesothelin; Interleukin 11 receptor alpha (IL-llRa); prostate stem cell antigen (PSCA);
vascular endothelial growth factor receptor 2 (VEGFR2); Lewis(Y) antigen; CD24; Platelet-
derived growth factor receptor beta (PDGFR-beta); Stage-specific embryonic antigen-4
(SSEA-4); CD20; Folate receptor alpha; Receptor tyrosine-protein kinase ERBB2 (Her2/neu);
Mucin 1, cell surface associated (MUC1); epidermal growth factor receptor (EGFR); neural
cell adhesion molecule (NCAM); carbonic anhydrase IX (CAlX); tyrosinase; Fucosyl GM1;
sialyl Lewis adhesion molecule (sLe); ganglioside GM3; transglutaminase 5 (TGS5); high
molecular weight-melanoma associated antigen (HMWMAA); claudin 6 (CLDN6); thyroid
stimulating hormone receptor (TSHR); G protein coupled receptor class C group 5, member D
(GPRC5D); chromosome X open reading frame 61 (CXORF61); CD97; CD179a; anaplastic
lymphoma kinase (ALK); mammary gland differentiation antigen (NY-BR-1); Wilms tumor
protein (WT1); Cancer/testis antigen 1 (NY-ESO-1); Melanoma-associated antigen 1 (MAGE-
A1); melanoma antigen recognized by T cells 1 (MelanA or MARTI); Rat sarcoma (Ras)
mutant; human Telomerase reverse transcriptase (hTERT); human papilloma virus E6 (HPV
E6); human papilloma virus E7 (HPV E7); CD79a; CD79b; CD72; Leukocyte-associated
immunoglobulin-like receptor 1 (LAIRl); C-type lectin domain family 12 member A
(CLEC12A); EGF-like module-containing mucin-like hormone receptor-like 2 (EMR2);
lymphocyte antigen 75 (LY75); Glypican-3 (GPC3); Fc receptor-like 5 (FCRL5);
immunoglobulin lambda-like polypeptide 1 (IGLLl); Biotin; c-MYC epitope Tag; CD34;
LAMP1 TROP2; GFRalpha4; CDH17; CDH6; CDH19; CD200R; Slea (CA19.9; Sialyl Lewis
Antigen) Fucosyl-GM1; PTK7; CDH1-CD324; DLL3; CD276/B7H3; IL11Ra; IL13Ra2;
CD179b-IGLl1; ALK, TCR-gamma-delta; NKG2D; CD32 (FCGR2A); CSPG4-HMW-MAA;
Tim1-/HVCR1; CSF2RA (GM-CSFR-alpha); TGFbetaR2; VEGFR2/KDR; Lewis Ag; TCR-
alpha chain, TCR-beta1 chain; TCR-beta2 chain; TCR-gamma chain; TCR-delta chain; FITC;
Leutenizing hormone receptor (LHR); Follicle stimulating hormone receptor (FSHR);
Chorionic Gonadotropin Hormone receptor (CGHR); CCR4; GD3; SLAMF6; SLAMF4; HIV1
envelope glycoprotein; HTLV1-Tax; CMV pp65; EBV-EBNA3c; influenza A hemagglutinin
(HA); GAD; PDL1; Guanylyl cyclase C (GCC); KSHV-K8.1 protein; KSHV-gH protein; auto
antibody to desmoglein 3 (Dsg3); autoantibody to desmoglein 1 (Dsg1); HLA-A2; HLA-
A2:01, HLA-B; HLA-C; HLA-DP; HLA-DM; HLA-DOA; HLA-DOB; HLA-DQ; HLA-DR;
HLA-G; IGE; CD99; Lym1; Lym2; RAS G12V; Tissue Factor 1 (TF1); AFP; GPRC5D;
claudin18.2 (CLD18A2 OR CLDN18A.2); STEAP1; STEAP2, LIV1; NECTIN-4; CRIPTO;
GPA33; BST1/CD157; low conductance chloride channel; TAJ/TNFRSF19, MPL (TPO-R),
KIR3DL2, CD32b, CD229, Toso, BAFF-R, OR2H1, p95-Her2, huTAG2, immunoglobulin
kappa light chain, immunoglobulin gamma light chain, SARS-cov2 spike glycoprotein, SARS-
cov2 Receptor binding domain, CSF1R, mutant p53, p53-R175H mutant, p53-R248Q mutant,
NPM1c, PRAMEl, Melanoma-associated antigen 4 (MAGE-A4), gp100, IL23R, MYCN, and
Myelin Oligodendrocyte Glycoprotein (MOG).

As used herein, the term “receptor” refers to a polypeptide, or portion thereof, present on a cell membrane that selectively binds one or more ligand.

As used herein, the terms “region” or “portion” when used in reference to a nucleic acid molecule refers to a set of linked nucleotides that is less than the entire length of the molecule, such as a CD3ζ signaling region described herein.

The term “retrovirus vector” refers to a vector derived from at least a portion of a retrovirus genome. Examples of retrovirus vector include MSCVneo or MSCVpac.

The term “SAR” or “Synthetic Antigen Receptor,” as used herein, refers to any non-native antigen binding receptor that is expressed on the surface of a cell (e.g., immune cell). The “Synthetic Antigen Receptor” or “SAR” is a non-naturally occurring receptor or a synthetic receptor that can be expressed on the surface of a cell and comprises at least one heterologous antigen binding domain and at least one membrane associated domain, wherein the membrane associated domain can be a transmembrane domain or a membrane anchoring domain (i.e., a GPI linked domain). The antigen binding domain of the SAR is heterologous to its membrane associated domain, i.e., the antigen binding domain is derived from a different source than the membrane associated domain. A SAR may further comprise a hinge domain, an extracellular ligand binding domain and/or an optional cytosolic domain. In an embodiment, a SAR comprises a polypeptide or a set of polypeptides, which when expressed in an effector cell, provides the cell with specificity for a target cell, typically a cancer cell, and with intracellular signal generation. A SAR can be single chain, two chains or more than two chains. A SAR can be uni-specific, bispecific, or multi-specific. A SAR may have one or more heterologous antigen binding domains. The term SAR comprises conventional CARs (e.g., 2^{nd} generation CARs comprising 41BB or CD28 costimulatory domains and CD3z activation domain) and also encompasses newer approaches to conferring antigen specificity onto cells, such as Antibody-TCR chimeric molecules or Ab-TCR (WO 2017/070608 A1 incorporated herein by reference), TCR receptor fusion proteins or TFP (WO 2016/187349 A1 incorporated herein by reference), Synthetic Immune Receptors (SIRs) (see, WO 2018/102795 A1, incorporated herein by reference), STAR (see, WO 2020/029774), HLA-independent TCR (see, WO2019157454A1), Tri-functional T cell antigen coupler (Tri-TAC or TAC) (see, WO 2015/117229 A1, incorporated herein by reference) and zSIR (see, PCT/US2019/035096, incorporated herein by reference). Bispecific and multi-specific SARs have been described in PCT/US2021/022641. The term “SAW” covers CAR as well as other antigen binding receptors, including but not limited to recombinant TCR. The SAR also comprises compositions comprising one or more regions derived from CD16A, CD16B, CD3ζ, DAP10, DAP12, FcRγ, TCRαβ and TCRγδ etc. and variants and fragments thereof. The current disclosure provides SARs comprising functional variants of the above genes and/or proteins include alternative spliced isoforms, hybrid chains and homologs from other species. The example regions or fragments of the above genes and proteins that can be used in the construction of the SARs of the disclosure are provided in Tables 7 and 22 of the provisional patent application. The SAR can be also constructed with polypeptides or fragments that have 70%, 75%, 80%, 85%, 90%, 95%, 98% or 99% homology to any of the fragments provided in Tables 7 and 22 of the provisional patent application. The nucleic acid and amino acid sequences of example additional components (e.g., vL, vH, scFv, vHH etc.) that can be used in the construction of SAR are provided in Tables 3-6. The SAR can be also constructed with polypeptides or fragments that have 70%, 75%, 80%, 85%, 90%, 95%, 98% or 99% homology in the framework regions of any of the fragments provided in Tables 3-6 and comprising light chain and heavy chain CDR regions with no more than one amino acid substitution of the CDR regions of the antigen binding fragments (e.g., vL, vH, scFv, vHH and FHVH etc.) fragments listed in Tables 3-6. The example SARs of the disclosure are provided in Tables 8-20 and 23-25 of the provisional patent application. SARs are modular in design and additional SARs can be constructed by swapping one module of the SAR with a different module. The expression and activity of these novel SARs can be tested using methods described in the disclosure to select the SARs with optimal functional activities.

The term “single-chain synthetic antigen receptor” or “single chain SAR” means a synthetic antigen receptor comprising a single polypeptide chain. Example such SARs are provided in SEQ ID NO: 1392-2234 and in Table 10 of the provisional patent application.

The term “double-chain synthetic antigen receptor” or “double chain SAR” means a synthetic antigen receptor comprising two polypeptide chains wherein each chain comprises at least one antigen binding domain and a signaling chain. Example double chain SARs include Synthetic Immune Receptors (SIRs) (see, WO 2018/102795 A1, incorporated herein by reference), and zSIR (see, PCT/US2019/035096, incorporated herein by reference). Bispecific and multi-specific SARs have been described in PCT/US2021/022641.

The term “One and half-chain synthetic antigen receptor” means a synthetic antigen receptor comprising two polypeptide chains wherein one chain comprises at least one antigen binding domain and a signaling chain (e.g., TCRα constant chain) and the other chain comprises a signaling chain (e.g., TCRβ constant chain) but lacks an antigen binding domain. Example one and a half chain SARs include Synthetic Immune Receptors (SIRs) (see, WO 2018/102795 A1, incorporated herein by reference). Example one and a half SAR are presented in SEQ ID NO: 50061 and 50062.

Typically, the term “SAR-T” is used, to refer to T-cells that have been engineered to express a Synthetic antigen receptor. The term “SAR-NK” refers to an NK cell that has been engineered to express a SAR.

The term “Synthetic Immune Receptor” or alternatively a “SIR” refers to a set of polypeptides, typically two in some embodiments, which when expressed in an effector cell, provides the cell with specificity for a target cell, typically a cancer cell, and with intracellular signal generation. SIRs represent next generation CAR platforms that are described in WO 2018/102795 A1 which is incorporated herein by reference. In a typical embodiment, a SIR comprises one or more antigen binding domains (e.g., antibody or antibody fragment, a ligand, or a receptor) that bind to antigens as described herein and are joined to one or more T cell receptor constant chains or regions via an optional linker. In some embodiments, the set of polypeptides are contiguous with each other. In some embodiments, a SIR comprises two or more sets of two or more polypeptides. The polypeptides of each set of SIRs are contiguous with each other (functional polypeptide unit 1) but are not contiguous with the polypeptides of the other set (functional polypeptide unit 2). In some embodiments, the T cell receptor constant chains (or regions) of the SIR is chosen from the constant chain of human T cell receptor-alpha (TCR-alpha or TCRα or TCRα or hTCR-alpha or hTCRα or hTCRa or Cα), human T cell receptor-beta1(TCR-beta1 or TCRβ1 or TCRb1 or hTCR-beta1 or hTCRβ1 or hTCRb1 or Cβ1), human T cell receptor-beta 2 (TCR-beta2 or TCRβ2 or TCRb2 or hTCR-beta2 or hTCRβ2 or hTCRb2 or Cβ2 also designated TCR-beta, TCRβ or TCRb or Cβ), human Pre-T cell receptor alpha ((preTCR-alpha or preTCRα or preTCRα or preCα), human T cell receptor-gamma (TCR-gamma or TCRγ or TCRg or hTCR-gamma or hTCRγ or hTCRg or hTCRγ1 or hTCRgamma1, or Cy), or human T cell receptor-delta (TCR-delta or TCRd or TCRδ or hTCR-delta or hTCRd or hTCRδ or Cδ). In some embodiments, the TCR constant chains of SIR are encoded by their wild-type nucleotide sequences while in other aspects the TCR constant chains of SIR are encoded by the nucleotide sequences that are not wild-type. In some embodiments, the TCR constant chains of SIR are encoded by their human codon optimized sequences. In some embodiments, the TCR constant chains of SIR encode for the wild-type polypeptide sequences while in other embodiments the TCR constant chains of SIR encoded for polypeptides that carry one or more mutations. In some embodiments, the TCR constant chains of SIR are encoded by their codon optimized sequences that carry one or more mutations.

The term “TCR constant chain” or “constant region of T cell receptor” is defined as the constant chain of TCRα/TCRa, TCRβ1/TCRb1, TCRβ2/TCRb2, TCRγ/TCRd, TCRδ/TCRd and pre-TCRα. Examples of TCR constant chains are listed in Table 9B of current application and Table 12 of the provisional patent application. A TCR constant chain can be divided into several subdomains such as Ig-like C1 domain (e.g., SEQ ID NO: 1168-1175; Table 13 of the provisional patent application), connecting peptide (e.g., SEQ ID NO: 1177-1184; Table 9B and Table 14 of the provisional patent application), transmembrane domain (SEQ ID NO:1187-1190; Table 9B of current application and Table 15 of the provisional patent application), and cytosolic domain (e.g., SEQ ID NO: 1193-1196; Table 16 of provisional). The cytosolic domains of TCRα, TCRβ1/β2, TCRγ and TCRδ chains are short and generally not believed to play any significant role in their signaling activities.

The term “single chain variable region” or “scFv” refers to a fusion protein comprising at least one antibody fragment comprising a variable region of a light chain and at least one antibody fragment comprising a variable region of a heavy chain.

As use herein, the term “specifically binds” or “is specific for” refers to measurable and reproducible interactions, such as binding between a target and an antibody or antibody moiety, that is determinative of the presence of the target in the presence of a heterogeneous population of molecules, including biological molecules.

The term “signaling domain” refers to the functional region of a protein which transmits information within the cell to regulate cellular activity via defined signaling pathways by generating second messengers or functioning as effectors by responding to such messengers.

The term “signaling module” refers to a molecule or molecular complex comprising one or more signaling mediators or signaling adaptors that is capable of initiating a cell signal.

The term “signaling mediator” or “signaling adaptor” refers to molecule that is capable of initiating or inhibiting a cell signal when recruited by a natural or a non-natural signaling receptor. In contrast to a signaling receptor, a signaling adaptor lacks its own antigen binding domain or ligand binding domain. Example signaling adaptors include CD3ζ (CD3z), FcRγ, DAP10, DAP12, CD3ε, CD3γ and CD3δ.

The term “signaling chain” or “signaling fragment” refers to a polypeptide comprising the transmembrane and/or intracellular region and optionally the extracellular hinge/connecting peptide regions of a cell signaling receptor. Example signaling chains include the constant chains of TCRα, TCRβ, TCRγ and TCRδ. Additional example signaling chains include chains comprising the transmembrane and/or intracellular regions of CD16, NKp30, NKp44, NKp46, DAP10, DAP12, DNAM-1, NKG2D, CD32, CD64, KIR3DL1, KIR2DS4, FcRγ and CD3z.

The term SVH domain as used herein refers to a single human Vn domain antibody (VH sdAb). These terms are thus used interchangeably. The term SVH is also used interchangeably with independent vH domains. An example of an SVH is a fully human vH domain (FHVH) presented in SEQ ID NO (DNA): 425-426 and SEQ ID NO (PRT): 8805-8806.

The term “subject” is intended to include living organisms in which an immune response can be elicited (e.g., any domesticated mammals or a human).

As used herein, the term “TCR” or “T cell receptor” refers to a dimeric heterologous cell surface signaling protein forming an alpha-beta or gamma-delta receptor typically involved in recognizing an antigen presented by an MHC molecule (i.e., antigen recognition in the context of an MHC molecule).

As used herein, the term “TCR constant chain” refers to the constant chain TCRα, TCRβ1, TCRβ2, TCRγ, TCRδ and pre-TCRα and functional variants, mutants, alternative spliced isoforms, and homologs from non-human species. A TCR constant chain lacks the variable antigen binding domain but comprises the Ig-like domain, connecting peptide (or hinge domain), transmembrane domain and an optional intracellular or cytosolic domain.

As used herein, the term “T lymphocyte” or “T cell” refers to a cell expressing CD3 (CD3+) and a T Cell Receptor (TCR+).

The term “non-T cell” refers to a cell that is not a T cell. In an embodiment, a non-T cell lacks the cell surface expression of CD3 and a T cell receptor. In an embodiment, a non-T cell does not respond to a T cell activating antibody, such as OKT3. In an embodiment, a non-T cell lacks surface expression of CD3. In an embodiment, a non-T cell lacks the expression of one or more of CD3 chains selected from the group of CD3ε, CD3γ and CD3δ. An example of a non-T cell includes an NK cell, a B cell, a macrophage, a granulocyte, a dendritic cell, and an epithelial cell. A non-T cell can be an immortalized cell line.

The term “T cell receptor module,” or “TCRM,” refers to a heterodimer comprising sequences derived from a T cell receptor. The TCRM comprises T cell receptor transmembrane domains and may further comprise all or a portion of T cell receptor connecting peptides and/or intracellular domains.

The term “canonical TCRM” refers to a TCRM that is formed by heterodimerization between canonical TCR chains, i.e., TCRα and TCRβ1 or TCRβ2 chains, TCRγ and TCRδ chains, and pre-TCRα and TCRβ1 or β2 chains. Further, a canonical TCRM refers to a TCRM that is formed between two TCR chains that belong to the same species (e.g., human, mouse etc.). For example, a canonical TCRM is formed by heterodimerization of a first polypeptide chain that comprises the connecting peptide, transmembrane and intracellular domains of human TCRα and a second polypeptide chain that comprises the connecting peptide, transmembrane and intracellular domains of human TCRβ l or TCRβ2. Similarly, a canonical TCRM is formed by heterodimerization of a first polypeptide chain that comprises the connecting peptide, transmembrane and intracellular domains of human TCRγ and a second polypeptide chain that comprises the connecting peptide, transmembrane and intracellular domains of human TCRδ.

The term “non-canonical TCRM” refers to a TCRM that is not formed by the heterodimerization between canonical TCR chains, i.e., TCRα and TCRβ1 or TCRβ2 chains, TCRγ and TCRδ chains, and pre-TCRα and TCRβ1 or 02 chains. A non-canonical TCRM can be formed by heterodimerization of TCRα and TCRγ chains, TCRβ and TCRδ chains. A non-canonical TCRM is also formed between variants of TCRα, β, γ and δ chains, including their deletion mutants and hybrid chains. For example, a non-canonical TCRM is formed by a HC-SAR (SEQ ID NO: 32272) that comprises a first chain containing the constant domain and connecting peptide of TCRβ fused in frame to the transmembrane and intracellular domain of TCRγ and the second chain that is TCRα chain (SEQ ID NO: 8838).

The term “interspecies non-canonical TCRM” refers to a TCRM that is formed by heterodimerization of two TCR chains that belong to different species (e.g., between human TCRα and mouse TCRβ chain). An interspecies non-canonical TCRM is also formed when a component of a hybrid chain is derived from a different species. For example, an interspecies non-canonical TCRM is formed by heterodimerization between human TCRα chain and a hybrid TCRβ chain containing the constant domain of human TCRβ and connecting peptide, transmembrane domain and intracellular domain of mouse TCRβ.

The term “TCR-Fv” or “Fv-TCR” of “fragment variable TCR” as used here refers to an antigen binding module that is formed by the variable domains of TCR chains. A TCR-Fv can be formed by the Vα and Vβ domains or by Vγ and Vδ domains.

The term “Fv” or “fragment variable” as used here refers to an antigen binding module that is formed by the variable domains of an antibody. A Fv can be formed by the vL and vH domains.

As used herein a “transmembrane module” or “TMM” refers to a molecule or a molecular complex comprising a transmembrane protein (e.g., TCRα, TCRβ, TCRγ, TCRδ, CD16A or CD3z).

The term “membrane associated module” or “MAM” refers to a molecule or a molecular complex comprising a transmembrane protein (e.g., CD16A, CD3ζ) or a membrane anchored protein (e.g., CD16B). The term encompasses transmembrane proteins, such as CD16A, CD3z (or CD3L) and GPI-linked proteins, such as CD16B. A MAM may further comprise all or portions of hinge domains and/or cytosolic domains.

“Therapeutic agents” as used herein refers to agents that are used to, for example, treat, inhibit, prevent, mitigate the effects of, reduce the severity of, reduce the likelihood of developing, slow the progression of and/or cure, a disease.

“Therapeutic Controls” as used herein refers to an element used for controlling the activity of a SAR expressing cell. Examples of therapeutic controls are provided in Table 24 of provisional application.

The term “therapeutic effect” refers to a biological effect which can be manifested by various means, including but not limited to, e.g., decrease in tumor volume, a decrease in the number of cancer cells etc.

The phrase “therapeutically effective amount” as used herein means a sufficient amount of the composition to treat a disorder, at a reasonable benefit/risk ratio applicable to any medical treatment.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic compounds, plasmids, and viruses.

“Transmembrane domain” (or TM domain) as used herein refers to the region of a receptor, (e.g., a SAR) which crosses the plasma membrane.

“Vector,” “cloning vector” and “expression vector” as used herein refer to the vehicle by which a polynucleotide sequence (e.g., a foreign gene) can be introduced into a host cell, so as to transform the host and promote expression (e.g., transcription and translation) of the introduced sequence. Vectors include plasmids, phages, viruses, etc.

The term “viral vector” refers to a vector obtained or derived from a virus.

The term “zeta” or alternatively “zeta chain”, “CD3-zeta” or “TCR-zeta” “CD3ζ” is defined as the protein provided as GenBank Ace. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation.

The Tables 9-20 and 23-25 of the provisional patent application summarize the target antigens, Clone IDs, SEQ ID (DNA), SEQ ID (PRT) and names of several example SARs described in this disclosure. These constructs were made in general by combining the antigen binding fragments described in Tables 3-6 and in PCT/US22/17177 with example signaling chains described herein (including variants as provided in Table 7 of the provisional patent application). The SARs are divided into different types based on their architecture or backbone; i.e. the type of signaling chain (e.g., TCR constant chain, CD16 chain, CD3z chain etc.) present in them. However, it is to be understood that the SAR are modular in design and the scope of this disclosure is not limited to the SARs described in the Tables 9-20 and 23-25 of the provisional patent application and it is possible to generate different SARs by switching the different modules. Thus, it is possible to combine the antigen binding domains with other variants of TCR constant chains, but which are not included in the SARs described in the Table 7 of the provisional patent application. It is also possible to design SAR using antigen binding domains not listed in Tables 3-6. It is also possible to add or replace or remove the different therapeutic and accessory modules to the SAR. Thus, while the Tables 9-20 and 23-25 of the provisional patent application contain several SARs with an antibiotic resistance gene (e.g., PAC), this module can be removed or replaced by other therapeutic and accessory modules (e.g., IL12f, K13, MC159, icaspase etc.). Finally, other SARs (e.g., CAR, TFP) can be expressed in combination with SARs described herein.

The provisional patent application of this disclosure provides SEQ ID NO of several SARs and their components in the forms of tables. These tables can be used to identify a SAR comprising a specific antigen binding domain and belonging to a particular architecture. Alternatively, the sequence of a SAR containing a particular antigen binding domain of this disclosure can be determined by homology searching of the sequence listing file accompanying this disclosure. Finally, since the SARs are modular in design, the DNA and amino acid sequence of a SAR containing a particular module can be generated by substituting the module with the new module.

TABLE 3
Table 3

| | | vL- | | vH- | | | |
|---|---|---|---|---|---|---|---|
| | | SEQ | | SEQ | | scFv- | scFv- |
| | | ID | vL-SEQ | ID | vH-SEQ | DNA | PRT |
| TARGET | | NO | ID NO | NO | ID NO | SEQ ID | SEQ |
| ANTIGEN | NAME | (DNA | (PRT) | (DNA) | (PRT) | NO | ID |
| CD19 | hu-mROO5-1 | 339 | 8719 | 363 | 8743 | 387 | 8767 |
| CD45 | CD45-CH | 340 | 8720 | 364 | 8744 | 388 | 8768 |
| Her2p95 | Hup95Her2-USC1 | 341 | 8721 | 365 | 8745 | 389 | 8769 |
| Hcr2p95 | mu-p95Her2-USC2 | 342 | 8722 | 366 | 8746 | 390 | 8770 |
| OR2H1 | OR2H1-usc1 | 343 | 8723 | 367 | 8747 | 391 | 8771 |
| OR2H1 | R2PA1-usc2 | 344 | 8724 | 368 | 8748 | 392 | 8772 |
| OR2H1 | R2PA1-usc2 | 345 | 8725 | 369 | 8749 | 393 | 8773 |
| CLDN-18-2 | CLDN182-163E12 | 346 | 8726 | 370 | 8750 | 394 | 8774 |
| CLDN-18-2 | hu163E12-USC2 | 347 | 8727 | 371 | 8751 | 395 | 8775 |
| DLL3 | hSC16-57-USC1 | 348 | 8728 | 372 | 8752 | 396 | 8776 |
| p53 | p53-R248Q-336- | 349 | 8729 | 373 | 8753 | 397 | 8777 |
| | USC1 | | | | | | |
| TAG72 | huTAG72-v59-v15 | 350 | 8730 | 374 | 8754 | 398 | 8778 |
| MSLN | MSLN-hu22A10 | 351 | 8731 | 375 | 8755 | 399 | 8779 |
| PSMA | hu-PSMA-J591 | 352 | 8732 | 376 | 8756 | 400 | 8780 |
| He2 | huMab4D5-H91A | 353 | 8733 | 377 | 8757 | 401 | 8781 |
| BCMA | huUSC82-S29G | 354 | 8734 | 378 | 8758 | 402 | 8782 |
| BCMA | BCMA-3-USC2 | 19766 | 20386 | 19785 | 20405 | 19804 | 20424 |
| BCMA | BCMA-57-USC3 | 19767 | 20387 | 19786 | 20406 | 19805 | 20425 |
| CD70 | CD70-AM13 | 19768 | 20388 | 19787 | 20407 | 19806 | 20426 |
| CD70 | CD70-AM16 | 19769 | 20389 | 19788 | 20408 | 19807 | 20427 |
| CD70 | Hu-CD70-2H5 | 19770 | 20390 | 19789 | 20409 | 19808 | 20428 |
| CD70 | huCD70-hu18E7 | 19771 | 20391 | 19790 | 20410 | 19809 | 20429 |
| CD70 | CD70-P08F09- | 19772 | 20392 | 19791 | 20411 | 19810 | 20430 |
| | USC1 | | | | | | |
| CSFIR | CSFIR-AXI | 19773 | 20393 | 19792 | 20412 | 19811 | 20431 |
| CSF1R | CSFIR-AXI-USC1 | 19774 | 20394 | 19793 | 20413 | 19812 | 20432 |
| STEAP2 | RGN-7814N-USC1 | 19775 | 20395 | 19794 | 20414 | 19813 | 20433 |
| STEAP2 | STEAP2-RGN- | 19776 | 20396 | 19795 | 20415 | 19814 | 20434 |
| | 1162-USC1 | | | | | | |
| ALK | Alk-48 | 32912 | 40759 | 33121 | 41030 | 33330 | 41301 |
| ALK | Alk-58 | 32913 | 40760 | 33122 | 41031 | 33331 | 41302 |
| BCMA | BCMA-huC12A3 | 32914 | 40761 | 33123 | 41032 | 33332 | 41303 |
| BCMA | BCMA-J6M0 | 32915 | 40762 | 33124 | 41033 | 33333 | 41304 |
| CD123 | CD123-CSL362 | 32916 | 40763 | 33125 | 41034 | 33334 | 41305 |
| CD19 | CD138 | 32917 | 40764 | 33126 | 41035 | 33335 | 41306 |
| CD19 | CD179b | 32918 | 40765 | 33127 | 41036 | 33336 | 41307 |
| CD19 | CD19Bu12 | 32919 | 40766 | 33128 | 41037 | 33337 | 41308 |
| CD19 | FMC63 | 32920 | 40767 | 33129 | 41038 | 33338 | 41309 |
| CD19 | huFMC63-11 | 32921 | 40768 | 33130 | 41039 | 33339 | 41310 |
| CD20 | CD20-2F2 | 32922 | 40769 | 33131 | 41040 | 33340 | 41311 |
| CD20 | CD20-GA101 | 32923 | 40770 | 33132 | 41041 | 33341 | 41312 |
| CD22 | CD22-h10F4 | 32924 | 40771 | 33133 | 41042 | 33342 | 41313 |
| CD276 | CD276-17 | 32925 | 40772 | 33134 | 41043 | 33343 | 41314 |
| CD30 | CD30-5F11 | 32926 | 40773 | 33135 | 41044 | 33344 | 41315 |
| CD30 | CD30-Ac10 | 32927 | 40774 | 33136 | 41045 | 33345 | 41316 |
| CD32 | CD32-Med9 | 32928 | 40775 | 33137 | 41046 | 33346 | 41317 |
| CD324 | CD324-hSC10-17 | 32929 | 40776 | 33138 | 41047 | 33347 | 41318 |
| CD324 | CD324-SC10-6 | 32930 | 40777 | 33139 | 41048 | 33348 | 41319 |
| CD33b | CD33-huMyc9 | 32931 | 40778 | 33140 | 41049 | 33349 | 41320 |
| CD33 | CD33-AF5 | 32932 | 40779 | 33141 | 41050 | 33350 | 41321 |
| CD34 | CD34-hu4C7 | 32933 | 40780 | 33142 | 41051 | 33351 | 41322 |
| CD5 | CD5-18 | 32934 | 40781 | 33143 | 41052 | 33352 | 41323 |
| CD5 | CD5-9 | 32935 | 40782 | 33144 | 41053 | 33353 | 41324 |
| CD70 | CD70-h1F6 | 32936 | 40783 | 33145 | 41054 | 33354 | 41325 |
| CD79b | CD79b-2F2 | 32937 | 40784 | 33146 | 41055 | 33355 | 41326 |
| CD79b | huMA79bv28 | 32938 | 40785 | 33147 | 41056 | 33356 | 41327 |
| CDH17 | PTA001A4 | 32939 | 40786 | 33148 | 41057 | 33357 | 41328 |
| CDH19 | CDH19-16A4 | 32940 | 40787 | 33149 | 41058 | 33358 | 41329 |
| CDH6 | CDH6-NOV710 | 32941 | 40788 | 33150 | 41059 | 33359 | 41330 |
| CDH6 | CDH6-NOV712 | 32942 | 40789 | 33151 | 41060 | 33360 | 41331 |
| CLEC5A | CLEC5A-3E12A2 | 32943 | 40790 | 33152 | 41061 | 33361 | 41332 |
| CLEC5A | CLEC5A-8H8F5 | 32944 | 40791 | 33153 | 41062 | 33362 | 41333 |
| CLL1 | CLL1-M26 | 32945 | 40792 | 33154 | 41063 | 33363 | 41334 |
| CLL1 | CLL1-M32 | 32946 | 40793 | 33155 | 41064 | 33364 | 41335 |
| CS1 | HuLuc64-[2] | 32947 | 40794 | 33156 | 41065 | 33365 | 41336 |
| CS1 | huLuc90 | 32948 | 40795 | 33157 | 41066 | 33366 | 41337 |
| CSF2RA | CSF2RA-Ab1 | 32949 | 40796 | 33158 | 41067 | 33367 | 41338 |
| CSF2RA | CSF2RA-Ab6 | 32950 | 40797 | 33159 | 41068 | 33368 | 41339 |
| DLL3 | DLL3-hSC16-13 | 32951 | 40798 | 33160 | 41069 | 33369 | 41340 |
| DLL3 | DLL3-hSC16-56 | 32952 | 40799 | 33161 | 41070 | 33370 | 41341 |
| EGFR | Cetuximab | 32953 | 40800 | 33162 | 41071 | 33371 | 41342 |
| EGFRviii | EGFRviii-2173 | 32954 | 40801 | 33163 | 41072 | 33372 | 41343 |
| EpCAM | EpCam1-D5K5 | 32955 | 40802 | 33164 | 41073 | 33373 | 41344 |
| EpCAM | Epcam1-MM1 | 32956 | 40803 | 33165 | 41074 | 33374 | 41345 |
| FLT3 | FLT3-NC7 | 32957 | 40804 | 33166 | 41075 | 33375 | 41346 |
| HIV1-gp | HIV1-N6 | 32958 | 40805 | 33167 | 41076 | 33376 | 41347 |
| FR1 | FR1-huMov19 | 32959 | 40806 | 33168 | 41077 | 33377 | 41348 |
| GD2 | GD2-hu14-18 | 32960 | 40807 | 33169 | 41078 | 33378 | 41349 |
| GD2 | GD2-hu3F8 | 32961 | 40808 | 33170 | 41079 | 33379 | 41350 |
| GD3 | GD3-KM-641 | 32962 | 40809 | 33171 | 41080 | 33380 | 41351 |
| GFR4 | GFRAlpha4-P4-6 | 32963 | 40810 | 33172 | 41081 | 33381 | 41352 |
| GM1 | GM1-5B2 | 32964 | 40811 | 33173 | 41082 | 33382 | 41353 |
| GPRC5D | ET150-18 | 32965 | 40812 | 33174 | 41083 | 33383 | 41354 |
| GPRC5D | GPRC5D-ET150-5 | 32966 | 40813 | 33175 | 41084 | 33384 | 41355 |
| Her2 | Her2-Hu4D5 | 32967 | 40814 | 33176 | 41085 | 33385 | 41356 |
| HIV1-gp100 | HIV1-PGT-128 | 32968 | 40815 | 33177 | 41086 | 33386 | 41357 |
| HIV1-gp100 | HIV1-X5 | 32969 | 40816 | 33178 | 41087 | 33387 | 41358 |
| IL11Ra | IL11Ra-8E2 | 32970 | 40817 | 33179 | 41088 | 33388 | 41359 |
| IL13Ra2 | IL13Ra2-hu107 | 32971 | 40818 | 33180 | 41089 | 33389 | 41360 |
| IL13Ra2 | IL13Ra2-Hu108 | 32972 | 40819 | 33181 | 41090 | 33390 | 41361 |
| L1CAM | L1CAM-9-3-HU3 | 32973 | 40820 | 33182 | 41091 | 33391 | 41362 |
| LAMP1 | LAMP1-humab1-2 | 32974 | 40821 | 33183 | 41092 | 33392 | 41363 |
| LAMP1 | LAMP1-Mb4 | 32975 | 40822 | 33184 | 41093 | 33393 | 41364 |
| Lym1 | Lym1 | 32976 | 40823 | 33185 | 41094 | 33394 | 41365 |
| Lym2 | Lym2 | 32977 | 40824 | 33186 | 41095 | 33395 | 41366 |
| MPL | MPL-111 | 32978 | 40825 | 33187 | 41096 | 33396 | 41367 |
| MPL | MPL-161 | 32979 | 40826 | 33188 | 41097 | 33397 | 41368 |
| MPL | MPL-161 | 32980 | 40827 | 33189 | 41098 | 33398 | 41369 |
| MPL | MPL-175 | 32981 | 40828 | 33190 | 41099 | 33399 | 41370 |
| TCRB1 | TCRB1-E09 | 32982 | 40829 | 33191 | 41100 | 33400 | 41371 |
| TCRB1 | TCRB1-Jovi1 | 32983 | 40830 | 33192 | 41101 | 33401 | 41372 |
| TCRB2 | TCRB2-D05 | 32984 | 40831 | 33193 | 41102 | 33402 | 41373 |
| TCRB2 | TCRB2-E05 | 32985 | 40832 | 33194 | 41103 | 33403 | 41374 |
| TCRgd | TCRgd-G5-4 | 32986 | 40833 | 33195 | 41104 | 33404 | 41375 |
| TnAg | TnAg | 32987 | 40834 | 33196 | 41105 | 33405 | 41376 |
| Tn-Muc1 | Tn-Muc1-hu5E5 | 32988 | 40835 | 33197 | 41106 | 33406 | 41377 |
| TROP2 | TROP2- | 32989 | 40836 | 33198 | 41107 | 33407 | 41378 |
| WT1/HLA2 | WT1-Ab13 | 32990 | 40837 | 33199 | 41108 | 33408 | 41379 |
| WT1/HLA | WT1-Ab15 | 32991 | 40838 | 33200 | 41109 | 33409 | 41380 |
| WT1/HLA | WT1-Ab5 | 32992 | 40839 | 33201 | 41110 | 33410 | 41381 |
| CD123 | CD123-1172 | 32993 | 40840 | 33202 | 41111 | 33411 | 41382 |
| CDH19 | CDH19-4B10 | 32994 | 40841 | 33203 | 41112 | 33412 | 41383 |
| FR-beta | FRbeta-m923 | 32995 | 40842 | 33204 | 41113 | 33413 | 41384 |
| B7J4 | B7H4-hu22C10 | 32996 | 40843 | 33205 | 41114 | 33414 | 41385 |
| B7H4 | B7H4-hu1D11 | 32997 | 40844 | 33206 | 41115 | 33415 | 41386 |
| CD23 | CD23-p5E8 | 32998 | 40845 | 33207 | 41116 | 33416 | 41387 |
| GCC | GCC-Ab229 | 32999 | 40846 | 33208 | 41117 | 33417 | 41388 |
| CD200R | CD200R-huDx182 | 33000 | 40847 | 33209 | 41118 | 33418 | 41389 |
| AFP/HLA-A2 | AFP-76 | 33001 | 40848 | 33210 | 41119 | 33419 | 41390 |
| AFP/HLA-A2 | AFP-79 | 33002 | 40849 | 33211 | 41120 | 33420 | 41391 |
| BCMA | BCMA-ET-03 | 33003 | 40850 | 33212 | 41121 | 33421 | 41392 |
| BCMA | BCMA-huC11.D5.3L1H3 | 33004 | 40851 | 33213 | 41122 | 33422 | 41393 |
| BCMA | BCMA-huC13-F12 | 33005 | 40852 | 33214 | 41123 | 33423 | 41394 |
| CD123 | CD123-DART-1 | 33006 | 40853 | 33215 | 41124 | 33424 | 41395 |
| CD123 | CD123-DART-2 | 33007 | 40854 | 33216 | 41125 | 33425 | 41396 |
| CD123 | CD123-1176 | 33008 | 40855 | 33217 | 41126 | 33426 | 41397 |
| CD123 | CD123-2B8 | 33009 | 40856 | 33218 | 41127 | 33427 | 41398 |
| CD123 | CD123-9D7 | 33010 | 40857 | 33219 | 41128 | 33428 | 41399 |
| CD123 | CD123-3B10 | 33011 | 40858 | 33220 | 41129 | 33429 | 41400 |
| CD19 | CD19-MOR0028 | 33012 | 40859 | 33221 | 41130 | 33430 | 41401 |
| CD19 | hu-mROO5-1 | 33013 | 40860 | 33222 | 41131 | 33431 | 41402 |
| CD20 | CD20-Ubli-v4 | 33014 | 40861 | 33223 | 41132 | 33432 | 41403 |
| CD20 | CD20-7D8 | 33015 | 40862 | 33224 | 41133 | 33433 | 41404 |
| CD33 | CD33-SGNh2H12 | 33016 | 40863 | 33225 | 41134 | 33434 | 41405 |
| CD99 | CD99-hu12E7 | 33017 | 40864 | 33226 | 41135 | 33435 | 41406 |
| CLL1 | CLL1-21C9-L2H3 | 33018 | 40865 | 33227 | 41136 | 33436 | 41407 |
| CLL1 | CLL1-6E7L4H1e | 33019 | 40866 | 33228 | 41137 | 33437 | 41408 |
| CLL1 | CLL1-hu1075-v1 | 33020 | 40867 | 33229 | 41138 | 33438 | 41409 |
| CLL1 | CLL1-hu1075-v2 | 33021 | 40868 | 33230 | 41139 | 33439 | 41410 |
| FITC | FITC-4M-53 | 33022 | 40869 | 33231 | 41140 | 33440 | 41411 |
| FITC | FITC-E2 | 33023 | 40870 | 33232 | 41141 | 33441 | 41412 |
| GPRC5D | GPRC5D-ET150-1 | 33024 | 40871 | 33233 | 41142 | 33442 | 41413 |
| GPRC5D | GPRC5D-ET150-2 | 33025 | 40872 | 33234 | 41143 | 33443 | 41414 |
| HLA-A2 | HLA-A2-3PB2 | 33026 | 40873 | 33235 | 41144 | 33444 | 41415 |
| Kappa-LC | Kappa-LC1 | 33027 | 40874 | 33236 | 41145 | 33445 | 41416 |
| CD19 | hCD19-EUK5-13 | 33028 | 40875 | 33237 | 41146 | 33446 | 41417 |
| Streptag | Streptag | 33029 | 40876 | 33238 | 41147 | 33447 | 41418 |
| MPL | Hu-161-2 | 33030 | 40877 | 33239 | 41148 | 33448 | 41419 |
| MPL | MPL-hu-175-2 | 33031 | 40878 | 33240 | 41149 | 33449 | 41420 |
| MPL | MPL-hu-111-2 | 33032 | 40879 | 33241 | 41150 | 33450 | 41421 |
| CD179a | CD179a-2460-B04 | 33033 | 40880 | 33242 | 41151 | 33451 | 41422 |
| CD179a | CD179a-2462-E07 | 33034 | 40881 | 33243 | 41152 | 33452 | 41423 |
| CD22 | CD22-HA22 | 33035 | 40882 | 33244 | 41153 | 33453 | 41424 |
| STEAP1 | STEAP1-hu120 | 33036 | 40883 | 33245 | 41154 | 33454 | 41425 |
| Liv1 | hLiv1-mAb2 | 33037 | 40884 | 33246 | 41155 | 33455 | 41426 |
| Ncctin4 | hu-Ncctin4-mAb1 | 33038 | 40885 | 33247 | 41156 | 33456 | 41427 |
| CRIPTO | hu-Cripto-L1H2 | 33039 | 40886 | 33248 | 41157 | 33457 | 41428 |
| gpA33 | hu-gpA33 | 33040 | 40887 | 33249 | 41158 | 33458 | 41429 |
| ROR1 | ROR1-DART4 | 33041 | 40888 | 33250 | 41159 | 33459 | 41430 |
| CLL1 | CLL1-24C8 | 33042 | 40889 | 33251 | 41160 | 33460 | 41431 |
| CLLI | CLL1-24C1 | 33043 | 40890 | 33252 | 41161 | 33461 | 41432 |
| FLT3 | FLT3-10E3 | 33044 | 40891 | 33253 | 41162 | 33462 | 41433 |
| FLT3 | FLT3-8B5 | 33045 | 40892 | 33254 | 41163 | 33463 | 41434 |
| IL1RAP | IL1RAP-IAPB57 | 33046 | 40893 | 33255 | 41164 | 33464 | 41435 |
| IL1RAP | IL1RAP-IAPB63 | 33047 | 40894 | 33256 | 41165 | 33465 | 41436 |
| IL1RAP | hu-IL1RAP- | 33048 | 40895 | 33257 | 41166 | 33466 | 41437 |
| MSLN | MSLN-7D9-v3 | 33049 | 40896 | 33258 | 41167 | 33467 | 41438 |
| MSLN | MSLN-hu22A10 | 33050 | 40897 | 33259 | 41168 | 33468 | 41439 |
| BST1 | hu-BST1-A1 | 33051 | 40898 | 33260 | 41169 | 33469 | 41440 |
| BST1 | hu-BST1-A2 | 33052 | 40899 | 33261 | 41170 | 33470 | 41441 |
| BST1 | hu-BST1-A3 | 33053 | 40900 | 33262 | 41171 | 33471 | 41442 |
| CD19 | CAT17 | 33054 | 40901 | 33263 | 41172 | 33472 | 41443 |
| CD22 | hu-HA22-1 | 33055 | 40902 | 33264 | 41173 | 33473 | 41444 |
| CD70 | CD70-AM13 | 33056 | 40903 | 33265 | 41174 | 33474 | 41445 |
| Her2 | Her2-XMT-1520 | 33057 | 40904 | 33266 | 41175 | 33475 | 41446 |
| Her2 | Her2-XMT-1518 | 33058 | 40905 | 33267 | 41176 | 33476 | 41447 |
| Her2 | huMab4D5-D98W | 33059 | 40906 | 33268 | 41177 | 33477 | 41448 |
| MSLN | MSLN-3-AM | 33060 | 40907 | 33269 | 41178 | 33478 | 41449 |
| MSLN | MSLN-5 | 33061 | 40908 | 33270 | 41179 | 33479 | 41450 |
| EGFRviii | EGFRviii-2-AM | 33062 | 40909 | 33271 | 41180 | 33480 | 41451 |
| EGFRviii | H2M1863N2 | 33063 | 40910 | 33272 | 41181 | 33481 | 41452 |
| DLL3 | DLL3-AM6 | 33064 | 40911 | 33273 | 41182 | 33482 | 41453 |
| DLL3 | DLL3-AM14 | 33065 | 40912 | 33274 | 41183 | 33483 | 41454 |
| Nectin4 | Nectin4-66 | 33066 | 40913 | 33275 | 41184 | 33484 | 41455 |
| MSLN | MSLN-237 | 33067 | 40914 | 33276 | 41185 | 33485 | 41456 |
| MSLN | MSLN-HuAM15 | 33068 | 40915 | 33277 | 41186 | 33486 | 41457 |
| MSLN | MSLN76923 | 33069 | 40916 | 33278 | 41187 | 33487 | 41458 |
| PRLR | PRLR-CN | 33070 | 40917 | 33279 | 41188 | 33488 | 41459 |
| EMR2 | EMR2-USC2- | 33071 | 40918 | 33280 | 41189 | 33489 | 41460 |
| CEA | CEA-USC14 | 33072 | 40919 | 33281 | 41190 | 33490 | 41461 |
| Her3 | Her3-USC14 | 33073 | 40920 | 33282 | 41191 | 33491 | 41462 |
| FOLR1 | FOLR1-USC14 | 33074 | 40921 | 33283 | 41192 | 33492 | 41463 |
| FOLR1 | FOLR1-USC24 | 33075 | 40922 | 33284 | 41193 | 33493 | 41464 |
| CLDN6 | CLDN6-USC1 | 33076 | 40923 | 33285 | 41194 | 33494 | 41465 |
| CLDN6 | CLDN6-USC2 | 33077 | 40924 | 33286 | 41195 | 33495 | 41466 |
| SLC34A2 | huMX35-LH4 | 33078 | 40925 | 33287 | 41196 | 33496 | 41467 |
| CD22 | CD22-INO | 33079 | 40926 | 33288 | 41197 | 33497 | 41468 |
| CD22 | CD22-hu-RFB4 | 33080 | 40927 | 33289 | 41198 | 33498 | 41469 |
| CD22 | huHA22-2 | 33081 | 40928 | 33290 | 41199 | 33499 | 41470 |
| CD19 | huCD19-USC3 | 33082 | 40929 | 33291 | 41200 | 33500 | 41471 |
| BCMA | BCMA-hu72 | 33083 | 40930 | 33292 | 41201 | 33501 | 41472 |
| MPL | hu-161-3 | 33084 | 40931 | 33293 | 41202 | 33502 | 41473 |
| BAFFR | hu-BAFFR-USC90 | 33085 | 40932 | 33294 | 41203 | 33503 | 41474 |
| BAFFR | hu-BAFFR-USC55 | 33086 | 40933 | 33295 | 41204 | 33504 | 41475 |
| BAFFR | MOR6654 | 33087 | 40934 | 33296 | 41205 | 33505 | 41476 |
| ROR1 | ROR1-JJ-67 | 33088 | 40935 | 33297 | 41206 | 33506 | 41477 |
| ROR1 | ROR1-JJ-78 | 33089 | 40936 | 33298 | 41207 | 33507 | 41478 |
| ROR1 | ROR1-JJ-76 | 33090 | 40937 | 33299 | 41208 | 33508 | 41479 |
| Her2 | Her2-FRP5 | 33091 | 40938 | 33300 | 41209 | 33509 | 41480 |
| CD19 | CEA-huMN14 | 33092 | 40939 | 33301 | 41210 | 33510 | 41481 |
| CEA | CEA-BW431-26 | 33093 | 40940 | 33302 | 41211 | 33511 | 41482 |
| Her2 | hMab4D5-H91A | 33094 | 40941 | 33303 | 41212 | 33512 | 41483 |
| Her2 | Her2-USC-1516 | 33095 | 40942 | 33304 | 41213 | 33513 | 41484 |
| TOSO | TOSO-6B10 | 33096 | 40943 | 33305 | 41214 | 33514 | 41485 |
| CD30 | CD30-HRS3 | 33097 | 40944 | 33306 | 41215 | 33515 | 41486 |
| CD229 | CD229-USC1-2D4 | 33098 | 40945 | 33307 | 41216 | 33516 | 41487 |
| CD229 | CD229-2A2 | 33099 | 40946 | 33308 | 41217 | 33517 | 41488 |
| CD229 | CD229-USC3-2D5 | 33100 | 40947 | 33309 | 41218 | 33518 | 41489 |
| CD229 | CD229-USC2-2D4 | 33101 | 40948 | 33310 | 41219 | 33519 | 41490 |
| EBV-gp350 | EBV-gp350-7A1 | 33102 | 40949 | 33311 | 41220 | 33520 | 41491 |
| EBV-gp350 | EBV-gp350-6G4 | 33103 | 40950 | 33312 | 41221 | 33521 | 41492 |
| INFA-A-NA | INFL-NA-1E01 | 33104 | 40951 | 33313 | 41222 | 33522 | 41493 |
| EBV-LMP1 | EBV-LMP1EA | 33105 | 40952 | 33314 | 41223 | 33523 | 41494 |
| PSMA | PSMA-J591-ds75 | 33106 | 40953 | 33315 | 41224 | 33524 | 41495 |
| PSMA | hu-PSMA-J591 | 33107 | 40954 | 33316 | 41225 | 33525 | 41496 |
| PSMA | hu-PSMA-J591 | 33108 | 40955 | 33317 | 41226 | 33526 | 41497 |
| PSMA | PSMA-J591-ds75 | 33109 | 40956 | 33318 | 41227 | 33527 | 41498 |
| MUC1 | huMUC1-MNE6 | 33110 | 40957 | 33319 | 41228 | 33528 | 41499 |
| MUCI | MUC1-star-MN-E6 | 33111 | 40958 | 33320 | 41229 | 33529 | 41500 |
| MUC1 | hu-MUC1-MN-C2 | 33112 | 40959 | 33321 | 41230 | 33530 | 41501 |
| gpA33 | hu-gpA33-C825 | 33113 | 40960 | 33322 | 41231 | 33531 | 41502 |
| MSLN | MSLN-7D10 | 33114 | 40961 | 33323 | 41232 | 33532 | 41503 |
| MSLN | MSLN-7D9 | 33115 | 40962 | 33324 | 41233 | 33533 | 41504 |
| MSLN | MSLN-7D9-V29L | 33116 | 40963 | 33325 | 41234 | 33534 | 41505 |
| MSLN | MSLN-hu22A10 | 33117 | 40964 | 33326 | 41235 | 33535 | 41506 |
| MSLN | hu22A10-N31S | 33118 | 40965 | 33327 | 41236 | 33536 | 41507 |
| BCMA | hu-USC82-S29G | 33120 | 40966 | 33329 | 41237 | 33538 | 41508 |
| EGFRviii | GC1-Y31F-USC1 | 32006 | 40967 | 32069 | 41238 | 32132 | 41509 |
| EGFRviii | EGFRviii-GC1 | 32007 | 40968 | 32070 | 41239 | 32133 | 41510 |
| EGFRviii | GC1-S51T-USC2 | 32008 | 40969 | 32071 | 41240 | 32134 | 41511 |
| EGFRviii | GC1-D29N-USC3 | 32009 | 40970 | 32072 | 41241 | 32135 | 41512 |
| EGFRviii | GC2 | 32010 | 40971 | 32073 | 41242 | 32136 | 41513 |
| EGFRviii | GC2-S25R | 32011 | 40972 | 32074 | 41243 | 32137 | 41514 |
| EGFRviii | GC2-S25N | 32012 | 40973 | 32075 | 41244 | 32138 | 41515 |
| EGFRviii | GC2-S31N | 32013 | 40974 | 32076 | 41245 | 32139 | 41516 |
| GD2 | GD2-hu3F8-USC1 | 32014 | 40975 | 32077 | 41246 | 32140 | 41517 |
| GD2 | GD2-hu3F8-USC2 | 32015 | 40976 | 32078 | 41247 | 32141 | 41518 |
| GD2 | hu-KM666-USC1 | 32016 | 40977 | 32079 | 41248 | 32142 | 41519 |
| GD2 | GD2-mu-KM666 | 32017 | 40978 | 32080 | 41249 | 32143 | 41520 |
| CLDN18.2 | hu-CLD18A2- | 32018 | 40979 | 32081 | 41250 | 32144 | 41521 |
| | 175D10-USC1 | | | | | | |
| CLDN18.2 | hu-CLD18A2- | 32019 | 40980 | 32082 | 41251 | 32145 | 41522 |
| | 175D10-USC2 | | | | | | |
| CLDN6 | hu-CLDN6-BNT- | 32020 | 40981 | 32083 | 41252 | 32146 | 41523 |
| | USC1 | | | | | | |
| CLDN6 | CLDN6-BNT | 32021 | 40982 | 32084 | 41253 | 32147 | 41524 |
| CLDN6 | CLDN6-BNT- | 32022 | 40983 | 32085 | 41254 | 32148 | 41525 |
| | USC1 | | | | | | |
| CLDN6 | CLDN6-G51A- | 32023 | 40984 | 32086 | 41255 | 32149 | 41526 |
| | AT1 | | | | | | |
| CD19 | hu-mROO5-1- | 32024 | 40985 | 32087 | 41256 | 32150 | 41527 |
| | A28T-AT1 | | | | | | |
| CD19 | hu-mROO5-1- | 32025 | 40986 | 32088 | 41257 | 32151 | 41528 |
| | Y32S-AT2 | | | | | | |
| CD19 | huCD19-USC3- | 32026 | 40987 | 32089 | 41258 | 32152 | 41529 |
| | AT1 | | | | | | |
| CD19 | hu-CD19-USC3- | 32027 | 40988 | 32090 | 41259 | 32153 | 41530 |
| | AT2 | | | | | | |
| CD79b | huMA79b-AT1 | 32028 | 40989 | 32091 | 41260 | 32154 | 41531 |
| CD79b | huMA79b-AT2 | 32029 | 40990 | 32092 | 41261 | 32155 | 41532 |
| CD79b | huMA79b-AT3 | 32030 | 40991 | 32093 | 41262 | 32156 | 41533 |
| CD79b | huCD79b-AT4 | 32031 | 40992 | 32094 | 41263 | 32157 | 41534 |
| CD79b | huCD79b-AT5 | 32032 | 40993 | 32095 | 41264 | 32158 | 41535 |
| CD79b | huCD79b-AT6 | 32033 | 40994 | 32096 | 41265 | 32159 | 41536 |
| CD79b | huMA79b-AT5 | 32034 | 40995 | 32097 | 41266 | 32160 | 41537 |
| CD79b | huCD79b-2F2-AT1 | 32035 | 40996 | 32098 | 41267 | 32161 | 41538 |
| CD79b | huCD79b-2F2-AT2 | 32036 | 40997 | 32099 | 41268 | 32162 | 41539 |
| CD79b | huCD79b-2F2-AT3 | 32037 | 40998 | 32100 | 41269 | 32163 | 41540 |
| CD79b | huCD79b-2F2-AT4 | 32038 | 40999 | 32101 | 41270 | 32164 | 41541 |
| CD79b | huCD79b-2F2-AT5 | 32039 | 41000 | 32102 | 41271 | 32165 | 41542 |
| CD79b | huCD79b-2F2-AT6 | 32040 | 41001 | 32103 | 41272 | 32166 | 41543 |
| CD79b | huCD79b-2F2-AT7 | 32041 | 41002 | 32104 | 41273 | 32167 | 41544 |
| CD79b | mu-MA79b | 32042 | 41003 | 32105 | 41274 | 32168 | 41545 |
| TAJ | KN5-USC1-R3D | 32043 | 41004 | 32106 | 41275 | 32169 | 41546 |
| GPRC5D | Ro-5F11-USC1 | 32044 | 41005 | 32107 | 41276 | 32170 | 41547 |
| GPRC5D | Ro-5E11-USC2 | 32045 | 41006 | 32108 | 41277 | 32171 | 41548 |
| GPRC5D | Ro-5F11-E11-USC | 32046 | 41007 | 32109 | 41278 | 32172 | 41549 |
| GPRC5D | 5E11-5F11-USC | 32047 | 41008 | 32110 | 41279 | 32173 | 41550 |
| GPRC5D | JJ-5B83-USC3 | 32048 | 41009 | 32111 | 41280 | 32174 | 41551 |
| FCRH5 | FCRH5-GN-USC2 | 32049 | 41010 | 32112 | 41281 | 32175 | 41552 |
| FR1 | huMov19-USC2 | 32050 | 41011 | 32113 | 41282 | 32176 | 41553 |
| p53-R175H | p53-R175H | 32051 | 41012 | 32114 | 41283 | 32177 | 41554 |
| p53-R175H | p53-R175H-AT1 | 32052 | 41013 | 32115 | 41284 | 32178 | 41555 |
| p53-R248Q | p53-R248Q-336 | 32053 | 41014 | 32116 | 41285 | 32179 | 41556 |
| Muc16 | huMuc16-3A5 | 32054 | 41015 | 32117 | 41286 | 32180 | 41557 |
| RORI | ROR1-JJ-67 | 32055 | 41016 | 32118 | 41287 | 32181 | 41558 |
| ROR1 | ROR1-JJ-67-AT1 | 32056 | 41017 | 32119 | 41288 | 32182 | 41559 |
| DLL3 | hSC16-57-USC1 | 32057 | 41018 | 32120 | 41289 | 32183 | 41560 |
| NPM1c | NPM1c-YG1 | 32058 | 41019 | 32121 | 41290 | 32184 | 41561 |
| IL23R | IL23R-SANG-11 | 32059 | 41020 | 32122 | 41291 | 32185 | 41562 |
| HLA-A2 | HLA-A2-SANG-76 | 32060 | 41021 | 32123 | 41292 | 32186 | 41563 |
| MOG01 | MOG01-USC1 | 32061 | 41022 | 32124 | 41293 | 32187 | 41564 |
| MOG301 | Y56N-USC2 | 32062 | 41023 | 32125 | 41294 | 32188 | 41565 |
| MOG | MOG473-USC3 | 32063 | 41024 | 32126 | 41295 | 32189 | 41566 |
| MOG | MOG01 | 32064 | 41025 | 32127 | 41296 | 32190 | 41567 |
| MOG | MOG301 | 32065 | 41026 | 32128 | 41297 | 32191 | 41568 |
| MOG | MOG-AZ17 | 32066 | 41027 | 32129 | 41298 | 32192 | 41569 |
| CSF1R | CSF1R-AXI | 32067 | 41028 | 32130 | 41299 | 32193 | 41570 |
| CSF1R | CSF1R-AXI-USC1 | 32068 | 41029 | 32131 | 41300 | 32194 | 41571 |

TABLE 4

| | | Va- | | | |
|---|---|---|---|---|---|
| | | SEQ | | Vb-SEQ | |
| | | ID NO | Va-SEQ ID | ID NO | Vb-SEQ ID |
| TARGET ANTIGEN | NAME | (DNA) | NO (PRT) | (DNA) | NO (PRT) |
| NY-ESO-1/HLA-A2 | IG4 | 305 | 8685 | 322 | 8702 |
| gp100/HLA-A2 | gp100 | 306 | 8686 | 323 | 8703 |
| Kras/HLA-A11 | IMMU | 307 | 8687 | 324 | 8704 |
| WT1/HLA-A2 | C4 | 308 | 8688 | 325 | 8705 |
| WT1/HLA-A2 | 37-45 | 309 | 8689 | 326 | 8706 |
| MAGE-A4/HLA-A2 | A4 | 310 | 8690 | 327 | 8707 |
| HPV-E6/HLA-A2 | E6-5859 | 311 | 8691 | 328 | 8708 |
| HPV-E6/HLA-A2 | E6-5843B14 | 312 | 8692 | 329 | 8709 |
| HPV-E7/HLA-A2 | HPV-E7 | 313 | 8693 | 330 | 8710 |
| PRAME/HLA-A2 | PRAME | 32214 | 40531 | 32216 | 40533 |
| PRAME/HLA-A2 | PRAME-USC1 | 32215 | 40532 | 32217 | 40534 |

TABLE 5
Table 5

| | | | | | | SEQ | SEQ |
|---|---|---|---|---|---|---|---|
| | | SEQ | SEQ | | | ID | ID |
| Target | Name of | ID NO | ID NO | Target | Name of | NO | NO |
| Antigen | Fragment | (DNA) | (PRT) | Antigen | Fragment | (DNA) | (PRT) |
| MTX | R304-MTX | 412 | 8792 | CD30 | hu-574-USC1 | 422 | 8802 |
| CLDN18-2 | C18.2-USC1 | 413 | 8793 | CD30 | hu-542-USC1 | 423 | 8803 |
| CLDN18-2 | C18.2-USC2 | 414 | 8794 | CD30 | hu-542-USC2 | 424 | 8804 |
| CLDN18-2 | C18.2-USC3 | 415 | 8795 | BCMA | BCMA-948 | 425 | 8805 |
| CD22 | hu-077-USC1 | 416 | 8796 | BCMA | BCMA-972 | 426 | 8806 |
| CD22 | hu-077-USC2 | 417 | 8797 | NYESO1 | sVb-TCR | 427 | 8807 |
| DLL3 | DLL3-USC1 | 418 | 8798 | NYESO1 | NsVb-TCR | 428 | 8808 |
| DLL3 | DLL3-USC4 | 419 | 8799 | MAGEA3 | A3-sVb | 429 | 8809 |
| DLL3 | DLL3-USC5 | 420 | 8800 | MAGEA3 | sVb-7-SP | 430 | 8810 |
| DLL3 | DLL3-USC3 | 421 | 8801 | BCMA | hu-E59D-US3 | 32204 | 41581 |
| CD20 | huCD20-AT1 | 32195 | 41572 | CD33 | hA873-G30S | 32205 | 41582 |
| CD20 | huCD20-AT2 | 32196 | 41573 | CD33 | AT264-S30G | 32206 | 41583 |
| BCMA | E59D-USC1 | 32197 | 41574 | CD33 | 264-V50A | 32207 | 41584 |
| BCMA | huE59D-AT2 | 32198 | 41575 | MSLN | AT2-K65R | 32208 | 41585 |
| CD19 | hA773-S58T | 32199 | 41576 | CD20 | hA253-S31N | 32209 | 41586 |
| CD19 | AT131-S58T | 32200 | 41577 | BCMA | S59Y-x-E59D | 32210 | 41587 |
| CD19 | CD19-083 | 32201 | 41578 | BCMA | E59DxS59Y | 32211 | 41588 |
| CD19 | AT83-N29S | 32202 | 41579 | BCMA | 355-S59Y | 32212 | 41589 |
| CD19 | CD19-083 | 32203 | 41580 | BCMA | BCMA-E59D | 32213 | 41590 |

TABLE 6

| | | SEQ ID | SEQ ID | | | SEQ ID | SEQ |
|---|---|---|---|---|---|---|---|
| Target | Name of | NO | NO | Target | Name of | NO | ID NO |
| Antigen | fragment | (DNA) | (PRT) | Antigen | fragment | (DNA) | (PRT) |
| Her2 | DARPIN-1 | 435 | 8815 | ULBP2R | NKG2D-AF | 445 | 8825 |
| BCMA | BCMA- | 436 | 8816 | ULBP2-S3 | NKG2D-YA | 446 | 8826 |
| | Centyrin | | | | | | |
| Ig-Fc | CD16A-V158- | 437 | 8817 | ULBP2R | NKG2D-AF- | 447 | 8827 |
| | ECD-v1 | | | | NKG2D-AF | | |
| NKG2 | CD8SP- | 438 | 8818 | ULBP2-S3 | NKG2D- | 448 | 8828 |
| D-L | NKG2D-GS- | | | | YA- | | |
| | NKG2D | | | | NKG2D-YA | | |
| MPL | mTPO(1-187) | 439 | 8819 | NKG2D-AF | ULBP2R | 449 | 8829 |
| BCMA | FHVH93- | 440 | 8820 | NKG2D-YA | ULBP2-S3 | 450 | 8830 |
| | ULBP2R | | | | | | |
| Ezip | RZIP | 441 | 8821 | K4 | E4 | 443 | 8823 |
| Rzip | EZIP | 442 | 8822 | E4 | K4 | 444 | 8824 |

TABLE 7
Peptide/MHC complexes targeted by SAR (uTCR-SAR)

| SEQ ID | | | |
|---|---|---|---|
| NO | | | |
| (PRT) | Name of peptide | SEQUENCE | MHC/HLA |
| 22780 | gp100 | IMDQVPFSV | HLA-A*02:01 |
| 22781 | gp100 | YLEPGPVTV | HLA-A*02:01 |
| 22782 | gp100 | KTWGQYWQV | HLA-A*02:01 |
| 22783 | MUC1-A7(130-138) | NLTISDVSV | HLA-A*02:01 |
| 22784 | MUC1-D6(13-21) | LLLTVLTVV | HLA-A*02:01 |
| 22785 | TAX(11-19) | LLFGYPVYV | HLA-A*02:01 |
| 22786 | hTERT(540-548) | ILAKFLHWL | HLA-A*02:01 |
| 22787 | hTERT(865-873) | RLVDDFLLV | HLA-A*02:01 |
| 22788 | HIV 1 gag(77-85) | SLYNTVATL | HLA-A*02:01 |
| 22789 | CMV-pp65(495-503) | NLVPMVATV | HLA-A*02:01 |
| 22790 | MART(26-35) | EAAGIGILTV | HLA-A*02:01 |
| 22791 | EBNA-3A(596-604) | SVRDRLARL | HLA-A*02:01 |
| 22792 | EBNA-3c | LLDFVRFMGV | HLA-A*02:01 |
| 22793 | PR1 | VLQELNVTV | HLA-A*02:01 |
| 22794 | Ras9-G12V | LVVVGAVGV | HLA-A*02:01 |
| 22795 | KRAS-7-16 | VVVGADGVGK | HLA-A*11:01 |
| 22796 | Ras9-G12V | LVVVGAGGV | HLA-A*02:01 |
| 22797 | NY-ESO-1-(155-163) | QLSLLMWIT | HLA-A*02:01 |
| 22798 | NY-ESO-1-(157-165) | SLLMWITQC | HLA-A*02:01 |
| 22799 | NY-ESO-1-(157-167) | SLLMWITQCFL | HLA-A*02:01 |
| 22800 | Mesothelin | KLLGPHVEGL | HLA-A*02:01 |
| 22801 | AFP-158 | FMNKFIYEI | HLA-A*02:01 |
| 22802 | MAGE-A3-1271-279 | FLWGPRALV | HLA-A*02:01 |
| 22803 | MAGE-A3- 112-220 | KVAEL VHFL | HLA-A*02:01 |
| 22804 | MAGE-A4-230-239 | GVYDGREHTV | HLA-A*02:01 |
| 22805 | WT1-37-45 | VLDFAPPGA | HLA-A*02:01 |
| 22806 | WT1-126-134 | RMFPNAPYL | HLA-A*02:01 |
| 22807 | HPV16-E6-29-38 | TIHDIILECV | HLA-A*02:01 |
| 22808 | HPV16-E7 | YMLDLQPET | HLA-A*02:01 |
| 22809 | EGFR-T790M | IMQLMPFGC | HLA-A*02:01 |
| 22810 | Human-HA1-H 153-161 | VLHDDLLEA | HLA-A*02:01 |
| 22811 | MyD88-L265P-264-273 | RPIPIKYKAM | HLA-B*07:02 |
| 22812 | EBV-BMLF1-259-267 | GLCTLVAM | HLA-A*02:01 |
| 22813 | EBV-LMP2-426-434 | CLGGLLTMV | HLA-A*02:01 |
| 22814 | Influenza-A-M1-58-66 | GILGFVFTL | HLA-A*02:01 |
| 22815 | human-GAD65-114-122 | VMNILLQYV | HLA-A*02:01 |
| 22816 | SARS-COV-2-S-958-996 | ALNTL VKQL | HLA-A*02:01 |
| 22817 | p53-R248Q-245-254 | GMNQRPILTI | HLA-A*02:01 |
| 22818 | p53-R175H-168-176 | HMTEVVRHC | HLA-A*02:01 |
| 40581 | PRAME | VLDGLDVLL | HLA-A*02:01 |

TABLE 8
Light chain and heavy chain CDR1-3 for select novel antigen binding domains

| | | vL- | vL- | vL- | vH- | vH- | vH- |
|---|---|---|---|---|---|---|---|
| Target Ag | vL/vH/scFv name | CDR1 | CDR2 | CDR3 | CDR1 | CDR2 | CDR3 |
| GD2 | GD2-hu3F8-USC1 | 41807 | 42078 | 42349 | 42620 | 42891 | 43162 |
| GD2 | hu3F8-USC2 | 41808 | 42079 | 42350 | 42621 | 42892 | 43163 |
| GD2 | hu-KM666-USC1 | 41809 | 42080 | 42351 | 42622 | 42893 | 43164 |
| GD2 | GD2-mu-KM666 | 41810 | 42081 | 42352 | 42623 | 42894 | 43165 |
| CLDN18.2 | huCLD18A2- | 41811 | 42082 | 42353 | 42624 | 42895 | 43166 |
| | 175D10-USC1 | | | | | | |
| CLDN18.2 | huCLD18A2- | 41812 | 42083 | 42354 | 42625 | 42896 | 43167 |
| | 175D10-USC2 | | | | | | |
| CLDN6 | hu-CLDN6-BNT- | 41813 | 42084 | 42355 | 42626 | 42897 | 43168 |
| | USC1 | | | | | | |
| CLDN6 | CLDN6-BNT | 41814 | 42085 | 42356 | 42627 | 42898 | 43169 |
| CLDN6 | CLDN6-BNT- | 41815 | 42086 | 42357 | 42628 | 42899 | 43170 |
| | USC1 | | | | | | |
| CLDN6 | CLDN6-G51A- | 41816 | 42087 | 42358 | 42629 | 42900 | 43171 |
| | AT1 | | | | | | |
| CD19 | hu-mROO5-1- | 41817 | 42088 | 42359 | 42630 | 42901 | 43172 |
| | A28T-AT1 | | | | | | |
| CD19 | hu-mROO5-1- | 41818 | 42089 | 42360 | 42631 | 42902 | 43173 |
| | Y32S-AT2 | | | | | | |
| CD19 | hu-CD19-USC3- | 41819 | 42090 | 42361 | 42632 | 42903 | 43174 |
| | AT1 | | | | | | |
| CD19 | hu-CD19-USC3- | 41820 | 42091 | 42362 | 42633 | 42904 | 43175 |
| | AT2 | | | | | | |
| CD79b | huMA79b-AT1 | 41821 | 42092 | 42363 | 42634 | 42905 | 43176 |
| CD79b | huMA79b-AT2 | 41822 | 42093 | 42364 | 42635 | 42906 | 43177 |
| CD79b | huMA79b-AT3 | 41823 | 42094 | 42365 | 42636 | 42907 | 43178 |
| CD79b | huCD79b-AT4 | 41824 | 42095 | 42366 | 42637 | 42908 | 43179 |
| CD79b | huCD79b-AT5 | 41825 | 42096 | 42367 | 42638 | 42909 | 43180 |
| CD79b | huCD79b-AT6 | 41826 | 42097 | 42368 | 42639 | 42910 | 43181 |
| CD79b | huMA79b-AT5 | 41827 | 42098 | 42369 | 42640 | 42911 | 43182 |
| CD79b | Hu-2F2-AT1 | 41828 | 42099 | 42370 | 42641 | 42912 | 43183 |
| CD79b | Hu-2F2-AT2 | 41829 | 42100 | 42371 | 42642 | 42913 | 43184 |
| CD79b | Hu-2F2-AT3 | 41830 | 42101 | 42372 | 42643 | 42914 | 43185 |
| CD79b | Hu-2F2-AT4 | 41831 | 42102 | 42373 | 42644 | 42915 | 43186 |
| CD79b | Hu-2F2-AT5 | 41832 | 42103 | 42374 | 42645 | 42916 | 43187 |
| CD79b | Hu-2F2-AT6 | 41833 | 42104 | 42375 | 42646 | 42917 | 43188 |
| CD79b | Hu-2F2-AT7 | 41834 | 42105 | 42376 | 42647 | 42918 | 43189 |
| CD79b | mu-MA79b | 41835 | 42106 | 42377 | 42648 | 42919 | 43190 |
| TAJ | KN5-USC1-R3D | 41836 | 42107 | 42378 | 42649 | 42920 | 43191 |
| GPRC5D | GPRC5D-Ro- | 41837 | 42108 | 42379 | 42650 | 42921 | 43192 |
| | 5F11-USC1 | | | | | | |
| GPRC5D | GPRC5D-Ro- | 41838 | 42109 | 42380 | 42651 | 42922 | 43193 |
| | 5E11-USC2 | | | | | | |
| GPRC5D | GPRC5D-Ro- | 41839 | 42110 | 42381 | 42652 | 42923 | 43194 |
| | 5F11-E11-USC | | | | | | |
| GPRC5D | GPRC5D-Ro- | 41840 | 42111 | 42382 | 42653 | 42924 | 43195 |
| | 5E11-5F11-USC | | | | | | |
| GPRC5D | GPRC5D-JJ-5B83- | 41841 | 42112 | 42383 | 42654 | 42925 | 43196 |
| | USC3 | | | | | | |
| FCRH5 | FCRH5-GN-USC2 | 41842 | 42113 | 42384 | 42655 | 42926 | 43197 |
| FR1 | huMov19-USC2 | 41843 | 42114 | 42385 | 42656 | 42927 | 43198 |
| p53- | p53-R175H | 41844 | 42115 | 42386 | 42657 | 42928 | 43199 |
| R175H | | | | | | | |
| p53- | p53-R175H-AT1 | 41845 | 42116 | 42387 | 42658 | 42929 | 43200 |
| R175H | | | | | | | |
| p53- | R248Q-336-USC1 | 41846 | 42117 | 42388 | 42659 | 42930 | 43201 |
| R248Q | | | | | | | |
| Muc16 | huMuc16-3A5 | 41847 | 42118 | 42389 | 42660 | 42931 | 43202 |
| ROR1 | ROR1-JJ-67 | 41848 | 42119 | 42390 | 42661 | 42932 | 43203 |
| ROR1 | ROR1-JJ-67-AT1 | 41849 | 42120 | 42391 | 42662 | 42933 | 43204 |
| DLL3 | hSC16-57-USC1 | 41850 | 42121 | 42392 | 42663 | 42934 | 43205 |
| NPM1c | NPM1c-YG1 | 41851 | 42122 | 42393 | 42664 | 42935 | 43206 |
| IL23R | IL23R-SANG-11 | 41852 | 42123 | 42394 | 42665 | 42936 | 43207 |
| HLA-A2 | HLA-A2-SANG76 | 41853 | 42124 | 42395 | 42666 | 42937 | 43208 |
| MOG01 | MOG01-USC1 | 41854 | 42125 | 42396 | 42667 | 42938 | 43209 |
| MOG301 | Y56N-USC2 | 41855 | 42126 | 42397 | 42668 | 42939 | 43210 |
| MOG | MOG473-USC3 | 41856 | 42127 | 42398 | 42669 | 42940 | 43211 |
| MOG | MOG01 | 41857 | 42128 | 42399 | 42670 | 42941 | 43212 |
| MOG | MOG301 | 41858 | 42129 | 42400 | 42671 | 42942 | 43213 |
| MOG | MOG-AZ17 | 41859 | 42130 | 42401 | 42672 | 42943 | 43214 |
| CSF1R | CSF1R-AXI | 41860 | 42131 | 42402 | 42673 | 42944 | 43215 |
| CSF1R | CSF1R-AXI- | 41861 | 42132 | 42403 | 42674 | 42945 | 43216 |
| | USC1 | | | | | | |
| CD70 | P08F09-USC1 | 21011 | 21046 | 21081 | 21116 | 21151 | 21186 |
| STEAP2 | STEAP2-RGN- | 21015 | 21050 | 21085 | 21120 | 21155 | 21190 |
| | 1162-HL-USC1 | | | | | | |

TABLE 9
Examples of diseases targeted by SARs.

| | EXAMPLES OF DISEASE TARGETED BY SARs (e.g., CD16 |
|---|---|
| SAR “X” | SAR, NKp30 SAR, NKp44 SAR, NKp46 SAR and DAP10 SAR |
| TARGET | etc.) |
| CD19 | ALL, CLL, lymphoma, lymphoid blast crisis of CML, multiple |
| | myeloma, immune disorders, lupus |
| ALK | Non-small Cell Lung Cancer (NSCLC), ALCL or neuroblastoma |
| CD45 | Blood cancers |
| BCMA | Myeloma, PEL, plasma cell leukemia, Waldenstrom's |
| | macroglobinemia, lupus, |
| CD5 | Blood cancer, T cell leukemia, T cell lymphoma |
| CD20 | Blood cancers, Leukemia, ALL, CLL, lymphoma, immune disorders |
| CD22 | Blood cancers, Leukemia, ALL, CLL, lymphoma, lymphoid blast crisis |
| | of CML, immune disorders |
| CD23 | Blood cancers, Leukemia, ALL, CLL, lymphoma, autoimmune |
| | disorders (lupus) |
| CD30 | Hodgkins's lymphoma, Cutaneous T cell lymphoma |
| CD32 | Solid tumors |
| CD33 or | Blood cancers, AML, MDS |
| CD34 | |
| CD70 | Blood cancers, Waldenstrom's macroglobulinemia, Kidney cancer |
| CD79b | Blood cancers, ALL, Lymphoma, autoimmune disorder (lupus) |
| CD123 | Blood cancers, AML, MDS |
| CD138 | Blood cancers, Myeloma, PEL, plasma cell leukemia, Waldenström's |
| | macroglobulinemia |
| CD179b | Blood cancers, ALL, Lymphoma |
| CD276/B7-H3 | Ewing's sarcoma, neuroblastoma, rhabdomyosarcoma, ovarian cancer |
| CD324 | Solid tumors, esophageal, prostate, colorectal, breast, lung cancers |
| CDH6 | Solid tumors, renal, ovarian, thyroid cancers |
| CDH17 | Adenocarcinomas, gastrointestinal, lung, ovarian, endometrial cancers |
| CDH19 | Solid tumor, Melanoma |
| EGFR | Colon cancer, lung cancer |
| CLEC5A | Blood cancers, Leukemia, AML |
| GR/LHR | Prostate cancer, ovarian cancer, or breast cancer |
| CLL1 | Blood cancer, Leukemia |
| CS1 | Blood cancers, myeloma, PEL, plasma cell leukemia |
| CSF2RA | AML, CML, MDS |
| CD123 | Blood cancers, AML, MDS |
| DLL3 | Melanoma, lung cancer or ovarian cancer |
| EGFR | Solid tumors, Colon cancer, lung cancer |
| EGFRvIII | Solid tumors, glioblastoma |
| EpCam1 | Gastrointestinal cancer |
| FLT3 | Blood cancers, AML, MDS, ALL |
| Folate Receptor | Ovarian cancer, NSCLC, endometrial cancer, renal cancer, or other |
| alpha | solid tumors |
| FSHR | Prostate cancer, ovarian cancer, or breast cancer |
| GD2 | Neuroblastoma |
| GD3 | Melanoma |
| GFRa4 | Cancer, thyroid medullary cancer |
| Fucosyl- | Small cell lung cancer |
| GM1(GM1) | |
| GPRC5D | Myeloma, PEL, plasma cell leukemia, waldenstrom's |
| | macroglobulinemia, autoimmune disorder (lupus) |
| gp100 | Melanoma |
| GPC3 | Solid tumors, Lung cancer |
| gpNMB | Melanoma, brain tumors, gastric cancers |
| GRP78 | Myeloma |
| Her2 | Solid tumors, breast cancer, stomach cancer |
| Her3 | Colorectal, breast cancer |
| IL11Ra | Blood cancers, AML, ALL, CML, MDS, sarcomas |
| IL6Ra | Solid tumors, Liver cancer |
| IL13Ra2 | Glioblastomas |
| LAMP1 | Blood cancers, AML, ALL, MDS, CLL, CML |
| LewisY | Cancers |
| L1CAM | Solid tumors, ovarian, breast, endometrial cancers, melanoma |
| LHR | Prostate cancer, ovarian cancer, or breast cancer |
| Lym1 | Blood cancer, Leukemia, Lymphoma |
| Lym2 | Blood cancer, Leukemia, Lymphoma |
| MART1/MHC I | Melanoma |
| Mesothelin | Mesothelioma, ovarian cancer, pancreatic cancer |
| Muc1/MHC I | Breast cancer, gastric cancer, colorectal cancer, lung cancer |
| Muc16 | Ovarian cancer |
| NKG2D | Leukemia, lymphoma, or myeloma |
| NYBR1 | Breast cancer |
| PSCA | Prostate cancer |
| PR1/MHC I | Blood cancer, Leukemia |
| Prolactin | Breast cancer, chromophobe renal cell cancer |
| Receptor | |
| PSMA | Prostate cancer |
| PTK7 | Melanoma, lung cancer or ovarian cancer |
| ROR1 | Blood cancer, B cell malignancy, lymphoma, CLL |
| SLea | Pancreatic cancer, colon cancer |
| SSEA4 | Pancreatic cancer |
| Tyrosinase | Melanoma |
| TCRB1 | T cell leukemias and lymphomas, autoimmune disorders |
| TCRB2 | T cell leukemias and lymphomas, autoimmune disorders |
| TCRgd | T cell leukemias and lymphomas, autoimmune disorders |
| hTERT | Solid tumors, blood cancers |
| TGFBR2 | Solid tumors, keloid |
| TIM1/HAVCR1 | Kidney cancer, liver cancer |
| TROP2 | Solid tumors, Breast cancer, prostate cancer |
| TSHR | Thyroid cancer, T cell leukemia, T cell Lymphoma |
| TSLPR | Blood cancers, Leukemias, AML, MDS |
| WT1/MHC I | Blood cancers, AML |
| Folate Receptorβ | AML, Myeloma |
| B7H4 | Breast cancer or ovarian cancer |
| GCC | Gastrointestinal cancer |
| CD200R | Blood cancers, AML, MDS |
| AFP/MHC I | Solid tumors, Liver cancer |
| CD99 | Liver cancer |
| GPRC5D | Myeloma, Waldenström's macroglobulinemia |
| HPV16- | HPV16 associated cancers, cervical cancer, head and neck cancers |
| E7/MHC I | |
| Tn-Muc1 | Solid tumors and blood cancers |
| Igk-Light Chain | Myeloma, plasma cell leukemia |
| CLD18A2 | Gastric, pancreatic, esophageal, ovarian, or lung cancer |
| (Claudin 18.2) | |
| CD43 | Blood cancers, AML |
| NY-ESO- | Myeloma |
| 1/MHC I | |
| MPL/TPO-R | Blood cancer, AML, MDS, CML, ALL, Myeloproliferative disorders, |
| | Polycythemia vera, Myelofibrosis, Essential Polycythemia |
| STEAP1 | Gastric or prostate cancer, or lymphoma |
| Liv1 (SLC39A6) | Breast or prostate cancer |
| Nectin4 | Bladder, renal, cervical, lung, head and neck or breast cancer |
| (PVRL4) | |
| Cripto (TDGF1) | Colorectal or endometrial or ovarian cancer |
| gpA33 | Colorectal or endometrial or ovarian cancer |
| FLT3 | Blood cancers, AML, ALL, MDS |
| BST1/CD157 | Blood cancers, AML, MDS |
| IL1RAP | Liver, colorectal, cervical, lung or ovarian cancer |
| Chloride channel | Glioma |
| IgE | Allergy |
| HLA-A2 | Graft vs host disease, tissue rejection (SIR Expressed in regulatory T |
| | cells) |
| Amyloid | Amyloidosis, Alzheimer's disease |
| HIV1-env | HIVI/AIDS and related conditions |
| CLDN6 | Ovarian cancer, cervical cancer, gastrointestinal cancer |
| STEAP2 | Prostate cancer |

TABLE 9B

| | SEQ | SEQ | | SEQ | |
|---|---|---|---|---|---|
| | ID | ID | FRAGMENT | ID | SEQ ID |
| FRAGMENT NAME | DNA | PRT | NAME | DNA | PRT |
| TCRa-Connecting-Peptide- | 615 | 8995 | TCRa-TM | 625 | 9005 |
| MD | | | | | |
| TCRa-Connecting-Peptide | 616 | 8996 | TCRb-TM | 626 | 9006 |
| TCRb-Connecting- | 617 | 8997 | TCRg-TM | 627 | 9007 |
| Peptide-MD | | | | | |
| TCRb-Connecting-Peptide | 618 | 8998 | TCRd-TM | 628 | 9008 |
| TCRg-Connecting- | 619 | 8999 | CD3z-TM | 629 | 9009 |
| Peptide-MD | | | | | |
| TCRg-Connecting-Peptide | 620 | 9000 | TCRa-CP | 631 | 9011 |
| TCRd-Connecting- | 621 | 9001 | TCRb-CP | 632 | 9012 |
| Peptide-MD | | | | | |
| TCRd-Connecting-Peptide | 622 | 9002 | TCRg-CP | 633 | 9013 |
| CD3z-connecting-peptide | 623 | 9003 | CD3z-CP | 634 | 9014 |
| (hinge) | | | | | |
| CD16A-F158V-Hinge-v1 | 862 | 9242 | CD8b-CP | 647 | 9027 |
| CD16A-F158V-TM-v1 | 764 | 9144 | CD8a-CP | 648 | 9028 |
| CD16A-F158V-cP-v1 | 765 | 9145 | CD4-CP | 649 | 9029 |
| CD8-Hinge-opt1 | 748 | 9128 | CD28-CP | 502 | 8882 |
| CD3zECDTMCP-opt | 500 | 8880 | 41BB-CP | 503 | 8883 |
| CD3zECDTMCP-opt2 | 507 | 8887 | FcRy-CP-opt1 | 877 | 9257 |
| hTCR-alpha-constant | 453 | 8836 | hTCR-b1-constant | 467 | 8847 |
| hTCRa-WT | 454 | 8838 | hTCR-b2-constant | 468 | 8848 |
| hTCRa-opt2 | 456 | 8835 | hTCRb-WT | 469 | 8849 |
| hTCRa-T48C-opt | 458 | 8835 | hTCRb-S57C-opt1 | 470 | 8850 |
| hTCRa-CSDVP | 455 | 8833 | hTCRb-KACIAH | 471 | 8851 |
| hTCRa-Med12 | 32218 | 40535 | hTCRb-opt2 | 472 | 8852 |
| TCRa-MED17 | 32219 | 40536 | TCRb-opt-Med13 | 40537 | 32220 |
| mTCRa-opt | 465 | 8845 | mTCRb-opt | 486 | 8866 |
| hTCR-gamma | 492 | 8872 | hTCR-Delta | 494 | 8874 |
| hTCR-Gamma-Opt | 493 | 8873 | hTCR-Delta-Opt | 495 | 8875 |
| mTCRg | 32222 | 40539 | hTCR-Delta | 494 | 8869 |
| mTCRd | 32221 | 40538 | | | |

Example diseases targeted by the SARs targeting different antigens are provided in PCT/2020/014237.

Patent application PCT/US22/17177 described a novel SAR design designated called a universal TCR-SAR (or uTCR-SAR), that confers T cell receptor like antigen binding specificity to any cell. In an embodiment, a uTCR-SAR comprises: a) a first polypeptide chain comprising a first antigen-binding domain and a first Membrane associated module (MAM); and b) a second polypeptide chain comprising a second antigen-binding domain and a second Membrane associated module (MAM), wherein the first antigen-binding domain and the second antigen-binding domain form a TCR-like (e.g., TCR-Fv) antigen-binding module that specifically binds to the target antigen, and wherein the first MAM and the second MAM form a non-T cell receptor module (NTCRM). In some embodiments, the first and the second MAM of a uTCR-SAR comprises of a transmembrane or membrane associated domain of a signaling adaptor. In an embodiment, the signaling adaptor is selected from, but not limited, to one or more of CD3ζ, FcRγ, DAP10 and/or DAP12 or variants or fragments thereof. In an embodiment, the MAM of a uTCR-SAR comprises of a non-TCR receptor (e.g., CD16).

The present disclosure provides a uTCR-SAR comprising the Vα (or Va or a) chain reference amino acid sequence of SEQ ID NO: 8685-8693, 40531-32 or a variant thereof and the Vβ (or Vb or b) chain reference amino acid sequence of SEQ ID NO: 8702-8710, 40533-34 a variant thereof, where the uTCR-SAR targets an antigen shown in Tables 4 and 7. The SEQ ID NOs of the example peptide antigens listed in Table 4 are provided in Table 7. A variant may have an amino acid sequence having at least 75%, at least 80%, at least 85%, at least 90%, at least 95%, at least 98% or at least 99% sequence identity to the reference amino acid sequence (for example, with respect to either a chain reference sequence and/or b chain reference sequence). The uTCR-SAR may be encoded by the α (or a) chain reference nucleotide sequence of SEQ ID NO: 305-313 or a variant thereof and the β (or b) chain reference nucleotide sequence of SEQ ID NO: 322-330 or a variant thereof. A variant may have a nucleotide sequence having at least 70%, at least 75%, at least 80%, at least 85%, at least 90%, at least 95%, at least 98% or at least 99% sequence identity to the reference nucleotide sequence (for example, with respect to either a chain reference sequence and/or b chain reference sequence).

According to the invention the uTCR-SAR may be an NY-ESO-1 uTCR-SAR which may comprise the Vα (or Va or a) chain reference amino acid sequence of SEQ ID NO: 8685 or a variant thereof and the Vβ (or Vb or b) chain reference amino acid sequence of SEQ NO: 8702 or a variant thereof. The NY-ESO-1 uTCR-SAR may be encoded by the α (or a) chain reference nucleotide sequence of SEQ ID NO: 305 or a variant thereof and the b chain reference nucleotide sequence of SEQ ID NO: 322 or a variant thereof. In an embodiment, the NY-ESO-1 uTCR-SAR binds to a peptide antigen (-SLLMWITQC- or -SLLMWITQCFL-) represented by SEQ ID NO: 22798-99, optionally in complex with HLA-A*02:01. Example uTCR-SAR targeting NY-ESO-1 peptide (SLLMWITQC or SLLMWITQCFL) and comprising different signaling chains are presented in SEQ ID NO:10621-10697 (Tables 11 and 12 the provisional patent application). In addition, nucleic acid and amino acid sequences of uTCR-SAR targeting NY-ESO-1 peptide in which one or both CD3z signaling chains (e.g., CD3zECDTMCP; e.g., SEQ ID NO: 8880) are replaced by a CD3z signaling chains comprising deletion of residue 101 (i.e., CD3zECDTMCP-dQ101; SEQ ID NO:9327) are presented in SEQ ID NO: 11567-11623 (Table 13). As uTCR-SAR are modular in format, the signaling chains can be replaced by different signaling chains (e.g., SEQ ID NO:9327-9340) or functional variants thereof to generate new uTCR-SAR with distinct properties. Similarly, the antigen binding domains (e.g. Vα, Vβ. Vγ, ∇6, vL, vH, vHH, svTCR, etc.) and linker domains of the example tuTCR-SAR constructs may be replaced by antigen binding domains targeting different antigens (e.g., MAGE-A3, MAGE-A4 etc.) or different linkers (e.g., Ig or Ig-like linkers) or functional variants thereof to generate new uTCR-SAR with distinct properties.

The disclosure provides uTCR-SAR targeting MAGGE-A4, WT-1, gp100, KRas, HPV-E6, HPV-E; 7 and PRAME. The Va and Vb fragments targeting these antigens are provided in Table 4. The nucleic acid and amino acid sequence of example uTCR-SAR targeting different peptide antigens and comprising different signaling chains are presented in Tables 11, 12 and 13 of the provisional patent application.

Patent application PCT/US22/17177 (incorporated in its entirety by reference herein) describes single and double chain Synthetic Antigen Receptors based on CD16 (CD16a and CD16b) isoforms. The current invention describes CD16 based SAR comprising mutant CD16 chains. In an embodiment, the mutant CD16 chains of the CD16-SAR of the present disclosure comprise deletion in the CD16 cytoplasmic domain. In an embodiment, the mutant CD16 chains of the CD16-SAR of the present disclosure lack the CD16 cytoplasmic domain. In an embodiment, the mutant CD16 chains of the CD16-SAR of the present disclosure comprise mutation in the CD16 transmembrane domain. In an embodiment, the mutant CD16 chains of the CD16-SAR of the present disclosure lacks the full length CD16.

The nucleic acid and amino acid sequences of connecting peptide, hinge, transmembrane (TM), cytoplasmic (CP) domains of CD16, CD3z, FcRy chains and coreceptors and costimulatory receptors that can be used in the construction of a SAR are provided in Table 9B. The SEQ ID Nos of different Ig linkers and TCR constant domains are provided in the provisional patent application. Example CD16 chains with deleted and mutated cytoplasmic domains that can be used in the construction of SAR are provided in SEQ ID NO (PRT): 8945-8948. In an embodiment, the SAR can be constructed using CD16 chains with at least 70% amino acid sequence identity to SEQ ID NO (PRT): 8945-8948 or functional variants thereof. Example SARs with deleted and mutated cytoplasmic domains are presented in SEQ ID NO (DNA): 1111-2234 and SEQ ID NO (PRT): 9491-10614 (see also Tables 9-10 of the provisional patent application). In an embodiment, the SAR can be constructed in which one or more antigen binding domains are attached to N-terminus or near the N-terminus of entire or partial extracellular domain of CD16 chains represented by SEQ ID NO (PRT): 8945-8948 or functional variants thereof.

The disclosure provides, single chain, double chain and double chain hetero-dimeric SARs comprising the partial or entire region of CD16 (FcγRIII). The disclosure provides SARs comprising CD16 or fragments thereof that have at least 70% identity to any of the CD16 sequences described herein (SEQ ID NO: 8945-8948) while retaining the biological activity. Example mutant CD16 nucleic acid and amino acid sequences that can be used in the construction of CD16-SARs of the disclosure are provided in SEQ ID NO (DNA): 565-568 and SEQ ID NO (PRT): 8945-8948 or equivalent residues (i.e., a homolog) from a non-human species, e.g., mouse, rodent, monkey, ape, and the like.

In some embodiments, the CD16 sequences that can be used in the construction of the CD16 SARs of the disclosure may include mutants and variants that increase the affinity of CD16 for immunoglobulin Fc region (e.g., CD16A-F158V) and, in addition, prevent its cleavage from cell surface (e.g., CD16A-F158V-S197P).

In certain embodiments, the nucleic acid sequence of the SAR molecule comprises the nucleic acid sequence of human CD16 as shown in SEQ ID NO: 565-568. In certain embodiments, the nucleotide sequence of the SAR comprises sequence that encodes for amino acid sequence of CD16 having at least one, five or ten modifications but not more than 20 modifications of an amino acid sequence of SEQ ID NO: 8945-8948, or a sequence with 70-99% homology to an amino acid sequence of SEQ ID NO: 8945-8948. In certain embodiments, SAR molecule comprises the amino acid sequence of SEQ ID NO: 8945-8948 or equivalent residues from a non-human species.

In an embodiment, the disclosure provides a single chain CD16 SAR comprising the partial or entire region of CD16 or a variant thereof. In an embodiment, the disclosure provides a single chain CD16 SAR comprising a partial or entire region of CD16 extracellular domain. Example CD16 extracellular domain sequences that can be used in the construction of a CD16-SAR of the disclosure are provided in SEQ ID NO (DNA): 759-761 and SEQ ID NO (PRT): 9139-9141 or the equivalent residues (i.e., a homolog) from a non-human species. In an embodiment, the disclosure provides a CD16 SAR comprising the partial or entire region of CD16 hinge domain. Example CD16 hinge domain sequences that can be used in the construction of a CD16-SAR of the disclosure are provided in SEQ ID NO (DNA):763 and SEQ ID NO (PRT): 9143 or the equivalent residues (i.e., a homolog) from a non-human species. In an embodiment, the disclosure provides a CD16 SAR comprising the partial or entire region of CD16 transmembrane domain. Example CD16 transmembrane sequences that can be used in the construction of CD16-SARs of the disclosure are provided in SEQ ID NO (DNA): 764 and SEQ ID NO (PRT): 9144 or the equivalent residues (i.e., a homolog) from a non-human species. In an embodiment, the disclosure provides a CD16 SAR comprising a partial or entire region of CD16 cytosolic domain. The disclosure also provides SARs comprising variants of CD16 or fragments thereof that retain at least one biological activity of the wild-type CD16 to which it has identity or homology.

In an embodiment, the CD16 SAR comprises the CD16 extracellular domain comprising both immunoglobulin-like domains (i.e., D1 and D2) that is attached via the CD16 hinge domain to CD16 transmembrane domain. In an embodiment, the CD16 transmembrane domain comprises a mutation. In an embodiment, the CD16 transmembrane domain comprises a S213Y mutation. An example such CD16 SAR targeting CD19 is represented by CD8SP-CD19-hu-mROO5-1-(vL-vH)-CD16A-F158V-S197P-v3-L639-S213Y (SEQ ID NO(DNA): 2057 and SEQ ID NO (PRT): 10437. Additional example such SARs comprising scFv, FHVH, vHH and non-immunoglobulin antigen binding scaffolds targeting different antigens are provided in SEQ ID NO (DNA): 1954-2234 and SEQ ID NO PRT): 10334-10614. Such a CD16 SAR also retains the ability to bind to the Fc region of an antibody, an antibody fragment or bispecific/tri-specific engager and mediate antibody dependent cytotoxicity. Thus, immune cells (e.g., T cells, NK cells, monocytes/macrophages, neutrophils etc.) expressing the SAR CD8SP-CD19-hu-mROO5-1-(vL-vH)-CD16A-F158V-S197P-v3-L639-S213Y can target CD19 expressing target cells through CD19-hu-mROO5-1 scFv region. In addition, such immune cells can be redirected to targeted Her2 expressing target cells in the presence of Herceptin. Alternatively, such immune cells (e.g., T or NK cells) can be redirected to targeted CD20 expressing target cells in the presence of Rituximab.

In an embodiment, the CD16 SAR comprises the CD16 extracellular domain comprising both immunoglobulin-like domains (i.e., D1 and D2) that is attached via the CD16 hinge domain to CD16 transmembrane domain but lacks a complete CD16 cytosolic domain. An example such a SAR is represented by CD8SP-CD19-hu-mROO5-1-(vL-vH)-CD16A-F158V-S197P-v3-L639 (SEQ ID NO (DNA): 1495 and SEQ ID NO (PRT): 9875. Additional example such CD16-based SARs targeting different antigens are provided in SEQ ID NO (DNA): 1111-1953 and SEQ ID NO (PRT): 9491-10333. Such CD16-based SARs also retain the ability to bind to the Fc region of an antibody, an antibody fragment or bispecific/tri-specific engager and mediate antibody dependent cytotoxicity. Thus, immune cells (e.g., T cells, NK cells, monocytes/macrophages, neutrophils etc.) expressing such a SAR can target cells through their exogenous antigen binding domains (e.g., scFv, vHH, FHVH etc.). In addition, such immune cells can be redirected to targeted Her2 expressing target cells in the presence of Herceptin. Alternatively, such immune cells (e.g., T or NK cells) can be redirected to targeted CD20 expressing target cells in the presence of Rituximab.

In an embodiment, the CD16 SAR comprises the partial or entire CD16 hinge domain that is attached to CD16 transmembrane domain. The CD16 transmembrane domain may comprise a mutation (e.g., S213Y). Such a CD16-SAR lacks the ability to bind to an antibody as it lacks both the D1 and D2 domains. In an embodiment, the CD16-based SAR comprises a heterologous hinge (spacer) domain that is present between the antigen binding domain (e.g., scFv, or AABD) and the hinge domain of CD16.

In an embodiment, the CD16 SAR comprises an AABD (e.g., a vHH, FHVH, chVH, centyrin, affibody etc.) that is inserted between the D2 domain and the hinge domain of CD16 with optional intervening linkers (e.g., Gly4-Ser linker). In an example embodiment, the different domains of such a CD16 SAR from amino to carboxy-terminal include an N-terminal signal peptide, CD16-D1 domain, CD16-D2 domain, optional linker, AABD (e.g., vHH, FHVH, centyrin, affibody etc.), optional linker, CD16-hinge domain, and CD16-transmembrane domain.

It is to be understood that the different CD16 domains (i.e., extracellular, D1, D2, hinge and transmembrane) that may be used in the construction of the SAR may comprise their entire sequence or a deletion mutant or a variant as long as the domain retains at least one of its functional properties. The CD16 domains may comprise their wild-type sequence or one or more of the high affinity (e.g., F158V) or high affinity non-cleavable (e.g., F158V/S197P or F158V/S197R) variants.

In an embodiment, the antigen binding domain of the CD16 SAR comprises a scFv, a vL, vH, Fv, Va, Vb, Vg, Vd, TCR-Fv, vHH, FHVH, a single domain antibody, a single chain TCR (scTCR), a single variable domain TCR (svd-TCR), a non-immunoglobulin antigen binding scaffold, a ligand (e.g., APRIL) or the extracellular domain of a receptor (e.g., PD1, NKG2D, NKp30, NKp44, NKp46 etc.). The chain of a single chain SAR may bind to one antigen or more than one antigen (e.g., two, three, four etc.). The chain of a single chain CD16 SAR may further comprise one or more adaptors (e.g., RZIP, EZIP, NKG2D-YA etc.).

In some embodiments, the CD16 SAR of the disclosure comprises a molecule of the general formula:
AABD(n)-optional CD16 D1 domain-optional CD16 linker domain-optional-CD16 D2 domain, CD16 hinge domain-CD16 transmembrane domain. In one embodiment, n is at least 2, for example 2, 3, 4 or 5. The AABD (autonomous antigen binding domain) forms the antigen binding domain and is located at the extracellular side when expressed in a cell.

In some embodiments, the CD16 SAR of the disclosure comprises a molecule of the general formula:
scFv(n)-optional CD16 D1-optional CD16 linker domain-optional-CD16 D2 domain, CD16 hinge domain-CD16 transmembrane domain, wherein n is 1 or more.

The nucleic acid and amino acid sequences of SARs comprising the CD16A-F158V-S197P-v3-L642 mutant in fusion with the different antigen binding domains targeting different antigens are presented in TABLE 9 of the provisional patent application. The nucleic acid and amino acid sequences of SARs comprising the other CD16 mutants in fusion with the different antigen binding domains targeting different antigens are presented in TABLE 10 of the provisional patent application. In an embodiment, an example SAR comprises the CD16 mutant chain sequence and comprising a vHH fragment or a FHVH fragment attached to an scFv targeting CD19. Example SARs comprising the CD16 mutant chain sequence and comprising an adaptor (SEQ ID NO: 1381-87) or a scTCR are also provided.

The nucleic acid and amino acid sequences of example SARs comprising the mutant CD16A in fusion with vHH and FHVH fragments targeting different antigens are represented by SEQ ID NO (DNA): 1300-1368 and SEQ ID NO (PRT): 9680-9748, respectively.

The nucleic acid and amino acid sequences of example SARs comprising the mutant CD16A in fusion with non-immunoglobulin antigen binding domains (e.g., DARPIN, Centyrin, affibodies), receptor extracellular domains (e.g., NKG2D), ligands/cytokines (e.g., TPO), adaptors (e.g., EZIP, K4, ULBP2R and ULBP2-S3 etc.) targeting different antigens are represented in Table 9 of the provisional patent application by SEQ ID NO (DNA):1369-1387 and SEQ ID NO (PRT): 9747-9767, respectively.

T cells expressing a single chain CD16-based SAR with mutant CD16 chain when exposed to a cell expressing the cognate target antigen can activate NFAT signaling, induce IL2 production, promote T cell proliferation, promote T cell activation, and exert cytotoxicity. In another example embodiment, NK cells expressing a single chain CD16-SAR with mutant CD16 chain when exposed to a cell expressing the cognate target antigen can induce IL2 production, promote NK cell proliferation, promote NK cell activation, or exert cytotoxicity. In another example embodiment, monocytes/macrophages expressing a single chain CD16-SAR with mutant CD16 chain when exposed to a cell expressing the cognate target antigen can induce phagocytosis of the target cells. In another example embodiment, granulocytes (e.g., neutrophils) expressing a single chain CD16-SAR with mutant CD16 chain when exposed to a cell expressing the cognate target antigen can induce phagocytosis of the target cells.

In certain embodiments, the disclosure provides a novel platform of synthetic antigen receptors, designated CD16-SARs, containing two chains wherein at least one chain comprises the partial or the entire sequence of a mutant CD16 chain or a variant thereof. In certain embodiments, the disclosure provides a novel platform of synthetic antigen receptors, designated CD16-SAR, containing two chains wherein at least one chain comprises the CD16 hinge domain and the CD16 transmembrane domain but lacks a cytosolic domain. In certain embodiments, the disclosure provides a novel platform of synthetic antigen receptors, designated CD16-SAR, containing two chains wherein at least one chain comprises a CD16 transmembrane domain but lacks a cytosolic domain. In certain embodiments, the disclosure provides a novel platform of synthetic antigen receptors, designated CD16-SAR, containing two chains wherein at least one chain comprises a CD16 transmembrane domain with one or more mutations but lacks a cytosolic domain. In certain embodiments, the disclosure provides a novel platform of synthetic antigen receptors, designated CD16-SAR, containing two chains wherein at least one chain comprises a CD16 hinge and transmembrane domain but lacks a cytosolic domain. In certain embodiments, the disclosure provides a novel platform of synthetic antigen receptors, designated CD16-SAR, containing two chains wherein at least one chain comprises a CD16 hinge domain and a CD16 transmembrane domain with one or more mutations but lacks a cytosolic domain. Example double chain CD16 SARs are presented in SEQ ID NO (DNA): 1388-1391 and SEQ ID NO (PRT): 9768-9771. The sequences of additional example double chain CD16 SARs are presented in Table 10 of the provisional patent application.

In an embodiment, the disclosure provides a double chain CD16-based SARs where at least one chain comprises a partial or entire region of CD16 extracellular domain and comprise CD16 transmembrane domain with a mutation (e.g., S213Y mutation). In an embodiment, the disclosure provides a double chain CD16-based SARs where at least one chain comprises a partial or entire region of CD16 extracellular domain and comprises CD16 transmembrane domain but lacks a cytoplasmic domain. Example CD16 extracellular domain, hinge domain and transmembrane domain sequences that can be used in the construction of double chain CD16-SARs of the disclosure are provided in the preceding sections.

The disclosure provides a double chain CD16 based SAR in which the vL or vH fragment of an antibody is functionally joined to a first chain comprising a CD16 transmembrane domain and the complementary vH or vL fragment can be joined to second chain comprising transmembrane domain of a signaling adaptor (e.g., CD3z, FcRy, DAP10, DAP12 etc.). When the two such chains (e.g., vL-CD16 and vH-CD3zECDTMCP or vL-CD16 and vH-FceRy1) are co-expressed in the same cell, the vL and vH fragments can bind their cognate antigen and transmit a cell signal. It is to be noted that the vL and vH fragments of such a SAR are not able to bind to the antigen on their own in the absence of the complementary fragment. It is to be noted that the antigen binding of a CD16 based SAR described may comprise of variable domains derived from a TCR (e.g., Vα, Vβ, Vγ or Vδ) rather than the vL and vH fragments of an antibody.

In an embodiment, the CD16 chain may be a mutant chain (e.g., comprised of S213Y mutation). In an embodiment, the CD16 chain may lack a cytoplasmic domain or contains a partial cytoplasmic domain. In an embodiment, the CD16 chain may contain a partial cytoplasmic domain comprising no more than 1, 2, 3, 4, 5, 6, 7, 10, 15, or 20 amino acid residues. In an example embodiment, T cells expressing such CD16-SAR when exposed to a cell expressing the cognate target antigen can activate NFAT signaling, induce IL2 production, promote T cell proliferation, promote T cell activation, and exert cytotoxicity. In another example embodiment, NK cells expressing such CD16-SAR when exposed to a cell expressing the cognate target antigen can induce IL2 production, promote NK cell proliferation, promote NK cell activation, or exert cytotoxicity. In another example embodiment, monocytes/macrophages expressing such CD16-SAR when exposed to a cell expressing the cognate target antigen can induce phagocytosis of the target cells. In another example embodiment, monocytes/macrophages expressing a double chain CD16-SAR when exposed to a cell expressing the cognate target antigen can induce phagocytosis of the target cells. In another example embodiment, granulocytes expressing a double chain CD16-SAR when exposed to a cell expressing the cognate target antigen can induce phagocytosis of the target cells.

The expression and activity of the double chain SAR (e.g., CD16-SAR, zSIR, uTCR-CAR, zSAR, FceRγ1-SAR etc.) can be further increased by incorporation of a linker between the vL/vH, Vα/Vβ or Vγ/Vδ and the signaling adaptors (e.g., CD3z, FceRγ1 etc.) and/or signaling chain (e.g., CD16). In particular, the IgCL (SEQ ID NO: 8961) and Ig-CH1 domains (SEQ ID NO: 8962-8976) derived from antibodies serve as useful linkers between the vL/vH and signaling adaptors or signaling chain (e.g., CD16). Additional Ig-like domains are known in the art (SEQ ID NO (DNA): 597-614, 21451-21456 and SEQ ID NO (PRT):8977-8994, 21466-21471 and can serve as useful linkers in alternate embodiment of the disclosure.

In an embodiment, the linker comprises the Ig-like constant domain of a TCR chain and further comprises a TCR connecting peptide. Example such long Ig-like linkers are provided in SEQ ID NO: 22827-22829, 22833-22835, 22839-22840, 22843-22844, respectively. The long Ig-like linkers also include functional variants and homologs which encodes for polypeptide with at least 75% sequence identity to polypeptides encoded by the above sequences. In an embodiment, the long Ig-like linker comprises N-terminal or C-terminal deletion mutants of in SEQ ID NO (DNA): 22827-22829, 22833-22835, 22839-22840, 22843-22844, respectively, in which between 1-40 (e.g., 1, 5, 10, 15, 20, 25, 30, 40) N-terminal or C-terminal encoded amino acid residues are deleted.

In an embodiment, a double chain CD16-SAR comprises antigen binding domains (e.g., vL/vH, Vα/Vβ or Vγ/Vδ, scFv, Fab, vHH, non-immunogloblulin antigen binding scaffolds, DARPIN, receptor, cytokine/ligands, adaptors etc.) attached to two chains via optional linkers wherein each chain comprises a CD16 transmembrane domain. In an embodiment, one or both chains lack a cytosolic domain. In an embodiment, one or both chains comprise a partial cytosolic domain.

In an embodiment, at least one chain of the double chain CD16 SAR comprises the CD16 extracellular domain comprising both immunoglobulin-like domains (i.e., D1 and D2) that is attached via the CD16 hinge domain to CD16 transmembrane domain. A double chain CD16 SAR may further comprise an AABD attached to the N-terminus or near the N-terminus of vL, vH, Vα, Vβ, Vγ or Vδ chains comprising the antigen binding domain of the SAR via optional linkers.

In an embodiment, the disclosure provides a double chain CD16 SAR comprising mutant CD16 chains (e.g., S213Y mutation and/or lacking cytoplasmic domain) where one or both chains comprise a partial or entire region of CD16. In an embodiment, the disclosure provides a double chain CD16-SARs comprising mutant CD16 chains (e.g., S213Y mutation and/or lacking cytoplasmic domain) where one or both chains comprise a partial or entire region of CD16 extracellular domain. In an embodiment, the disclosure provides a double chain CD16 SARs comprising mutant CD16 chains (e.g., S213Y mutation and/or lacking cytoplasmic domain) where one or both chains comprise a partial or entire region of CD16 D1 domain. In an embodiment, the disclosure provides a double chain CD16 SARs comprising mutant CD16 chains (e.g., S213Y mutation and/or lacking cytoplasmic domain) where one or both chains comprise a partial or entire region of CD16 D2 domain or a functional variant or homolog thereof. In an embodiment, the disclosure provides a double chain CD16 SARs comprising mutant CD16 chains (e.g., S213Y mutation and/or lacking cytoplasmic domain) where one or both chains comprise a partial or entire region of CD16 hinge domain or a functional variant or homolog thereof. In an embodiment, the disclosure provides a double chain CD16 SARs comprising mutant CD16 chains (e.g., S213Y mutation and/or lacking cytoplasmic domain) where one or both chains comprise a CD16 transmembrane domain or a functional variant or homolog thereof.

In an embodiment, one or both chains of the double chain CD16 SAR comprise the CD16 extracellular domain comprising both immunoglobulin-like domains (i.e., D1 and D2) that is attached via the CD16 hinge domain to a mutant CD16 transmembrane domain. In an embodiment, the CD16 transmembrane domain comprises S213Y mutation. In an embodiment, one or both chains of the double chain CD16 SAR comprises the CD16 extracellular domain comprising both immunoglobulin-like domains (i.e., D1 and D2) that is attached via the CD16 hinge domain to a CD16 transmembrane domain but lacks a CD16 cytoplasmic domain. In an embodiment, one or both chains of a double chain CD16 SAR with mutant CD16 transmembrane domain and/or lacking a cytoplasmic domain also retains the ability to bind to the Fc region of an antibody, an antibody fragment or bispecific/tri-specific engager and mediate antibody dependent cytotoxicity. In an embodiment, one or both chains of the double chain CD16 SAR with mutant CD16 transmembrane domain and/or lacking a cytoplasmic domain comprises the partial CD16 extracellular domain comprising the 2nd immunoglobulin like domains (i.e., D2) that is attached via CD16 hinge domain to CD16 transmembrane domain. In an embodiment, one or both chains of such double chain CD16 SAR with mutant CD16 transmembrane domain and/or lacking a cytoplasmic domain lacks the ability to bind to the Fc portion of an antibody or an antibody fragment as it contains only the D2 domain of CD16 and lacks the D1 domain. In an embodiment, one or both chains of the double chain CD16 SAR with mutant CD16 transmembrane domain and/or lacking a cytoplasmic domain comprises the partial or entire CD16 hinge domain that is attached to CD16 transmembrane domain. In an embodiment, one or both chains of such double chain CD16 SAR lacks the ability to bind to the Fc portion of an antibody or an antibody fragment as it lacks both the D1 and D2 domains.

In an embodiment, both chains of a double chain CD16 SAR with mutant CD16 transmembrane domain and/or lacking a cytoplasmic domain comprise an antigen binding domain. In an embodiment, only one of the chains of a double chain CD16 SAR with mutant CD16 transmembrane domain and/or lacking a cytoplasmic domain comprise an antigen binding domain. In an embodiment, one of the chains of a double chain CD16 SAR with mutant CD16 transmembrane domain and/or lacking a cytoplasmic domain comprise a non-natural antigen binding domain and the second chain binds to Fc portion of an antibody or antibody fragment or a bispecific/trispecific engager via the CD16 extracellular domain.

In an embodiment, one chain of a double chain CD16 SAR with mutant CD16 transmembrane domain and/or lacking a cytoplasmic domain comprises an antigen binding domain consisting of a vL domain and the second chain of the double chain CD16 SAR with mutant CD16 transmembrane domain and/or lacking a cytoplasmic domain comprises an antigen binding domain consisting of a vH domain. In an embodiment, both chains of a double chain CD16 SAR with mutant CD16 transmembrane domain and/or lacking a cytoplasmic domain comprise an antigen binding domain of the same class (i.e., scFv, vHH, FHVH, a single domain antibody, a non-immunoglobulin antigen binding scaffold, a ligand, or a receptor etc.). In an embodiment, each chain of a double chain CD16 SAR with mutant CD16 transmembrane domain and/or lacking a cytoplasmic domain comprise a vHH domain. In an embodiment, each chain of a double chain CD16 SAR with mutant CD16 transmembrane domain and/or lacking a cytoplasmic domain comprise a FHVH domain. In an embodiment, both chains of a double chain CD16 SAR with mutant CD16 transmembrane domain and/or lacking a cytoplasmic domain comprise an antigen binding domain of different classes (i.e., scFv, vHH, FHVH, a single domain antibody, a non-immunoglobulin antigen binding scaffold, a ligand, or a receptor etc.). In an example embodiment, one chain of a double chain CD16 SAR with mutant CD16 transmembrane domain and/or lacking a cytoplasmic domain comprises an antigen binding domain derived from vHH domain while the second chain comprises an antigen binding domain derived from a FHVH domain.

The two chains of a double chain CD16 SAR with mutant CD16 transmembrane domain and/or lacking a cytoplasmic domain may target the same antigen (e.g., CD19) or different antigens (e.g., CD19 and CD20). The two chains of a double chain CD16 SAR with mutant CD16 transmembrane domain and/or lacking a cytoplasmic domain may target two different epitopes of a single antigen (e.g., CD19) or two different antigens (e.g., CD19 and CD20). Each chain of a double chain SAR may bind to one antigen or more than one antigen (e.g., two, three, four etc.). Each chain of a double chain CD16 SAR may further comprise adaptors (e.g., RZIP, EZIP, NKG2D-YA, NKG2D-FA etc.).

In another embodiment, one or both of the CD16 chain(s) of a double chain CD16-SAR with mutant CD16 transmembrane domain and/or lacking a cytoplasmic domain may further comprise a cytosolic costimulatory domain. In another embodiment, one or both of the CD16 chain(s) of a double chain CD16-SAR with mutant CD16 transmembrane domain and/or lacking a cytoplasmic domain may further comprise a cytosolic coreceptor domain. Example costimulatory domains include costimulatory domains of CD8a, CD8b and CD4 etc. In another embodiment, one or both of the CD16 chain(s) of a double chain CD16-SAR with mutant CD16 transmembrane domain and/or lacking a cytoplasmic domain may further comprise a cytosolic signaling molecule. Example signaling molecule include LAT-200-262del (SEQ ID NO: 50029), Lck, Lck-T3161 (SEQ ID NO: 50031), SLP-76-224-244del (SEQ ID: 50030), ZAP-70 and mutants and variants thereof. In an embodiment, a SAR is a z-CD16-SAR comprising one chain comprising a vL, Vα, or Vδ domain operably linked via an Ig-like linker (e.g., IgCL or TCRβ constant domain), to a polypeptide encoding CD3z hinge, transmembrane and cytosolic domain and a second polypeptide linker comprising a vH, Vβ, or Vγ domain operably linked via an Ig-like linker (e.g., IgG1-CH1 or TCRα constant domain), to a polypeptide encoding CD16 hinge and transmembrane domain and a cytosolic domain comprising a costimulatory domain (e.g., 4-1BB, CD28 etc.) or a coreceptor domain (e.g., CD8a, CD8b, CD4 etc.) or a signaling molecule (e.g., Lck, Lek-T3161, LAT-200-262del, SLP-76-224-244del or ZAP70 etc.). In an embodiment, the cytosolic costimulatory, coreceptor or signaling molecules are attached to the C-terminal of the CD16 transmembrane domain. In an embodiment, the cytosolic costimulatory, coreceptor or signaling molecules are attached to the C-terminal of the CD16 cytosolic domain. It is to be understood that the antigen binding domains can be switched. Thus, vL, Vα and Vδ could be attached to the second chain and similarly the vH, Vβ and Vγ domains could be attached to the first chain of such a double chain SAR. In another aspect, the first chain may also comprise a cytosolic costimulatory domain (e.g., 41BB), coreceptor (e.g., CD8) or signaling molecules (e.g., Lck or LAT etc.) that is present C-terminal to the CD3z transmembrane domain. In another embodiment, the CD3z chain is replaced by the FcRγ. Example zSAR and zCD16 SARs are provided in Tables 2A-2E. In an embodiment, the Va and Vb in the constructs in Table 2A-2E can be replaced with vL and vH fragments derived from antibodies. Similarly, vL and vH fragments can be replaced by Va and Vb fragments. The order of Va, Vb, vL and vH can be switched. Similarly, one of more CD3z fragments can be replaced by corresponding FcRy fragments. The Ig linker domains can be derived from immunoglobulins or TCR constant chains. The one or both CD3z cytosolic domains can have the dQ101 mutation.

The two chains of CD16A-SARs with mutant CD16 transmembrane domain and/or lacking a cytoplasmic domain described herein may be encoded by a single polynucleotide chain and translated into a single polypeptide chain, which is subsequently cleaved into different proteins. The two chains of CD16A-SARs with mutant CD16 transmembrane domain and/or lacking a cytoplasmic domain described herein may be expressed using two distinct promoters and encoded by two separate polynucleotide chains. The two chains of CD16A-SARs with mutant CD16 transmembrane domain and/or lacking a cytoplasmic domain described herein may be encoded by a single vector. The two chains of CD16A-SARs with mutant CD16 transmembrane domain and/or lacking a cytoplasmic domain described herein may be encoded by a two different vector. The nucleic acid molecule encoding a CD16-SAR with mutant CD16 transmembrane domain and/or lacking a cytoplasmic domain can comprise one or more leader sequences (also known as a signal peptide). In one embodiment, each functional unit (e.g., an antigen binding domain joined to a CD16 chain plus Furine-SGSG-cleavable linker) of a CD16A-SAR can be preceded by a leader sequence which directs the CD16A-SAR to the cell surface as a type I transmembrane protein. In one embodiment, the antigen-binding domain of CD16-SAR is extracellular-facing. In some embodiments, the leader sequence comprises the nucleic acid sequence of any of SEQ ID NO: 301-303 and amino acid sequences of SEQ ID NO: 8681 to 8683. In some embodiments, short nucleic acid sequences (3-9 nucleic acids) comprising restriction enzyme sites are located between the different subunits of a CD16A-SAR with mutant CD16 transmembrane domain and/or lacking a cytoplasmic domain, e.g., between a signal sequence and the antigen binding domain of the CD16-SAR or between the antigen binding and the CD16 chain.

The different SARS of this disclosure are modular in design. Therefore, the sequence encoding one of the CD16 mutant chain or signaling adaptor comprising a CD16 SAR may be replaced by a sequence encoding different signaling module. Example signaling modules are provided in TABLE 7 of the provisional patent application. Similarly, the antigen binding domains can be replaced by other antigen binding domains.

In certain embodiments, the disclosure provides a novel platform of synthetic antigen receptors, designated CD16-SARs, containing two chains, one of which incorporates the partial or entire region of CD16 with mutant CD16 transmembrane domain and/or lacking a cytoplasmic domain.

In alternate embodiment, the disclosure provides a double chain CD16 SARs with mutant CD16 transmembrane domain and/or lacking a cytoplasmic domain where one or both of the chains comprise a partial or entire region of CD16 extracellular domain. In an embodiment, the disclosure provides double chain CD16 SARs with mutant CD16 transmembrane domain and/or lacking a cytoplasmic domain where one or both of the chains comprise a partial or entire region of CD16 hinge domain. In an embodiment, the disclosure provides a double chain CD16 SAR with mutant CD16 transmembrane domain and/or lacking a cytoplasmic domain where one or both of the chains comprise a CD16 transmembrane domain. In an embodiment, the disclosure provides a double chain CD16 SAR where one or both of the chains comprises a partial or entire deletion of CD16 cytosolic domain.

The disclosure provides that the vL fragment of an antibody can be joined to a CD16 chain with mutant CD16 transmembrane domain and/or lacking a cytoplasmic domain and the vH fragment can be joined to the another signaling chain, such as CD3z, FcRγ. NKp30, NKp44, NKp46, TCRα constant chain, TCRβ constant chain, TCRγ constant chain or TCRδ constant chain etc. Alternatively, the disclosure provides that the vH fragment of an antibody can be joined to a CD16 chain with mutant CD16 transmembrane domain and/or lacking a cytoplasmic domain and the vL fragment can be joined to the another signaling chain, such as CD3z, FcRγ, NKp30, NKp44, NKp46, TCRα constant chain, TCRβ constant chain, TCRγ constant chain or TCRδ constant chain etc. When the two such chains (e.g., vL-mutant CD16 and vH-CD3z) are co-expressed in the same cell, the vL and vH fragments can bind their cognate antigen and transmit a T cell signal. In particular, T cells expressing such CD16-hererodimeric SAR with mutant CD16 transmembrane domain and/or lacking a cytoplasmic domain when exposed to a cell line expressing the cognate target antigen can activate NFAT signaling, induce IL2 production, promote T cell proliferation, promote T cell activation, and exert cytotoxicity. In another example embodiment, NK cells expressing such CD16-SAR with mutant CD16 transmembrane domain and/or lacking a cytoplasmic domain when exposed to a cell line expressing the cognate target antigen can induce IL2 production, promote NK cell proliferation, promote NK cell activation, or exert cytotoxicity. The expression and activity of the CD16-heterodimeic SAR can be further increased by incorporation of a linker between the vL/vH and the CD16 and the other signaling chains (e.g., CD3z, FcRγ, NKp30, NKp44, NKp46 etc.). In particular, the IgCL and Ig-CH1 domains derived from antibodies serve as useful linkers between the vL/vH and CD16 fragments with mutant CD16 transmembrane domain and/or lacking a cytoplasmic domain. Additional Ig-like domains are known in the art and can serve as useful linkers in alternate embodiment of the disclosure. The disclosure also provides that the vL/vH fragments in the above SAR can be replaced by Vα and Vβ domains derived from a TCR to generate uTCR-SAR.

Also provided herein are clonal iPSCs genetically engineered to comprise, among other editing as contemplated and described herein, a CD16 SAR with mutant CD16 transmembrane domain and/or lacking a cytoplasmic domain. In an embodiment, the CD16 SAR with mutant CD16 transmembrane domain and/or lacking a cytoplasmic domain is a high affinity CD16 SAR or a high-affinity non-cleavable CD16 SAR (hnCD16-SAR). The genetically engineered iPSCs are capable of differentiating into effector cells comprising the CD16-SAR with mutant CD16 transmembrane domain and/or lacking a cytoplasmic domain (e.g., high affinity CD16 SAR or hnCD16-SAR) introduced to the iPSCs. In some embodiments, the derived effector cells comprising CD16-SAR with mutant CD16 transmembrane domain and/or lacking a cytoplasmic domain are NK cells. In some embodiments, the derived effector cells comprising CD16-SAR with mutant CD16 transmembrane domain and/or lacking a cytoplasmic domain are T cells. In an embodiment, the CD16-SAR with mutant CD16 transmembrane domain and/or lacking a cytoplasmic domain (e.g., high affinity CD16 SAR or hnCD16-SAR) expressed in iPSC or derivative cells thereof binds to not only ADCC antibodies or fragments thereof, but also to bi-, tri-, or multi-specific engagers or binders that recognize the CD16 or CD64 extracellular binding domains of said CD16 SAR. As such, the present application provides a derivative effector cell or a cell population thereof, preloaded with one or more pre-selected ADCC antibody through binding with the extracellular domain of the CD16-SAR expressed on the derivative effector cell, in an amount sufficient for therapeutic use in a treatment of a condition, a disease, wherein said CD16-SAR with mutant CD16 transmembrane domain and/or lacking a cytoplasmic domain comprises an extracellular binding domain of CD64, or of CD16 having FI76V and S197P. In an embodiment, the antigen binding domain of the CD16-SAR with mutant CD16 transmembrane domain and/or lacking a cytoplasmic domain comprises an AABD, a scFv, Fv, extracellular domain of a receptor, ligand, or another non-immunoglobulin antigen binding module. In an embodiment, the CD16-SAR with mutant CD16 transmembrane domain and/or lacking a cytoplasmic domain comprises an antigen binding domain attached to or near the N-terminus of the Fc binding domain of CD16 or CD64. In an embodiment, the CD16-SAR with mutant CD16 transmembrane domain and/or lacking a cytoplasmic domain further comprises an antigen binding domain (e.g., AABD, e.g., FHVH, chVH, aVH, vHH, Darpin, centyrin, affibody etc.) attached to or near the N-terminus of the Fc binding domain of CD16 or CD64.

In an embodiment, the CD16-SAR of the disclosure comprise the Fc binding region of CD32 or CD64 fused in frame to CD16 with mutant transmembrane and/or cytoplasmic domain or variant thereof. In an example embodiment, the order of different modules in such a CD16 SAR with mutant CD16 transmembrane domain and/or lacking a cytoplasmic domain may comprise from NH2 to C-terminus the following:
Antigen binding domain(n)-CD32-Fc binding domain-CD16 transmembrane domain; where n=1, 2, 3, or more.
Antigen binding domain(n)-CD32-Fc binding domain-CD16 mutant transmembrane domain-CD16 cytoplasmic domain; where n=1, 2, 3, or more.
Antigen binding domain(n)-CD32-Fc binding domain-CD16 mutant transmembrane domain; where n=1, 2, 3, or more.

In an example embodiment, the order of different modules in such a CD16 SAR with mutant CD16 transmembrane domain and/or lacking a cytoplasmic domain may comprise from NH2 to C-terminus the following:
Antigen binding domain(n)-CD64-Fc binding domain-CD16 transmembrane domain; where n=1, 2, 3, or more.
Antigen binding domain(n)-CD64-Fc binding domain-CD16 mutant transmembrane domain-CD16 cytoplasmic domain; where n=1, 2, 3, or more.
Antigen binding domain(n)-CD64-Fc binding domain-CD16 mutant transmembrane domain; where n=1, 2, 3, or more.

Unlike primary NK cells, mature T cells from a primary source (i.e., natural/native sources such as peripheral blood, umbilical cord blood, or other donor tissues) do not express CD16. It was unexpected that mature T cells expressing the exogeneous CD16-SAR construct with mutant CD16 transmembrane domain and/or lacking a cytoplasmic domain show cell surface expression of the CD16 SAR and are capable of transmitting a cell signal (e.g., NFAT signaling) when exposed to the target antigen expressing cells.

The disclosure provides a derivative T cell comprising an exogenous CD16-based SAR with mutant CD16 transmembrane domain and/or lacking a cytoplasmic domain. In some embodiment, the CD16-based SAR with mutant CD16 transmembrane domain and/or lacking a cytoplasmic domain comprise the wild-type sequence of CD16. In some embodiments, the hnCD16 with mutant CD16 transmembrane domain and/or lacking a cytoplasmic domain comprise in the derivative T cell comprises F176V (158V) and S197R (or S197P). In some other embodiments, the hnCD16 with mutant CD16 transmembrane domain and/or lacking a cytoplasmic domain comprised in the derivative T cell comprises a full or partial ectodomain originated from CD64 or may further comprises at least one of non-native transmembrane domain, stimulatory domain and signaling domain.

In addition to primary NK and T cells, the CD16 SARs with mutant CD16 transmembrane domain and/or lacking a cytoplasmic domain of the disclosure can be expressed in immortalized cell lines. Example immortalized cell lines suitable for expression of the CD16 SARs with mutant CD16 transmembrane domain and/or lacking a cytoplasmic domain of the disclosure include NK92 and NK92MI cell lines. Additionally, CD16 SARs with mutant CD16 transmembrane domain and/or lacking a cytoplasmic domain of the disclosure can be expressed in pluripotent hematopoietic stem cells (e.g., CD34+ stem cells), which can be differentiated to generate CD16 SAR expressing blood cells belonging to different lineages.

The cells expressing the with mutant CD16 transmembrane domain and/or lacking a cytoplasmic domain may also express accessory modules encoding cytokines (e.g., membrane anchored IL2, membrane anchored IL15 etc.), suicide switches, and survival switches. Example accessory modules are provided in SEQ ID NO(DNA): 656-667, 904-928, 968-969 and SEQ ID NO (PRT): 9036-9047, 9284-9308, 9348-9349.

The Synthetic Immune Receptor (SIR) architecture comprises vL fragment of an antibody attached to one TCR constant chain and the vH fragment attached to a second complementary TCR constant chain that can form a heterodimer with the first TCR constant chain. A major problem with the SIR design is steric hinderance between the vL/vH domains and specific residues present in the TCR constant chains since the vL/vH domains are artificially grafted on the TCR constant chains. This steric hinderance between the TCR constant chains and vL/vH domains can interfere in the interaction between the vL and vH domains of the SIR resulting in reduced binding affinity and signaling through the receptor. As the steric hinderance is due to the interaction between specific residues of the vL and vH fragments and specific residues of the TCR constant chains, it varies depending on the sequence of the vL and vH fragments used to generate the SIR. This problem is also seen with other non-TCR antigen binding domains that can be used in the construction of the SIR.

On-target/off-tumor toxicity, cytokine release syndrome and neurotoxicity are major problems of cell therapy and can be fatal. A potential solution is the generation of affinity tuned SIR with varying levels of affinity for the target antigen. A standard approach to generation of affinity tuned CAR constructs is the use of CDR mutagenesis to select low affinity antigen binding domains (e.g., scFv). However, this approach is expensive and time consuming. The CDR mutagenesis of antigen-binding domains can also lead to acquisition of new binding affinities, resulting in on-target off tumor toxicity.

The current invention provides a solution to the above problems by providing a panel of hybrid chains that can be used in the construction of a SAR (e.g., a SIR). One or two of these hybrid chains can replace one or both of the wild-type TCR chains in case of steric hinderance between the antigen binding domain (e.g., vL, vH, scFv, vHH etc.) being used in the construction of the SAR (e.g., SIR) and one or both of the wild type TCR chains. The hybrid TCR chains can be also used to rapidly generate a large panel of SAR (e.g., SIR) constructs with varying level of affinity, thereby saving cost, time and labor. Furthermore, the HC-SAR approach does not require the need to generate a large panel of new antigen binding domains, thereby obviating the risk of acquisition of new antigen binding specificities and off-tumor toxicities.

Finally, the HC-SAR (e.g., HC-SIR) approach also provide a strategy for preventing TCR mispairing that exploits our understanding of TCR biogenesis. Without being limited by any theory, functional surface expression of the TCR complex typically involves the recruitment of accessory CD3 proteins by invariant domains of the two polypeptide chains of the TCR. Disruption of the TCR-CD3 interactions can ablate both surface expression and signaling of the TCR complex. Accordingly, it can be useful to prevent mispairing of introduced and endogenous chains entirely, thereby eliminating the risk of autoimmunity and maximizing surface expression of the therapeutic TCR heterodimer. Additionally, it can be useful for modifications made to the introduced TCR chains to minimize or avoid foreign sequences, at least in that it is contemplated that the absence of foreign sequences can minimize immunogenicity and avoid development of antibodies against the introduced TCRs. It is noted that hybrid-chain SIRs in accordance with some embodiments herein can minimize or prevent mispairing, while also comprising host organism sequences (e.g., fully human TCR's for a human host) so as to minimize immunogenicity against the HC-SAR.

A number of suitable HC-SAR configurations are provided in accordance with some embodiments herein.

HC-SAR (e.g., HC-SIR) chains can comprise various domains, including the constant domain (C), connecting peptide (ConnP), transmembrane domain (TM), and cytoplasmic tail (CP or IC). The constant domain of a SIR comprises immunoglobulin like (Ig-like) linker domains. Example Ig-like linker domains derived from constant domains of TCR and immunoglobulins are provided in SEQ ID NO (DNA): 581-614; 19751-19756) and SEQ ID NO (PRT): 8960 to 8994, 20371-20376. In an embodiment, the TCR constant domains and antibody constant domains used in the construction of the HC-SAR (e.g., HC-SIR) comprise N-terminal and C-terminal deletion mutants of the forgoing that are missing between 1-50 amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50). In some embodiments, the constant domain of TCR chains comprise mutations that result in formation of disulfide bond between the two TCR chains. Example mutations include T48C in TCRα constant domain (Ig-like linker) and S57C in TCRb constant domain (Ig-like linker domain).

A number of configurations for the chains of a HC-SAR (e.g., HC-SIR) in accordance with some embodiments herein are set forth in Tables 2F-2J, herein. It is contemplated that synthetic versions and variants of any or all of the indicated domains can be useful in HC-SIRs in accordance with some embodiments herein. As such, unless stated otherwise, C, ConnP, TM, and CP(IC) domains are contemplated to encompass naturally occurring and synthetic versions of the indicated domains. In some embodiments, the HC-SIR comprises synthetic variants of naturally occurring human domains. In some embodiments, the HC-SIR is a chimera of synthetic variants of naturally occurring human domains and non-human species. In some embodiments, the HC-SAR (e.g., HC-SIR) comprises synthetic variants of naturally occurring murine domains. In some embodiments, the HC-SAR (e.g., HC-SIR) is a chimera of synthetic variants of naturally occurring human domains and murine domains. In some embodiments, the HC-SIR is a chimera of synthetic variants of naturally occurring human domains and non-human species.

In accordance with some embodiments herein, HC-SAR (e.g., HC-SIR) can be entirely of one organism. Optionally, the HC-SAR (e.g., HC-SIR) can be chimeric, comprising a combination of domains from two different organisms (for example, human and murine). For example, chimeric SIR comprising murinized human TCR constant chains are described in WO 2018/102795 A1, which is hereby incorporated by reference in its entirety. Without being limited by any theory, it is contemplated that fully human and/or chimeric HC-SARs (e.g., HC-SIRs) are less likely to be immunogenic in a human host than murine HC-SIRs. In some embodiments, a chimeric HC-SAR (e.g., HC-SIR) is provided. In some embodiments, the domains of the HC-SIR are fully human. In some embodiments, the domains of the HC-SAR (e.g., HC-SIR) are fully derived from a non-human species. In some embodiments, the domains of the HC-SIR are fully murine. In some embodiments, the HC-SAR is a chimera of human domains and non-human domains. In some embodiments, the HC-SAR is a chimera of human domains and murine domains.

In accordance with Alternative 1, the HC-SAR comprises two polypeptide chains: in the first chain, the vL domain of an antibody and constant domain (or Ig-like linker domain) of the alpha chain (TCR α chain), and the connecting peptide, transmembrane domain and cytoplasmic tail of the beta chain (TCR β chain), and reciprocally, in the second chain, the vH domain of an antibody and constant domain (or Ig-like linker domain) of the beta chain, and the connecting peptide, transmembrane domain and cytoplasmic tail of the alpha chain. Schematically, this can be diagrammed as: first chain: vL-Cₐₗₚₕₐ-ConnP_{beta}-TM_{beta}-CP_{beta}; second chain: vH-C_{beta}-ConnPₐₗₚₕₐ-TMₐₗₚₕₐ-CPₐₗₚₕₐ.

In accordance with Alternative 2, the HC-SAR comprises two polypeptide chains: in the first chain, the vL domain of an antibody, constant domain (or Ig-like linker domain) and connecting peptide of the alpha chain and the transmembrane domain and cytoplasmic tail of the beta chain, and reciprocally, in the second chain, the vH domain of an antibody, constant domain (or Ig-like linker domain) and connecting peptide of the beta chain combined with the transmembrane domain and cytoplasmic tail of the alpha chain. Schematically, this can be diagrammed as: first chain: vL-Cₐₗₚₕₐ-ConnPₐₗₚₕₐ-TM_{beta}-CP_{beta}; second chain: vH-C_{beta}-ConnP_{beta}-TMₐₗₚₕₐ-CPₐₗₚₕₐ.

In accordance with Alternative 3, the HC-SAR comprises two polypeptide chains: in the first chain, the vL domain, constant domain (or Ig-like linker domain) of the gamma chain (TCR γ chain) and the connecting peptide, transmembrane domain and cytoplasmic tail of the delta chain (TCR δ chain), and reciprocally, in the second chain, vH domain, constant domain (or Ig-like linker domain) of the delta chain and the connecting peptide, transmembrane domain and cytoplasmic tail of the gamma chain. Schematically, this can be diagrammed as: first chain: vL-C_{gamma}-ConnP_{delta}-TM_{delta}-CP_{delta}; second chain: vH-C_{delta}-ConnP_{gamma}-TM_{gamma}-CP_{gamma}.

In accordance with Alternative 4, the HC-SAR comprises two polypeptide chains: in the first chain, the vL, constant domain (or Ig-like linker domain) and connecting peptide of the gamma chain and the transmembrane domain and cytoplasmic tail of the delta chain, and reciprocally, in the second chain, the vH, constant domain (or Ig-like linker domain) and connecting peptide of the delta chain and the transmembrane domain and cytoplasmic tail of the gamma chain. Schematically, this can be diagrammed as: first chain: vL-C_{gamma}-ConnP_{gamma}-TM_{delta}-CP_{delta}; second chain: VH-C_{delta}-ConnP_{delta}-TM_{gamma}-CP_{gamma}.

In accordance with Alternative 5, the HC-SAR comprises two polypeptide chains: in the first chain, the vL domain of an antibody and constant domain (or Ig-like linker domain) of the alpha chain, and the connecting peptide, transmembrane domain and cytoplasmic tail of the delta chain, and reciprocally, in the second chain, the vH domain of an antibody and constant domain (or Ig-like linker domain) of the beta chain, and the connecting peptide, transmembrane domain and cytoplasmic tail of the gamma chain. Schematically, this can be diagrammed as: first chain: vL-Cₐₗₚₕₐ-ConnP_{delta}-TM_{delta}-CP_{delta}; second chain: vH-C_{beta}-ConnP_{gamma}-TM_{gamma}-CP_{gamma}.

In accordance with Alternative 6, the HC-SAR comprises two polypeptide chains: in the first chain, the vL domain of an antibody and constant domain (or Ig-like linker domain) of the alpha chain, and the connecting peptide, transmembrane domain and cytoplasmic tail of the delta chain, and reciprocally, in the second chain, the vH domain of an antibody and constant domain (or Ig-like linker domain), the connecting peptide, transmembrane domain and cytoplasmic tail of the gamma or beta chain. Schematically, this can be diagrammed as: first chain: vL-Cₐₗₚₕₐ-ConnP_{delta}-TM_{delta}-CP_{delta}; second chain: vH-C_{gamma}-ConnP_{gamma}-TM_{gamma}-CP_{gamma} or vH-C_{beta}-ConnP_{beta}-TM_{beta}-CP_{beta}.

In accordance with Alternative 7, the HC-SAR comprises two polypeptide chains: in the first chain, the vL domain of an antibody and constant domain (or Ig-like linker domain) of the delta chain, and the connecting peptide, transmembrane domain and cytoplasmic tail of the alpha chain, and reciprocally, in the second chain, the vH domain of an antibody and constant domain (or Ig-like linker domain), the connecting peptide, transmembrane domain and cytoplasmic tail of the gamma or beta chain. Schematically, this can be diagrammed as: first chain: vL-C_{delta}-ConnPₐₗₚₕₐ-TMₐₗₚₕₐ-CPₐₗₚₕₐ; second chain: vH-C_{gamma}-ConnP_{gamma}-TM_{gamma}-CP_{gamma} or vH-C_{beta}-ConnP_{beta}-TM_{beta}-CP_{beta}.

In accordance with Alternative 8, the HC-SAR comprises two polypeptide chains: in the first chain, the vL domain of an antibody and constant domain (or Ig-like linker domain) of the beta chain, and the connecting peptide, transmembrane domain and cytoplasmic tail of the gamma chain, and reciprocally, in the second chain, the vH domain of an antibody and constant domain (or Ig-like linker domain), the connecting peptide, transmembrane domain and cytoplasmic tail of the alpha or delta chain. Schematically, this can be diagrammed as: first chain: vL-C_{beta}-ConnP_{gamma}-TM_{gamma}-CP_{gamma}; second chain: vH-Cₐₗₚₕₐ-ConnPₐₗₚₕₐ-TMₐₗₚₕₐ-CPₐₗₚₕₐ or vH-C_{delta}-ConnP_{delta}-TM_{delta}-CP_{delta}

In accordance with Alternative 9, the HC-SAR comprises two polypeptide chains: in the first chain, the vL domain of an antibody and constant domain (or Ig-like linker domain) of the gamma chain, and the connecting peptide, transmembrane domain and cytoplasmic tail of the beta chain, and reciprocally, in the second chain, the vH domain of an antibody and constant domain (or Ig-like linker domain), the connecting peptide, transmembrane domain and cytoplasmic tail of the alpha or delta chain. Schematically, this can be diagrammed as: first chain: vL-C_{gamma}-ConnP_{beta}-TM_{beta}-CP_{beta}; second chain: vH-Cₐₗₚₕₐ-ConnPₐₗₚₕₐ-TMₐₗₚₕₐ-CPₐₗₚₕₐ or vH-C_{delta}-ConnP_{delta}-TM_{delta}-CP_{delta}

In accordance with Alternative 10, the HC-SAR comprises two polypeptide chains: in the first chain, the vL domain of an antibody and constant domain (or Ig domain) of an antibody chain (e.g., IgCL), the connecting peptide of gamma chain, transmembrane domain and cytoplasmic tail of the beta chain, and reciprocally, in the second chain, the vH domain of an antibody, constant domain (or Ig domain) of an antibody (e.g., IgG1-CH1 or IgG1-CH4), the connecting peptide of delta chain, transmembrane domain and cytoplasmic tail of the alpha chain. Schematically, this can be diagrammed as: first chain: vL-IgCL-ConnP_{gamma}-TM_{beta}-CP_{beta}; second chain: vH-IgG1-CH1-ConnP_{delta}-TMₐₗₚₕₐ-CPₐₗₚₕₐ.

In accordance with Alternative 11, the HC-SAR comprises two polypeptide chains: in the first chain, the vL domain of an antibody and constant domain (or Ig domain) of an antibody chain (e.g., IgCL), the connecting peptide of gamma chain, transmembrane domain and cytoplasmic tail of the beta chain, and reciprocally, in the second chain, the vH domain of an antibody, constant domain (or Ig domain) of an antibody (e.g., IgG1-CH1 or IgG1-CH4), the connecting peptide, transmembrane domain and cytoplasmic tail of the alpha chain. Schematically, this can be diagrammed as: first chain: vL-IgCL-ConnP_{gamma}-TM_{beta}-CP_{beta}; second chain: vH-IgG11-CH1-ConnPₐₗₚₕₐ-TMₐₗₚₕₐ-CPₐₗₚₕₐ.

In accordance with Alternative 12, the HC-SAR comprises two polypeptide chains: in the first chain, the vL domain of an antibody and constant domain (or Ig domain) of an antibody chain (e.g., IgCL), the connecting peptide of gamma chain, transmembrane domain and cytoplasmic tail of the beta chain, and reciprocally, in the second chain, the vH domain of an antibody, constant domain (or Ig domain) of an antibody (e.g., IgG1-CH1 or IgG1-CH4), the connecting peptide, transmembrane domain and cytoplasmic tail of the delta chain. Schematically, this can be diagrammed as: first chain: vL-IgCL-ConnP_{gamma}-TM_{beta}-CP_{beta}; second chain: vH-IgG1-CH1-ConnP_{delta}-TM_{delta}-CP_{delta}.

In accordance with Alternative 13, the HC-SAR comprises two polypeptide chains: in the first chain, the vL domain of an antibody and constant domain (or Ig domain) of an antibody chain (e.g., IgCL), the connecting peptide of beta chain, transmembrane domain and cytoplasmic tail of the gamma chain, and reciprocally, in the second chain, the vH domain of an antibody, constant domain (or Ig domain) of an antibody (e.g., IgG1-CH1 or IgG1-CH4), the connecting peptide, transmembrane domain and cytoplasmic tail of the alpha chain or delta chain. Schematically, this can be diagrammed as: first chain: vL-IgCL-ConnP_{beta}-TM_{gamma}-CP_{gamma}; second chain: vH-IgG-CH1-ConnPₐₗₚₕₐ-TMₐₗₚₕₐ-CPₐₗₚₕₐ or vH-IgG1-CH1-ConnP_{delta}-TM_{delta}-CP_{delta}.

It is understood that the vL and vH domains in the above designs can be switched, so that the first chain comprises vH domain and the second chain comprises vL domain. The vL and vH domains come together to create a Fv domain that bind to a target antigen with high specificity. It is understood that the IgCL and IgG1-CH1 domains in the above designs can be switched. Additionally, a number of IgG-CH domains are known (e.g., SEQ ID NO:8963-8976) and can substitute for the IgG1-CH1 domain (SEQ ID NO (PRT): 8962). It is also understood that the TCR constant domain can switch one or both Ig linker domains. Thus, first chain my comprise a TCR constant domain (e.g., SEQ ID NO:8977-8994) and the second chain may comprise an Ig linker domain (e.g., IgG1-CH1)

It is also understood that the CP domains in any of the above designs can be switched. Thus, CP_{delta} may be switched for a CP_{beta}.

In an embodiment, the vL and vH antibody domains of the above-described HC-SAR are replaced by one or more non-TCR antigen binding domains such as scFv, vHH, single variable domain antibody, FHVH (fully human vH domain), SVH (single vH domain), SVL (single vL domain), non-immunoglobulin antigen binding scaffold (e.g., centyrin, affibody, DARPIN, D domain etc.), ligand binding domain of a receptor, receptor binding domain of a ligand, autoantigen, adaptor binding domain, epitope and mimotope etc.

In an embodiment, the disclosure provides at least one recombinant polynucleotide encoding at least one hybrid chain synthetic antigen receptor (HC-SIR), or its functional variant, the at least one SAR or its functional variant comprising a heterodimer of T-cell receptor (TCR) constant chains, the heterodimer having one or more non-TCR antigen binding domains. In an embodiment, at least one TCR constant chain of the HC-SAR is a hybrid TCR chain. In an embodiment, the hybrid TCR chain comprises at least one domain selected from the group of TCR chain constant domain (C), TCR chain connecting peptide (ConnP), TCR chain transmembrane (TM) domain and TCR chain cytoplasmic domain (CP) that is heterologous. In an embodiment, the hybrid TCR chain comprises at least one domain selected from the group of TCR chain constant domain (C), TCR chain connecting peptide (ConnP), and TCR chain cytoplasmic domain (CP) that is heterologous to the TCR chain transmembrane (TM) domain. In an embodiment, the hybrid TCR chain comprises at least one domain selected from the group of TCR chain constant domain (C), TCR chain transmembrane (TM) domain and TCR chain cytoplasmic domain (CP) that is heterologous to the TCR chain connecting peptide (ConnP). In an embodiment, the HC-SAR lacks a TCR chain variable domain.

In an embodiment, at least one domain of the HC-SAR that is heterologous to the TD of the hybrid TCR chain is derived from a) a TCRα, TCRβ, TCRγ, TCRδ or pre-TCRcz chain that lacks the TCR chain transmembrane domain present in the hybrid TCR chain; b) TCR chain with a transmembrane domain with less than 50% sequence identity to the TM of the hybrid TCR chain; c) a TCR constant chain or a functional variant from a different species; d) any combination of (a), (b) and (c).

In an embodiment, more than one domain selected from the group of constant domain (C), connecting peptide (ConnP) and cytoplasmic domain (CP) is heterologous to the transmembrane (TM) domain. In an embodiment, ConnP is heterologous to a) TM domain; b) constant domain (C); c) both a) and b)

In an embodiment, the TM domain of the HC-SAR encodes a peptide with a sequence selected from SEQ ID NO: 31985-88 and 31992, and/or a peptide with a sequence selected from the group of transmembrane domains with SEQ ID NO: 40606 to 40670 and 40737-40758 or a functional variant or a homolog with 1, 2, 3, 4, 5 or 6 amino acid substitutions except at residues 7, 12, 17 and 21; and/or b) transmembrane domain with SEQ ID NO: 40671-40708 or a functional variant or a homolog with 1, 2, 3, 4, 5 or 6 amino acid substitutions except at residues 6, 12, and 16.

In an embodiment, the hybrid chain constant domain (C) is derived from an immunoglobulin. In an embodiment, constant domain of at least one hybrid chain is derived from an immunoglobulin. In an embodiment, constant domains of both hybrid chains are derived from an immunoglobulin

In an embodiment, both TCR constant chains of the HC-SAR are hybrid chains.

In an embodiment, the first hybrid chain of the HC-SAR comprises a first TCR chain transmembrane domain, a non-TCR antigen binding domain and a second TCR chain constant domain or a functional variant or a fragment thereof but does not comprise a first TCR chain constant domain. In an embodiment, the second hybrid TCR chain comprises a second TCR chain transmembrane domain, an optional non-TCR antigen binding domain and a first TCR chain constant domain or a functional variant or a fragment thereof but does not comprise a second TCR chain constant domain. In an embodiment, the first TCR chain constant domain comprises an TCR alpha or gamma chain constant domain or a functional variant or fragment thereof. In an embodiment, the first TCR chain transmembrane domain comprises an TCR alpha or gamma chain transmembrane domain or a functional variant thereof. In an embodiment, the second TCR chain constant domain comprises a TCR beta or delta chain constant domain or a functional variant or fragment thereof. In an embodiment, the second TCR chain transmembrane domain comprises a TCR beta or delta chain transmembrane domain or a variant thereof. In an embodiment, the first TCR chain constant domain comprises an TCR alpha or delta chain constant domain or a functional variant or fragment thereof. In an embodiment, the first TCR chain transmembrane domain comprises a TCR alpha or delta chain transmembrane domain or a functional variant thereof. In an embodiment, the second TCR chain constant domain comprises a TCR beta or gamma chain constant domain or a functional variant or a variant thereof. In an embodiment, the second TCR chain transmembrane domain comprises a TCR beta or gamma chain transmembrane domain or a variant thereof.

In an embodiment, the first hybrid TCR chain further comprises a second TCR chain connecting peptide or a functional variant or fragment thereof but does not comprise a first TCR chain connecting peptide. In an embodiment, the second TCR hybrid chain further comprises a first TCR chain connecting peptide or a functional variant or fragment thereof but does not comprise a second TCR chain connecting peptide. In an embodiment, the first TCR chain connecting peptide comprises an TCR alpha or gamma chain connecting peptide or a functional variant or fragment thereof and the second TCR chain connecting peptide comprises a TCR beta chain or delta connecting peptide or a functional variant or fragment thereof. In an embodiment, the first TCR chain connecting peptide comprises an TCR alpha or delta chain connecting peptide or a functional variant or fragment thereof and the second TCR chain connecting peptide comprises a TCR beta chain or gamma connecting peptide or a functional variant or fragment thereof.

In an embodiment, the HC-SAR comprises: a) a first polypeptide chain comprising a first antigen-binding domain comprising a vH antibody domain, a TCR constant domain or a functional variant or fragment thereof and a first T cell receptor domain (TCRD) comprising a first transmembrane domain of a first TCR subunit; and b) a second polypeptide chain comprising a second antigen-binding domain comprising a vL antibody domain, a TCR constant domain or a functional variant or fragment thereof and a second TCRD comprising a second transmembrane domain of a second TCR subunit, wherein the vH antibody domain of the first antigen-binding domain and the vL antibody domain of the second antigen-binding domain form an antigen-binding module that specifically binds to a target antigen. In an alternate embodiment, the HC-SAR comprises a) a first polypeptide chain comprising a first antigen-binding domain comprising a vL antibody domain, a TCR constant domain or a functional variant or fragment thereof of a first TCR subunit and a T cell receptor domain (TCRD) comprising a first transmembrane domain of a second TCR subunit; and b) a second polypeptide chain comprising a second antigen-binding domain comprising a vH antibody domain, a TCR constant domain or a functional variant or fragment thereof of the second TCR subunit and a second TCRD comprising a second transmembrane domain of the first TCR subunit. In an embodiment, the vH antibody domain of the first antigen-binding domain and the vL antibody domain of the second antigen-binding domain form an antigen-binding module that specifically binds to a target antigen. In an embodiment, (i) the first TCR subunit is a TCR γ chain and the second TCR subunit is a TCR δ or TCR α chain; or (ii) the first TCR subunit is a TCR δ chain and the second TCR subunit is a TCR γ or TCR β chain; or (iii) the first TCR subunit is a TCR α chain and the second TCR subunit is a TCR β or TCRγ chain; or (iv) the first TCR subunit is a TCR β chain, and the second TCR subunit is a TCR α or TCR δ chain. In an embodiment, the first polypeptide chain further comprises a TCR connecting peptide or a functional variant or fragment thereof and the second polypeptide chain further comprise a TCR connecting peptide or a functional variant or fragment thereof. In an embodiment, one or both connecting peptides are heterologous to a) transmembrane domain; b) constant domain; c) both a) and b). In an embodiment, the first TCRD and the second TCRD form a T cell receptor module (TCRM) that is capable of recruiting at least one TCR-associated signaling module. In an embodiment, the TCRM is a non-canonical TCRM. In an embodiment, the TCRM is an inter-species non-canonical TCRM.

In an embodiment, the disclosure provides a HC-SAR that specifically binds to a target antigen, comprising: a) a first polypeptide chain comprising a first antigen-binding domain comprising a vH antibody domain, a constant antibody domain or a functional variant or fragment thereof and a first T cell receptor domain (TCRD) comprising a first transmembrane domain of a first TCR subunit; and b) a second polypeptide chain comprising a second antigen-binding domain comprising a vL antibody domain, a TCR constant domain or a functional variant or fragment thereof and a second TCRD comprising a second transmembrane domain of a second TCR subunit, wherein the VH antibody domain of the first antigen-binding domain and the VL antibody domain of the second antigen-binding domain form an antigen-binding module that specifically binds to a target antigen, or
c) a first polypeptide chain comprising a first antigen-binding domain comprising a vL antibody domain, a constant antibody domain or a functional variant or fragment thereof and a T cell receptor domain (TCRD) comprising a first transmembrane domain of a first TCR subunit; and d) a second polypeptide chain comprising a second antigen-binding domain comprising a vH antibody domain, a TCR constant domain or a functional variant or fragment thereof and a second TCRD comprising a second transmembrane domain of a second TCR subunit. In an embodiment, the vH antibody domain of the first antigen-binding domain and the vL antibody domain of the second antigen-binding domain form an antigen-binding module that specifically binds to a target antigen. In an embodiment, (i) the first TCR subunit is a TCR γ chain, and the second TCR subunit is a TCR δ or TCR α chain; or (ii) the first TCR subunit is a TCR δ chain, and the second TCR subunit is a TCR γ or TCR β chain; or (iii) the first TCR subunit is a TCRα chain, and the second TCR subunit is a TCRβ or TCR γ chain; or (iv) the first TCR subunit is a TCRβ chain, and the second TCR subunit is a TCRα or TCR δ chain. In an embodiment, the first polypeptide chain further comprises a TCR connecting peptide or a functional variant or fragment thereof and the second polypeptide chain further comprise a TCR connecting peptide or a functional variant or fragment thereof. In an embodiment, one or both connecting peptides are heterologous to a) transmembrane domain; b) constant domain; c) both a) and b). In an embodiment, the first TCRD and the second TCRD form a T cell receptor module (TCRM) that is capable of recruiting at least one TCR-associated signaling module. In an embodiment, the TCRM is a non-canonical TCRM. In an embodiment, the TCRM is an inter-species non-canonical TCRM.

In an embodiment, the disclosure provides a HC-SAR where both TCR constant chains comprise antibody constant domains that are joined in frame to connecting peptide (ConnP), TM (transmembrane) and CP (Cytoplasmic, also referred to as intracellular or IC) domains of TCR chains. In an embodiment, the ConnP, TM and IC domains of such HC-SAR are heterologous to each other, i.e., they are not derived from the same TCR constant chain.

In an embodiment, the disclosure provides a HC-SAR that specifically binds to a target antigen, comprising: a) a first polypeptide chain comprising a first antigen-binding domain comprising a vH antibody domain, a first constant antibody domain or a functional variant or fragment thereof and a first T cell receptor domain (TCRD) comprising a first ConnP, TM and CP domains of a first TCR subunit; and b) a second polypeptide chain comprising a second antigen-binding domain comprising a vL antibody domain, a second constant antibody domain or a functional variant or fragment thereof and a second TCRD comprising a second ConnP, TM and CP domains of a second TCR subunit wherein the vH antibody domain of the first antigen-binding domain and the vL antibody domain of the second antigen-binding domain form an antigen-binding module that specifically binds to a target antigen, or c) a first polypeptide chain comprising a first antigen-binding domain comprising a vL antibody domain, a first constant antibody domain or a functional variant or fragment thereof and a first T cell receptor domain (TCRD) comprising a first ConnP, TM and CP domains of a first TCR subunit; and d) a second polypeptide chain comprising a second antigen-binding domain comprising a vH antibody domain, a second constant antibody domain or a functional variant or fragment thereof and a second TCRD comprising a second ConnP, TM and CP domains of a second TCR subunit, wherein the vH antibody domain of the first antigen-binding domain and the vL antibody domain of the second antigen-binding domain form an antigen-binding module that specifically binds to a target antigen. In an embodiment, the first TCRD and the second TCRD form a T cell receptor module (TCRM) that is capable of recruiting at least one TCR-associated signaling module. In an embodiment, the TCRM is a non-canonical TCRM. In an embodiment, the TCRM is an inter-species non-canonical TCRM.

In an embodiment, the disclosure provides a HC-TCR where one or both TCR constant chains comprise antibody constant domains that are joined in frame to connecting peptide (ConnP), TM (transmembrane) and CP (Cytoplasmic, also referred to as intracellular or IC) domains of TCR chains. In an embodiment, the ConnP, TM and IC domains of such HC-TCR are heterologous to each other, i.e., they are not derived from the same TCR constant chain.

In an embodiment, the disclosure provides a HC-TCR that specifically binds to a target peptide antigen, comprising: a) a first polypeptide chain comprising a first TCR antigen-binding domain comprising a Vα or Vγ domain, a first constant antibody domain or a functional variant or fragment thereof and a first T cell receptor domain (TCRD) comprising a first ConnP, TM and CP domains of a first TCR subunit; and b) a second polypeptide chain comprising a second TCR antigen-binding domain comprising a Vβ or Vδ domain, a second constant antibody domain or a functional variant or fragment thereof and a second TCRD comprising a second ConnP, TM and CP domains of a second TCR subunit. In an embodiment, the Vα or Vγ TCR domain of the first antigen-binding domain and the Vβ or Vδ antibody domain of the second antigen-binding domain form an antigen-binding module that specifically binds to a target peptide antigen in an MHC dependent manner, or independent manner.

In an embodiment, the first TCRD and the second TCRD form a T cell receptor module (TCRM) that is capable of recruiting at least one TCR-associated signaling module. In an embodiment, the TCRM is a non-canonical TCRM. In an embodiment, the TCRM is an inter-species non-canonical TCRM. Example TCR antigen binding domains are variable domains (e.g., Vα and Vβ) of TCRs that are capable of binding peptides derived from NY-ESO-1, MAGE-A4, WT-1, PRAME in the presence of HLA-A2 molecule (Table 4). Example such HC-TCR are provided in SEQ ID NO (DNA): 40512-40513.

In an example embodiment, Ig-like linker domains derived from constant domains of TCR and antibodies (immunoglobulins) are provided in SEQ ID NO (DNA): 581-614; 19751-19756) and SEQ ID NO (PRT): 8960 to 8994, 20371-20376. In an embodiment, the antibody constant domains comprise functional variants and fragments of the forgoing with at least 75% (e.g., 75%, 80%, 85%, 90%, 95%, 98%, 99%, 99.9% etc.) sequence identity at the protein level including variants from non-human species. In an embodiment, the first or the second constant antibody domain is an Ig-liker domain (e.g., IgG1-CH1. SEQ ID NO:582-596) and the second or the first constant antibody domain is an Ig linker domain (e.g., IgG-CL; SEQ ID NO: 581). In an embodiment, the TCR constant domain is an Ig-like linker domain represented by SEQ ID NO (DNA): 597-614; 19751-19756 and SEQ ID (PRT): 8977-8994, 20371-20376. In an embodiment, the antibody constant domains comprise functional variants and fragments of the forgoing with at least 75% (e.g., 75%, 80%, 85%, 90%, 95%, 98%, 99%, 99.9% etc.) sequence identity at the protein level including variants from non-human species. In an embodiment, the antibody and TCR constant domains comprise N-terminal and C-terminal deletion mutants of the forgoing that are missing between 1-50 amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50).

In an embodiment, the connecting peptide is derived from TCRα, TCRβ, TCRγ, TCRδ or pre-TCRα. Example connecting peptides are represented by SEQ ID NO (PRT): 8995-9002.

In an embodiment, the connecting peptide of HC-SAR is derived from TCRα, TCRβ, TCRγ, TCRδ or pre-TCRα. Example connecting peptides are represented by SEQ ID NO (PRT): 8995-9002. In an embodiment, the connecting peptide is derived from the same TCR chain as the transmembrane domain. For example, both the connecting peptide and the transmembrane domain are derived from human TCRα chain. In an embodiment, the connecting peptide is derived from the same TCR chain as the TCR constant domain. For example, both the connecting peptides and the TCR constant domain are derived from human TCRα chain. In an embodiment, the connecting peptide is derived from a different TCR chain as the transmembrane domain. For example, the connecting peptide is derived from human TCRβ chain and the transmembrane domain is derived from human TCRα chain. In an embodiment, the connecting peptide is derived from a different TCR chain as the TCR constant domain. For example, the connecting peptide is derived from human TCRβ chain and the TCR constant domain is derived from human TCRα chain.

In an embodiment, the ConnP, TM and CP domains of at least one TCR subunits are heterologous. In an embodiment, the ConnP and TM domains of at least one TCR subunits are heterologous. In an embodiment, the ConnP, TM and CP domains of both TCR subunits are heterologous. In an embodiment, the ConnP and TM domains of both TCR subunits are heterologous.

In an embodiment, the first TCR subunit comprises the ConnP of TCRβ and TM of TCRγ and the second TCR subunit is TCRα or TCRδ. In an embodiment, the first TCR subunit comprises the ConnP of TCRγ and TM of TCRβ and the second TCR subunit is TCRα or TCRδ. In an embodiment, the first TCR subunit comprises the ConnP of TCRα and TM of TCRδ and the second TCR subunit is TCRβ or TCRγ. In an embodiment, the first TCR subunit comprises the ConnP of TCRδ and TM of TCRα and the second TCR subunit is TCRβ or TCRγ.

In an embodiment, the first TCR subunit comprises the ConnP of TCRβ and TM of TCRγ and the second TCR subunit comprises the ConnP of TCRα and TM of TCRδ or the ConnP of TCRδ and TM of TCRα. In an embodiment, the first TCR subunit comprises the ConnP of TCRγ and TM of TCRβ and the second TCR subunit comprises ConnP of TCRα and TM of TCRδ or the ConnP of TCRδ and TM of TCRα. In an embodiment, the first TCR subunit comprises the ConnP of TCRα and TM of TCRδ and the second TCR subunit comprises ConnP of TCRβ and TM of TCRγ or ConnP of TCRγ and TM of TCRβ. In an embodiment, the first TCR subunit comprises the ConnP of TCRδ and TM of TCRα and the second TCR subunit comprises ConnP of TCRβ and TM of TCRγ or ConnP of TCRγ and TM of TCRβ.

In an embodiment, TM domain of TCR α, β, γ, δ and preTCRα is represented by SEQ ID NO (PRT): 31985-88 and 31992, respectively. In an embodiment, a TM domain of TCRα, TCRδ and preTCRα is a peptide with SEQ ID NO (PRT): 40606-40638 and 40639-40670, 40737-40758 respectively, or a functional variant or a homolog with 1, 2, 3, 4, 5 or 6 conservative amino acid substitutions except at residues 7, 12, 17 and 21. In an embodiment, a TM domain of TCRβ and TCRγ is a peptide with a sequence selected from SEQ ID NO: 40671-40708 and 40709-40736, respectively or a functional variant or a homolog with 1, 2, 3, 4, 5 or 6 amino acid substitutions except at residues 6, 12, and 16.

Example HC-SAR are provided in SEQ ID NO (DNA): 40162-40171 and 55078-55335.

In an embodiment, the vL and vH antibody domains of the above-described HC-SAR are replaced by one or more non-TCR antigen binding domains such as scFv, vHH, single variable domain antibody, FHVH (fully human vH domain), SVH (single vH domain), SVL (single vL domain), non-immunoglobulin antigen binding scaffold (e.g., cenyrin, affibody, DARPIN, D domain etc.), ligand binding domain of a receptor, receptor binding domain of a ligand, autoantigen, adaptor binding domain, epitope and mimotope etc.

In an embodiment, the disclosure provides a SAR comprising a heterodimer of two chains wherein each chain comprises a C, TM, ConnP, and CP and wherein at least one domain of the SAR is replaced by a corresponding domain from another species (e.g., human, mouse, monkey etc.). In an embodiment, the SAR comprises at least one non-TCR antigen binding domain which is attached to at least one of the two chains. In an embodiment, at least one chain of SAR comprises at least one transmembrane domain selected from SEQ ID NO: 31985-88 and 31992. In an embodiment, both chains of SAR comprise a transmembrane domain selected from SEQ ID NO: 31985-88 and 31992. In an embodiment, at least one domain of HC-SAR is of human origin. In an embodiment, at least one domain of HC-SAR is of non-human origin.

In some embodiments, the domains of any of the HC-SAR architectures described herein are fully human. In some embodiments, the domains of the HC-SAR are fully derived from a non-human species. In some embodiments, the domains of the HC-SAR are fully murine. In some embodiments, the HC-SAR is a chimera of human domains and non-human domains. In some embodiments, the HC-SAR is a chimera of human domains and murine domains.

In an embodiment, the HC-SAR forms a T cell receptor module (TCRM) that is capable of recruiting at least one TCR-associated signaling module when expressed in a T cell.

In an embodiment, the HC-SAR forms a non-canonical TCRM. Example non-canonical TCRM comprise modules that comprise signaling chains comprising transmembrane domains and/or connecting peptides of a) TCRα and TCRγ; and b) TCRβ and TCRδ. Example HC-SAR that form non-canonical TCRM are provided in SEQ ID NO: 32254-32265, 32268-69, 40142, 40145, 40146-40161. In an embodiment, the HC-SAR forms an interspecies non-canonical TCRM. Example such HC-SAR that form non-canonical TCRM are provided in SEQ ID NO: 32266-67.

In an embodiment, the TCR constant chain comprising any of the SAR of the disclosure (e.g., HC-SAR, SIR, Ab-TCR, zSIR, zCD16-SIR etc.) comprises one or more of the following features: (i) mutations that enhance the dimerization of the constant chains and reduce their pairing with the endogenous T cell receptor chains; (ii) one or both TCR constant chains are human codon optimized; and/or (iii) TCR constant chains are of human, mouse or dog origin.

In an embodiment, non-TCR antigen binding domain comprising any of the SAR of the disclosure (e.g., HC-SAR, SIR, Ab-TCR, zSIR, zCD16-SIR etc.) is selected from the group of an antibody; an antibody fragment selected from a Fv, a Fab, and a (Fab′)2; a heavy chain variable region of an antibody (vH domain); a light chain variable region of an antibody (vL domain); a single chain variable fragment (scFv); a single domain antibody (SDAB); a camelid VHH domain; a monomeric variable region of an antibody; a non-immunoglobulin antigen binding scaffold optionally selected from a DARPIN, D-domain (DD) an affibody, an affilin, an adnectin, an affitin, an obodies, a repebody, a fynomer, an alphabody, an avimer, an atrimer, a centyrin, a pronectin, an anticalin, a kunitz domain, an Armadillo repeat protein or a binding fragment thereof; the extracellular domain of receptor or a binding fragment thereof; a ligand or a binding fragment thereof; a bispecific-antibody; an autoantigen; am HLA molecule or a fragment thereof, β2M molecule or a fragment thereof and HLA/peptide complex

In an embodiment, the SAR of the disclosure, including HC-SAR and SIR, comprises variable regions of a heavy and light chains of an antibody or fragments thereof specific for a predefined target antigen, such that, when expressed, one of said heavy and light chains of the antibody or fragments thereof is attached to one of said two chains of said T-cell constant chains and the other of said heavy and light chains of the antibody or fragments thereof is attached to the other of said two chains of said T-cell constant chains.

In an embodiment, any of the SARs of the disclosure (e.g., HC-SAR, SIR, Ab-TCR, zSIR, zCD16-SIR etc.) further comprises one or more autonomous antigen binding domains (AABD) or fragments thereof that are operationally linked to the N-terminus or near the N-terminus of one or more non-TCR antigen binding domain via one or more optional linkers. In an embodiment, the AABD is located N-terminal to the one or more non-TCR antigen binding domains.

In an embodiment, one or more autonomous antigen binding domains (AABDs) or fragments thereof are selected from the group of: a single vH domain (SVH) or a fragment thereof; a single vL domain (SVL) or a fragment thereof; a vHH domain or a fragment thereof; a single domain antibody or a fragment thereof; a single variable domain of a TCR (svd-TCR) or a fragment thereof; a non-immunoglobulin antigen binding scaffold or a fragment thereof; a ligand-binding domain of a receptor or a fragment thereof; a receptor-binding domain of a ligand; an autoantigen or a fragment thereof; an adaptor binding domain or a fragment thereof; an adaptor or a fragment thereof; an epitope or a fragment thereof; and an Fe binding domain or a fragment thereof.

In an embodiment, non-immunoglobulin antigen binding scaffold is selected from the group of a DARPIN, a D domain (DD), an affibody, an affilin, an adnectin, an affitin, an obodies, a repebody, a fynomer, an alphabody, an avimer, an atrimer, a centyrin, a pronectin, an anticalin, a kunitz domain, and an Armadillo repeat protein.

In an embodiment, the one or more non-TCR antigen binding domain(s) and/or AABD comprising any of the SARs of the disclosure (e.g., HC-SAR, SIR, Ab-TCR, zSIR, zCD16-SIR etc.) bind to one or more of disease-associated antigens selected from the group shown in Table B.

In an embodiment, the one or more non-TCR antigen binding domain(s) and/or AABD comprising any of the SAR of the disclosure (e.g., HC-SAR, SIR, Ab-TCR, zSIR, zCD16-SIR etc.) are listed in Tables 3, 5 and 6.

In an embodiment, the non-TCR antigen binding domain(s) and/or AABD comprising any of the SAR of the disclosure (e.g., HC-SAR, SIR, Ab-TCR, zSIR, zCD16-SIR etc.) are selected from one or more of the (i) a light chain variable region (vL) encoded by a polynucleotide having a sequence of any one of SEQ ID NO 339-354, 19766-19776, 32912-33120, and 32006-32068 or sequences with at least 75% identity thereto in the framework regions and contains the complementarity determining regions (CDRs) of any of the forgoing polypeptides and which encodes a polypeptide that binds to its antigen and a complementary heavy chain variable region (vH) encoded by a polynucleotide having a sequence of any one of SEQ ID NO: 363-378, 19785-19795, 33121-33329, and 32069-32131 or sequences with at least 75% identity thereto in the framework regions and contains the CDRs of any of the forgoing polypeptides and which encodes a polypeptide that binds to its antigen;
(ii) a single chain variable fragment (scFv) encoded by a polynucleotide having a sequence of any one of SEQ ID NO 387-402, 19804-19814, 33330-33538 and 32132-32194 or sequences with at least 75% identity thereto in the framework regions and contains the CDRs of any of the forgoing polypeptides and which encodes a polypeptide that binds to its antigen; (iii) a camelid VHH domain encoded by a polynucleotide having a sequence of any one of SEQ ID NO 412-426, 32195-32213 or sequences with at least 75% identity thereto in the framework regions and contains the CDRs of any of the forgoing polypeptides and which encodes a polypeptide that binds to its antigen;
(iv) a non-immunoglobulin scaffold encoded by a polynucleotide having a sequence of any one of SEQ ID NO 435-450 or sequences with at least 75% identity thereto and which encodes a polypeptide that binds to its antigen.
(v) a receptor encoded by a polynucleotide having a sequence of any one of SEQ ID NO 437, 438, 445-448 or sequences with at least 75% identity thereto and which encodes a polypeptide that binds to its cognate;
(vi) a ligand encoded by a polynucleotide having a sequence of any one of SEQ ID NO 439 or sequences with at least 75% identity thereto and which encodes a polypeptide that binds to its cognate; and
(vii) a polynucleotide encoding a light chain variable region (vL) that comprise one or more of light chain complementary determining regions 1-3 (LC-CDR1-3) for a selected target antigen as set forth in any of SEQ ID Nos: 20989-21015, 41591-41861; 21024-21050, 41862-42132; and 21059-21085, 42133-42403 and a polynucleotide encoding a complementary heavy chain variable region (vH) that comprises one or more of heavy chain complementary determining regions 1-3 (HC-CDR1-3) for a selected target antigen as set forth in any of SEQ ID Nos: 21094-21120 and 42404-42674; 21129-21155, 42675-42945; and 21164-21190, 42946-43216.

In an embodiment, the non-TCR antigen binding domain(s) and/or AABD comprising any of the SAR of the disclosure (e.g., HC-SAR, SIR, Ab-TCR, zSIR, zCD16-SIR etc.) are selected from one or more of the (i) a variable light (vL) domain comprising a sequence of any one of SEQ ID Nos: 8719-8734, 20386-20396, 40759-41029 or a sequence with at least 85% identity thereto in the framework region or a sequence having up to 10 conservative amino acid substitutions in the framework region and contains the CDRs of any of the forgoing polypeptides and a complementary variable heavy (vH) domain comprising a sequence of any one of SEQ ID Nos: 8743-8758, 20405-20415, 41030-41300 or a sequence with at least 85% identity thereto in the framework region or a sequence having up to 10 conservative amino acid substitutions in the framework region and contains the CDRs of any of the forgoing polypeptides wherein the non-natural TCR antigen binding domain binds to its antigen;
(ii) a single domain antibody, a vHH domain, a SVH, and/or FHVH domain comprising a sequence as set forth in any one of SEQ ID NO: 8792-8806, 41572-41590, 43217-43318 or a sequence with at least 85% identity thereto in the framework region or a sequence having up to 10 conservative amino acid substitutions in the framework regions and contains the CDRs of any of the forgoing polypeptides and which bind to its antigen;
(iii) a non-immunoglobulin antigen binding domains having a sequence as set forth in any of SEQ ID NOs: 43364-43375, 43406-43410 or a sequence with at least 85% identity thereto or a sequence having up to 10 conservative amino acid substitutions and which bind to its antigen; (iv) an scFv domains comprising light chain complementary determining regions 1-3 (LC-CDR1-3) for a selected target antigen as set forth in any of SEQ ID Nos: 20989-21015, 41591-41861; 21024-21050, 41862-42132; and 21059-21085, 42133-42403 and complementary heavy chain variable region (vH) that comprises one or more of heavy chain complementary determining regions 1-3 (HC-CDR1-3) for a selected target antigen as set forth in any of SEQ ID Nos: 21094-21120 and 42404-42674; 21129-21155, 42675-42945; and 21164-21190, 42946-43216;
(v) an scFv fragment having a sequence selected from the group consisting of SEQ ID NO: 8767-8782 and 20424-20434, 41301-41571 or a sequence with at least 85% identity thereto in the framework region or a sequence each having up to 10 conservative amino acid substitutions in the framework regions and contains the CDRs of any of the forgoing polypeptides and which bind to its antigen;
(vi) one or more receptors comprising of amino acid sequences of any of SEQ ID Nos: 43377-43392 or a sequence with at least 85% identity thereto or sequence having up to 10 conservative amino acid substitutions;
(vii) one or more ligands comprising a sequence of any of SEQ ID NOs:43394-43404 or a sequence with at least 85% identity thereto or sequence having up to 10 conservative amino acid substitutions;
(viii) an extracellular domain of CD16A, NKG2D, CD4, PD1, desmoglein 3 (Dsg3) (ix) an extracellular domain of one or more of hTPO, mTPO, CGHα chain, CGHβ chain, FHβ chain, LHβ chain, TSHβ chain, APRIL, or combination thereof;
(x) a light chain variable region (vL) that comprise one or more of light chain complementary determining regions 1-3 (LC-CDR1-3) for a selected target antigen as set forth in any of SEQ ID Nos: 20989-21015, 41591-41861; 21024-21050, 41862-42132; and 21059-21085, 42133-42403 and a complementary heavy chain variable region (vH) that comprises one or more of heavy chain complementary determining regions 1-3 (HC-CDR1-3) for a selected target antigen as set forth in any of SEQ ID Nos: 21094-21120 and 42404-42674; 21129-21155, 42675-42945; and 21164-21190, 42946-43216.
(xi) any combination of (i)-(x).

A number of other suitable HC-SAR Alternatives in accordance with some embodiments herein are listed in Tables 2F-2J. In some embodiments, the HC-SAR is in accordance with any of Alternatives listed in Tables 2F-2J.

In an embodiment, the disclosure provides a SAR (e.g., HC-SAR, SIR, Ab-TCR, zSIR, zCD16-SIR etc.) polynucleotide that encodes for a polypeptide which comprises an autonomous antigen binding domain (AABD) or a fragment thereof that is joined in frame to the N-terminus or near the N-terminus of the vL and/or vH domain of the SAR via one or more optional linkers

In some embodiments, the invention provides a SAR (e.g., hybrid Synthetic Antigen Receptor or a hybrid-SAR or HC-SAR) in which at least one of the TCR chains comprises a transmembrane domain that is a hybrid of two different TCR chains. In an embodiment, the first TCR chain comprises a TCRβ chain Ig-like domain and TCRβ connecting peptide but comprises a transmembrane domain that is a hybrid of TCRβ and TCRγ chains and further comprises a TCRγcytosolic domain and the second TCR chain is a TCRα constant chain (i.e., comprising a TCRα Ig-like domain, TCRα connecting peptide, TCRα transmembrane domain and TCRα cytosolic domain) or a functional fragment or a functional variant thereof, including homolog from a non-human species. Example TCRβγ hybrid chains are provided in SEQ ID NO (DNA): 23228-23229 and SEQ ID NO (PRT): 23674-23675, 32223-32224, 32230-32239. Example complementary TCRα chains are provided in SEQ ID NOs: 453-463, 465-466, 19758, 21199-21224, 22848-22860, 22862-22962, 22974-23019, 23072-23074 and SEQ ID NO (PRT): 8833-8843, 8844-45, 20378, 20378, 21961-21986, 23294-23306, 23307-23408, 23420-23465, 23518-23520, 23558-23560, 23579-23581, 23587-89, 23595-97, 23603-05, 23611-13, 23620-22, 23631-33, 23642-44, 23653-55, 23672-73, 23679, 23681, 27936-27946, 27948-49, 27982-83, 27997, 27999, 28003, 28005, 28149-28194, 28217-28317, 28647-28672, 28686-28843, 28864-66, 29277-29286, 29888-89 and functional variants, fragments and homologs thereof. Example SARs with hybrid TCRβγ chains are provided in SEQ ID NO (DNA): 23251-23257, 32254-32258 and SEQ ID NO (PRT): 23697-23703. The hTCRa-T48C-C94S chain in the above construct can be substituted with any of the TCRα constant chains described above to generate novel SARs. The TCR chains in any of the above construct may also be a hybrid of two different species. In an embodiment, a TCR chain may comprise the Ig-like domain of human TCRα, a connecting peptide of mouse TCRα and transmembrane and cytosolic domain of human TCRα. In another example embodiment, a TCR chain may comprise the Ig-like domain of human TCRβ, a connecting peptide of mouse TCRγ and a transmembrane domain of human TCRγ and a cytosolic domain of human TCRγ. In an embodiment, the Ig-like domain of a TCR chain can be derived from an antibody. In an example embodiment a TCR chain may comprise the IgCL domain derived from an antibody that is joined in frame to the connecting peptide of TCRβ, transmembrane domain of TCRγ and cytosolic domain of human TCRγ. Example such TCR chain is represented by SEQ ID NO: 32230-32234 Similarly, the antigen binding domains (e.g., vL and vH) fragments can be substituted by antigen binding domains (e.g., vL, vH, scFv, non-immunoglobulin antigen binding scaffolds, receptor, ligands etc.) targeting different antigens to generate one and half chain or double chain SARs. Additionally, the [hTCRb-S57C-opt-L447-Ig-ConnP-hTCRg-opt-U427-TMCP chain](SEQ ID NO: 23674) can be substituted by hTCRb-S57C-opt-L435-Ig-ConnP-hTCRg-opt-U415-TMCP (SEQ ID NO: 23675) or functional variants, fragments, or homologs from non-human species.

In an embodiment, the first TCR chain comprises a TCRα chain Ig-like domain and TCRα connecting peptide but comprises a transmembrane domain that is a hybrid of TCRα and TCRδ chains and further comprises a TCRδ cytosolic domain and the second TCR chain is a TCRβ constant chain (i.e., comprising a TCRβ Ig-like domain, TCRβ connecting peptide, TCRβ transmembrane domain and TCRβ cytosolic domain) or a functional fragment or a functional variant thereof, including homolog from a non-human species. Example TCRαδ hybrid chains are provided in SEQ ID NO (DNA): 23223-23225, 32240-32249 and SEQ ID NO (PRT): 23669-23671. Example complementary TCRβ chains are provided in SEQ ID NOs: 467-487, 19759, 21225-21237, 23228-23229, 23234-23235 and SEQ ID NO (PRT): 8847-8867, 20379, 23674-23675, 23680-23681, 7950-27970, 27980-81, 27984-87, 27998, 28000, 28006, 28008, 28013-28058, 28127-28137, 28318-28444, 28673-28685, 28844-46, 28854-56, 28874-76, 28884-86, 29290-29310. Example SARs with hybrid TCRαδ chains are provided in SEQ ID NO (DNA): 23258-23264, 32259-32263 and SEQ ID NO (PRT): 23704-23710. The [hTCRb-S57C-opt-L447-Ig-ConnP-hTCRg-opt-U427-TMCP]chain in the above construct can be substituted with any of the TCRβ constant chains described above to generate novel SARs. Similarly, the antigen binding domains (e.g., vL and vH) fragments can be substituted by antigen binding domains (e.g., vL, vH, scFv, non-immunoglobulin antigen binding scaffolds, receptor, ligands etc.) targeting different antigens to generate one and half chain or double chain SARs. Additionally, the [hTCRa-T48C-opt-L351-Ig-ConnP-hTCRd-opt-U391-TMCP (SEQ ID NO: 23669) can be substituted by SEQ ID NO: 23670 or 23671 or functional variants, fragments, or homologs from non-human species.

In an embodiment, the first TCR chain comprises a TCRγ chain Ig-like domain and TCRγ connecting peptide but comprises a transmembrane domain that is a hybrid of TCRγ and TCRβ chains and further comprises a TCRβ cytosolic domain and the second TCR chain is a TCRδ constant chain (i.e., comprising a TCRδ Ig-like domain, TCRδ connecting peptide, TCRδ transmembrane domain and TCRδ cytosolic domain) or a functional fragment or a functional variant thereof, including homolog from a non-human species. Example TCRγβ hybrid chains are provided in SEQ ID NO (DNA): 23230-23231, 32250-32251 and SEQ ID NO (PRT): 23676-23677. Example complementary TCRδ chains are provided in SEQ ID NOs: 494, 23226-27, 32221 and SEQ ID NO (PRT): 8874, 23672-73.

In an embodiment, the first TCR chain comprises a TCRδ chain Ig-like domain and TCRδ connecting peptide but comprises a transmembrane domain that is a hybrid of TCRδ and TCRα chains and further comprises a TCRα cytosolic domain and the second TCR chain is a TCRγ constant chain (i.e., comprising a TCRγIg-like domain, TCRγ connecting peptide, TCRγ transmembrane domain and TCRγcytosolic domain) or a functional fragment or a functional variant thereof, including homolog from a non-human species. Example TCRδα hybrid chains are provided in SEQ ID NO (DNA): 23226-23227, and SEQ ID NO (PRT): 23672-23673. Example complementary TCRγ chains are provided in SEQ ID NO (DNA): 493, 23230-31 and SEQ ID NO (PRT): 8873, 8900-8903, 18351-18372, 18441-18462, 18474-18484, 18713-18813, 23676-77, 28105-28115, 28138-28148, 27975-76, 27977-79, 27988-93, 28002, 28010, 28012, 28195-28216, 28445-28545, 28847-51, 28857-61, 28867-28871, 28877-81, 28887-91, 29315-16 and functional variants, fragments, and homologs thereof. Example hybrid chain SAR are provided in SEQ ID NO: 32268-32269.

In an embodiment, the invention provides a SAR (e.g., HC-SAR) comprising TCR chains of fragments thereof represented by SEQ ID NO: 32223-32253 or functional variants or homologs which encodes for polypeptides with at least 75% (e.g., 75%, 80%, 85%, 90%, 95%, 98%, 99% etc.) sequence identity to polypeptides encoded by sequences SEQ ID NO: 32223-32253.

In an embodiment, the invention provides a one and a half or a double chain SAR (e.g., SAR or HC-SAR) comprising TCRα, TCRβ, TCRγ, TCRδ or preTCRα chains selected from any of the TCRα, TCRβ, TCRγ, TCRδ or preTCRα chains or fragments provided herein (e.g., Tables 7 and 22 of the provisional application) or functional fragments or variants thereof.

In an embodiment, the invention provides one and a half or a double chain SAR (e.g., SIR or HC-SAR etc.) comprising a TCRα chain with sequences selected from the group of SEQ ID NO (DNA): 453-463, 465-466, 19758, 16902-16958, 17003-17103, 21199-21224, 22848-22860, 22862-22962, 22974-23019, 23072-23074 and SEQ ID NO (PRT): 8833-8843, 8844-45, 20378, 20378, 18384-18440, 18485-18585, 21961-21986, 23294-23306, 23307-23408, 23420-23465, 23518-23520, 23558-23560, 23579-23581, 23587-89, 23595-97, 23603-05, 23611-13, 23620-22, 23631-33, 23642-44, 23653-55, 23672-73, 23679, 23681, 27936-27946, 27948-49, 27982-83, 27997, 27999, 28003, 28005, 28149-28194, 28217-28317, 28647-28672, 28686-28843, 28864-66, 29277-29286, 29888-89, 32218-32219 or a functional variant, fragment or a homolog which encodes for polypeptides with at least 70% (e.g., 70%, 75%, 80%, 85%, 90%, 95%, 98%, 99%, or 100%) sequence identity to polypeptides encoded by the above sequences.

In an embodiment, the invention provides a (e.g., SIR or HC-SAR etc.) comprising a TCRα chain with a sequence selected from the group of SEQ ID NO (PRT): 31963, 31969, 31971, 31973, 31975, 31977, 31979, 31981, 31983 or a functional variant, fragment or a homolog which encodes for polypeptides with at least 70% (e.g., 70%, 75%, 80%, 85%, 90%, 95%, 98%, 99%, or 100%) sequence identity thereto.

In an embodiment, the invention provides one and a half or a double chain SAR (e.g., SIR or HC-SAR etc.) comprising a TCRβ chain with a sequence selected from the group of SEQ ID NO (PRT): 31964, 31965-31968, 31970, 31972, 31974, 31976, 31978, 31980, 31982 and 31984 or a functional variant, fragment or a homolog which encodes for polypeptides with at least 70% (e.g., 70%, 75%, 80%, 85%, 90%, 95%, 98%, 99%, or 100%) sequence identity thereto.

In an embodiment, the invention provides a one and a half or a double chain SAR (e.g., SIR or HC-SAR etc.) comprising a TCRβ chain with sequences selected from the group of SEQ ID NO (DNA):467-487, 16777-16822, 16891-16901, 17104-17230, 21225-21237, 23228-23229, 23234-23235 and SEQ ID NO (PRT): 8847-8867, 18259-18304, 18373-18383, 18586-18712, 23674-23675, 23680-23681, 7950-27970, 27980-81, 27984-87, 27998, 28000, 28006, 28008, 28013-28058, 28127-28137, 28318-28444, 28673-28685, 28844-46, 28854-56, 28874-76, 28884-86, 29290-29310, and 32220 or a functional variant, fragment or a homolog thereof with at least 70% (e.g., 70%, 75%, 80%, 85%, 90%, 95%, 98%, 99%, or 100%) sequence identity thereto.

In an embodiment, the invention provides a one and a half or a double chain SAR (e.g., SIR or HC-SAR etc.) comprising a TCRγ chain with sequences selected from the group of SEQ ID NO (DNA): 493, 17231-17331, 16869-16879, 16959-16980, 16992-17002, 23230-31 and SEQ ID NO (PRT): 8900-8903, 18351-18372, 18441-18462, 18474-18484, 18713-18813, 23676-77, 28105-28115, 28138-28148, 27975-76, 27977-79, 27988-93, 28002, 28010, 28012, 28195-28216, 28445-28545, 28847-51, 28857-61, 28867-28871, 28877-81, 28887-91, 29315-16 and 32222 or a functional variant, fragment or a homolog which encodes for polypeptides with at least 70% (e.g., 70%, 75%, 80%, 85%, 90%, 95%, 98%, 99%, or 100%) sequence identity to polypeptides encoded by the above sequences.

In an embodiment, the invention provides a one and a half or a double chain SAR (e.g., SIR or HC-SAR etc.) comprising a TCRδ chain with sequences selected from the group of SEQ ID NO (DNA): 495, 16823-16868, 16880-16890, 16981-16991, 17332-17432, 23226-27 and SEQ ID NO (PRT): 8875, 18305-18350, 18362-18372, 18463-18473, 18814-18914, 23672-73, 32221 or a functional variant, fragment or a homolog which encodes for polypeptides with at least 70% (e.g., 70%, 75%, 80%, 85%, 90%, 95%, 98%, 99%, or 100%) sequence identity to polypeptides encoded by the above sequences.

The disclosure provides one and a half or a double chain SAR (e.g., SIR or HC-SAR etc.) comprising an antigen binding domain (e.g., scFv, vHH, FHVH, non-immunoglobulin antigen binding scaffold, receptor, ligand, adaptor, epitope tag, autoantigen etc.) that is operationally joined to one hybrid TCR constant chain or variant thereof and is co-expressed with a complementary hybrid TCR constant chain or variant thereof. In an embodiment, the antigen binding domain of SAR (e.g., SIR) is a non-TCR antigen binding domain (e.g., vL, vH, single domain antibody, scFv, vHH, FHVH, SVH, SVL, non-immunoglobulin antigen binding scaffold, receptor, ligand, adaptor, epitope tag, autoantigen etc.). A non-TCR antigen binding domain is an antigen binding domain that is structurally distinct from a TCR found in nature. In an example embodiment, a non-TCR antigen binding domain lacks the variable domains of a TCR, e.g., Vα, Vβ, Vγ and/or Vδ domains. The example hybrid TCR constant chains are provided in SEQ ID NO (DNA): 529-544 and SEQ ID NO (PRT): 8909-8924, 22112-22576, 23420-23677, 27936-31955. The disclosure also provides SAR comprising hybrid TCR constant chains with at least 70% (e.g., 70%, 75%, 80%, 90%, 95%, 98%, 99% or 100%) amino acid identity to hybrid TCR chains provided in SEQ ID NO (PRT): 8909-8924, 22112-22576, 23420-23677, 27936-31955 and functional variants and mutants thereof, including homologs from non-human species. Example one and half chain (OHC) SAR (e.g., SIR) comprising hybrid TCR constant chains and non-TCR antigen binding domains targeting various antigens are provided in SEQ ID NO (DNA): 8106-8393 and SEQ ID NO (PRT):16487-16774 (see also Tables 19 and 20 of the provisional application). In an embodiment, the OHC SAR (e.g., SIR) further comprises one or more autonomous antigen binding domains (AABD) that are operationally linked to the N-terminus or near the N-terminus of the non-TCR antigen binding domains via one or more optional linkers.

In an embodiment, a TCRα chain comprising SAR (e.g., SIR or HC-SAR etc.) is a chain comprising the transmembrane domain of TCRα represented by SEQ ID NO (PRT): 9005 or 31985 or homologs or functional variants thereof, including homologs from non-human species. In an embodiment, a TCR α chain comprising a SAR is a chain comprising a transmembrane domain with at least 70% (e.g., 70%, 75%, 80%, 90%, 95%, 98%, 99% or 100%) amino acid identity to SEQ ID NO (PRT): 9005. In an embodiment, a TCRα chain comprising a SIR is a chain comprising the connecting peptide (or hinge domain) of TCRα (SEQ ID NO (PRT): 8995-6) or homologs or functional variants thereof with at least 70% (e.g., 70%, 75%, 80%, 90%, 95%, 98%, 99% or 100%) amino acid identity thereto, including homologs from non-human species. In an embodiment, a TCRα chain comprising a SIR is a chain comprising the connecting peptide (or hinge domain) and transmembrane domains of TCRα or functional variants thereof, including homologs from non-human species. In an embodiment, the SAR comprises a TCR chain comprising the sequence with SEQ ID NO: 40606-40638. In an embodiment, the invention provides a SAR comprising a TCR chain comprising the sequence with SEQ ID NO: 31985. In an embodiment, a TCRδ chain comprising a SAR is a chain with the sequence -LSVIGFRILLLKVAGFNLLMTLRLWS-(SEQ ID NO: 40606) or a functional variant or a homolog with 1, 2, 3, 4, 5 or 6 amino acid substitutions except residues at positions 7, 12, 17 and 21 are Y, K, Y and T, respectively. In an embodiment, a TCRα chain comprising a SAR is a chain that comprises a fragment of TCRα (SEQ ID NO: 8834) and is able to dimerize with a TCRβ chain or fragment thereof.

In an embodiment, a preTCRα chain comprising a SAR is a chain comprising the transmembrane domain of pre-TCRα or homologs or functional variants thereof, including homologs from non-human species. In an embodiment, a preTCRα chain comprising a SAR is a chain comprising a transmembrane domain with at least 70% (e.g., 70%, 75%, 80%, 90%, 95%, 98%, 99% or 100%) amino acid identity to SEQ ID NO (PRT): 40737. In an embodiment, a TCRα chain comprising a SAR is a chain comprising the connecting peptide (or hinge domain) of preTCRα or homologs or functional variants thereof with at least 70% (e.g., 70%, 75%, 80%, 90%, 95%, 98%, 99% or 100%) amino acid identity thereto, including homologs from non-human species. In an embodiment, a TCRα chain comprising a SAR is a chain comprising the connecting peptide (or hinge domain) and transmembrane domains of preTCRα or functional variants thereof, including homologs from non-human species. In an embodiment, the SAR comprises a TCR chain comprising the sequence with SEQ ID NO: 40737-40758. In an embodiment, the invention provides a SAR comprising a TCR chain comprising the sequence with SEQ ID NO: 31992. In an embodiment, a TCRδ chain comprising a SAR is a chain with the sequence -LWLGVLRLLLFKLLLFDLLLTCSCL- (SEQ ID NO: 40737) or a functional variant or a homolog with 1, 2, 3, 4, 5 or 6 amino acid substitutions except residues at positions 7, 12, 17 and 21 are Y, K, Y and T, respectively. In an embodiment, a preTCRα chain comprising a SAR is a chain that comprises a fragment of preTCRα (SEQ ID NO:8871) and is able to dimerize with a TCRβ chain or fragment thereof.

In an embodiment, a TCRβ chain comprising a SAR is a chain comprising the transmembrane domain of TCRβ (SEQ ID NO (PRT): 9006) or homologs or functional variants thereof, including homologs from non-human species. In an embodiment, a TCRα chain comprising a SAR is a chain comprising a transmembrane domain with at least 70% (e.g., 70%, 75%, 80%, 90%, 95%, 98%, 99% or 100%) amino acid identity to SEQ ID NO (PRT): 9006. In an embodiment, a TCRβ chain is a chain comprising the connecting peptide (or hinge domain) of TCRβ (SEQ ID NO (PRT): 8997-8) or homologs or functional variants thereof with at least 70% (e.g., 70%, 75%, 80%, 90%, 95%, 98%, 99% or 100%) amino acid identity thereto, including homologs from non-human species. In an embodiment, a TCRβ chain is a chain comprising the connecting peptide (or hinge domain) and transmembrane domains of TCRβ or functional variants thereof. In an embodiment, a TCRβ chain is a chain that comprises a fragment of TCRβ (SEQ ID NO: 8847-48) and is able to dimerize with a TCRα chain or fragment thereof. In an embodiment, the invention provides a SAR comprising a TCR chain comprising the sequence with SEQ ID NO:31986. In an embodiment, a TCRβ comprising a SAR is a chain with the sequence SATILYEILLGKATLYAVLVSALVLM (SEQ ID NO: 40671) or a functional variant or a homolog with 1, 2, 3, 4, 5 or 6 amino acid substitutions except residues at positions 6, 12, and 16 are Y, K and Y, respectively.

In an embodiment, a TCRγ chain comprising a SAR is a chain comprising the transmembrane domain of TCRγ (SEQ ID NO (PRT): 9007) or homologs or functional variants thereof with at least 70% (e.g., 70%, 75%, 80%, 90%, 95%, 98%, 99% or 100%) amino acid identity to SEQ ID NO (PRT): 9007. In an embodiment, a TCRγ chain is a chain comprising the connecting peptide (or hinge domain) of TCRγ (SEQ ID NO (PRT): 8999-9000) or homologs or functional variants thereof with at least 70% (e.g., 70%, 75%, 80%, 90%, 95%, 98%, 99% or 100%) amino acid identity to SEQ ID NO (PRT): 8999-9000. In an embodiment, a TCRγ chain is a chain comprising the connecting peptide (or hinge domain) and transmembrane domains of TCRγ or functional variants thereof. In an embodiment, a TCRγ chain is a chain that comprises a fragment of TCRγ (SEQ ID NO: 8872-73) and is able to dimerize with a TCRδ chain or fragment thereof. In an embodiment, the invention provides a SAR comprising a TCR chain comprising the sequence with SEQ ID NO:31987. In an embodiment, a TCRγ chain comprising a SAR is a chain with the sequence -SAYYMYLLLLLKSVVYFAIITCCLLR- (SEQ ID NO: 40709) or a functional variant or a homolog with 1, 2, 3, 4, 5 or 6 amino acid substitutions except residues at positions 6, 12, and 16 are Y, K and Y, respectively.

In an embodiment, a TCRδ chain comprising a SAR is a chain comprising the transmembrane domain of TCRδ (SEQ ID NO (PRT): 9008) or homologs or functional variants thereof with at least 70% (e.g., 70%, 75%, 80%, 90%, 95%, 98%, 99% or 100%) amino acid identity to SEQ ID NO (PRT):9008. In an embodiment, a TCRδ chain is a chain comprising the connecting peptide (or hinge domain) of TCRδ (SEQ ID NO (PRT): 9001-2) or homologs or functional variants thereof. In an embodiment, a TCRδ chain is a chain comprising the connecting peptide (or hinge domain) and transmembrane domains of TCRδ or functional variants thereof with at least 70% (e.g., 70%, 75%, 80%, 90%, 95%, 98%, 99% or 100%) amino acid identity to SEQ ID NO: 9001-2. In an embodiment, a TCRδ chain is a chain that comprises a fragment of TCRδ (SEQ ID NO: 8874-75) and is able to dimerize with a TCRγ chain or fragment thereof. In an embodiment, the invention provides a SAR comprising a TCR chain comprising the sequence with SEQ ID NO:31988. In an embodiment, a TCRδ chain comprising a SAR is a chain with the sequence LTVLGLRMLFAKTVAVNFLLTAKLFF (SEQ ID NO: 40639) or a functional variant or a homolog with 1, 2, 3, 4, 5 or 6 amino acid substitutions except residues at positions 7, 12, 17 and 21 are Y, K, Y and T, respectively.

A number of nucleic acid arrangements encoding the first and second hybrid chains are suitable in accordance with some embodiments herein

In some embodiments, a first nucleic acid encodes the first hybrid chain comprising the constant domain of the alpha chain combined with the connecting peptide, transmembrane domain and cytoplasmic tail of the beta chain, and reciprocally, a second nucleic acid encodes the second hybrid chain comprising the constant domain of the beta chain combined with the connecting peptide, transmembrane domain and cytoplasmic tail of the alpha chain.

In some embodiments, a first nucleic acid encodes the first hybrid chain comprising the constant domain and connecting peptide of the alpha chain combined with the transmembrane domain and cytoplasmic tail of the beta chain, and reciprocally, a second nucleic acid encodes the second hybrid chain comprising the constant domain and connecting peptide of the beta chain combined with the transmembrane domain and cytoplasmic tail of the alpha chain.

In some embodiments, a first nucleic acid encodes the first hybrid chain comprising the constant domain of the gamma chain combined with the connecting peptide, transmembrane domain and cytoplasmic tail of the delta chain, and reciprocally, a second nucleic acid encodes the second hybrid chain comprising the constant domain of the delta chain combined with the connecting peptide, transmembrane domain and cytoplasmic tail of the gamma chain.

In some embodiments, a first nucleic acid encodes the first hybrid chain comprising the constant domain and connecting peptide of the gamma chain combined with the transmembrane domain and cytoplasmic tail of the delta chain, and reciprocally, a second nucleic acid encodes the second hybrid chain comprising the constant domain and connecting peptide of the delta chain combined with the transmembrane domain and cytoplasmic tail of the gamma chain.

In some embodiments, the different domains of a SAR are joined by one or more linkers. In an example embodiment, the non-TCR antigen binding domain and the TCR constant domains of a SAR (e.g., SIR, HC-SIR, zSIR, uSAR-TCR, zCD16 SAR etc.) are joined by a linker. In an embodiment, the linker is a short peptide linker. In an embodiment, the linker is of any length. In some embodiment, the linker is between 2-50 amino acids in length or between 5-100 amino acids in length.

In some embodiments, the first nucleic acid and second nucleic acid are part of the same construct (e.g., a vector). Optionally, the first and second nucleic acid can be under the control of different promoters. Optionally, the first and second nucleic acid can be under the control of the same promoter. In an embodiment, at least one of the nucleic acid is under the control of a promoter driven by a SynNotch receptor. Optionally, the first and second nucleic acid can be separated by a 2A polypeptide-encoding polynucleotide sequence.

A number of promoters are suitable for driving expression of the first and/or second nucleic acid in accordance with some embodiments herein. The promoter can be naturally occurring or non-naturally occurring. Examples of promoters include, but are not limited to, viral promoters, plant promoters and mammalian promoters. Examples of viral promoters include, but are not limited to, MND3 promoter (SEQ ID NO: 25-26) and cytomegalovirus (CMV) immediate early promoter. In some embodiments, a cell-type specific promoter can be used (e.g., CD4 vs CD8 vs Treg), depending on the type of cell to be genetically engineered. In some embodiments, a promiscuous promoter can be used.

In some embodiments, the SAR (e.g., HC-SAR) is targeted to an endogenous T cell gene locus (e.g., TRAC, TRBC, TRDC, TRGC etc.). In some embodiments, the SAR (e.g., HC-SAR) is expressed under the promoter of an endogenous TCR gene (e.g., TRAC, TRBC, TRDC, TRGC). Example constructs for targeting a SAR (e.g., HC-SAR) to the TRAC locus are provided in SEQ ID NO: 8396-8404. In an embodiment, the SAR (e.g., HC-SAR) are targeted to an endogenous locus using a gene targeting system known in the art, such as CRISP/Cas9, ARCUS, Zn finger nucleases etc.

In an embodiment, the expression of one or both chains of a SAR and/or accessory modules and/or therapeutic controls is under the transcriptional control of a synthetic notch (synNotch) receptor polypeptide that comprises from N to C terminus: an extracellular antigen binding domain, a Notch core domain comprising one or more proteolytic cleavage sites, and an intracellular domain comprising a transcriptional activator comprising a DNA binding domain and a transactivation domain, wherein binding of the extracellular antigen binding domain to a target antigen (e.g., CD19, MSLN etc.) induces cleavage of the Notch core domain at the one or more proteolytic cleavage sites, thereby releasing the intracellular domain and the transcriptional regulator. In embodiments the transcriptional regulator is a transcriptional activator.

In some embodiments, the disclosure provides vectors expressing the SAR (e.g., HC-SAR). In an embodiment, the SAR vector is selected from the group consisting of a DNA vector, an RNA vector, a plasmid, a lentivirus vector, an adenoviral vector, a retrovirus vector, a baculovirus vector, a sleeping beauty transposon vector, and a piggyback transposon vector.

In some embodiments, a vector provided herein includes a gene for a selectable marker that is effective in a eukaryotic cell. Typical selectable marker genes encode proteins that confer resistance to an antibiotic or toxin (e.g., neomycin, methotrexate, gentamycin, zeocin) or complement auxotrophic deficiencies.

In some embodiments, the SAR (e.g., HC-SAR) is expressed in a CD4 T cell. In some embodiments, the HC-SAR is expressed in a CD8 T cell. In an embodiment, the SAR (e.g., HC-SAR) of the invention is co-expressed with a therapeutic control and/or an accessory module. In an embodiment, the accessory modules and therapeutic controls are expressed using the same vector as the SAR (e.g., HC-SAR) or from a separate vector.

According to the present invention, the modified immunoresponsive cells expressing or presenting a SAR (e.g., uTCR-SAR, SIR, HC-SAR, zSIR, zCD16-SAR etc.) may further express or present a heterologous co-receptor (e.g., the cell is transduced with or engineered to comprise a nucleic acid sequence encoding a co-receptor, for example by gene knock in). The heterologous co-receptor may be a CD8 co-receptor and/or a CD4 co-receptor. The CD8 co-receptor may comprise a dimer or pair of CD8 chains which comprises a CD8-α, and CD8-β chain or a CD8-α and CD8-α chain. Preferably, the CD8 co-receptor is a CD8αα co-receptor comprising a CD8-α and CD8-α chain. A CD8α co-receptor may comprise the amino acid sequence of at least 80% identity to SEQ I) NO: 9025, SEQ ID NO: 9025 or a variant thereof. A CD8β co-receptor may comprise the amino acid sequence of at least 80% identity to SEQ ID NO: 9024, SEQ ID NO: 9024 or a variant thereof. The CD8α co-receptor may be a homodimer. Preferably the modified immunoresponsive cells expressing or presenting a heterologous SAR (e.g., uTCR-SAR, SIR, zSIR etc.) may further express or present a heterologous co-receptor (e.g., the cell is transduced with or engineered to comprise a nucleic acid sequence encoding a co-receptor, for example by gene knock in) which is a CD8 co-receptor as defined herein.

CD8 co-receptor may comprise the reference amino acid sequence of SEQ IID NO: 9024-9025 or may be a variant thereof. The CD8 co-receptor may be encoded by the reference nucleotide sequence of SEQ ID NO: 644-645 or may be a variant thereof. According to the invention the heterologous co-receptor may comprise a CD8 co-receptor in which, in the Ig-like V-type domain comprises CDRs having the sequence comprising: (i) amino acids 45-53 of SEQ ID NO: 9025, (ii) amino acids 72-79 of SEQ ID NO: 9025, (iii) amino acids 80-117 of SEQ ID NO: 9025, or sequences having at least 70% sequence identity thereto.

According to the invention the heterologous CD8 co-receptor may comprise a CD8 co-receptor which comprises or in-which, in the Ig-like V-type domain comprises, residues 22-135 of the amino acid sequence of SEQ ID No: 9025, or an amino acid sequence in which amino acid residues 22-44, 54-71, 80-117, 124-135 thereof have at least 70%, 75%, 80%, 85%, 90% or 95% identity to the sequence of amino acid residues 22-44, 54-71, 80-117, 124-135, CDR 1, CDR 2, CDR 3, respectively of SEQ ID No: 9025 and in which amino acid residues 45-53, 72-79 and 118-123 have at least 70%, 75%, 80%, 85%, 90% or 95% identity to the sequence of amino acid residues 45-53, 72-79 and 118-123 respectively of SEQ ID No: 9025.

According to the invention the CD8 co-receptor may comprise a CD8 co-receptor in which, or in which in the Ig-like V-type domain, the sequence of: (i) amino acid residues 22-44 thereof may have (a) at least 70%, 75%, 80%, 85%, 90% or 95% identity to the sequence of amino acid residues 22-44 of SEQ 11) NO: 9025 or (b) may have one, two or three amino acid residues inserted or deleted relative to residues 22-44 of SEQ ID NO: 9025, (ii) amino acid residues 45-53 is amino acids 45-53 of SEQ ID NO: 9025, (iii) amino acid residues 54-71 thereof may have (a) at least 70%, 75%, 80%, 85%, 90% or 95% identity to the sequence of amino acid residues 54-71 of SEQ ID NO. 9025 or (b) may have one, two or three amino acid residues inserted or deleted relative to the sequence of amino acid residues 54-71 of SEQ ID NO: 9025, (iv) amino acid residues 72-79 may be amino acids 72-79 of SEQ ID NO: 9025, (v) amino acid residues 80-117 thereof may have at least 70%, 75%, 80%, 85%, 90% or 95% identity to the sequence of amino acid residues 80-117 of SEQ I) NO: 9025 or may have one, two or three insertions, deletions or substitutions relative to the sequence of amino acid residues 80-117 of SEQ ID NO: 9025; (vi) amino acids 118-123 may be amino acids 80-117 of SEQ ID NO: 9025, (vii) amino acid residues 124-135 thereof may have at least 70%, 75%, 80%, 85%, 90% or 95% identity to the sequence of amino acid residues 124-135 of SEQ ID NO: 9025 or may have one, two or three insertions, deletions or substitutions relative to the sequence of amino acid residues 124-135 of SEQ ID NO: 9025.

According to the invention the CD4 co-receptor may comprise the reference amino acid sequence of SEQ ID NO: 9026 or may be a variant thereof. The CD4 co-receptor may be encoded by the reference nucleotide sequence of SEQ ID NO: 646 or may be a variant thereof.

The modified immunoresponsive cells (e.g. T, NK, monocyte cell etc.) that express heterologous CD8 and/or CD4 co-receptor may demonstrate improved affinity and/or avidity and/or improved cellular activation, as determinable by the assays disclosed herein, towards or on stimulation by antigenic peptide, tumor, or cancer antigen optionally when presented on HLA.

The disclosure also provides immunoresponsive cells expressing an accessory module comprising IL12, IL12 fusion protein or a variant thereof that can be co-expressed with the SARs (e.g., uTCR-SAR, SIR, HC-SAR, zCD16-SAR etc.) of the disclosure. Example IL12 and IL12 fusion proteins (e.g., IL12f and IL12f-CD28-Hinge-TM) are provided in Table 25 of the provisional patent application (SEQ ID NO (DNA): 968-969 and SEQ ID NO (PRT): 9348-9349). Example SARs (e.g., uTCR-SAR) comprising IL12f and IL12f-CD28-Hinge-TM are also provided in Table 25 of the provisional patent application (SEQ ID NO: 967, 970-980; and SEQ ID NO (PRT): 9347, 9350-9360). The disclosure provides immunoresponsive cells expressing a SAR and an accessory module comprising IL12 and IL12 fusion proteins that have at least 80% identity to IL12 and IL12 fusion proteins (e.g., IL12f and IL12f-CD28-Hinge-TM) provided in SEQ ID NO (DNA): 968-969 and SEQ ID NO (PRT): 9348-9349. In an embodiment, an immunoresponsive cells expressing a SAR and an IL12 or an IL12f fusion protein shows increased sensitivity to a target antigen expressing cell as compared to an immunoresponsive cells expressing a SAR without an IL12 or an IL12f fusion protein.

According to the invention, the modified immunoresponsive cells (e.g., T, NK, monocyte, macrophage etc.) may have an improved or increased expression of CD40L, cytokine production, cytotoxic activity, induction of dendritic cell maturation or induction of dendritic cell cytokine production, optionally in response to cancer and/or tumor antigen or peptide antigen thereof optionally as presented by tumor of cancer cell or tissue, in comparison to immunoresponsive cells lacking the heterologous CD8 or CD4 co-receptor.

In an embodiment, the disclosure also provides polypeptides encoded by any of the SAR (e.g., HC-SAR) polynucleotides described herein.

In accordance with some embodiments, methods of making T cells expressing HC-SARs are provided. The SAR, including HC-SAR, SAR and Ab-TCR, of the disclosure can be also expressed in cells other than T cells (e.g., NK, macrophage etc.) that have been engineered to exogenously express one or more of CD3ε, CD3γ, CD3δ and CD3ζ chains. In an embodiment, cells other than T cells (e.g., NK, macrophage etc.) have been engineered to exogenously express CD3ε, CD3γ, and CD3δ chains. In an embodiment, cells other than T cells (e.g., NK) have been engineered to express CD3ε, CD3γ, CD3δ and CD3ζ chains.

In an embodiment, an ex vivo or in vivo method of making a SAR (e.g., HC-SAR)-expressing immune effector cell (e.g., T, NK, NKT, stem cell etc.) is described. In an embodiment, the method comprises introducing a vector encoding a recombinant polynucleotide of claim 1 into an immune effector cell or a hematopoietic stem cell or progenitor cell that can give rise to an immune effector cell, under conditions such that the HC-SAR polypeptide is expressed. In an embodiment, the method involves introducing in vitro transcribed RNA or RNAs or synthetic RNA or RNAs into a cell or population of cells, where the RNA or RNAs comprises a recombinant polynucleotide or polynucleotides encoding the SAR (e.g., HC-SAR) and/or any accessory module or therapeutic control.

T cells can be isolated from a subject or a donor. Optionally, T cells can be provided from a cell line. T cells can be genetically engineered to express a HC-SAR. T cells can be genetically modified using any of a number of approaches known to the skilled artisan. The T cells can be contacted with/transfected with an expression vector or collection of expression vectors encoding a HC-SAR as described herein. In some embodiments, two expression vectors, one vector encoding a first chain of a HC-SAR and one vector encoding a second chain of a HC-SAR, are contacted with the isolated T cells. In some embodiments, the expression vector carries both the first and second nucleic acids and therefore only one expression vector is contacted with the isolated T cells. The vectors express HC-SAR in accordance with any of the HC-SAR configurations.

Optionally, following contact with a single vector or two vectors, a T cell is placed in a selective culture medium. In some embodiments, the vector comprises a selectable marker gene which encodes a protein that confers resistance to an antibiotic or toxin present in the selective culture medium. Optionally, the selectable marker gene encodes a protein that complements an auxotrophic deficiency and enables the T cell to survive in a selective culture medium. Optionally, a selection step can be performed wherein T cells contacted with/transfected with a HC-SAR expression vector are sorted by flow cytometry to select for T cells that express the HC-SAR and separate them from the T cells that do not express the HC-SAR. Optionally, sorting by flow cytometry is used to select for T cells expressing relatively high levels of HC-SAR. Optionally, the expression of endogenous TCR is knocked down or eliminated in the transfected T cell. Optionally, the effect of knockdown or elimination of endogenous TCR is measured, for example, by determining messenger RNA levels and/or polypeptide levels of endogenous TCR.

In some embodiments, the expression of an endogenous TCR is repressed or eliminated prior to sorting by flow cytometry of HC-SAR-expressing T cell and prior to administering a HC-SAR-expressing T cell to a subject.

Optionally, the genetically modified T cells are subsequently used, for example, to induce an immune response in a subject with a cancer or an infectious disease. Optionally, genetically modified T cells are preserved for later use, for example cryogenically preserved. For example, allogeneic T cells may be preserved in a bank for later use is a suitable subject.

In some embodiments, the genetically modified T cell comprises a first nucleic acid encoding the first hybrid chain and a second nucleic acid encoding the second hybrid chain of any of the SARs. In some embodiments, the first nucleic acid and the second nucleic acid encode the first hybrid chain and the second hybrid chain of any of a SAR. In some embodiments, the first nucleic acid and the second nucleic acid encode the first hybrid chain and the second hybrid chain. In some embodiments, the first nucleic acid and the second nucleic acid encode the first hybrid chain and the second hybrid chain. In some embodiments, the first nucleic acid and the second nucleic acid encode the first hybrid chain and the second hybrid chain. In some embodiments, the first nucleic acid and the second nucleic acid encode the first hybrid chain and the second hybrid chain. In some embodiments, the first nucleic acid and the second nucleic acid encode the first hybrid chain and the second hybrid chain.

In some embodiments, the cell has one or more of the following gene modifications and/or characteristics: a) lacks the expression of a functional endogenous TCR on its surface; b) decreased expression of a functional endogenous TCR on its surface; c) lacks the expression of or has decreased expression of one or more subunits that comprise a functional endogenous TCR; d) lacks the expression of or has decreased expression of one or more TCR chains selected from the group of TCRα, TCRβ1, TCRβ2, TCRγ, TCRδ and pre-TCRα; e) express a substantially impaired endogenous TCR; f) is deficient in the expression of one or more target antigens of the SAR (e.g., HC-SAR; g) have a deletion or mutation in one or more target antigens of the SAR (e.g., HC-SAR to a form that is no longer recognized by the SAR (e.g., HC-SAR; h) lacks the expression of or has low expression of a functional HLA; i) lacks the expression of β2 microglobulin; j) lacks the expression of diaglycerol kinase (DGK) or Ikaros; k) expresses an agent that enhances the activity of SAR (e.g., HC-SAR)-expressing cell; 1) expresses an agent that inhibits an inhibitory molecule; m) expresses an agent that inhibits the expression of one or more target antigens of SAR; n) expresses an agent that provides co-stimulation to an SAR-expressing cell; o) expresses a cytokine or a chemokine; p) expresses an agent that that promotes the proliferation, persistence, expansion and activation of a SAR (e.g., HC-SAR)-expressing cell; q) expresses a soluble receptor; r) expresses a second SAR (e.g., HC-SAR; s) expresses a Chimeric Antigen Receptor (CAR); t) expresses an inhibitory CAR that comprise an antigen binding domain, a transmembrane domain and an intracellular domain of an inhibitory molecule; u) expresses a CAR comprising an antigen binding domain, a transmembrane domain and a primary signaling domain; v) expresses a CAR comprising an antigen binding domain, a transmembrane domain, a costimulatory signaling domain but no primary signaling domain; w) expresses a CAR comprising an antigen binding domain, a transmembrane domain, a primary signaling domain and a costimulatory domain; x) expresses a coreceptor, wherein the coreceptor is CD8a, CD8b, CD4 or a combination or variant thereof; y) expresses IL12 or a variant thereof; z) expresses membrane anchored or soluble forms of IL2 or IL15; ab) expresses an accessory module selected from the group of 41BBL, CD40L, CD80, CD86, K13, MC159, cFLIP-L/MRITα, cFLIP-p22, HTLV1 Tax, HTLV2 Tax, HTLV2 Tax-RS mutant, FKBPx2-K13, FKBPx2-HTLV2-Tax, FKBPx2-HTLV2-Tax-RS, IL6R-304-vHH-Alb8-vHH, IL12f, PD1-4H1 scFV, PD1-5C4 scFV, PD1-4H1-Alb8-vHH, PD1-5C4-Alb8-vHH, CTLA4-Ipilimumab-scFv, CTLA4-Ipilimumab-Alb8-vHH, IL6-19A-scFV, IL6-19A-scFV-Alb8-vHH, sHVEM, sHVEM-Alb8-vHH, hTERT, Fx06, CD3z, CD3z-GGGS-41BB, CD3-BBz, CD3-CD28z, CD3-CD28-Lck fusion protein, shRNA targeting Brd4, hTERT, heparinase, cytokine, chemokine, IL2, IL-7, IL-15, IL12f, IL-21, a costimulatory agent, soluble receptor, or combination thereof; y) expresses a therapeutic control; and/or ac) expresses a therapeutic control selected from the group of a truncated epidermal growth factor receptor (tEGFR), truncated epidermal growth factor receptor viii (tEGFRviii), truncated CD30 (tCD30), truncated BCMA (tBCMA), truncated CD19 (tCD19), CD34, thymidine kinase, cytosine deaminase, nitroreductase, xanthine-guanine phosphoribosyl transferase, human caspase 8, human caspase 9, inducible caspase 9 (icaspase9), purine nucleoside phosphorylase, linamarase/linamarin/glucose oxidase, deoxyribonucleoside kinase, horseradish peroxidase (HRP)/indole-3-acetic (IAA), Gamma-glutamylcysteine synthetase, CD20/alphaCD20, CD34/thymidine kinase chimera, dox-dependent caspase-2, mutant thymidine kinase (HSV-TKSR39), AP1903/Fas system, a chimeric cytokine receptor (CCR), a selection marker, dihydroxyfolate receptor (DHFR), mutant DHIFR, methylated-DNA-protein-cysteine methyltransferase, inosine monophosphate dehydrogenase II (IMDHP2), puromycin acetyle transferase (PAC), blasticidin-resistance gene, mutant calcinueurin a/b (Can/b), CNa12, CNb30, a suicide gene and combinations thereof.

In some embodiments, a CD4 T cell comprises any of the HC-SAR configurations of Tables 2F-2J. In some embodiments, a CD8 T cell comprises any of the HC-SAR configurations of Tables 2F-2J. In some embodiments, a Treg T cell comprises any of the HC-SAR configurations of Tables 2F-2J

In accordance with some embodiments herein, methods of inducing an immune response in a subject in need thereof are provided. An immune cell (T, NK, NKT etc.) comprising nucleic acids encoding any of the SAR configurations discussed herein, can be administered to the subject. Optionally, the immune cell (T, NK, NKT etc.) expresses any of the SAR configurations described herein. Optionally, the immune cell (T, NK, NKT etc.) encodes any of the SAR configurations described herein and can be subsequently induced to express them. In some embodiments, the immune cell (T, NK, NKT etc.) is autologous. In some embodiments, the immune cell is allogeneic.

In an embodiment, the SAR (HC-SAR) expressing cells are administered with a stem cell in which one or more target antigens of the SAR have been deleted or mutated to a form that is no longer recognized by the SAR (e.g., HC-SAR). In an embodiment, the one or more target antigens are selected from the antigens listed in Table B. In an embodiment, the one or more target antigens are selected from the group of CD19, CD20, CD22, CD30, CD33, CD38, CD45, CD70, CD79b, CD123, CD138, CD157, CD179b, CD200R, CD276, CD324, MPL, FLT3, Lym1, Lym2, CST, BCMA, CD138, GPRC5D, CLL1, CSF2RA, LAMP1, TSHR, TnAg, NKG2D, CXCR4, TCRβ1, TCRβ2, BST1, IL1RAP, ALK, Folate Receptor 1, ROR1 and TCRgd. In an embodiment, the stem cell is an autologous hematopoietic stem cell or an allogeneic hematopoietic stem cell.

Without being limited by any theory, it is contemplated that co-administration of the two genetically engineered T cells can boost the immune response.

In some embodiments a vector encoding a SAR is administered directly to a subject. The vector can provide nucleic acids encoding the chains of a SAR to a T cell in the subject, so as to provide a SAR in vivo.

The effective amount immune cells administered in accordance with some embodiments herein can depend on a variety of factors, for example characteristics of the subject, the type of disease state being treated, characteristics of the SAR, activity levels of the SAR-expressing cell, and/or the level of immune response desired. Optionally, the amount of immune cells administered is determined by the skilled artisan. In some embodiments, at least about 10 SAR-expressing T cells are administered to the subject, for example, at least about 10³, 10⁴, 10⁵, 10⁶, 10⁷, 10⁸, 10⁹, or 10¹⁰ immune cells, including ranges between any two of the listed values.

In some embodiments, the SAR-expressing cell is administered via intramuscular injection, intravaginal injection, intravenous injection, intraperitoneal injection, subcutaneous injection, epicutaneous administration or intradermal administration.

The methods described in the preceding sections for generating T cells expressing HC-SARs and using them for inducing immune response can be also used for other SAR described in this disclosure.

The present invention also features novel chimeric receptors for engineering redirected cells. For example, the present invention features an engineered cell co-expressing on its surface a synthetic antigen receptor (MHC-SAR) comprising a major histocompatibility complex (MHC) portion (derived from a MHC protein) directly or indirectly fused to a non-T cell receptor (TCR) portion (derived from a non-TCR protein); wherein the non-TCR portion is not part of an MHC protein.

In an embodiment, the non-T cell receptor comprises a non-T cell receptor module (NTCRM). In some embodiments, the non-TCR portion comprises a transmembrane domain of the non-TCR signaling receptor or a signaling adaptor and the MIC portion comprises an extracellular domain of the MHC protein. In some embodiments, the non-TCR portion comprises at least a portion of a transmembrane domain of the non-TCR signaling receptor or a signaling adaptor and the MHC portion comprises at least a portion of an extracellular domain of the MHC protein. In some embodiments, the non-TCR portion comprises at least a portion of a transmembrane domain and at least a portion of a cytoplasmic domain of a non-TCR signaling receptor or a signaling adaptor, and the MHC portion comprises at least a portion of an extracellular domain of the MHC protein.

In an embodiment, the MHC portion lacks entirely or partially the Ig-like C1 domain. In an embodiment, the MHC portion comprises the N-terminal region of its extracellular domain (i.e., MHC-Domain 1 or MHC-D1) and comprises a deletion in the C-terminal Ig-like C1 region (i.e., MHC domain 2 or MHC-D2) of the extracellular domain. In an embodiment, the MHC portion comprises the N-terminal region of the extracellular domain (i.e., MHC-Domain 1 or MHC-D1) that is linked to a TCR portion, a non-TCR portion, a signaling adaptor or a NTCRM via a linker. In an embodiment, the linker is an Ig-like linker. In an embodiment, the linker is an Ig-like linker. In an embodiment, the Ig-like linker is derived from a protein other than the MHC protein from which the N-terminal region is derived. In an embodiment, the linker region is derived from an MHC protein other than the MHC protein from which the N-terminal region is derived. In an embodiment, the linker is derived from an immunoglobulin or a TCR constant chain. Example linkers are shown in Table 7 of the provisional patent application. In an embodiment, the MHC portion is a hybrid of two or more MHC proteins; i.e., the MHC portion comprises the N-terminal region (i.e., MHC-Domain 1 or MHC-D1) derived from a first MHC Protein or variant thereof and C-terminal region (i.e., MHC-Domain 1 or MH-IC-D1) derived from a second MHC protein, wherein the first and the second MHC proteins are different (e.g., first MHC protein is ILA-DRA and second MHC protein is HLA-DPA etc.). In an embodiment, the MHC portion is a hybrid of two or more proteins; i.e., the MHC portion comprises the N-terminal region derived from an MHC Protein or variant thereof and C-terminal region derived from an immunoglobulin or a TCR constant chain.

In some embodiments, the MHC portion of the MHC-SAR is N-terminal to the TCR or non-TCR portion of the M-IC-SAR. In some embodiments, the MHC portion is directly fused to a TCR portion (e.g., TCR constant chain), or a non-TCR signaling receptor (e.g., CD16), a signaling adaptor (e.g., CD3z, FCRG) or a NTCRM. In some embodiments, the MHC portion is indirectly linked to a TCR portion (e.g., TCR constant chain), or a non-TCR signaling receptor (e.g., CD16), a signaling adaptor (e.g., CD3z, FCRG) or a NTCRM via a linker. In some embodiments, the MHC-SAR further comprises one or more autonomous antigen binding domains (e.g., AABD, e.g., vL, vH, vHH, FHVH, DARPIN, epitope, ligand, receptor, adaptor, peptide antigen etc.) that is operationally linked to the N-terminus or near the N-terminus of the MHC portion via an optional linker. In an embodiment, the AABD is a peptide antigen. In some embodiments, the MHC-SAR further comprises a peptide antigen integrated into the MHC portion, or directly or indirectly fused to the MHC portion. In some embodiments, the peptide antigen is linked to the MHC portion via a linker. In some embodiments, the linker comprises a glycine-rich peptide. In some embodiments, the SCR further comprises a transmembrane domain positioned in between the cell surface receptor ligand portion and the signaling molecule portion. In some embodiments, the MHC-SAR further comprises a peptide antigen integrated into the MHC portion, or directly or indirectly fused to the MHC portion are mammalian proteins (e.g., human, mouse, cat, dog, etc.

In some embodiments, the MHC protein comprises HILA-A, HLA-B, HLA-C, ILA-DPA1, HLA-DPB1, HLA-DQA1, HLA-DQB1, HLA-DRA, HLA-DRB, H2-Aa, H2-B1, H2-K1, H2-E8 beta, H2-EK alpha, H2-EK beta, a variant thereof, a fragment thereof, or a combination thereof. In some embodiment, the TCR is selected from the group of TCRα, TCRβ1, TCRβ2, TCRγ1, TCRγ2, TCRδ, or preTCRα, or a fragment thereof, or a combination thereof. In some embodiments, the non-TCR signaling receptor is a naturally occurring receptor. In some embodiment, the signaling adaptor is selected from the group consisting of CD3ζ, FceRγ (FCRG), DAP10, DAP12 a variant thereof, a fragment thereof, or a combination thereof. The present invention also features a chimeric receptor (MHC-SAR) as described above. For example, the MHC-SAR may comprise a major histocompatibility complex (MHC) portion derived from a MHC protein directly or indirectly linked to a TCR portion or a non-T cell receptor (TCR) portion derived from a non-TCR signaling receptor or a signaling adaptor, wherein the MHC-SAR is adapted to bind to a TCR of a target cell.

The present invention also features a method of eliminating a target cell or reprogramming a target cell (the target cell comprising a TCR) in some embodiments, the method comprises introducing a genetically engineered cell that expresses on its surface a chimeric receptor (MHC-SAR) according to the present invention to the target cell, wherein the MHC-SAR is specific for the TCR of the target cell, wherein upon binding of the MHC-SAR to the TCR the genetically engineered cell (a) initiates a signaling cascade that eliminates the target cell, or (b) instructs the target cell to differentiate to a specific effector function. In some embodiments, the method is for immunotherapy, in some embodiments, the target cell is an autoreactive T cell. In some embodiments, the method is for immunotherapy, in some embodiments, the genetically engineered cell is surgically introduced to a host (e.g., a mammal). In some embodiments, the target cell is an autoreactive T cell.

Example MHC-SAR comprising RQ13 peptide and HLA-DRB-B1 and HLA-DRA-A1 domains attached to different signaling chains are provided in SEQ ID NO (DNA): 23780-23794 and SEQ ID NO (PRT): 23877-23891. Additional MHC-SARs targeting different TCR can be generated by using different peptides and MHC D1 domains.

The MHC portion may comprise one or more MHC proteins (e.g., HLA-A, HLA-B, HLA-C, HLA-DPA1, HLA-DPB1, HLA-DQA1, HLA-DQ81, HLA-DRA, HLA-DRB1), one or more fragments thereof, or combinations thereof.

In some embodiments, the MHC-SAR comprises a peptide antigen. A non-limiting example of a peptide antigen that may be used with the MHC-SAR is RQ13 peptide RFYKTLRAEQ (SEQ ID NO: 23892

In some embodiments, the MHC-SAR comprises at least a portion of a MHC molecule that allows for binding to an appropriate TCR. in some embodiments, the MHC-SAR comprises at least a portion of a MHC molecule that allows for binding to an appropriate TCR and at least a portion of a TCR molecule (e.g., a portion of a TCR molecule that allows for appropriate signaling and/or complexing subunits such as CD3 subunits) or a non-TCR molecule (e.g., a portion of CD16A, CD16B, NKp30, NKp44, NKp46, CD3z, FcRy, DAP10 etc.).

There is a need for viral vectors enabling transduction of quiescent HSCs and limiting the risk of multi-copy integration. There is an important need for viral vectors enabling transduction of resting T, B and NK cells. Gene transduction into NK cells using VSVG pseudotyped lentiviral vectors also suffer from the problem of low efficiency.

In order to deliver genes by a lentiviral vector in hematopoietic cells (e.g., CD34+ stem cell, NK cells, naïve T and B cells etc.), an “entry” envelope protein allowing an efficient vector-cell fusion needs to be presented at its surface. The process of incorporating a heterologous envelope glycoprotein on the core of a lentiviral vector is called “pseudotyping”. For a long time, VSV-G associated with viral cores derived from HIV-1 has been used. Nevertheless, there are disadvantages in using VSV-G-LVs. Toxicity is associated with long-term expression of VSV-G which makes generation of stable cell lines difficult. In addition, VSV-G-LVs are sensitive to human complement, which makes them unsuited for in vivo use. Only high VSV-G-LVs doses (multiplicity of infection=MOI of 50-100) allow efficient hCD34+ cell transduction increasing the risk of multi-copy integration and thus genotoxicity (Di Nunzio et al. (2007) Hum. Gene Ther. 18:81 1-820). Human beings may also develop strong immune responses against VSV-G which may reduce the efficacy of a second administration of VSV-G-LVs. All these results limit ex vivo and in vivo use of VSV-G-LVs.

The chimeric envelope glycoprotein comprising the extracellular and transmembrane domains of the RD1 14 feline leukemia virus envelope glycoprotein fused to the cytoplasmic tail (designated TR) of the murine leukemia virus A (MLV-A) envelope glycoprotein, called herein after RD1 14/TR and described in the international application WO 03/091442) appeared to be a quite good alternative envelope glycoprotein. However, transduction levels stay way below those of VSV-G-LVs and titers of RD1 14 are much lower than for VSV-G-LVs. Additionally, RD1 14/TR glycoprotein cannot enter efficiently into murine cells. So, this receptor specificity limits the potentialities of work with this LV since the majority of preclinical disease models eligible for gene therapy are mice models.

The patent application WO2013045639 describes lentiviral vectors pseudotyped with either a chimeric envelope glycoprotein which comprises or consists in a fusion of the transmembrane and extracellular domain of a baboon endogenous retrovirus (BaEV) envelope glycoprotein and the cytoplasmic tail domain of a murine leukemia virus (MLV) envelope glycoprotein (BaEV/TR); or a modified BaEV envelope glycoprotein wherein the cytoplasmic tail domain is devoid of the fusion inhibitory R peptide (BaEVRless). These envelope glycoproteins display transduction properties particularly suitable for gene transfer into hematopoietic cells, including HSC and resting T and B cells. The BaEV/TR envelope glycoprotein is represented by SEQ ID NO (DNA): 113 and SEQ ID NO: (PRT): 8493, respectively. The modified BaEV envelope glycoprotein wherein the cytoplasmic tail domain is devoid of the fusion inhibitory R peptide (BaEVRless) as described in WO2013045639 is represented by SEQ ID NO (DNA): 37 and SEQ ID NO: (PRT): 8417, respectively.

The patent application WO2013045639 described that new LV pseudotypes can transduce very efficiently and stably hCD34⁺ cells up to 70% at low vector doses and upon mild cytokine stimulation.

Subsequently BaEV-LVs were shown to more efficiently transduce NK cells and NK cell lines as compared to VSV-G-LV. In one aspect, the present invention is based on the unexpected and surprising discovery that the modified BaEV envelope glycoprotein described in WO2013045639 whose cytoplasmic tail domain is devoid of the fusion inhibitory R peptide (BaEVRless) has very poor to negligible gene transduction efficiency into hematopoietic cells, including NK cells and T cells. The modified BaEV envelope glycoprotein whose cytoplasmic tail domain is devoid of the fusion inhibitory R peptide (BaEVRless) as described in WO2013045639 is represented by SEQ ID NO (DNA): 37 and SEQ ID NO: (PRT): 8417, respectively.

In another aspect the present invention is based on the unexpected discovery that the modified BaEV envelope glycoprotein wherein the cytoplasmic tail domain is devoid of the fusion inhibitory R peptide (BaEVRless) as described in WO2013045639 but carries additional amino acid sequences at the carboxy-terminus display transduction properties suitable for gene transfer into hematopoietic cells, including HSC, NK cells and resting T and B cells.

In an embodiment, the additional amino acid sequences at the carboxy-terminus are not derived from the cytoplasmic tail (designated TR) of the murine leukemia virus A (MLV-A) envelope glycoprotein. The cytoplasmic tail (designated TR) of the murine leukemia virus A (MLV-A) envelope glycoprotein is represented by SEQ ID NO (DNA): 114 and SEQ ID NO (PRT): 8494. In an embodiment, the modified envelope glycoprotein of the present disclosure is not the envelope glycoprotein of RD114 retrovirus (SEQ ID NO: 8431).

In an embodiment, the modified envelope glycoprotein of the present disclosure does not comprise the cytoplasmic tail (designated TR) of the murine leukemia virus A (MLV-A) envelope glycoprotein. In an embodiment, the modified envelope glycoprotein of the present disclosure does not comprise the TR domain of MLV-A envelope glycoprotein, which is represented by SEQ ID NO (DNA): 114 or SEQ ID NO (PRT): 8494. In an embodiment, the modified envelope glycoprotein of the present disclosure does not consist of the TR domain of MLV-A envelope glycoprotein, which is represented by SEQ ID NO (DNA): 114 or SEQ ID NO (PRT): 8494. In an embodiment, the modified envelope glycoprotein of the present disclosure lacks SEQ ID NO (PRT): 8494.

In an embodiment, the modified BaEV envelope glycoprotein of the present disclosure is not the BaEV/TR envelope glycoprotein, which is represented by SEQ ID NO (DNA): 113 and SEQ ID NO: (PRT): 8493, respectively. In an embodiment, the modified BaEV envelope glycoprotein of the present disclosure lacks one or more (e.g., 1, 2, 3, 4, 5, 6, 7, 8, 9, 10, 11, 12, 13 etc.) carboxy-terminal amino acid residues of SEQ ID NO: (PRT): 8493.

In an embodiment, the modified BaEV envelope glycoprotein of the present disclosure adds at least one (e.g., 1, 2, 3, 4, 5 6, 7, 8, 9, 10, 11, 12, 13, 14, 15, 20, 25 etc.) amino acid to the carboxy terminus of the BaEVRless envelope (SEQ ID NO (PRT): 8417) described in WO2013045639. In an embodiment, the modified BaEV envelope glycoprotein of the present disclosure adds at least two amino acids to the carboxy terminus of the BaEVRless envelope (SEQ ID NO (PRT): 8417). In an embodiment, the modified BaEV envelope glycoprotein of the present disclosure adds at least three amino acids to the carboxy terminus of the BaEVRless envelope (SEQ ID NO (PRT): 8417). In an embodiment, the modified BaEV envelope glycoprotein of the present disclosure adds at least four amino acids to the carboxy terminus of the BaEVRless envelope (SEQ ID NO (PRT): 8417).

In an embodiment, the modified BaEV envelope glycoprotein of the present disclosure adds more than one amino acid residues (e.g., 2, 5, 10, 50, 99 amino acid residues) to the carboxy terminus of the BaEVRless envelope glycoprotein (SEQ ID NO (PRT): 8417).

In an embodiment, the modified BaEV envelope glycoprotein of the present disclosure is at least one amino acid longer than the BaEVRless envelope glycoprotein (SEQ ID NO (PRT): 8417). In an embodiment, the modified BaEV envelope glycoprotein of the present disclosure has a cytoplasmic domain that is at least one amino acid longer than the cytoplasmic domain of BaEVRless envelope glycoprotein (SEQ ID NO (PRT): 8417). In an embodiment, the modified BaEV envelope glycoprotein of the present disclosure is more than one amino acid (e.g., 2, 3, 4, 5, 6, 7, 8, 9, 10, 15, 20, or 99 amino acid residues) longer than the BaEVRless envelope glycoprotein (SEQ ID NO (PRT): 8417). In an embodiment, the modified BaEV envelope glycoprotein of the present disclosure has a cytoplasmic domain that is at least one amino acid longer than the cytoplasmic domain of BaEVRless envelope glycoprotein (SEQ ID NO (PRT): 8417). In an embodiment, the modified envelope glycoprotein of the present disclosure does not comprise the TR domain, which is represented by SEQ ID NO (PRT): 8494.

In an embodiment, the first amino acid of the cytoplasmic domain is calculated from Glycine at residue 525 of SEQ ID NO (PRT): 8416 or 8417.

In an embodiment, the modified BaEV envelope glycoprotein of the present disclosure has a cytoplasmic domain that has a non-aromatic amino acid, a basic, an acid, a neutral, an aromatic, a polar, a negative charged, or a positively charged residue at position 23 of the cytoplasmic domain. In an embodiment, the modified BaEV envelope glycoprotein of the present disclosure has a cytoplasmic domain that has any amino acid residue at position 23 of the cytoplasmic domain.

In an embodiment, the modified BaEV envelope glycoprotein of the present disclosure has a cytoplasmic domain that is at least 23 amino acid residues in length and does not comprise the TR domain, which is represented by SEQ ID NO (PRT): 8494. In an embodiment, the modified BaEV envelope glycoprotein of the present disclosure has a cytoplasmic domain that is longer than 22 amino acid residues in length and does not comprise the TR domain represented by SEQ ID NO (PRT): 8494.

In an embodiment, the modified BaEV envelope glycoprotein of the present disclosure has a cytoplasmic domain that is at least 23 (e.g., 23, 24, 25, 50, 100, 110 or more) amino acid residues in length

In an embodiment, the modified BaEV envelope glycoprotein of the present disclosure has a cytoplasmic domain that is at least 23 amino acid residues in length.

In an embodiment, the modified BaEV envelope glycoprotein of the present disclosure has a cytoplasmic domain that comprise between 23 to 35 amino acid residues of the cytoplasmic domain of the wild-type BaEV envelope glycoprotein.

In an embodiment, the modified BaEV envelope glycoprotein of the present disclosure has a cytoplasmic domain that has the N-terminal 31 amino acid residues of the cytoplasmic domain of the wild-type BaEV envelope glycoprotein. In an embodiment, the modified BaEV envelope glycoprotein of the present disclosure has a cytoplasmic domain that comprises or consists of N-terminal 31, 30, 29, 28, 27, 26, 25, 24 or 23 amino acid residues of the cytoplasmic domain of the wild-type BaEV envelope glycoprotein. The cytoplasmic domain of the wild-type BaEV envelope glycoprotein is represented by SEQ ID NO (PRT): 8444.

In an embodiment, the modified BaEV envelope glycoprotein of the present disclosure has a cytoplasmic domain that lacks the carboxy-terminal 1, 2, 3, 4, 5, 6, 7, 8, 9, 10, 11, 12, 13, 14, 15, 16, amino acid residues of the wild-type BaEV envelope glycoprotein. In an embodiment, the modified BaEV envelope glycoprotein of the present disclosure has a cytoplasmic domain that lacks at least the carboxy-terminal 1, 2, 3, 4, 5, 6, 7 or 8 amino acid residues of the wild-type BaEV envelope glycoprotein.

In an embodiment, the modified BaEV envelope glycoprotein of the present disclosure has a cytoplasmic domain that lacks the carboxy-terminal 8 or more (e.g., 8, 9, 10, 11, 12, 13) amino acid residues of the wild-type BaEV envelope glycoprotein.

In an embodiment, the modified BaEV envelope glycoprotein of the present disclosure comprises a sequence represented by SEQ ID NO: 50005-50010 or a functional variant or a homolog with at least 75% (e.g., 75%, 80%, 85%, 90%, 95%, 99%) sequence identity thereof. In an embodiment, the modified BaEV envelope glycoprotein comprises a cytosolic domain with a sequence represented by SEQ ID NO: 50011-50013 or a functional variant or a homolog with at least 75% (e.g., 75%, 80%, 85%, 90%, 95%, 99%) sequence identity to a sequence with SEQ ID NO: 50011-13 in the cytosolic domain.

In an embodiment, the modified BaEV envelope glycoprotein of the present disclosure is encoded by a nucleic acid sequence that is codon optimized for the species (e.g., human, mouse, rat, hamster, monkey etc.) whose cells are used to generate the lentiviral particles. In an embodiment, the modified BaEV envelope glycoprotein is human codon optimized. An example human codon optimized modified BaEV envelope glycoprotein are provided in SEQ ID NO (DNA): 70-96, 98, 115-122, and 264-275.

In an embodiment, the polynucleotide encoding the modified BaEV envelope glycoprotein comprises a nucleic acid sequence with SEQ ID NO (DNA): 70-96, 98, 115-122, and 264-275 or sequence with at least 75%, sequence identity to SEQ ID NO (DNA): 70-96, 98, 115-122, and 264-275.

In an embodiment, the polynucleotide encoding the modified BaEV envelope glycoprotein comprises a nucleic acid sequence that encode for a polypeptide with SEQ ID NO (PRT): 8450-8476, 8478, 8495-8502 and 8644-8655 or sequence with at least 70% sequence identity to SEQ ID NO (PRT): 8450-8476, 8478, 8495-8502 and 8644-8655.

In an embodiment, the polynucleotide encoding the modified BaEV envelope glycoprotein comprises a nucleic acid sequence that encode for a polypeptide with an extracellular domain with SEQ ID NO (PRT): 8442 or a polypeptide with an extracellular domain with at least 70, 75%, 80%, 85%, 90%, 95%, 98%, 99% or 99.9% sequence identity to SEQ ID NO (PRT): 8442. In an embodiment, the polynucleotide encoding the modified BaEV envelope glycoprotein comprises a nucleic acid sequence that encode for a polypeptide with an extracellular domain with SEQ ID NO (PRT): 8442 or a polypeptide with an extracellular domain with up to 100 (e.g., 100, 90, 80, 70, 60, 50, 45, 40, 35, 30, 25, 20, 15, 10, 5, 2 or 1) amino acid modifications to SEQ ID NO (PRT): 8442.

In an embodiment, the polynucleotide encoding the modified BaEV envelope glycoprotein comprises a nucleic acid sequence that encode for a polypeptide with a transmembrane domain with SEQ ID NO (PRT): 8443 or a polypeptide with a transmembrane domain with at least 70, 75%, 80%, 85%, 90%, 95%, 98%, 99% or 99.9% sequence identity to SEQ ID NO (PRT): 8443. In an embodiment, the polynucleotide encoding the modified BaEV envelope glycoprotein comprises a nucleic acid sequence that encode for a polypeptide with a transmembrane domain with SEQ ID NO (PRT): 8443 or a polypeptide with a transmembrane domain with up to 10 (e.g., 10, 9, 8, 7, 6, 5, 4, 3, 2 or 1) amino acid modifications to SEQ ID NO (PRT): 8443.

In an embodiment, the polynucleotide encoding the modified BaEV envelope glycoprotein comprises a nucleic acid sequence that encode for a polypeptide with SEQ ID NO (PRT): 8450-8476, 8478, 8495-8502 and 8644-8655 or a polypeptide with up to 50 (e.g., 50, 45, 40, 35, 30, 25, 20, 15, 10, 5, 2 or 1) amino acid modifications in a polypeptide with SEQ ID NO (PRT): 8450-8476, 8478, 8495-8502, and 8644-8655.

In an embodiment, the modified BaEV envelope glycoprotein comprises a sequence with SEQ ID NO (PRT): 8450-8476, 8478, 8495-8502, and 8644-8655 or sequence with at least 70, 75%, 80%, 85%, 90%, 95%, 98%, 99% or 99.9% sequence identity to SEQ ID NO (PRT): 8450-8476, 8478, 8495-8502, and 8644-8655.

In an embodiment, the modified BaEV envelope glycoprotein comprises an amino acid sequence with at least 70% (e.g., 70%, 75%, 80%, 85%, 90%, 95%, 98%, 99% or 99.9%) sequence identity to any of the SEQ ID NO (PRT): 8450-8476, 8478, 8495-8502, and 8644-8655 and which retain one or more biological properties of the envelope glycoprotein encoded by SEQ ID NO (PRT): 8450-8476, 8478, 8495-8502, and 8644-8655. The example biological properties of the envelope glycoprotein include the ability to induce cell fusion, form trimers, bind to cell surface receptors, get expressed on cell surface, pseudotype a virus particle and bind hASCT1 and/or hASCT2 transporters etc.

In an embodiment, the modified BaEV envelope glycoprotein comprises a polypeptide with an extracellular domain represented by SEQ ID NO (PRT): 8442 or a functional variant thereof, including homologs from other species. In an embodiment, the modified BaEV envelope glycoprotein comprise a polypeptide with an extracellular domain with at least 70 (e.g., 70%, 75%, 80%, 85%, 90%, 95%, 98%, 99% or 99.9%) sequence identity to SEQ ID NO (PRT): 8442 or a polypeptide with an extracellular domain with up to 50 (e.g., 50, 45, 40, 35, 30, 25, 20, 15, 10, 5, 2 or 1) amino acid modifications to SEQ ID NO (PRT): 8442.

In an embodiment, modified BaEV envelope glycoprotein of the present disclosure does not encode for an envelope glycoprotein that consists of SEQ ID NO (PRT): 8417 or 8493. In an embodiment, the modified BaEV envelope glycoprotein of the present disclosure does not encode for an envelope glycoprotein that comprises a cytosolic domain that consists of or comprises of an amino acid sequence represented by SEQ ID NO (PRT): 8493 or 8494. In an embodiment, the variant extracellular domain of the modified BaEV envelope glycoprotein of the present disclosure retains one or more biological properties of the extracellular domain of envelope glycoprotein encoded by SEQ ID NO (PRT): 8442. Example biological properties include the ability to induce cell fusion, form trimers, bind to cell surface receptors, get expressed on cell surface, pseudotype a virus particle and bind hASCT1 and/or hASCT2 transporters etc.

In an embodiment, the modified BaEV envelope glycoprotein comprises a transmembrane domain with SEQ ID NO (PRT): 8443 or a transmembrane domain with at least 70% (e.g., 70%, 75%, 80%, 85%, 90%, 95%, 98%, 99% or 99.9%) sequence identity to SEQ ID NO (PRT): 8443. In an embodiment, the modified BaEV envelope glycoprotein comprises a transmembrane domain with SEQ ID NO (PRT): 8443 or a transmembrane domain with up to 10 (e.g., 10, 5, 2 or 1) amino acid modifications to SEQ ID NO (PRT): 8443.

In an embodiment, the modified BaEV envelope glycoprotein comprises an amino acid sequence with at least 70% (e.g., 70%, 75%, 80%, 85%, 90%, 95%, 98%, 99% or 99.9%) identity to any of the SEQ ID NO (PRT): 8450-8476, 8478, 8495-8502, and 8644-8655. In an embodiment, the modified BaEV envelope glycoprotein comprises an amino acid sequence with at least 70% (e.g., 70%, 75%, 80%, 85%, 90%, 95%, 98%, 99% or 99.9%) identity to any of the SEQ ID NO (PRT): 8450-8476, 8478, 8495-8502, and 8644-8655 and which contain one or more of amino acid motifs represented by SEQ ID NO: 16775-16780, 31989-31991. In an embodiment, the modified envelope glycoprotein of the present disclosure is not the BaEV/TR envelope glycoprotein (SEQ ID NO (PRT): 8493) or the BaEVRless envelope glycoprotein (SEQ ID NO (PRT): 8417), respectively.

In an embodiment, the modified BaEV envelope glycoprotein comprises an extracellular domain with SEQ ID NO: 8442 or a functional variant with at least 70% (e.g., 70%, 75%, 80%, 85%, 90%, 95%, 98%, 99% or 99.9%) sequence identity to SEQ ID NO: 8442 and which lacks a cytosolic domain that consists of or comprises of an amino acid sequence with SEQ ID NO (PRT): 8493 or 8494. In an embodiment, the modified BaEV envelope glycoprotein comprises a transmembrane domain with SEQ ID NO: 8443 or a functional variant with at least 70% sequence identity to the SEQ ID NO: 8443 and which lacks a cytosolic domain that consists of or comprises of an amino acid sequence with SEQ ID NO (PRT): 8493 or 8494.

In an embodiment, the modified envelope glycoprotein of the present disclosure is not the BaEV/TR envelope glycoprotein (SEQ ID NO (PRT): 8493) or the BaEVRless envelope glycoprotein (SEQ ID NO (PRT): 8417), respectively.

In an embodiment, the modified envelope glycoprotein of the present disclosure comprises in its extracellular domain one or more of sequences selected from the group of-SDGGG-(SEQ ID NO: 16775), LDLLTAE-(SEQ ID NO: 16776), SLAEVVLQNRR-(SEQ ID NO: 16777), SLAAVVLQNRR-(SEQ ID NO: 16778), SLAEVVLQNRRGLDLLTAE-(SEQ ID NO: 16779), SLAAVVLQNRRALDLLTAE-(SEQ ID NO: 16780), SLAGVVLQNRRALDLLTAE-(SEQ ID NO: 31990), VVLQNRR-(SEQ ID NO: 31991 or a variant with 1 or 2 amino acid substitutions or mutations to any of the above sequences. In an embodiment, the modified envelope glycoprotein of the present disclosure is not the BaEV/TR envelope glycoprotein (SEQ ID NO (PRT): 8493) or the BaEVRless envelope glycoprotein (SEQ ID NO (PRT): 8417), respectively.

In an embodiment, the modified BaEV envelope glycoprotein comprises a sequence represented by SEQ ID NO: 31989 in its extracellular domain or a variant with 1, 2, 3, 4 or 5 amino acid substitutions or mutations to the SEQ ID NO: 31989. In an embodiment, the modified envelope glycoprotein of the present disclosure is not the BaEV/TR envelope glycoprotein (SEQ ID NO (PRT): 8493) or the BaEVRless envelope glycoprotein (SEQ ID NO (PRT): 8417), respectively.

In an embodiment, the modified envelope glycoprotein does not comprise a sequence -SDGGG-(SEQ ID NO: 16775) in its extracellular domain. In an embodiment, the modified envelope glycoprotein comprises at least one exogenous antigen binding domain (e.g., scFv, vHH, FHVH, non-immunoglobulin antigen binding domains etc.) that is capable of binding to a distinct antigen. Example antigens include CD3, CD4, CD8, CD34, CD45, CD33, CD13, CD19, NKp30, NKG2D, NKp40, NKp44 etc.

In an embodiment, the modified envelope glycoprotein comprises one or more amino acid substitutions in the sequence-SDGGG-(SEQ ID NO: 16775). In an embodiment, the modified envelope glycoprotein comprises at least one exogenous antigen binding domain (e.g., scFv, vHH, FHVH, non-immunoglobulin antigen binding domains etc.) that is capable of binding to a distinct antigen. Example antigens include CD3, CD4, CD8, CD34, CD45, CD33, CD13, CD19, NKp30, NKG2D, NKp40, NKp44 etc. In an embodiment, the modified envelope glycoprotein lacks the ability to bind to ASCT2 or had reduced binding to ASCT2.

In an embodiment, the disclosure provides a modified HERV-W1 envelope glycoprotein that can be used for pseudotyping of viral vectors, including lentiviral vectors.

In an embodiment, the modified HERV-W1 envelope glycoprotein of the present disclosure comprise a non-native N-terminal signal peptide sequence. Non-native signal peptide sequence refers to a signal peptide sequence that is not part of the wild-type HERV-W1 signal peptide sequence. The wild-type HERV-W signal peptide sequence is represented by SEQ ID NO (DNA): 124 and SEQ ID NO (PRT): 8504. An example non-native signal peptide is murine Igκ signal peptide (mIgk-SP) and is represented SEQ ID NO (DNA): 130 and SEQ ID NO (PRT): 8510. Other example non-native signal peptides are known in the art and include human CD8a signal peptide (SEQ ID NO: 301) and human Ig signal peptide (SEQ ID NO: 302).

In an embodiment, the modified HERV-W1 envelope glycoprotein of the present disclosure lacks the complete cytoplasmic domain of the wild-type HERV-W1 envelope glycoprotein represented by SEQ ID NO (DNA): 127 and SEQ ID NO (PRT): 8507. In an embodiment, the modified HERV-W1 envelope glycoprotein has a partial or complete deletion of the inhibitory R fusion peptide. In an embodiment, the modified HERV-W1 envelope glycoprotein comprise a mutation in cytosolic domain. In an embodiment, the modified HERV-W1 envelope glycoprotein comprises a partial or complete replacement of the endogenous cytoplasmic domain of HERV-W1 with a non-endogenous (or foreign) peptide sequence. In an embodiment, the non-endogenous peptide sequence is derived from the cytoplasmic domain of a different envelope glycoprotein. In an example embodiment, the non-endogenous peptide sequence is represented by SEQ ID NO: 8494 or 8446 or deletion mutants of the foregoing.

In an embodiment, the modified HERV-W1 envelope glycoprotein comprises or contains a cytoplasmic domain that is at least 5 amino acid residue long or is longer than 5 amino acid residues. The first amino acid of the cytoplasmic domain is calculated from Glycine at residue 465 of SEQ ID NO: 8503 (HERV-W1-wt protein).

In an embodiment, the modified HERV-W1 envelope glycoprotein of the present disclosure comprises a cytoplasmic domain that comprises at least 5 (e.g., 6, 7, 8, 9, 10, 15, 25, 50, or 74) amino acid residues of the cytoplasmic domain of the wild-type HERV-W1 envelope glycoprotein. In an embodiment, the modified HERV-W1 envelope glycoprotein has a cytoplasmic domain that comprises the N-terminal 51 amino acid residues of the cytoplasmic domain of the wild-type HERV-W1 envelope glycoprotein (SEQ ID NO: 8503).

In an embodiment, the modified HERV-W1 envelope glycoprotein of the present disclosure has a cytoplasmic domain that lacks the carboxy-terminal 1-69 amino acid residues of the wild-type HERV-W1 envelope glycoprotein represented by SEQ ID NO: 8503.

In an embodiment, the modified HERV-W1 envelope glycoprotein has a cytoplasmic domain that lacks at least the carboxy-terminal 1, 2, 3, 4, 5, 6, 7 or 8 amino acid residues of the wild-type HERV-W1 envelope glycoprotein represented by SEQ ID NO:8503. In an embodiment, the modified HERV-W1 envelope glycoprotein has a cytoplasmic domain that lacks at least the carboxy-terminal 1-23 amino acid residues of the wild-type HERV-W1 envelope glycoprotein represented by SEQ ID NO:8503

In an embodiment, the modified HERV-W1 envelope glycoprotein has a cytoplasmic domain that lacks the carboxy-terminal 23 amino acid residues of the wild-type HER V-W1 envelope glycoprotein represented by SEQ ID NO:8503. In an embodiment, the nucleic acid modified HERV-W1 envelope glycoprotein comprises a sequence with SEQ ID NO (DNA): 140-151, 159-164 and 201 and SEQ ID NO (PRT): 8520-8531, 8539-8544 and 8581.

In an embodiment, the polynucleotide encoding the modified HERV-W1 envelope glycoprotein comprises a nucleic acid sequence that encode for a polypeptide with SEQ ID NO (PRT): 8520-8531, 8539-8544 and 8581 or a polypeptide with up to 100 (e.g., 100, 90, 80, 70, 60, 50, 45, 40, 35, 30, 25, 20, 15, 10, 5, 2 or 1) amino acid modifications in a polypeptide with SEQ ID NO (PRT): 8520-8531, 8539-8544 and 8581.

In an embodiment, the modified HERV-W1 envelope glycoprotein is encoded by a nucleic acid sequence that is codon optimized. Example human codon optimized modified HERV-W1 envelope glycoprotein are provided in SEQ ID NO (DNA): 140-151, 159-164 and 201. In an embodiment, the polynucleotide encoding the modified HERV-W1 envelope glycoprotein comprise a nucleic acid sequence with SEQ ID NO (DNA): 140-151, 159-164, 201 or sequence with at least 70% (e.g., 75%, 80%, 85%, 90%, 95%, 98%, 99%) sequence identity to the forgoing. In an embodiment, the polynucleotide encoding the modified HER V-W1 envelope glycoprotein comprise a nucleic acid sequence that encode for a polypeptide with SEQ ID NO (PRT): 8520-8531, 8539-8544 and 8581 or sequence with at least 70% sequence identity to SEQ ID NO (PRT): 8520-8531, 8539-8544 and 8581.

In an embodiment, the polynucleotide encoding the modified HERV-W1 envelope glycoprotein comprise a nucleic acid sequence that encode for a polypeptide with an extracellular domain with SEQ ID NO (PRT): 8505 or 8511 or a polypeptide with an extracellular domain with at least 70% sequence identity to SEQ ID NO (PRT): 8505 or 8511 or with up to 100 amino acid modifications to SEQ ID NO (PRT): 8505 or 8511. In an embodiment, the polynucleotide encoding the modified HERV-W1 envelope glycoprotein does not comprise the sequence of the wild-type HERV-W1, which is represented by SEQ ID NO (DNA): 8503. In an embodiment, the polynucleotide encoding the modified HERV-W1 envelope glycoprotein does not encode for an envelope glycoprotein that comprises a signal peptide domain that consists of or comprises of an amino acid sequence with SEQ ID NO (PRT): 8504. In an embodiment, the polynucleotide encoding the modified HERV-W1 envelope glycoprotein does not encode for an envelope glycoprotein that comprises a cytosolic domain that consists of an amino acid sequence with SEQ ID NO (PRT): 8507.

In an embodiment, the polynucleotide encoding the modified HERV-W1 envelope glycoprotein comprise a nucleic acid sequence that encode for a polypeptide with a transmembrane domain with SEQ ID NO (PRT): 8506 or 8512 a polypeptide with a transmembrane domain with at least 70, 75%, 80%, 85%, 90%, 95%, 98%, 99% sequence identity to SEQ ID NO (PRT): 8506 or 8512. In an embodiment, the polynucleotide encoding the modified HERV-W1 envelope glycoprotein comprise a nucleic acid sequence that encode for a polypeptide with a transmembrane domain with SEQ ID NO (PRT): 8506 or 8512 or a polypeptide with a transmembrane domain with up to 10 (e.g., 10, 7, 5, 2 or 1) amino acid modifications to SEQ ID NO (PRT): 8506 or 8512.

In an embodiment, the modified HERV-W1 envelope glycoprotein comprise an amino acid sequence with at least 70% (e.g., 70%, 75%, 80%, 85%, 90%, 95%, 98%, 99% or 99.9%) sequence identity to any of the SEQ ID NO (PRT): 8520-8531, 8539-8544 and 8581 and which retain one or more biological properties of the envelope glycoprotein encoded by the above. The example biological properties of the envelope glycoprotein include the ability to induce cell fusion, form trimers bind to cell surface receptors, get expressed on cell surface, pseudotype a virus particle and bind hASCT1 and/or hASCT2 transporters etc.

In an embodiment, the modified HERV-W1 envelope glycoprotein is not encoded by nucleic acid sequence represented by SEQ ID NO: 139 or amino acid sequence represented by SEQ ID NO: 8519.

In an embodiment, the modified HERV-W1 envelope glycoprotein comprises a transmembrane domain with SEQ ID NO:8506 or a functional variant with at least 70% sequence identity to SEQ ID NO: 8506 and which does not comprise a signal peptide with SEQ ID NO: 8504 or which lacks a cytosolic domain that consists of or comprises of an amino acid sequence with SEQ ID NO (PRT): 8507 or which lacks a cytosolic domain that consists of or comprise of an amino acid sequence (GPCIFNLLVNFVSSRIEAVKL) with SEQ ID NO (PRT): 20384. In an embodiment, the cytosolic domain of the modified HERV-W1 envelope glycoprotein of the disclosure is not-GPCIFNLLVNFVSSRIEAVKL-(SEQ ID NO (PRT): 20384). In an embodiment, the carboxy-terminal domain of the modified HERV-W1 envelope glycoprotein of the disclosure is not GPCIFNLLVNFVSSRIEAVKL (SEQ ID NO (PRT): 20384. In an embodiment, the carboxy-terminal domain of the modified HERV-W1 envelope glycoprotein of the disclosure does not end in the sequence GPCIFNLL VNFVSSRIEAVKL with SEQ ID NO (PRT): 20384.

In an embodiment, the modified envelope HERV-W1 glycoprotein comprises a sequence-SDGGG-(SEQ ID NO: 16775) in its extracellular domain or a variant with 1 or 2 amino acid substitutions or mutations thereto that retains the ability to bind to hASCT2. In an embodiment, the modified envelope glycoprotein does not comprise a sequence-SDGGG-(SEQ ID NO: 16775) in its extracellular domain. In an embodiment, the modified envelope glycoprotein comprises at least one exogenous antigen binding domain that is capable of binding to a distinct antigen. In an embodiment, the modified envelope glycoprotein comprises one or more amino acid substitutions in the sequence-SDGGG-(SEQ ID NO: 16775). In an embodiment, the modified envelope glycoprotein lacks the ability to bind to ASCT2 or has reduced binding to ASCT2.

In an embodiment, the modified HERV-W1 glycoprotein comprises in its extracellular domain one or more of the sequences selected from the group of 1) LDLLTAE-(SEQ ID NO: 16776), 2) SLAEVVLQNRR (SEQ ID NO: 16777), 3) SLAAVVLQNRR (SEQ ID NO: 16778), 4)-SLAEVVLQNRRGLDLLTAE-(SEQ ID NO: 16779), 5) SLAAVVLQNRRALDLLTAE (SEQ ID NO: 16780), 6) SEQ ID NO: 31989 or a variant with 1, 2, or 3 amino acid substitutions or mutations to the forgoing. In an embodiment, the modified envelope glycoprotein is not the HERV-W1 glycoprotein envelope glycoprotein (SEQ ID NO (PRT): 8503) or the HERV-W1 envelope glycoprotein (SEQ ID NO (PRT): 8419), respectively. In an embodiment, the modified HERV-w1 envelope glycoprotein is not the BaEV/TR envelope glycoprotein (SEQ ID NO (PRT): 8493) or the BaEVRless envelope glycoprotein (SEQ ID NO (PRT): 8417), respectively.

In an embodiment, the modified envelope glycoprotein are resistant to complement.

In an embodiment, the modified envelope glycoprotein are further mutated so that they lose their ability to bind to hASCT1 and/or hASCT2 transporters. In an embodiment, the mutated modified envelope glycoproteins that lose their ability to bind to hASCT1 and/or hASCT2 comprise one or more exogenous antigen binding domains (e.g., scFv, vHH, FHVH etc.) that allow them to bind to one or more different antigens (e.g., CD3, CD4, CD8, CD34, NKp30, NKp44, NKp46, CD16, NKG2D, CD13, CD33 etc.)

In an embodiment, the modified envelope glycoprotein is surface engineered to express antigen binding domains (e.g., scFv, vHH, FHVH, non-immunoglobulin antigen binding scaffold etc.) and cytokines that bind to specific antigens and/or receptors. In certain example embodiments, the targeting moiety is selected from the group consisting of Stem Cell Factor protein (SCF, KIT-ligand, KL, or steel factor), TPO (thrombopoietin), or a moiety that binds to cKit (CD117), MPL, CD4, CD8, CD3, CD5, CD6, CD7, CD2, TCR alpha, TCR beta, TCR gamma, TCR delta, CD10, CD16, NKp44, NKp46, NKp30, NKG2D, CD34, CD110, CD33, CD14, CD68, CCR7, CD62L, CD25, CCR2, CCR3, CCR4, CCR5, CCR6, CCR7, and CXCR3.

Accordingly, in certain aspects, the present disclosure provides an engineered viral particle comprising an engineered envelope harboring a mutated fusion protein, a chimeric gag protein, an engineered targeting moiety for binding to a target cell, wherein the mutated fusion protein does not bind to its natural receptor; and a nucleic acid encoding a polypeptide of interest. In certain example embodiments, the chimeric gag protein is xHIV gag protein as described in WO2021183761 (incorporated in its entirety by reference herein). In certain example embodiments, the targeting moiety is fused to the mutated fusion protein.

The disclosure also provides a vector encoding the modified envelope glycoprotein. In an embodiment, the vector is a plasmid vector.

In an embodiment, the modified envelope glycoprotein is expressed using transfection of an mRNA.

In an embodiment, the modified envelope glycoprotein are expressed in the packaging cells using transient transfection. In an embodiment, the modified envelope glycoproteins are expressed in the packaging cells stably. In an embodiment, the modified envelope glycoproteins are packaged using a capsid derived from an endogenous human retrovirus. In an embodiment, the modified envelope glycoproteins are packaged using gag and pol proteins derived from an endogenous human retrovirus.

In an embodiment, the modified envelope glycoprotein (e.g., modified BaEV or HERV-W1 glycoprotein) is used to pseudotype a virus. In an embodiment, the virus is a recombinant virus or a viral vector. In an embodiment, the viral vector is a retroviral vector, e.g., a γ retroviral vector or a lentiviral vector. In an embodiment, the viral vector encodes for a therapeutic protein. In an embodiment, the viral vector encodes for a synthetic antigen receptor (SAR). In an embodiment, the vector encodes for a chimeric antigen receptor (CAR). In an embodiment, the viral vector encodes for a next generation CAR. In an embodiment, the pseudotyped viral vector encodes a therapeutic protein, such as β-globin, ADA etc. In an embodiment, the pseudotyped viral vector encodes for a gene editing system (e.g., CRISP/Cas9, Zn finger nuclease etc.).

In an embodiment, the viral vector pseudotyped by the modified envelope glycoprotein is used for ex vivo gene delivery. In an embodiment, the viral vector pseudotyped by the modified envelope glycoprotein is used for in vivo gene delivery. In an embodiment, the viral vector pseudotyped by the modified envelope glycoprotein is administered to a subject. In an embodiment, the viral vector pseudotyped by the modified envelope glycoprotein is administered to a subject by any route, including but not limited to, intravenous route, intra-arterial route, intradermal route, intra-peritoneal route, intra-pleural route, intra-nodal route (e.g., injection into lymph node), intratumor route, intra-lesional route, intra-thecal route etc. In an embodiment, the viral vector pseudotyped by the modified envelope glycoprotein is administered to a subject by injection into an organ or a tissue, such as spleen, liver, heart, lung, eye, brain, muscle, bone, kidney, thyroid, fat, pancreas, etc.

In an embodiment, the subject receives an agent prior to, concurrent with or after the in vivo administration of the vector. In an embodiment, the agent is selected from the group of (e.g., CXCR4 antagonist, e.g., Plerixafor, BL-8040, BPRCX714, BPRCX807 etc.), a cytokine (e.g., G-CSF, GM-CSF, Neulasta or Pegfilgastrim, IL2, IL15), a bispecific or multi-specific T- or NK-activating antibody, a small molecule drug (e.g., a tyrosine kinase inhibitor, e.g., dasatinib), an immunosuppressive drug (e.g., rituximab, steroids, calcineurin inhibitor), an IL6 antagonist (e.g., tocilizumab), an IL1 antagonist (e.g., anakinra), an anti-histamine agent (e.g., Diphenhydramine), an antipyretic agent (e.g., paracetamol) or a combination.

The present invention thus concerns a pseudotyped viral vector particle for transferring biological material into cells. In an embodiment, the said vector particle comprises at least a modified BaEV or HERV-W1 envelope protein of the disclosure.

In a preferred embodiment, the targeted hematopoietic cell is not prestimulated with at least one cytokine. In particular it is not prestimulated with SCF, TPO and/or FI.3-L. In another preferred embodiment, the targeted hematopoietic cell is only prestimulated with SCF or TPO. In still another preferred embodiment, the targeted hematopoietic cell is only prestimulated with SCF and TPO.

The lentiviral vector particles pseudotyped with modified viral envelope glycoproteins (e.g., mBaEV, mHERV-W1 or mSyncetin-1) also permit highly efficient transduction of resting B cells, NK cells and thymocytes.

In an embodiment, freshly isolated total NK cells are prestimulated with IL2 and subsequently transduced with lentiviral vector particles pseudotyped with modified viral envelope glycoproteins (e.g., mBaEV, mHERV-W1 or mSyncetin-1) or functional variants thereof, including homologs from other species, of the present disclosure.

In another embodiment, after activation primary human NK cells can be efficiently transduced by using the lentiviral vector particles pseudotyped with modified viral envelope glycoproteins (e.g., mBaEV, mHERV-W1 or mSyncetin-1) or functional variants thereof, including homologs from other species, of the present disclosure, even without the need for inhibitors of NK cells anti-viral defense mechanisms whereas resting NK cells cannot be efficiently transduced by these vector particles.

Preferentially, NK cells are activated by stimulating agents, either in soluble form or surface-bound or by feeder cells, including parts of feeder cells, such as membrane particles. More preferentially, NK cells are activated by at least one growth factor such as a cytokine or combinations of growth factors such as cytokines, e.g., common-gamma chain cytokines including but not limited to IL-2, IL-7, IL-15, IL-21 or IL-1 family cytokines including but not limited to IL-1alpha, IL1beta, IL-18, IL-33, 11-36, IL37 and IL38.

The transduction of cells (e.g., hematopoietic stem cell, NK cells, T cells, B cells, dendritic cells, liver cells, spleen cells, heart cells, brain cells etc.) can be carried out by in vivo administration of viral particles pseudotyped with the modified viral envelope glycoproteins (e.g., mBaEV, mHERV-W1 or mSyncetin-1) or functional variants thereof of the present disclosure. The in vivo administration of viral particles can be carried out by methods known in the art. Example routes of administration include, intravenous, intra-arterial, subcutaneous, intra-cavitary, intra-pleural, intrathecal, intrapericardial, intra-tumoral. Alternatively, the viral particles can be directly injected into organs or tissues, such as the splenic, hepatic, cardiac etc.

The modified viral envelope glycoproteins (e.g., HERV-W1 or syncetin-1) have an advantage for in vivo administration as they are derived from endogenous human retroviruses and therefore are not immunogenic.

In an embodiment, the subject is administered a single dose of the viral particles pseudotyped with the modified viral envelope glycoproteins (e.g., HERV-W1 or syncetin-1) of the disclosure. In an embodiment, the subject is administered a multiple doses of the viral particles pseudotyped with the modified viral envelope glycoproteins (e.g., HERV-W1 or syncetin-1) of the disclosure.

The present invention also relates to a method for producing a pseudotyped viral vector particle comprising: a) transfecting a cell with: (i) at least one first nucleic acid sequence comprising a packaging competent retroviral derived genome; (ii) at least one second nucleic acid sequence comprising a cDNA encoding core proteins from said retrovirus, and (iii) at least one third nucleic acid sequence comprising a cDNA encoding a modified viral envelope glycoprotein of the disclosure. to yield a producer cell; b) maintaining the producer cell in culture for sufficient time to allow expression of the cDNAs to produce the encoded proteins; and c) allowing the encoded proteins to form viral vector particles of the invention.

In an embodiment, the disclosure provides a surface engineered viral particle or virus like particle comprising the modified viral glycoproteins described herein and a reporter. In an embodiment, the reporter is a luciferase, e.g., a marine luciferase, e.g., Nluc or Gluc. In an embodiment, the reporter is expressed on the surface of the viral particle or virus like particle.

The disclosure also provides a method of measuring the titer of the viral particle or virus like particle described herein by measuring the activity of the reporter. In an embodiment, the titer of the viral particle or virus like particle is measured by addition of a substrate. In an embodiment, the substrate is coelenterazine or its derivative or D-luciferin or its derivative.

In an embodiment, the packaging cell line lacks the expression of ASCT1 and/or ASCT2 or have reduced expression of ASCT1 and/or ASCT2. In an embodiment, the packaging cell line is exposed to suppressyn. In an embodiment, the packaging cell line expresses suppressyn (SEQ ID NO:31994) (Sugimoto J et al, Scientific Reports, 2013). In an embodiment, the packaging cell line expresses a receptor that binds to an antibody. In an embodiment, the receptor is an Fc receptor or a functional fragment or a variant or homolog thereof. In an embodiment, the Fc receptor is selected from the group of CD64, CD16A, CD16B and CD32 or a functional fragment, variant or a homolog thereof which retains the ability to bind to Fc region or an antibody. In an embodiment, the Fc receptor comprises a transmembrane or membrane anchoring domain. In an embodiment, the Fc receptor comprises at least one exogenous (non-native) antigen binding domain (e.g., scFv, vHH, FHVH, non-immunoglobulin antigen binding scaffold etc.) targeting an antigen. In an embodiment, the antigen is selected from the group of CD3, CD4, CD8, CD45, CD34, CD33, CD13, CD19, CD20, CD22, CD56, NKG2D, NKp30, NKp40, NKp46 or a combination thereof. In an embodiment the packaging cell line is engineered to express more than one Fc receptor. In an embodiment, the Fc receptor is not part of the viral envelope glycoprotein. In an embodiment, the Fc receptor is expressed on the cell membranes of the packaging cells that are used to produce the viral vector. In an embodiment, the Fc receptor comprises a membrane anchoring domain (e.g., a GPI linker). In yet another embodiment, the Fc receptor comprises a secretory signal. Example Fc receptors (CD64) are provided in SEQ ID NO (DNA): 19762-19763 and SEQ ID NO (PRT): 20382-20383.

In yet another embodiment, the Fc receptor is expressed in the packaging cells stably. In still another embodiment, the Fc receptor is expressed in the packaging cells transiently.

In an embodiment, the disclosure provides a parental cell for the generation of enveloped viral particles producer or packaging cell lines, wherein the parental cell is genetically engineered to decrease expression of MHC-I, MHC-II, 2M on the surface of the cell and/or is genetically engineered to overexpress CD47 or a Fc receptor (e.g., CD64, CD16) including truncated forms. In an embodiment, the parental cell line is engineered to overexpress a cytokine/ligand (e.g., CD137L), a cytokine receptor or a targeting moiety (e.g., CD3 scFv, CD28 scFv, CD4 scFv, CD8 scFv etc.).

In an embodiment, the packaging cell line lacks expression of β2M and CIITA. In an embodiment, the packaging cell line overexpresses CD47. In an embodiment, the packaging cell line lacks expression of β2M (β2 macroglobulin), CIITA and overexpresses CD47 and CD64 (β2M−/−CIITA−/−CD47/CD64). In an embodiment, the packaging cell line lacks expression of β2M (B2 macroglobulin), CIITA and overexpresses CD47 and CD16A/CD16B (β2M−/−CIITA−/−CD47/CD16A/CD16B).

In an embodiment, the viral particle or virus like particle lacks expression of β2M and CIITA. In an embodiment, the viral particle or virus like particle overexpresses CD47. In an embodiment, the p viral particle or virus like particle lacks expression of β2M (B2 microglobulin), CIITA and overexpresses CD47 and CD64 (β2M−/−CIITA−/−CD47/CD64). In an embodiment, the packaging cell line lacks expression of β2M (B2 macroglobulin), CIITA and overexpresses CD47 and CD16A/CD16B (β2M−/−CIITA−/−CD47/CD16A/CD16B).

In an embodiment, the invention provides an enveloped viral particle producer or packaging cell, wherein the cell is genetically engineered to decrease expression of CD47 on the surface of the cell. In an embodiment, the cell comprises a genetically engineered disruption of a gene encoding β2-microglobulin and/or a genetically engineered disruption of one or more genes encoding an MHC-I a chain and/or CIITA and over-expresses or under-expresses CD47 and overexpresses CD64 and/or CD16A/CD16B

In an embodiment, the packaging cell line expresses on its surface (i) a first mitogenic T-cell activating transmembrane protein which comprises a mitogenic domain which binds CD3; and (ii) a second mitogenic T cell activating transmembrane protein which comprises a mitogenic domain which optionally binds CD28. In an embodiment, both the first and the second protein comprise a transmembrane domain. In an embodiment, both the first and the second protein comprise a membrane anchoring domain (e.g., GPI linked domain) that is not a transmembrane domain. In an embodiment, the first or the second protein comprise a transmembrane domain. In an embodiment, the first or the second protein comprise a membrane anchoring domain.

In an embodiment, a viral vector (e.g., retroviral or lentiviral vector) is provided having a viral envelope which comprises: (i) a first mitogenic T-cell activating transmembrane protein which comprises a mitogenic domain which binds CD3; and (ii) a second mitogenic T cell activating transmembrane protein which comprises a mitogenic domain which optionally binds CD28. In an embodiment, both the first and the second protein comprise a transmembrane domain. In an embodiment, both the first and the second protein comprise a membrane anchoring domain (e.g., GPI linked domain) that is not a transmembrane domain. In an embodiment, the first or the second protein comprise a transmembrane domain. In an embodiment, the first or the second protein comprise a membrane anchoring domain.

For the purpose of transfection, said first, second and third nucleic acid sequences may be carried on a same vector, or on two or three separated vectors. In an embodiment, gag, pol, rev, env genes (e.g., mBaEV, mHERV-W1), and viral genome cassettes carrying target nucleic acid fragments are stably inserted into the genome of the host cell (for example, 293T cells) by means of a sleeping beauty (SB) transposon system in combination with a Piggy BAC (Pb) transposon system, and a viral production system capable of inducing regulation and control is developed in combination with an induced expression system (for example, a TET-ON system and/or a Cumate system) to generate stable producer cell lines.

In one aspect of the invention, the GAG and POL genes (in embodiments in which the GAG POL represents the GAG gene and the POL gene are located in the same construct and translated in a frameshift manner), the transcription of the REV gene, the coding sequence of the viral envelope glycoprotein (e.g. mBaEV or mHERV-W1), and the viral genome transcription cassette carrying the target nucleic acid fragment is controlled, preferably by placing the gene or sequence under the control of an inducible expression system, preferably by placing the gene or sequence under the control of a TET-ON and/or CUMATE inducible expression system.

The present invention also relates to a medicament comprising a pseudotyped viral vector particle as defined above as active ingredient, in particular a pseudotyped viral vector particle as defined above further comprising a biological material which is preferably one or more nucleic acids.

It also relates to a pharmaceutical composition comprising a pseudotyped viral vector particle as defined above, in particular a pseudotyped viral vector particle as defined above further comprising a biological material which is preferably one or more nucleic acids, and a pharmaceutically acceptable carrier.

The present invention also concerns a method for treating a subject in need thereof comprising administering a therapeutically effective amount of a pseudotyped viral vector particle as defined above, in particular a pseudotyped viral vector particle as defined above further comprising a biological material which is preferably one or more nucleic acids, to the subject in need thereof. For intravenous administration, a unit dose from about 5×10⁸ to about 10⁹ pseudotyped viral vector particles as defined above can be used, whereas for medullar administration a unit dose from about 10⁸ to about 5×10⁸ pseudotyped viral vector particles as defined above can be used.

In an embodiment, the pseudotyped viral vector particle of the invention is for use in the treatment of a hematopoictic disorder or an autoimmune disease. In an embodiment, the pseudotyped viral vector particle of the invention is used for adoptive cellular therapy. In an example embodiment, the pseudotyped viral vector particle of the invention is for generation of chimeric antigen receptor (CAR) modified T, NK, or macrophage cells. In an embodiment, the CAR is a next generation CAR.

In an embodiment, the cells (e.g., CD34+ HSC, T, NK, NKT, or macrophage cells are transduced with the pseudotyped viral vector particle of the invention and then expanded ex vivo. In an embodiment, the cells are expanded ex vivo for between 1-100 days. In an embodiment, the cells are not expanded ex vivo. In an embodiment, the cells (e.g., CD34+ HSC, T, NK) are transduced with the pseudotyped viral vector particle of the invention in vivo.

In an embodiment, the T, NK, NKT, or macrophage cells are transduced with the viral vector particle pseudotyped with modified envelope glycoproteins of the invention show better disease modifying activity (e.g., antitumor activity, anti-inflammatory activity) as compared to the T, NK, NKT, or macrophage cells are transduced with the viral vector particles pseudotyped with the VSVG envelope glycoprotein when equal number of both cell types are administered to a subject in need. In an embodiment, the cells are exposed to an agent that increases the expression of ASCT1 and/or ASCT2.

In an embodiment, the present disclosure provides surface-engineered lentiviral particles that can be generated using multicistronic vectors designed to express a plurality of polypeptides, namely a modified baboon envelope glycoprotein or HERV-W1 envelope glycoprotein or functional variant thereof and one or more non-viral proteins capable of viral surface display. In the multicistronic vectors of the disclosure, the plurality of polypeptides are joined by linkers comprising peptides capable of inducing ribosome skipping or self-cleavage. 2A peptides may be used (e.g., T2A, P2A, E2A, and F2A).

In one aspect, the disclosure provides a multicistronic vector for surface-engineering lentiviral particles, comprising a polynucleotide operatively linked to a promoter, wherein the polynucleotide encodes a plurality of polypeptides joined by linkers comprising peptides capable of inducing ribosome skipping or self-cleavage, and wherein the plurality of polypeptides comprise a modified baboon envelope glycoprotein (mBaEV) or HERV-W1 envelope glycoprotein (mHERV-W1) or functional variant thereof and one or more non-viral proteins capable of viral surface display.

In some embodiments, the linkers comprise 2A peptides. In some embodiments, the plurality of polypeptides comprises 2, 3, 4, 5, 6, 7, 8, 9, 10, 11, 12, 13, 14, 15, 16, 17, 18, 19 or 20 proteins capable of viral surface display.

In some embodiments, the non-viral proteins capable of viral surface display comprise one or more non-viral proteins selected from a transmembrane-domain fusion of a single chain variable fragment (scFv) specific for human CD3 (anti-CD3 scFv), human CD86, and human CD137L, or functional variants thereof.

In some embodiments, the viral fusion glycoprotein is modified baboon envelope glycoprotein or a modified HERV-W1 envelope glycoprotein or a functional variant thereof, and the plurality of polypeptides comprises the anti-CD3 scFv, the human CD86, and human CD137L, or functional variants thereof. In some embodiments, the plurality of polypeptides consists of a modified baboon envelope glycoprotein (mBaEV) or a modified HERV-W1 envelope glycoprotein (mHERV-W1), the anti-CD3 scFv, the human CD86, and the human CD137L, or functional variants thereof. Non-limiting examples of multicistronic vectors of the disclosure include SEQ ID NOs (DNA): 31 and 32.

In some embodiments, the mutlicistronic vector is a lentiviral envelope plasmid capable of generating a lentiviral particle pseudotyped with mBaEV or mHERV-W1 when co-transfected with a transfer plasmid and a packaging plasmid into a packaging cell line. Example lentiviral envelope plasmids are provided in SEQ ID NO: 31 and 32.

In some embodiments, the titer of surface-engineered lentiviral particle is at least about 1×10⁶, 1×10⁷, 2×10⁷, 4×10⁷, 6×10⁷, 8×10⁷, or 1×10⁸ IU/ml.

In an embodiment, the subject is administered the viral vector (encapsulated in a hydrogel. In an embodiment, the subject is administered the packaging cell line generating the viral vector encapsulated in a hydrogel.

In an embodiment, the disclosure provides a method of in vivo gene delivery comprising administering an engineered viral particle to a subject in need of delivery, wherein the engineered viral particle is any viral particle or VLP, wherein the administration of the engineered viral particle or VLP induces an in vivo activity in a target cell associated with the polypeptide of interest (e.g., a SAR).

In some embodiments, the lentiviral particle comprises a targeting agent or the nucleic acid vector encodes a targeting agent. Example targeting agents include antibodies, chimeric antigen receptor (CAR), synthetic antigen receptors (“SAR”) and recombinant TCR.

The present disclosure provides methods of transducing immune cells in a subject in need thereof, comprising a) administering a multi-specific antibody to render immune cells in the subject more transducible; and b) administering a vector, optionally a viral vector. In some embodiments, the vector is a lentiviral vector. In some embodiments, the viral vector is pseudotyped with an endogenous human virus (e.g., endogenous human retrovirus, e.g., HERV-W1, HERV-FRD etc.) envelope protein or a variant thereof. In some embodiments, the viral vector is pseudotyped with mBaEV, mHERV-W1 and/or VSVG envelope proteins. In some embodiments, the viral vector is pegylated. In some embodiments, the method transduces the immune cells. In some embodiments, the immune cells are T cells. In some embodiments, the vector encodes a SAR selected from a CAR, SIR, Ab-TCR, MHC-SAR, zSIR, TFP, uTCR-SAR, hybrid chain SIR, hybrid chain Ab-TCR, CD16-SAR (e.g., CD16-based SAR), NKp44 SAR, NKp46 SAR, NKp30 SAR, NKG2D-SAR.

In some embodiments, the multi-specific antibody comprises a T-cell antigen-specific binding domain. In some embodiments, the T-cell antigen is CD3, CD4, CD8, or TCR. In some embodiments, the multi-specific antibody comprises a NK-cell antigen-specific binding domain. In some embodiments the NK cell antigen is CD16A, NKp44, NKp46, NKp30, or NKG2D.

In some embodiments, the multi-specific antibody comprises a second antigen-specific binding domain. In some embodiments, the second antigen is CD19.

In some embodiments, the lentiviral vector is pseudotyped with a mBaEV, mHERV-W1 and/or VSVG envelope protein. In some embodiments, the lentiviral vector is pseudotyped with a Nipah virus envelope protein.

In some embodiments, the multi-specific antibody binds specifically to CD3 and CD19, wherein the vector is a lentiviral vector pseudotyped with a mBaEV or mHERV-W1 virus envelope protein, and wherein the vector comprises a polynucleotide encoding an anti-CD 19 chimeric antigen receptor and optionally a transgene encoding an accessory module.

The present disclosure provides combination therapies for use in transducing immune cells in vivo, comprising a multi-specific antibody and a vector, optionally a viral vector, optionally a viral vector pseudotyped with a mBaEV or mHERV-W1 virus envelope protein.

CARs can recognize antigen in an MHC independent manner. Current methods for generation of chimeric antigen receptor expressing cells (e.g., CAR-T, CAR-NK etc.) involve ex vivo gene transduction with a CAR encoding vector (e.g., lentiviral, γ retroviral, transposon etc.) or mRNA transcript, followed by ex vivo expansion and subsequent administration of the CAR-expressing cells to the subject in need thereof. However, in vivo delivery of CAR construct/mRNA transcript is being considered to reduce cost and to prevent delay associated with manufacturing. These studies are generally being conducted with single chain 2^{nd} generation CAR constructs comprising an antigen binding domain (e.g., scFv, vHH domain etc.) attached via a hinge region to a transmembrane domain (e.g., CD8 or CD28 transmembrane domain) and comprising a cytosolic costimulatory domain (e.g., CD28 or 41BB costimulatory domain) and an activation domain (e.g., CD3z or FcRy). The inventor has made the surprising discovery that the single chain 2^{nd} generation CAR constructs, as currently being tested, are not suitable for in vivo gene delivery of CAR constructs for the purpose of generating CAR-T cells. The current application describes for the first time that the in vivo delivery of single chain CAR constructs (e.g., first and 2^{nd} generation CAR, and TFPs) for the purpose of generating CAR cells in vivo is not safe and has the potential of causing disease relapse. The current application further describes that zSIR constructs are also not ideal for in vivo delivery for the purpose of generating CAR expressing cells (e.g., CAR-T, CAR-NK, CAR-monocytes etc.). The current application further describes that in vivo delivery of single chain CAR constructs (e.g., first and 2^{nd} generation CAR, and TFPs) and zSIR can result in their ectopic insertion and/or expression in malignant cells. The current application further describes that in vivo delivery of single chain CAR constructs (e.g., first and 2^{nd} generation CAR, and TFPs) and zSIR can result in their expression in normal healthy cells/tissues other than the intended target cell (e.g., T, NK, macrophage etc.), which has the potential of causing side effects.

The disclosure provides a safe, economical, and efficient method for in vivo generation of CAR/SAR-T cells for the treatment of a variety of disease conditions, including cancer, autoimmune, degenerative, and infectious disorders. In contrast to the current methods that are focused on targeting the expression of the CAR construct to the T cells, the method of the present invention involves the use of SAR (CAR) constructs of optimal design that show selective expression only in T cells. Therefore, the method of the present invention can be used to achieve selective expression of the SAR polypeptide in T cells without the need for using special T cell directed vector.

The method of the present invention involves the use of unique SAR designs that are superior for in vivo SAR generation. In an embodiment, the method involves the use of a SAR design suitable that is a double chain SAR and comprises two transmembrane domains. In an embodiment, the SAR of the method of the invention comprises at least one polypeptide chain that comprises at least one fragment encoding the transmembrane and cytosolic domain that is selected from the group of SEQ ID NO: 31985-31988 and 31992. In an embodiment, the SAR of the method of invention for in vivo generation of SAR-T cells is a SAR which encodes for two polypeptide chains each of which comprises at least one fragment selected from the group of SEQ ID NO: 31985-31988 and 31992. In an embodiment, the disclosure provides a SAR for in vivo generation of SAR-T cells that comprises at least one fragment with at least 70%, 75%, 80%, 85%, 90%, 95% or 99% sequence identity to a sequence represented by SEQ ID NO: 40606-40758. In an embodiment, the disclosure provides a SAR for in vivo generation of SAR-T cells that comprises at least two fragments with at least 70%, 75%, 80%, 85%, 90%, 95% or 99% sequence identity to a sequence represented by SEQ ID NO: 40606-40758. In an embodiment, the disclosure provides a SAR for in vivo generation of SAR-T cells that comprises a connecting peptide (hinge domain) derived from a TCRα, β, γ, δ or preTCRα chain or a functional variant or a fragment thereof. In an embodiment, the disclosure provides a SAR for in vivo generation of SAR-T cells that comprises a connecting peptide (hinge domain) and a transmembrane domain derived from TCRα, β, γ, δ or preTCRα chain or a functional variant or a fragment thereof. In an embodiment, the disclosure provides a SAR for in vivo delivery that comprises a T cell receptor module. In an embodiment, the SAR for in vivo generation of SAR-T cells is not a TCR. In an embodiment, the disclosure provides a SAR for in vivo delivery that is a hybrid chain SAR. In an embodiment, the disclosure provides a SAR for in vivo delivery that is a double chain SAR or a one and a half chain SAR. In an embodiment, the disclosure provides a SAR for in vivo generation of SAR-T cells that comprises chains that interact with CD3ε, CD3γ αnd/or CDδ chains. In an embodiment, the disclosure provides a SAR for in vivo generation of SAR-T cells that comprises at least one signaling chain that interact with a TCR constant chain selected from the group of TCRα, TCRβ, TCRγ, TCRδ and preTCRα chain. In an embodiment, the disclosure provides a SAR for in vivo generation of SAR-T cells that lacks a transmembrane domain derived from CD8, CD28, FcRy or CD3z. In an embodiment, the method involves the use of SAR constructs for in vivo generation of SAR-T cells that can recognize antigen in an MHC-independent manner. In an embodiment, the method involves the use of SAR constructs for in vivo generation of SAR-T cells that can recognize antigen in an MHC-dependent manner. In an embodiment, the method involves use of SAR construct that comprises non-TCR antigen binding domains, i.e., antigen binding domains that are not derived from a TCR. In an embodiment, the SAR comprises antigen binding domains that are derived from antibodies, antibody fragments (e.g., vL, vH, scFv etc.), vHH, non-immunoglobin antigen binding domains (e.g., DARPIN, Centyrin etc.). In an embodiment, the SAR for in vivo generation of SAR-T cells comprises at least one linker derived from immunoglobulins (e.g., IgCL, IgG-CH1 etc.). In an embodiment, the SAR for in vivo generation of SAR-T cells lacks at least one linker derived from immunoglobulins (e.g., IgCL, IgG-CH1 etc.). In an embodiment, the SAR for in vivo generation of SAR-T cells lacks two linkers derived from immunoglobulins (e.g., IgCL, IgG-CH1 etc.). In an embodiment, the SAR for in vivo generation of SAR-T cells is not an Ab-TCR. In an embodiment, a SAR suitable for in vivo delivery for the generation of SAR-T cells in vivo comprises one or more non-TCR antigen binding domain (e.g., antibody, antibody fragment, Fab, Fab2, scFv, vL, vH, vHH, FHVH, non-immunoglobulin antigen binding scaffold, extracellular domain of a receptor, extracellular domain of a ligand/cytokine, autoantigen, adaptor, epitope etc.) attached via optional linkers to at least one TCR constant chain or functional fragment or functional variant or homolog thereof, including homolog from non-human species. In an embodiment, the TCR constant chain or functional fragment or functional variant or homolog thereof comprises a T cell receptor module that is capable of recruiting at least one TCR-associated signaling module when expressed in a T cell. In an embodiment, the method teaches that the following SAR designs show expression in only T cell and do not express in non-T cells: OHC and double chain SIR, SIR with hybrid TCR chains, OHC and double chain Ab-TCR, OHC and double chain Ab-TCR with hybrid TCR chain, OHC and double chain cTCR, OHC and double chain cTCR with at least one hybrid TCR chain. In an embodiment, at least one chain of the SAR is a hybrid chain. In an embodiment, the disclosure provides a SAR for in vivo generation of SAR-T cells that comprises a transmembrane domain derived from TCRα, β, γ, δ or preTCRα chain or a functional variant or a fragment thereof.

In an embodiment, the SAR shows less than 1% (e.g., 1%, 0.5%, 0.2%, 0.1%, 0.05%, 0.02%, 0.01%, 0%) cell surface expression at protein level in cells other than T cells (e.g., non-T cells) when delivered in vivo. In an embodiment, the SAR shows less than 1% (e.g., 1%, 0.5%, 0.2%, 0.1%, 0.05%, 0.02%, 0.01%, 0%) cell surface expression at protein level in cancer cells when delivered in vivo. In an embodiment, the cancer is of non-T cell lineage, e.g., B cell lymphoma, B cell ALL, AML, prostate cancer, breast cancer etc. In an embodiment, the cancer cells do not express one or more CD3 chains selected from the group of CD3ε, CD3γ and/or CD3δ. In an embodiment, the SAR shows less than 1% (e.g., 1%, 0.5%, 0.2%, 0.1%, 0.05%, 0.02%, 0.01%, 0%) cell surface expression at protein level in healthy cells when delivered in vivo. In an embodiment, the healthy cells comprise one or more of B cell, macrophage, neutrophils, liver cells, spleen cells, heart cells, lung cells, kidney cells, breast cell, ovarian cells, epithelial cells, brain cells, skin cells, gastrointestinal cells, muscle cells etc. In an embodiment, the cell surface expression of SAR protein in T cells is at least 2-fold (e.g., 2-fold, 5-fold, 10-fold, 50-fold, 100-fold, 1000-fold, 10,000-fold, 100,000-fold, million-fold, billion-fold, or a trillion-fold) higher than its expression in non-T cells. In an embodiment, the SAR shows less than 1% (e.g., 1%, 0.5%, 0.2%, 0.1%, 0.05%, 0.02%, 0.01%, 0%) incidence of second T cell cancers when delivered in vivo. In an embodiment, the SAR construct is delivered in vivo using a standard vector. In an embodiment, the SAR construct is delivered in vivo using a vector that is not specifically targeted to T cells. In an embodiment, the SAR construct is delivered in vivo using a vector that is not modified to reduce its insertion into cells other than T cells. In an embodiment, the SAR for in vivo delivery is delivered using a viral vector. In an embodiment, the SAR for in vivo delivery is delivered using a viral like particle. In an embodiment, the SAR for in vivo delivery is delivered using lipid nanoparticle. In an embodiment, the SAR for in vivo delivery is delivered using vector that are pseudotyped with standard envelope proteins. In an embodiment, the SAR is delivered via a vector that is not surface engineered. In an embodiment, the SAR is delivered in vivo using a vector that is pseudotyped with a wild-type envelope glycoprotein. In an embodiment, the SAR is delivered in vivo using a vector that is pseudotyped with an envelope glycoprotein that is still capable of binding to its native receptor. In an embodiment, the SAR is delivered in vivo using a vector that is pseudotyped with an envelope glycoprotein selected from one or more of VSV-G, modified Baboon envelope glycoprotein, modified HERV-W1 envelope glycoprotein, gibbon ape envelope glycoprotein or Cocal glycoprotein. In an embodiment, the SAR is delivered in vivo using a DNA vector. In an embodiment, the SAR is delivered in vivo using an RNA vector. In an embodiment, the SAR is delivered in vivo using a circular RNA. In an embodiment, the SAR is delivered in vivo using an in vitro transcribed RNA. In an embodiment, the SAR is delivered in vivo using transposase vector.

In one embodiment the vector is a retroviral vector, the target is a T cell and the transgene is a SAR (e.g., a SIR, cTCR, Ab-TCR, HC-SAR etc.). In another embodiment, the vector is retroviral vector, the target is a cell that has been activated by administration of an external agent and the transgene encodes a therapeutic activity. In this and other embodiments a retroviral vector can be replicating (RRV) or non-replicating (RNV) and can be derived from any integrating virus such as a foamy virus, a lentivirus, an alpha, beta, or gamma retrovirus or CRISPR elements and also includes non-viral integrating vector such as those based on transposons such as “Piggy-bac” (Saito et al, Cytother. 16:1257-1269, 2014) or “Sleeping Beauty”. In another embodiment, the vector is a retroviral vector, the activating agent is granulocyte colony stimulating factor (GCSF) and the target is CD34+ cells. In a further embodiment, the vector is a gamma retroviral non-replicating vector, the target population is naturally activated T cells, and the transgene is a SAR (e.g., a SIR, cTCR, Ab-TCR, HC-SAR etc.). In a further embodiment, the vector is a gamma retroviral vector, the target cell population is a naturally activated cell population and the transgene encodes an immune activating agent. In yet another embodiment, the vector is a gamma retroviral vector, the target population is naturally activated T cells, and the transgene is a SAR (e.g., a SIR, cTCR, Ab-TCR, HC-SAR etc.). In a further embodiment, the SAR comprises a binding domain that targets one or more of antigens listed in Table B.

In one aspect, the present disclosure provides a viral particle comprising a vector genome comprising a polynucleotide sequence encoding a SAR, wherein the SAR selectively expresses in T cells in vivo. In some embodiments, the viral particle is a lentiviral particle. In an embodiment, the vector is pseudotyped with an envelope glycoprotein selected from one or more of VSV-G, modified Baboon envelope glycoprotein, modified HERV-W1 envelope glycoprotein, gibbon ape envelope glycoprotein or Cocal glycoprotein. In some embodiments, the viral particle comprises a viral envelope comprising one or more immune cell-activating proteins exposed on the surface and/or conjugated to the surface of the viral envelope. In some embodiments, the viral envelope comprises an anti-CD3 single-chain variable fragment exposed on the surface and/or conjugated to the surface of the viral envelope. In some embodiments, the viral envelope comprises an envelope glycoprotein that is exposed on the surface and/or conjugated to the surface of the viral envelope. In some embodiments, the viral envelope comprises a Cocal glycoprotein exposed on the surface and/or conjugated to the surface of the viral envelope, optionally wherein the Cocal glycoprotein comprises the R354Q mutation.

In an embodiment, the disclosure provides an in vivo method for generating a SAR-expressing T cell or a stem cell that can differentiate into a T cell. In an embodiment, the in vivo method results in selective expression of the SAR only in T cells. In an embodiment, the in vivo method results in selective expression of the SAR only in cells that express at least one T cell chain selected from the group of T cell receptor constant chain α, β, γ, δ or preTCRα. In an embodiment, the in vivo method involves a SAR that comprises a sequence represented by SEQ ID NO: 31985-31988 and 31992. In an embodiment, the in vivo method for generation of SAR-T cells involves a SAR that comprises a sequence represented by SEQ ID NO: 40606-40758. In an embodiment, the SAR targets one or more antigens in an MHC-independent manner. In an embodiment, the SAR targets one or more antigens in an MHC-dependent manner. In an embodiment, the method comprises in vivo delivery of a recombinant polynucleotide encoding a SAR. In an embodiment, the SAR comprises one or more non-TCR antigen binding domain attached via optional linkers to at least one TCR constant chain or functional fragment or functional variant or homolog thereof, including homolog from non-human species. In an embodiment, the TCR constant chain or functional fragment or functional variant or homolog thereof comprises a T cell receptor module that is capable of recruiting at least one TCR-associated signaling module when expressed in a T cell. In an embodiment, the SAR comprises a vL fragment derived from an antibody attached via an optional linker to a first TCR chain or fragment thereof and a vH fragment derived from that antibody attached via an optional linker to a complementary TCR chain or fragment thereof so that when expressed in a T cell the vL and vH fragments associate to generate a fragment variable (Fv) that is capable of specifically binding to a target antigen and optionally recruiting at least one T cell receptor signaling module. In an embodiment, the SAR comprises one or more autonomous antigen binding domain (AABD) that are attached via an optional linker to the N-terminus or near the N-terminus of the non-TCR antigen binding domain. In an embodiment, the recombinant polynucleotide encodes a SAR selected from the group of a OHC SAR, a double chain SAR, a OHC chain chimeric T cell receptor (cTCR), a double cTCR, an OHC Ab-TCR, and a double chain Ab-TCR. In an embodiment, the SAR, cTCR and Ab-TCR comprise of hybrid TCR chains.

In an embodiment, the method results in SAR showing less than 1% (e.g., 1%, 0.5%, 0.2%, 0.1%, 0.05%, 0.02%, 01%, 0%) cell surface expression at protein level in cells other than T cells (e.g., non-T cells) when delivered in vivo. In an embodiment, the method results in SAR showing less than 1% (e.g., 1%, 0.5%, 0.2%, 0.1%, 0.05%, 0.02%, 0.01%, 0%) cell surface expression at protein level in cancer cells when delivered in vivo. In an embodiment, the cancer is of non-T cell lineage, e.g., B cell lymphoma, B cell ALL, AML, prostate cancer, breast cancer etc. In an embodiment, the cancer cells do not express one or more CD3 chains selected from the group of CD3ε, CD3γ and/or CD3δ. In an embodiment, the method results in SAR showing less than 1% (e.g., 1%, 0.5%, 0.2%, 0.1%, 0.05%, 02%, 01%, 0%) cell surface expression at protein level in healthy cells when delivered in vivo. In an embodiment, the healthy cells comprise one or more of B cell, macrophage, neutrophils, liver cells, spleen cells, heart cells, lung cells, kidney cells, breast cell, ovarian cells, epithelial cells, brain cells, skin cells, gastrointestinal cells, muscle cells etc. In an embodiment, the method results in cell surface expression of SAR protein in T cells that is at least 2-fold (e.g., 2-fold, 5-fold, 10-fold, 50-fold, 100-fold, 1000-fold, 10,000-fold, 100,000-fold, million fold, billion fold, or a trillion-fold) higher than its expression in non-T cells. In an embodiment, the method results in SAR showing less than 1% (e.g., 1%, 0.5%, 0.2%, 0.1%, 0.05%, 0.02%, 0.01%, 0%) incidence of second T cell cancers when delivered in vivo. In an embodiment, the method delivers the SAR construct in vivo using a standard vector.

In an embodiment, the method delivers the SAR construct in vivo using a vector that is not specifically targeted to T cells. In an embodiment, the method delivers the SAR construct in vivo using a vector that is not modified to reduce its insertion into cells other than T cells. In an embodiment, the method delivers the SAR for in vivo delivery using a viral vector. In an embodiment, the method delivers the SAR for in vivo delivery using a viral like particle. In an embodiment, the SAR for in vivo delivery is delivered using lipid nanoparticle. In an embodiment, the SAR for in vivo delivery is delivered using vector that are pseudotyped with standard envelope proteins. In an embodiment, the SAR is delivered via a vector that is not surface engineered. In an embodiment, the SAR is delivered in vivo using a vector that is pseudotyped with a wild-type envelope glycoprotein. In an embodiment, the SAR is delivered in vivo using a vector that is pseudotyped with an envelope glycoprotein that is still capable of binding to its native receptor. In an embodiment, the SAR is delivered in vivo using a vector that is pseudotyped with an envelope glycoprotein selected from one or more of VSV-G, modified Baboon envelope glycoprotein, modified HERV-W1 envelope glycoprotein, gibbon ape envelope glycoprotein. In an embodiment, the SAR is delivered in vivo using a DNA vector or an RNA vector. In an embodiment, the SAR is delivered in vivo using a circular RNA. In an embodiment, the SAR is delivered in vivo using an in vitro transcribed RNA. In an embodiment, the SAR is delivered in vivo using transposase vector.

In an embodiment, the disclosure provides a vector for in vivo generation of a SAR-expressing T cell or a stem cell that can differentiate into a T cell. In an embodiment, the vector results in selective expression of the SAR only in T cells. In an embodiment, the vector results in selective expression of the SAR only in cells that express at least one T cell chain selected from the group of T cell receptor constant chain α, β, γ, δ or preTCRα. In an embodiment, the SAR targets one or more antigens in an MHC-dependent and/or MHC-independent manner. In an embodiment, the vector comprises a recombinant polynucleotide encoding a SAR, where the SAR comprises one or more non-TCR antigen binding domain attached via optional linkers to at least one TCR constant chain or functional fragment or functional variant or homolog thereof, including homolog from non-human species. In an embodiment, the TCR constant chain or functional fragment or functional variant or homolog thereof comprises a T cell receptor module that is capable of recruiting at least one TCR-associated signaling module when expressed in a T cell. In an embodiment, the SAR comprises a sequence represented by SEQ ID NO: 31985-31988 and 31992. In an embodiment, the SAR comprises a sequence represented by SEQ ID NO: 40606-40758. In an embodiment, the SAR comprises a vL fragment derived from an antibody attached via an optional linker to a first TCR chain or fragment thereof and a vH fragment derived from that antibody attached via an optional linker to a complementary TCR chain or fragment thereof so that when expressed in a T cell the vL and vH fragments associate to generate a fragment variable (Fv) that is capable of specifically binding to a target antigen and optionally recruiting at least one T cell receptor signaling module. In an embodiment, the SAR comprises one or more autonomous antigen binding domain (AABD) that are attached via an optional linker to the N-terminus or near the N-terminus of the non-TCR antigen binding domain. In an embodiment, the recombinant polynucleotide encodes a SAR selected from the group of a OHC SAR, a double chain SAR, a OHC chain chimeric T cell receptor (cTCR), a double cTCR, an OHC Ab-TCR, and a double chain Ab-TCR. In an embodiment, the SIR, cTCR and Ab-TCR comprise of hybrid TCR chains. In an embodiment, the vector is selected from the group of a viral vector (e.g., lentiviral vector, γ retroviral vector, adenoviral vector, AAV vector, vaccinia vector, herpes simplex, baculoviral vector etc.), a non-viral vector (e.g., virus like particle, lipid nano-particle etc.), a DNA vector, an RNA vector, a transposon (e.g., sleeping beauty transposon or piggyback transposon). In an embodiment, the viral vector is pseudotyped with mBaEV, BaEV-TR, mHERV-W1, VSVG, gibbon ape envelop, measles virus fusion protein, nipah virus fusion protein. In an embodiment, the viral vector is used as a vaccine.

In an embodiment, the disclosure provides a recombinant system for generating a SAR-expressing T cell or a stem cell that can differentiate into a T cell. In an embodiment, the recombinant system results in selective expression of the SAR only in T cells. In an embodiment, the recombinant system vector results in selective expression of the SAR only in cells that express at least one T cell chain selected from the group of T cell receptor constant chain α, β, γ, δ or preTCRα. In an embodiment, the SAR targets one or more antigens in an MHC-dependent and/or MHC-independent manner. In an embodiment, the recombinant system comprises a recombinant polynucleotide encoding a SAR, where the SAR comprises one or more non-TCR antigen binding domain (e.g., antibody, antibody fragment, Fab, Fab2, scFv, vL, VH, vHH, FHVH, non-immunoglobulin antigen binding scaffold, extracellular domain of a receptor, extracellular domain of a ligand/cytokine, autoantigen, adaptor, epitope etc.) attached via optional linkers to at least one TCR constant chain or functional fragment or functional variant or homolog thereof, including homolog from non-human species. In an embodiment, the TCR constant chain or functional fragment or functional variant or homolog thereof comprises a T cell receptor module that is capable of recruiting at least one TCR-associated signaling module when expressed in a T cell. In an embodiment, the recombinant system comprises a SAR that comprises a sequence represented by SEQ ID NO: 31985-31988 and 31992. In an embodiment, the recombinant system comprises a SAR that comprises a sequence represented by SEQ ID NO: 40606-40758. In an embodiment, the recombinant polynucleotide encodes a SAR selected from the group of a OHC SIR, a double chain SIR, a OHC chain chimeric T cell receptor (cTCR), a double cTCR, an OHC Ab-TCR, and a double chain Ab-TCR. In an embodiment, the SIR, cTCR and Ab-TCR comprise of hybrid TCR chains. In an embodiment, the recombinant system comprises a viral vector, a non-viral vector (e.g., virus like particle, lipid nanoparticle etc.), a DNA vector, an RNA vector, a transposon (e.g., sleeping beauty transposon or piggyback transposon). In an embodiment, the recombinant system results in the expression of the SIR, Ab-TCR or cTCR from the promoter of an endogenous T cell gene. In an embodiment, the recombinant system results in the insertion of the polynucleotide encoding a SIR, Ab-TCR or cTCR at an endogenous T cell gene locus, wherein optionally the endogenous T cell gene locus is TRAC, TRBC, TRGC, TRDC locus or a combination.

In an embodiment, the disclosure provides a T cell or stem cell that can give rise to a T cell engineered in vivo to express a SAR. In an embodiment, the vector results in selective expression of the SAR only in cells that express at least one T cell chain selected from the group of T cell receptor constant chain α, β, γ, δ or preTCRα. In an embodiment, the SAR targets one or more antigens in an MHC-dependent and/or MHC-independent manner. In an embodiment, the cell expresses a recombinant polynucleotide encoding SAR or a fragment thereof. In an embodiment, the SAR comprises one or more non-TCR antigen binding domain attached via optional linkers to at least one TCR constant chain or functional fragment or functional variant or homolog thereof, including homolog from non-human species. In an embodiment, the TCR constant chain or functional fragment or functional variant or homolog thereof comprises a T cell receptor module that is capable of recruiting at least one TCR-associated signaling module when expressed in a T cell. In an embodiment, the SAR comprises a sequence represented by SEQ ID NO: 31985-31988 and 31992. In an embodiment, the SAR comprises a sequence represented by SEQ ID NO: 40606-40758. In an embodiment, the SAR comprises a vL fragment derived from an antibody attached via an optional linker to a first TCR chain or fragment thereof and a vH fragment derived from that antibody attached via an optional linker to a complementary TCR chain or fragment thereof so that when expressed in a T cell the vL and vH fragments associate to generate a fragment variable (Fv) that is capable of specifically binding to a target antigen and optionally recruiting at least one T cell receptor signaling module. In an embodiment, the SAR comprises one or more autonomous antigen binding domain (AABD) that are attached via an optional linker to the N-terminus or near the N-terminus of the non-TCR antigen binding domain. In an embodiment, the T cell or the stem cell that can give rise to a T cell expresses a recombinant polynucleotide that encodes a SAR selected from the group of a OHC SIR, a double chain SIR, a OHC chain chimeric T cell receptor (cTCR), a double cTCR, an OHC Ab-TCR, and a double chain Ab-TCR. In an embodiment, the SIR, cTCR and Ab-TCR comprise of one or more (i.e., two) hybrid TCR chains. In an embodiment, the cell is engineered using a recombinant system. In an embodiment, the recombinant system comprises a vector selected from the group of a viral vector (e.g., lentiviral vector, γ retroviral vector, adenoviral vector, AAV vector, vaccinia vector, baculoviral vector etc.), a non-viral vector (e.g., virus like particle, lipid nano-particle etc.), a DNA vector, an RNA vector, a transposon (e.g., sleeping beauty transposon or piggyback transposon). In an embodiment, the viral vector is pseudotyped with mBaEV, BaEV-TR, mHERV-W1, VSVG, gibbon ape envelop, measles virus fusion protein. In an embodiment, the cell is engineered using a recombinant system that results in the expression of the SIR, Ab-TCR or cTCR from the promoter of an endogenous T cell gene. In an embodiment, the recombinant system results in the insertion of the polynucleotide encoding a SIR, Ab-TCR or cTCR at an endogenous T cell gene locus, wherein optionally the endogenous T cell gene locus is TRAC, TRBC, TRGC, TRDC locus or a combination. In an embodiment, the T cell is a CD4, CD8, helper T cell, cytotoxic T cell, Treg, stem like T, NKT, memory T cell, naïve T cell, effector T cell or synthetic T cell.

A pharmaceutical composition that results in in vivo generation of SAR-expressing T cells. In an embodiment, the pharmaceutical composition does not result in expression of SAR in non-T cells. In an embodiment, the pharmaceutical composition comprises a SAR described in the preceding sections.

In an embodiment, the composition comprises a SAR that comprises a sequence represented by SEQ ID NO: 31985-31988 and 31992. In an embodiment, the composition comprises a SAR that comprises a sequence represented by SEQ ID NO: 40606-40758. In an embodiment, the composition comprises a SAR that comprises a vL fragment derived from an antibody attached via an optional linker to a first TCR chain or fragment thereof and a vH fragment derived from that antibody attached via an optional linker to a complementary TCR chain or fragment thereof so that when expressed in a T cell the vL and vH fragments associate to generate a fragment variable (Fv) that is capable of specifically binding to a target antigen and optionally recruiting at least one T cell receptor signaling module. In an embodiment, the composition comprises a SAR that comprises one or more autonomous antigen binding domain (AABD) that are attached via an optional linker to the N-terminus or near the N-terminus of the non-TCR antigen binding domain. In an embodiment, the composition comprises a SAR that comprises a recombinant polynucleotide that encodes a SAR selected from the group of a OHC SIR, a double chain SIR, a OHC chain chimeric T cell receptor (cTCR), a double cTCR, an OHC Ab-TCR, and a double chain Ab-TCR. In an embodiment, the composition comprises a SAR that comprises SIR, cTCR and Ab-TCR comprising of one or more (i.e., two) hybrid TCR chains. In an embodiment, the composition comprises a recombinant system that comprises a vector selected from the group of a viral vector, a non-viral vector (e.g., virus like particle, lipid nanoparticle etc.), a DNA vector, an RNA vector, a transposon (e.g., sleeping beauty transposon or piggyback transposon). In an embodiment, the viral vector is pseudotyped with mBaEV, BaEV-TR, mHERV-W1, VSVG, gibbon ape envelop, measles virus fusion protein. In an embodiment, the cell is engineered using a composition comprising a recombinant system that results in the expression of the SIR, Ab-TCR or cTCR from the promoter of an endogenous T cell gene. In an embodiment, the recombinant system results in the insertion of the polynucleotide encoding a SIR, Ab-TCR or cTCR at an endogenous T cell gene locus, wherein optionally the endogenous T cell gene locus is TRAC, TRBC, TRGC, TRDC locus or a combination. In an embodiment, the T cell is a CD4, CD8, helper T cell, cytotoxic T cell, Treg, stem like T, NKT, memory T cell, naïve T cell, effector T cell or synthetic T cell.

In an embodiment, the invention provides a method for providing anti-disease immunity in a subject comprising administering to the subject in vivo an effective amount of a recombinant polynucleotide or a recombinant system or a vector that results in the selective cell surface expression of a SAR protein only in T cells. In an embodiment, the subject has cancer, an autoimmune disease, an allergic disease, or a degenerative disease. In an embodiment, the subject receives a single dose recombinant polynucleotide or a recombinant system or a vector. In an embodiment, the subject receives multiple doses of recombinant polynucleotide or a recombinant system or a vector. In an embodiment, the recombinant polynucleotide or a recombinant system or the vector for in vivo generation of SAR-T cells is administered to the subject by any suitable route, including intravenous, intranodal, intraperitoneal, intralymphatic, intrasplenic, intra-arterial, subcutaneous etc. In another aspect, the present disclosure provides a method of treating a disease or disorder in a subject in need thereof, comprising administering a therapeutically effective amount of a viral particle to the subject by intraperitoneal, subcutaneous, or intranodal injection, wherein the viral particle transduces immune cells in vivo. In some embodiments, the viral particle is administered by intra-nodal injection, optionally via inguinal lymph node.

In some embodiments, at least 2 million, at least 4 million, at least 6 million, at least 8 million or at least 10 million transducing units of viral particle are administered to the subject. In some embodiments, the viral particle is administered as a single injection. In some embodiments, the viral particle is administered as at least 1, at least 2, at least 3, at least 4, at least 5, at least 6, at least 7, at least 8, at least 9, or at least 10 injections.

In an embodiment, the present disclosure provides a method of treating a disease or disorder, transducing immune cells in vivo, and/or generating an immune cell expressing a SAR in a subject in need thereof, comprising administering the viral particle of the present disclosure to the subject. In some embodiments, the method of the disclosure eliminates the need for pre-activation of the immune cells prior to administration of the viral particle. In some embodiments, the method comprises no pre-activation of the immune cells in the subject prior to administration of the viral particle (e.g., no pre-activation within about 1, 2, 3, 4, 5, 6, or 7 days, or within about 1, 2, 3, 4, 5, 6, 7, 8, 9, 10, 11, or 12 weeks prior to administration of the viral particle). In some embodiments, pre-activation of the immune cells comprises activating the CD3 and/or CD28 signaling in the immune cells (e.g., T cells), optionally by administering anti-CD3 and/or anti-CD28 antibodies, respectively. Accordingly, in some embodiments, the method of the disclosure does not comprise administering separate CD3 and/or CD28 activating agents prior to administration of the viral particle.

In some embodiments, a polynucleotide encoding a SAR is administered to the subject which allows the production of the SAR in vivo. In some embodiments, the administration of such polynucleotide generates similar effect in vivo as direct administration of the SAR. In some embodiments, the administration of such polynucleotide improves the in vivo transduction efficiency of a particle. In some embodiments, the polynucleotide is an mRNA.

In some embodiments, in vivo delivery of such polynucleotides generates SAR expression over time (e.g., starting within hours and lasting several days). In some embodiments, in vivo delivery of such polypeptides results in desirable pharmacokinetics, pharmacodynamics and/or safety profile of the encoded SAR. In some embodiments, the polynucleotide may be optimized by one or more means to prevent immune activation, increase stability, reduce any tendency to aggregate, such as over time, and/or to avoid impurities. Such optimization may include the use of modified nucleosides, modified, and/or particular 5′ UTRs, 3′UTRs, and/or poly (A) tail modifications for improved intracellular stability and translational efficiency (see, e.g., Stadler et al., 2017, Nat. Med.). Such modifications are known in the art.

Strategies for in vivo delivery of polynucleotides (e.g., mRNA) are known in the art. For a summary of strategies, see Mol. Ther. 2019 Apr. 10; 27 (4): 710-728, which is incorporated herein by reference in its entirety.

In some embodiments, the viral particle is administered via a route selected from the group consisting of parenteral, intravenous, intramuscular, subcutaneous, intratumoral, intraperitoneal, and intralymphatic. In some embodiments, the viral particle is administered multiple times.

In some embodiments, the lentiviral particle of the present disclosure is a replication incompetent, self-inactivating (SIN) lentiviral vector (LVV) particle. In an embodiment, the lentiviral particle comprises a VSV-G, modified BaEV or a modified HERV-W1 envelope glycoprotein. In an embodiment, the lentiviral particle comprises a surface-engineered viral envelope that includes expression of a membrane-bound anti-CD3 single-chain variable fragment (scFv) and the Cocal glycoprotein.

In some embodiments, the retroviral particles and/or lentiviral particles of the disclosure comprise a polynucleotide comprising a sequence encoding a receptor that specifically binds to a hapten. In some embodiments, a sequence encoding a receptor that specifically binds to the hapten is operatively linked to a promoter. Illustrative promoters include, without limitation, a cytomegalovirus (CMV) promoter, a CAG promoter, an SV40 promoter, an SV40/CD43 promoter, an EF-1a promoter, and a MND promoter.

In some embodiments, the retroviral particles comprise transduction enhancers. In some embodiments, the retroviral particles comprise a polynucleotide comprising a sequence encoding a T cell activator protein. In some embodiments, the retroviral particles comprise a polynucleotide comprising a sequence encoding a hapten-binding receptor. In some embodiments, the retroviral particles comprise tagging proteins.

In some embodiments, the retroviral particles comprise a cell surface receptor that binds to a ligand on a target host cell, allowing host cell transduction.

In some embodiments, the viral envelope glycoprotein is a VSV G protein from the Cocal strain (Cocal glycoprotein) including variant containing the R354Q mutation. Various fusion glycoproteins can be used to pseudotype lentiviral particles

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, anti-CD3 scFv, (CD3), Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis

New Jersey virus (VSNJV), Bas-Congo Virus (BASV). In some embodiments, the fusion glycoprotein or functional variant thereof is the Cocal virus G protein.

In some embodiments, the viral particle is a Nipah virus (NiV) envelope pseudotyped lentivirus particle (“Nipah envelope pseudotyped vector”). In some embodiments, the Nipah envelope pseudotyped vector is pseudotyped using Nipah virus envelope glycoproteins NiV-F and NiV-G. In some embodiments, the NiV-F and/or NiV-G glycoproteins on such Nipah envelope pseudotyped vector are modified variants. In some embodiments, the NiV-F and/or NiV-G glycoproteins on such Nipah envelope pseudotyped vector are modified to include an antigen binding domain. In some embodiments, the antigen is EpCAM, CD4, or CD8.

In some embodiments, the glycoprotein on an envelope pseudotyped viral particle is modified to include an antigen binding domain. In some embodiments, the antigen is CD3 In some embodiments, the antigen binding domain is an anti-CD3 single chain variable fragment (scFv). In some embodiments, the antigen binding domain is an CD3 humanized murine scFv.

In some embodiments, the envelope pseudotyped viral particle is modified to include a fusion glycoprotein or functional variant thereof and an antigen binding domain or functional variant thereof. In some embodiments, the envelope pseudotyped viral particle is modified to include the Cocal virus G protein or functional variant thereof and an anti-CD3 scFv or functional variant thereof.

In some embodiments, the retroviral vector particle is surface-engineered. Illustrative methods of surface-engineering a retroviral vector particle are provided in, e.g., WO 2019/200056, PCT/US2019/062675, and US 62/916,110, each of which is incorporated herein by reference in its entirety.

In some embodiments, the retroviral particle is surface-engineered to include a fusion glycoprotein or functional variant thereof and an antigen binding domain or functional variant thereof. In some embodiments, the retroviral particle is surface-engineered to include the Cocal virus G protein or functional variant thereof and an anti-CD3 scFv or functional variant thereof.

Various non-viral proteins capable of viral surface display are provided by the present disclosure. In some embodiments, the non-viral proteins are co-stimulatory molecules.

In some embodiments, the T-cell activation or co-stimulation molecule may be selected from the group consisting of an anti-CD3 antibody, CD28 ligand (CD28L), and 41bb ligand (41BBL or CD137L).

In some embodiments, the T-cell activation or co-stimulation molecule is selected from the group consisting of an anti-CD3 antibody, a ligand for CD28 (e.g., CD28L), and 41bb ligand (41BBL or CD137L). CD86, also known as B7-2, is a ligand for both CD28 and CTLA-4. In some embodiments, the ligand for CD28 is CD86. CD80 is an additional ligand for CD28. In some embodiments, the ligand for CD28 is CD80. The non-viral protein may be a cytokine, optionally selected from the group consisting of IL-15, IL-7, and IL-2.

In some embodiments, the surface-engineered vector comprises a transmembrane protein comprising a mitogenic domain and/or cytokine-based domain.

The mitogenic domain may comprise all or part of an antibody or other molecule which specifically binds a T-cell surface antigen. In some embodiments, the vector comprises an anti-CD3s antibody, or antigen-binding fragment thereof, coupled to a transmembrane domain. In some embodiments, the vector comprises a ligand for 4-1BB, or functional fragment thereof, coupled to its native transmembrane domain or a heterologous transmembrane domain.

The mitogenic transduction enhancer and/or cytokine-based transduction enhancer may comprise a “spacer sequence” to connect the antigen-binding domain with the transmembrane domain.

The viral particle of the present invention may comprise a cytokine-based transduction enhancer in the viral envelope. The cytokine-based transduction enhancer may comprise a cytokine domain and a transmembrane domain. The cytokine domain may comprise a T-cell activating cytokine, such as from IL2, IL7 and IL15, or a functional fragment thereof.

A viral particle may be used to infect cells in vivo at an any effective dosage. In some embodiments, the viral particle is administered to a subject in vivo, by direct injection to the cell, tissue, organ, or subject in need of therapy. Viral particles may also be delivered according to viral titer (TU/mL). In some embodiments, the viral titer delivered is about 10⁵ to 10⁶, about 10⁵ to 10⁷, 10⁷ to 10⁹, about 10⁸ to 10¹⁰.

In some embodiments, the viral particle is an adeno-associated virus (AAV) particle. In an embodiment, the SAR comprises a sequence represented by SEQ ID NO: 31985-31988 and 31992. In an embodiment, the SAR comprises a sequence represented by SEQ ID NO: 40606-40758. In an embodiment, the SAR comprises a sequence represented by SEQ ID NO: 33546-40513, 50054-55347.

The formulations and compositions of the present disclosure may comprise a combination of any number of viral particles, and optionally one or more additional pharmaceutical agents (polypeptides, polynucleotides, compounds etc.) formulated in pharmaceutically acceptable or physiologically acceptable compositions for administration to a cell, tissue, organ, or an animal, either alone, or in combination with one or more other modalities of therapy. In some embodiments, the one or more additional pharmaceutical agent further increases transduction efficiency of vectors.

In some embodiments, the composition further comprises other agents, such as, e.g., cytokines, growth factors, hormones, small molecules, or various pharmaceutically active agents.

In some embodiments, compositions and formulations of the viral particles used in accordance with the present disclosure may be prepared for storage by mixing a viral particle having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. In some embodiments, one or more pharmaceutically acceptable surface-active agents (surfactant), buffers, isotonicity agents, salts, amino acids, sugars, stabilizers and/or antioxidant are used in the formulation.

Suitable pharmaceutically acceptable surfactants comprise but are not limited to polyethylene-sorbitan-fatty acid esters, polyethylene-polypropylene glycols, polyoxyethylene-stearates and sodium dodecyl sulphates. Suitable buffers comprise but are not limited to histidine-buffers, citrate-buffers, succinate-buffers, acetate-buffers, and phosphate-buffers.

Isotonicity agents are used to provide an isotonic formulation. In some embodiments, isotonicity agents are generally used in a total amount of about 5 mM to about 350 mM.

In an embodiment, SAR expressing cells are generated in vivo or ex vivo using lipid nanoparticles (LNP). In an embodiment, SAR expressing cells are generated in in vivo or ex vivo using SAR encoding RNA. In an embodiment, RNA is a circular RNA (e.g., oRNA™). In an embodiment, circular RNA is delivered to the cells in vivo or ex vivo after packaging in LNP. Methods and compositions for generation of CAR in vivo using circular RNA are provided in WO2023250375 which is incorporated in their entirety by reference herein.

In an embodiment, the SAR expressing cells are generated in vivo or ex vivo using genome targeting. In an embodiment, the SAR construct is targeted to an endogenous T cell gene locus in vivo or ex vivo using genome targeting approaches known in the art. In an example embodiment, the endogenous T cell gene locus is TRAC, TRBC, TCDC, and/or TRGC gene locus. In an embodiment, the SAR construct is expressed from the promoter of an endogenous TCR gene (e.g., TCRα, TCRβ1, TCRβ2, TCRγ, TCRδ, CD3ζ, CD3ε, CD3γ or CDδ gene. A number of techniques for genome targeting are known in art, including but not limited to CRISP/Cas9, Zn finger nuclease, ARCUS, single-stranded DNA etc.

Additional methods and compositions for in vivo generation of SAR-expressing cells (e.g., SAR-T cells) are provided in WO2022164935, WO2022109162, WO2020142780, WO2020106992, WO2021154839, WO2023215848, WO2019219836, WO2009013324A1, WO2016009326, WO2023250375 and Xin, Tianqing, Cheng, Li et. al., Frontiers in Oncology vol 12, 2022, which are incorporated in their entirety by reference herein.

The disclosure further provides a method of controlling the expression and activity of a receptor in vitro and in vivo by controlling the expression and/or activity of one or more of its components and/or signaling mediator. The method can be useful in the treatment of various diseases involving the use of cells expressing the receptor.

In one embodiment, a region of the target gene of interest is targeted by a guide RNA and Cas9 in order to insert (knock-in) an expression cassette for drug inducible protein-destabilization domain (DIPDD) present in a homologous recombination (HR) targeting vector. The HR targeting vector contains homology arms at the 5′ and 3′ end of the expression cassette homologous to the genomic DNA surrounding the targeting gene of interest locus. By fusing drug inducible protein-destabilization domain (DIPDD) in frame with the target gene of interest, the resulting fusion protein upon expression will be made susceptible to proteasome mediated degradation upon treatment with a small molecule compound that binds to DIPDD. Example DIPDD include dTAG and ICBD (IMiD Cerebelon binding domain). Example small molecule compounds that can bind to dTAG and ICBD and induce proteasome mediated degradation include dTAG-13 and IMiD (e.g., pomalidomide).

### Examples

### Cloning of Baboon, HERV-W1 and HERV-FRD Envelopes

The mammalian expression vectors pcDNA3, pCDNA3.1 (+) and pSectagA vectors were obtained from Invitrogen (ThermoFisher Scientific). The gene fragments encoding the different envelopes were ordered from IDT and cloned in the mammalian expression vectors using standard molecular biology techniques.

### Generation of Lentiviral Vectors

The SAR constructs were cloned in the lentiviral, retroviral, or sleeping beauty transposon vectors. The methods for generation of SAR (e.g., 2^{nd} generation CARs, SIRs, Ab-TCR and TFP etc.), the generation and use of GGS-NLuc fusion proteins, and the generation and use of luciferase (e.g., GLuc and Luc146-1H2) reporter cell lines for measurement of cellular cytotoxicity using the Matador assays have been described (PCT/US2017/024843, PCT/US2017/025602, PCT/US2017/052344, PCT/US2017/064379, PCT/2020/014237 and PCT/US2018/53247), which are incorporated in their entirety by reference herein. For generation of baboon and HERV-W1 enveloped viruses, 10 μg of transfer plasmid was transfected into each p100 plate along with 1 μg of baboon or HERV-W1 envelope encoding plasmids and 6 μg of psPAX2 packaging plasmid. For pseudotyping with VSVG envelope, 10 μg of transfer plasmid, 3 μg of pLP/VSVG plasmid and 6 μg of psPAX2 packaging plasmid were used per p100 plate. Transfection was done into 293 FT cells using polyethylene amine (PEI) essentially as described previously (Natarajan et al, Scientific Reports, 10:2318) and PCT/US2018/53247. 293 FT cells were grown in DMEM with 10% FCS (hereby referred to as DMEM-10). Approximately 48-72 hr. post-transfection, all media was collected, pooled, and centrifuged at 1000 rpm for 1 minute to remove any cell debris and non-adherent cells. The cell-free supernatant was filtered through 0.45 μm syringe filter. In some cases, the supernatant was further concentrated by centrifugation at 18500 rpm for 2 hours at 4° C. The viral pellet was re-suspended in 1/10 of the initial volume in XVIVO medium. The virus was either used fresh to infect the target cells or stored frozen in aliquots at −80° C.

### Infection of T Cells, NK Cells and PBMC

Isolation of T cells and infection with lentivirus encoding SAR was done essentially as described in PCT/US2018/53247. Blood from a healthy donor was used to isolate NK cells using NK cell isolation kit (Miltenyi). NK92 cells were obtained from ATCC. NK Primary and NK92 cells were cultured in Minimum Essential Medium (MEM) Alpha without ribonucleosides and deoxy ribonucleosides supplemented with 20% Fetal bovine serum, 0.2 mM Inositol, 0.1 mM 2-Mercaptoethanol, 2 mM L-Glutamine, 1.5 g/L Sodium bicarbonate, 0.02 mM Folic Acid. For NK92 cells, medium was further supplemented with 200 IU/ml IL2. NK primary cells were cultured and activated with 500 IL/ml of IL2 for 7 days before infections. Lentiviral infections were done with concentrated lentivirus supernatant by spin infection in 6-well plates. For primary NK cells, approx. 4 million NK cells in 1.5 ml culture medium supplemented with 500 IU/ml IL2 and infected with 500 μl concentrated virus without polybrene. For NK92 cells, 6 ug/ml polybrene was used. The plates were centrifuged at 2,800 rpm for 90 min at 32° C. for 5 hours. The medium was changed after 5 hours, and the infection was repeated next day.

Unless specified otherwise, primary T cells were infected with lentiviral vectors pseudotyped with VSVG and primary NK cells were infected with lentiviral vectors pseudotype with modified baboon envelop, such as 072622-SyCD2 (SEQ ID NO: 95).

For detection of SARs using Protein L staining was used. FACS was done using FACSVerse analyzer from BD Biosciences. To measure cell death, Matador and Matador Glo assays were utilized as described in PCT/US2017/052344 and Choi S et al, Scientific Reports volume 11, 10002 (2021).

Infection of NK92 Cells with Baboon Envelope Virus

1 million NK92 cells (ATCC) were plated in 100 μl NK92 medium in each well in a 24-well non-tissue culture plate along with 400 μl of concentrated virus and 6 μg/ml polybrene. Plates were centrifuged at 2,800 RPM for 90 min at 32° C. followed by incubation at 37° C. for 5 hours. Culture medium was changed after 5 h and cells were expanded for 3 days. GFP expression was checked 3 days post-infection.

Experiment: NK92, JNG and primary NK cells were infected with a lentivirus encoding EGFP that was pseudotype with BaEVRless envelope constructs represented by SEQ ID NO: 100-104. In most experiments, the virus was concentrated prior to infection. Surprisingly, despite repeated attempts, no EGFP expression in the target cells was seen in any of the cells when the EGFP lentivirus was pseudotyped with the BaEVRless Baboon envelopes represented by SEQ ID NO: 100-104. The constructs with SEQ ID NO: 100 and 101 were generated in pcDNA3.1 vector and were expressed using CMV promoter. The constructs with SEQ ID NO: 102 was cloned in PsectagA vector and was also expressed using CMV promoter. Finally, the constructs with SEQ ID NO: 103 and 104 were expressed using pRC vector under RSV promoter. However, EGFP expression as seen when even a single amino acid was added to the C-terminus of the BaEVRless constructs represented by SEQ ID NO: 100-104. These constructs with an additional amino acid added to the C-terminus of the BaEVRless constructs are represented by SEQ ID NO (DNA): 86-91 and 115 and SEQ ID NO (PRT): 8466-8471 and 8495. The results are shown in Table 10. In addition, successful EGFP expression in the target cells was seen upon transduction with lentiviral vectors pseudotyped with modified baboon envelopes represented by SEQ ID NO:70-85 and 91-96 and 98. In an experiment, the constructs with SEQ ID NO: 72-78 showed EGFP expression in 22.0%, 33%, 8.2%, 50%, 25.8% and 61.3% of NK92 cells, respectively (Table 10). Very efficient EGFP expression was also seen using modified BaEV envelopes represented by SEQ ID NO: 95 and 98. In addition, modified BaEV envelopes represented by SEQ ID NO (DNA): 86-94 and 115-122 showed varying levels (between 2.7% to 50%) of EGFP transfer into target cells. A comparative ability of lentivirus vectors encoding EGFP and pseudotyped with the indicated modified BaEV envelopes in two separate experiments is shown in the following Table. The constructs with SEQ ID NO: 86-115

TABLE 10

| SEQ ID NO | GFP % NK92 | SEQ ID NO | GFP % NK92 |
|---|---|---|---|
| 70 | 4.23 | 86 | 18.91 |
| 72 | 22.00 | 87 | 11.96 |
| 73 | 33.20 | 88 | 5.45 |
| 74 | 8.23 | 89 | 36.43 |
| 75 | 25.89 | 90 | 28.77 |
| 78 | 61.35 | 91 | 11.17 |
| 98 | 43.14 | 115 | 7.44 |
| 78 | 12.02 | 70 | 18.75 |
| 76 | 15.04 | 71 | 14.35 |
| | | 78 | 40.13 |
| | | 95 | 35.73 |

NK92 cells were infected with a lentivirus encoding EGFP that was pseudotyped with the HERV-W1 envelope constructs. EGFP expression was measured by flow cytometry 48-72 h after infection. The nucleic acid and amino acid SEQ ID NOs of the different constructs (indicated by clone code) are provided in Table 2. In the case of HERV-W1 envelope, it was observed that EGFP lentiviruses pseudotyped with the HERV-W1 envelope comprising the wild-type signal peptide of HERV-W1 were not able to transduce GFP in NK92 or Jurkat cells. These constructs carried different C-terminal deletions in their cytosolic domain. These constructs are represented by SEQ ID NO: 8514-8518. In contrast, EGFP lentiviruses pseudotyped with the HERV-W1 envelopes comprising an exogenous signal peptide derived from murine Igκ chain were able to transfer EGFP expression to target cells successfully. Example such HERV-W1 envelopes with an exogenous signal peptide are represented by SEQ ID NO: 141-148 and 150-151, 201 and showed successful EGFP gene transfer. These constructs carried different C-terminal deletions in their cytosolic domain. It is to be understood that other exogenous signal peptides (e.g., CD8 signal peptide, human IgH signal peptide etc.) can substitute for murine Igκ signal peptide to allow HERV-W1 envelope to successfully pseudotype a viral vector. High level gene transfer of EGFP was seen with HERV-W1 constructs represented by SEQ ID NO:145, 150, 159 and 201. The results demonstrated that several modified baboon and HERV-W1 pseudotyped viruses were able to successfully infect NK92 cells. However, surprisingly, the BaEVRless envelope was not able to transfer EGFP when used to pseudotype an EGFP encoding lentiviral vector. In addition, surprisingly, HERV-W1 envelopes with the wild-type (native) signal peptide were not successful in transferring EGFP expression. Thus, HERV-W1 with exogenous signal peptide and a truncated cytosolic domain is unique among endogenous human retroviral envelopes in the ability to pseudotype viral vectors

Experiment. KG-1, a CD34+ stem cell leukemia cell line, was infected with a GFP encoding lentiviral vector. Flow cytometry analysis confirmed effective induction of KG-1 cells with baboon pseudotyped virus (SEQ ID NO: 95). In contrast, very poor GFP induction was seen with GFP lentiviral vector pseudotyped with VSVG.

### Experiment; Safety of In Vivo Generated SAR-T Cells

Female NSG MHCI/II^{DKO} mice are humanized with healthy human donor PBMCs. Animals are given 1 million NALM6-Luc cells by tail vein injection followed by tail vein administration of lentiviral particles (25-100×10⁸ titerable units (TU)) pseudotyped with VSVG, baboon (SEQ ID NO: 78), HERV-W1 (SEQ ID NO:159) and Cocal or aCD3-Cocal envelopes and containing a payload comprising different CD19 SAR. In vivo generation of SAR-T is demonstrated by flow cytometry using Protein L and/or Topanga assay on peripheral blood 1 week after viral administration. Flow cytometry demonstrates lack of expression of SARs with SEQ ID NO: 3250-3263, 3272-3274, 3931, 4967, 6003, 7039, 8106, 8160, 8178, 8196, 8124, 8142, 8214, 8232, 8250, 8268, 8286, 8304, 8322, 8340, 8358, 8376, 17437, 17462, 17433, 17463, 17511, 17581, 17610, 17640, 17669, 17699, 17728, 17758, 17787, 17817, 17846, 17876, 17905, 17935, 17994, 18023, 18053, 18082, 18112, 18141, 18171, 18200, 18230 in NALM6 cells or peripheral blood B lymphocytes. Similarly, lack of expression of the above constructs is seen in any of the normal tissues (e.g., heart, lung, liver, intestine, kidney etc.). In contrast, expression of SAR with SEQ ID NO: 3246-3249, 3264-3271, 3275-3282 is observed in NALM6, B lymphocytes and to varying extent in different healthy tissues.

### Screening of Antigen Binding Domains for Specific Binding Using Malibu Glo Assay

A number of novel antigen binding domains targeting different antigens are generated (SEQ ID NO: 388-398, 19804-19814), 32132-32194, 412-426, 32195-32213). The fragments are cloned into a lentiviral vector to generate Malibu-Glo reagents as described in PCT/US2017/025602. The Malibu Glo reagent supernatant containing the Nluc fusion proteins is used to test specific binding to cell lines expressing the target antigen using the Malibu Glo assay. The fold increase in Luc activity with example binding domains are shown in Table 12. The selected binders can be used to generate SAR constructs (e.g., SEQ ID NO:33860-40139, 40172-40511, 50054-50558) and tested for in vitro and in vivo assays. It is to be understood that SAR are modular in format and the various modules can be substituted. Therefore, based on the antigen binding domains and signaling chains provided in this disclosure, it is possible for one with ordinary skills in the art to develop additional SARs with different combinations of antigen binding domains, linkers and signaling chains, including hybrid chains. We envision these broad panel of SARs can be used to provide a diverse immune response. Furthermore, from this broad panel of SARs, it is possible to select SARs with specific properties (e.g., cytotoxicity, cytokine secretion, cell surface expression, in vivo activity etc.) using assays described in this disclosure.

TABLE 12

| | SEQ | Control | Target | | | SEQ | control | Target | |
|---|---|---|---|---|---|---|---|---|---|
| | ID | cell | cell | Fold | | ID | cell | cell | Fold |
| TARGET | NO | line | line | Change | TARGET | NO | line | line | Change |
| TAJ | 32169 | 293FT | LNCaP | 4.35 | CD19 | 32150 | Jurkat | RAJI | 903 |
| CD22 | 416 | K562 | RAJI | 4.32 | CD19 | 33431 | Jurkat | RAJI | 933 |
| DLL3 | 420 | K562 | NCI-H82 | 1.70 | CD19 | 32151 | Jurkat | RAJI | 270 |
| STEAP2 | 19814 | K562 | LNCaP | 27.37 | CD19 | 32199 | Jurkat | RAJI | 117 |
| IL23R | 32185 | K562 | L363 | 2.37 | CD19 | 32202 | Jurkat | RAJI | 160 |
| BCMA | 32197 | K562 | H929 | 2.57 | CD33 | 32205 | Jurkat | HL60 | 17 |
| BCMA | 32198 | K562 | H929 | 2.99 | CD33 | 32206 | Jurkat | HL60 | 28 |
| CD20 | 32195 | K562 | RAJI | 15.58 | BCMA | 32204 | Jurkat | L363 | 2. |
| MSLN | 32208 | Jurkat | SKOV3 | 11.52 | | | | | |

Induction of NFAT promoter driven GFP expression. Jurkat-NFAT-GFP (JNG) cells are infected with lentiviral vectors encoding different SAR constructs and optionally selected with puromycin. The control JNG cells and SAR-expressing JNG cells are cocultured for approximately 24 hours with different target cell lines expressing their cognate antigen(s). Thus, JNG cells expressing SARs targeting CD19 are co-cultured with CD19 antigen-expressing cell line RAJI and their ability to bind to the target antigen and induce cell signaling is assayed by measuring induction of GFP expression using Flow Cytometry. RAJI cells lacking CD19 (RAJI-CD19-KO) are used as negative controls in some cases. The induction of GFP expression is quantified as 1+, 2+, 3+, etc. depending on % of SAR expressing cells that induce GFP over the control cells. Thus, 1.6+ means that approximately 16% of JNG cells expressing the SAR showed GFP induction upon coculture with the target cells above the level seen with control JNG parental cells. In some cases, the cells are pulsed with the indicated peptides. The results in the following Table 13 demonstrate activation of NFAT induced GFP expression upon expression of different single chain and double chain SAR constructs. These results demonstrate that several HC-SAR (SAR) constructs targeting different antigens are highly active in the NFAT-GFP assay. These constructs comprise different antigen binding domains and one or two hybrid chains. Furthermore, they differ in the types of hybrid chains and the location of the junction between the two chains comprising the hybrid chain. The results further demonstrate the ability of HC-SAR to activate NFAT is not limited to constructs comprising vL and vH fragments but is also seen in constructs comprising different types of antigen binding domains (e.g., vHH, DARPIN, etc.). Furthermore, bispecific and multi-specific HC-SAR constructs are active (e.g., SEQ ID NO: 32851-76).

TABLE 13

| SEQ ID | | | |
|---|---|---|---|
| NO | SAR- | | |
| (DNA) | TYPE | Target Antigen | NFAT-GFP induction in Target cell line |
| 981 | SIR | MSLN | SKOV-3 5+ |
| 982 | FcRySIR | CD19 | RAJI 4.5+ |
| 983 | zSAR | NYESO-1/HLA-A2 | T2 -ve, T2 + 10 μM NYESO-pep 3+ |
| 984 | zSAR | NYESO-1/HLA-A2 | T2 -ve, T2 + 10 μM NYESO-peptide 3+ |
| 985 | zSAR | NYESO-1/MAGE- | T2 -ve, T2 + 10 μM NYESO-peptide 1.5+ |
| | | A3/HLA-A2 | |
| 986 | zSAR | BCMA; NYESO- | T2 +, T2 + 10 μM NYESO-peptide+ |
| | | 1/HLA-A2 | |
| 987 | zSAR | NYESO-1/HLA-A2 | T2 -ve, T2 + 10 μM NYESO-peptide 6+ |
| 988 | zSAR | NYESO-1/HLA-A2 | T2 -ve, T2 + 10 μM NYESO-peptide 6.5+, |
| 989 | zSAR | NYESO-1/HLA-A2 | T2 -ve, T2 + 10 μM NYESO-peptide 7+ |
| 991 | zSAR | NYESO-1/HLA-A2 | T2 -ve, T2 + 10 μM NYESO-peptide 2.5+, |
| 992 | zSAR | NYESO-1/HLA-A2 | T2 -vc, T2 + 10 μM NYESO-peptidc 6+ |
| 993 | CAR | CD19 | RAJI 4.5+ |
| 994 | zSIR | CD19 | RAJI 1.5+ |
| 995 | SIR | PSMA | LnCaP 4.5+, MDA-PCa-2b 3+ |
| 996 | zSIR | CD19 | RAJI 6+ |
| 997 | zSAR | NYESO-0/HLA-A2 | T2 -ve, T2 + 10 μM NYESO-peptide 1.5+ |
| 998 | zSAR | CD19 | RAJI 6+ |
| 999 | zSIR | CD19 | RAJI 2.5+ |
| 1000 | zSAR | NYESO-1/HLA-A2 | T2 -ve, T2 + 1 μM NYESO-peptide 2+ |
| 1001 | ySIR | CD19 | RAJI 4.5+ |
| 1002 | zSIR | CD19 | RAJI 2+ |
| 1003 | zSIR | CD19 | RAJI + |
| 1004 | zSIR | CD19 | RAJI 3+ |
| 1005 | zSAR | NYESO-1/HLA-A2 | T2 -ve, T2 + 1 μM NYESO-peptide 2+ |
| 1006 | zSIR | CD19 | RAJI 3+ |
| 1007 | zSIR | CD19 | RAJI 3+ |
| 1008 | zSIR | CD19 | RAJI + |
| 1010 | zSIR | CD19 | RAJI 2.5+ |
| 1012 | zSIR | CD19 | RAJI + DMSO 1.5+, RAJI + 1 μM Pom +/−, RAJI + |
| | | | 10 μM Len +/− |
| 1013 | CAR | CD70 | A-704 3+, 786-O 2.5+, ACHN 1.5+, THP-1 -ve |
| 1014 | CAR | CD72 | RAJI + |
| 1015 | SIR | KLK2 | LnCaP +/−, MDA-PCa-2b -ve |
| 1016 | SIR | KLK2 | LnCaP +/−, MDA-PCa-2b -ve |
| 1018 | zSIR | PSMA | LnCaP + |
| 1019 | zCD16-SIR | PSMA | LNCaP + |
| 1020 | zSIR | PSMA | LnCaP +/− |
| 1021 | zSIR | MSLN | SKOV-3 +/− |
| 1023 | zSIR | MSLN | SKOV-3 + |
| 1024 | zSIR | CD19 | RAJI + |
| 1025 | zSIR | CD19 | RAJI + |
| 1026 | zSIR | CD19 | RAJI + |
| 1027 | zSIR | CD19 | RAJI 2+ |
| 1028 | zSIR | CD19 | RAJI + |
| 1029 | zSIR | CD19 | RAJI 1.5+ |
| 1030 | zSIR | CD19 | RAJI +/− |
| 1031 | zSIR | CD19 | RAJI + DMSO +, RAJI + 1 μM Pom +/−, RAJI + 10 μM |
| | | | Len +/− |
| 1032 | zSIR | CD19 | RAJI 2+ |
| 1033 | zSIR | CD19 | RAJI 2+ |
| 1038 | SIR | CD20, CD19, CD72 | RAJI 3+ |
| 1039 | SIR | BCMA, CD19, CD72 | RAJI +/−, L363 +/−, U266 +/− |
| 1040 | zSIR | CD19 | RAJI + DMSO 2.5+, RAJI + 100 nM dTAG 1.5+ |
| 1041 | zSIR | CD19 | RAJI + DMSO 3+, RAJI + 1 μM Pom 1.5+, RAJI + |
| | | | 10 μM Len 2+ |
| 1042 | zSIR | CD19 | RAJI + DMSO 3.5+, RAJI + 1 μM Pom 3+, RAJI + |
| | | | 10 μM Len 3+ |
| 1043 | zSIR | MPL | HEL +/− |
| 1044 | zSIR | DART1 | L428 1.5+ |
| 1045 | zSIR | CD123 | L428 +/− |
| 1046 | zSIR | CD19 | RAJI 3.5+ |
| 1047 | zSIR | CD19 | RAJI 2.5+ |
| 1048 | zSIR | CD19 | RAJI 4+ |
| 1049 | zSIR | CD19 | RAJI + |
| 1050 | zSIR | CD19 | RAJI 4+ |
| 1051 | zSIR | CD19 | RAJI 3+ |
| 1052 | zSIR | CD19 | RAJI 3+ |
| 1053 | zSIR | CD19 | RAJI 3.5+ |
| 1054 | zSIR | CD19 | RAJI 3.5+ |
| 1055 | zSIR | CD19 | RAJI 2+ |
| 1056 | zSIR | CD19 | RAJI 4+ |
| 1057 | zSIR | CD19 | RAJI 4+ |
| 1058 | zSIR | CD19 | RAJI 3.5+ |
| 1059 | zSIR | CD19 | RAJI 3.5+ |
| 1060 | zSIR | CD19 | RAJI 3+ |
| 1061 | SIR | PSMA | LnCaP 2+, MDA-PCa-2b 2+ |
| 1062 | HC-SAR | CD19 | RAJI 4+ |
| 1063 | HC-SAR | CD19 | RAJI 2+ |
| 1064 | HC-SAR | CD19 | RAJI 3.5+ |
| 1065 | HC-SAR | CD19 | RAJI 3.5+ |
| 1066 | HC-SAR | CD19 | RAJI 3+ |
| 1067 | zSIR | CD19 | RAJI 1.5+ |
| 1068 | zSIR | CD19 | RAJI 2+ |
| 1069 | zSIR | CD19 | RAJI + |
| 1070 | SIR | CD72, CD19 | RAJI + |
| 1071 | SIR | CD72, CD19 | RAJI 2+ |
| 1072 | SIR | p53/HLA-A2 | T2 +/−, T2-p53WT +/−, T2-p53R175H 1.5+ |
| 1073 | SIR | CD70 | THP-1 +, A-704 7+, 786-O 4+, ACHN 4.5+ |
| 1074 | zSIR | CD70 | THP-1 +, A-704 6+, 786-O 4+, ACHN 3+ |
| 1075 | zSIR | CD70 | THP-1 +, A-704 6.5+, 786-O 4+, ACHN 4+, K562 -ve |
| 1076 | zSIR | CD70 | THP-1 +/−, A-704 5+, 786-O 2.5+, ACHN 3+, |
| 1077 | zSIR | CD70 | THP-1 +, A-704 3. 5+, 786-O 1.5+, ACHN 2.5+, |
| 1078 | zSIR | CD19 | RAJI 1.5+ |
| 1079 | zSIR | CD19 | RAJI 1.5+ |
| 1080 | zSIR | CD19 | RAJI +/− |
| 1081 | FcyR-SIR | CD19 | RAJI +/− |
| 1082 | zSAR | NYESO-1/HLA-A2 | T2 -ve, T2 + 10 μM NYESO-peptide +/− |
| 1083 | zSAR | NYESO-1/HLA-A2 | T2 -ve, T2 + 10 μM NYESO-peptide +/− |
| 1084 | zSIR | CD19 | RAJI + |
| 1085 | zSAR | NYESO-1/HLA-A2 | T2 -ve, T2 + 10 μM NYESO-peptide +/− |
| 1086 | FcyR-SIR | CD19 | RAJI + |
| 1087 | FcyR-SIR | CD19 | RAJI 2.5+ |
| 1088 | zCD16-SIR | PSMA | LnCaP 4+ |
| 1089 | zSIR | PSMA | LnCaP +/− |
| 1090 | zSIR | CD33 | Molm-13 +/−, Molm-14 +/−, L428 +/− |
| 1091 | zSIR | CD19 | RAJI +/− |
| 1092 | zSIR | CD19 | RAJI 1.5+ |
| 1093 | SIR | CD19 | RAJI-DMSO +/−, RAJI-Pom +/−, RAJI-Len +/− |
| 1094 | SIR | CD19 | RAJI-DMSO 1.5+, RAJI-dTAG 1.5+ |
| 1095 | SIR | CD19 | RAJI-DMSO 1.5+, RAJI-dTAG 1.5+ ↓ |
| 1096 | zSIR | CD19 | RAJI-DMSO 1.5+, RAJI-Pom + ↓, RAJI-Len +/− ↓ |
| 1097 | CAR | CLDN-18-2 | NUGC-4 +/−, KATOIII +/− |
| 1098 | SIR | DLL3, CD19, CD20 | RAJI +, NCI-H2171 +/−, NCI-H82 +/−, |
| 1099 | SIR | BCMA, CD19, | RAJI 1.5+, L363 +, U266 +, NUGC-4 +/− |
| | | CLDN18-2 | |
| 1100 | SIR | BCMA, CD19, CD22 | RAJI +, L363 +, U266 + |
| 1101 | SIR | CD20, CD19, | RAJI +/−, NUGC-4 +/−, KATOIII +/− |
| | | CLDN18-2 | |
| 1102 | SIR | CD22, CD19, CD20 | RAJI 1.5+ |
| 22695 | CAR | CD70 | A-704 +, ACHN +, 786-O + |
| 22696 | CAR | CD70 | A-704 +/−, ACHN +/−, 786-O +/− |
| 22697 | CAR | CD70 | A-704 +/−, ACHN +/−, 786-O +/− |
| 22698 | SIR | CD70 | A-704 +, ACHN +, 786-O + |
| 22699 | zSIR | CD70 | A-704 +/−, ACHN +/−, 786-O +/− |
| 22700 | SIR | BCMA, DLL3, CD19 | RAJI + , RAJI-CD19-KO +, RAJI-CD20-KO +, L363 +, |
| | | | U266 +, NCI-H82 +/ |
| 22701 | SIR | DLL3, CD19, CD20 | RAJI +, RAJI-CD19-KO +, NCI-H82 +/−, SHP-77 +/−, |
| | | | SK-MEL5 +/− |
| 22703 | zCD16-SIR | PSMA | LnCaP +/− |
| 22704 | zSIR | PSMA | LnCaP +/− |
| 22705 | SIR | CD19 | RAJI 3+, RAJI + 10 μM Chlorpromazine 2+, |
| 22706 | zSAR | NYESO-1/HLA-A2 | T2 -ve, T2 + 10 μM NYESO-peptide +/− |
| 22707 | zSAR | NYESO-1/HLA-A2 | T2 -ve, T2 + 10 μM NYESO-peptide +/− |
| 22708 | STR | CD30, CD19, CD72 | RAJI +/−, L428 +/− |
| 22709 | CAR | CD19 | RAJI 4+, RAJI + 10 μM Chlorpromazine 3+, RAJI + 10 |
| | | | μM Nifedipine 3.5+ |
| 22710 | SIR | CD22, CD19, CD20 | RAJI +, RAJI-CD19-KO 1.5+, RAJI-CD20-KO 1.5+, |
| | | | RAJI-CD22-KO 1.5+, RAJI-CD19-CD22-KO 1.5+, |
| 22711 | zSIR | GD2 | SH-Sy5y +/−, SK-MEL-5 -ve, A375 -ve |
| 22712 | | GD2 | SH-Sy5y +, SK-MEL-5 +/−, A375 +/− |
| 22713 | zSIR | GD2 | SH-Sy5y +/−, SK-MEL-5 -ve, A375 -ve |
| 22714 | zCD16-SIR | GD2 | SH-Sy5y +, SK-MEL-5 -vc, A375 -vc |
| 22715 | zCD16-SIR | GD2 | SH-Sy5y +, SK-MEL-5 +/−, A375 +/− |
| 22716 | SIR | GD2 | SH-Sy5y +, SK-MEL-5 +/−, A375 -ve |
| 22717 | | CD19 | RAJI + |
| 22718 | CD16-SAR | CD19 | RAJI 1.5+ |
| 22719 | SIR | CD20, CD19 | RAJI 1.5+, RAJI-CD19-KO 2+, RAJI-CD20-KO 2+, |
| | | | RAJI-CD19-CD20-KO +/− |
| 22720 | zSIR | CLDN-18-2 | NUGC-4 +/−, KATOIII +/− |
| 22721 | SIR | CD22, CD19, BCMA | RAJI 3+, RAJI-CD19-KO 2+, RAJI-CD20-KO 2+, RAJI- |
| | | | CD19-CD20-KO 2+, L363 3.5+, U266 3+ |
| 22722 | zSIR | CD22, CD19 | RAJI 3+, RAJI-CD19-KO +, RAJI-CD22-KO 2+, RAJI- |
| | | | CD19-CD22-KO 1.5+ |
| 22723 | zSIR | CD22, CD19 | RAJI 3+, RAJI-CD19-KO +, RAJI-CD22-KO 1.5+, |
| | | | RAJI-CD19-CD22-KO 1.5+ |
| 22724 | zSIR | CD22, CD19, BCMA | RAJI +, RAJI-CD19-KO +/−, RAJI-CD22-KO +/−, RAJI- |
| | | | CD19-CD22-KO +/−, L363 -ve, U266 +/− |
| 22725 | zSIR | CD22, CD19, CD20 | RAJI 2+, RAJI-CD19-KO 1.5+, RAJI-CD20-KO 1.5+, |
| | | | RAJI-CD22-KO 2+, RAJI-CD19-CD22-KO 1.5+ |
| 22726 | zSIR | CD123 | L428 +/−, Molm-13 +/− |
| 22727 | zSIR | CD123 | L428 +/−, Molm-13 +/− |
| 22728 | zSIR | CD123 | L428 2+ Molm-13 +/− |
| 22729 | zCD16-SIR | CD123 | L428 2.5+ Molm-13 + |
| 22730 | zSIR | CD123 | L428 2+ Molm-13 + |
| 22731 | zSIR | CD123 | L428 +/−, Molm-13 +/− |
| 22732 | zCD16-SIR | CD123 | L428 3+ Molm-13 + |
| 22733 | HC-SAR | CD123 | L428 +/−, Molm-13 -ve |
| 32428 | SIR | BCMA | L363 (+1), MM1S (+1), OPM2 (+1.5) |
| 32429 | SIR | BCMA | L363 (+/−), MM1S (+1), OPM2 (+/−) |
| 32430 | SIR | BCMA | L363 (+/−), MM1S (+/−), OPM2 (+/−) |
| 32431 | SIR | BCMA | L363 (+/−), MM1S (+1.5) |
| 32432 | SIR | BCMA | L363 (+1), MM1S (+2.5), OPM2 (+1.5) |
| 32433 | SIR | BCMA | L363 (+/−), MM1S (+/−), OPM2 (+/−) |
| 32434 | SIR | BCMA | L363 (+/−), Daudi (+/−), RPMI-8266 (+/−), NCI-H929 (+/−) |
| 32437 | SIR | BCMA | L363 (+/−), MM1S (+/−), OPM2 (+/−), Daudi (+/−), |
| 32438 | SIR | BCMA | L363 (+/−), MM1S (+/−), OPM2 (+/−), RPMI-8266 (+/−) |
| 32439 | SIR | BCMA | MM1S (+1), OPM2 (+/−), Daudi (+/−), RPMI-8266 (+1) |
| 32441 | SIR | BCMA | RAJI (+4), NCI-H929 (+1), L363 (+3), MM1S (+4.5), |
| | | | RPMI-8226 (+5) |
| 32442 | zSIR | BCMA | NCI-H929 (+/−), MM1S (+/−), RPMI-8226 (+/−) |
| 32443 | zCD16- | BCMA | MM1S (+1), L363 (+/−), U266 (+/−), RPMI-8226 (+/−) |
| | SAR | | |
| 32444 | z16SIR | BCMA | NCI-H929 (+1), MM1S (+1), RPMI-8226 (+1) |
| 32445 | z16SIR | BCMA | RAJI (+/−), Jcko1 (+/−). MM1S (+2), RPMI-8226 (+1) |
| 32446 | HC-SAR | BCMA | MM1S (+2), NCI-H929 (+2), L363 (+1.5), U266 (+2), |
| | | | RPMI-8226 (+2) |
| 32448 | SIR | CD19 | RAJI (+4) |
| 32449 | SIR | CD19 | RAJI (+1) |
| 32450 | SIR | CD19 | RAJI (+3) |
| 32451 | SIR | CD19 | RAJI (+3) |
| 32452 | SIR | CD19 | RAJI (+/−) |
| 32453 | SIR | CD19 | RAJI (+4) |
| 32454 | SIR | CD19 | RAJI (+4) |
| 32455 | SIR | CD19 | RAJI (+2) |
| 32456 | SIR | CD19 | RAJI (+2) |
| 32457 | SIR | CD19 | RAJI (+1.5) |
| 32458 | SIR | CD19 | RAJI (+2) |
| 32459 | SIR | CD19 | RAJI (+2) |
| 32460 | SIR | CD19 | RAJI (+2) |
| 32461 | SIR | CD19 | RAJI (+1.5), RAJI-CD19-KO (−) |
| 32462 | SIR | CD19 | RAJI (−), RAJI-CD19-KO (−) |
| 32463 | SIR | CD19 | RAJI (+2) |
| 32464 | SIR | CD19 | RAJI (+/−) |
| 32465 | SIR | CD19 | RAJI (+2.5) |
| 32466 | SIR | CD19 | RAJI (+2.5) |
| 32467 | SIR | CD19 | RAJI (+2.5) |
| 32468 | SIR | CD19 | RAJI (+2) |
| 32469 | SIR | CD19 | RAJI (+3.5) |
| 32470 | SIR | CD19 | RAJI (+2.5) |
| 32471 | SIR | CD19 | RAJI (+1) |
| 32472 | SIR | CD19 | RAJI (+1) |
| 32473 | SIR | CD19 | RAJI (+/−) |
| 32474 | SIR | CD19 | RAJI (+2.5) |
| 32475 | SIR | CD19 | RAJI (+6.5) |
| 32476 | SIR | CD19 | RAJI (+5) |
| 32477 | CAR | CD19 | RAJI (+2.5) |
| 32478 | CAR | CD19 | RAJI (+2.5) |
| 32479 | CAR | CD19 | RAJI (+2.5) |
| 32480 | CAR | CD19 | RAJI (+/−) |
| 32481 | zSIR | CD19 | RAJI (+/−) |
| 32482 | zSIR | CD19 | RAJI (+/−) |
| 32483 | zSIR | CD19 | RAJI (+1.5) |
| 32484 | zSIR | CD19 | RAJI (+/−) |
| 32485 | zSIR | CD19 | RAJI (+6) |
| 32486 | zSIR | CD19 | RAJI (+1) |
| 32487 | zSIR | CD19 | RAJI (+2.5) |
| 32488 | zSIR | CD19 | RAJI (+1) |
| 32489 | zSIR | CD19 | RAJI (+/−) |
| 32490 | zSIR | CD19 | RAJI (+1) |
| 32491 | zSIR | CD19 | RAJI (+2.5) |
| 32492 | zSIR | CD19 | RAJI (+/−) |
| 32493 | zSIR | CD19 | RAJI (+1) |
| 32494 | zSIR | CD19 | RAJI (+1) |
| 32495 | zSIR | CD19 | RAJI (+1.5) |
| 32496 | zSIR | CD19 | RAJI (+1) |
| 32497 | zSIR | CD19 | RAJI (−) |
| 32498 | zSIR | CD19 | RAJI (+1) |
| 32499 | zSIR | CD19 | RAJI (+1.5) |
| 32500 | zSIR | CD19 | RAJI (+/−) |
| 32501 | zSIR | CD19 | RAJI (+1.5) |
| 32502 | zSIR | CD19 | RAJI (+1.5) |
| 32503 | zSIR | CD19 | RAJI (+2) |
| 32504 | zSIR | CD19 | RAJI (+/−) |
| 32505 | zSIR | CD19 | RAJI (+2) |
| 32506 | zSIR | CD19 | RAJI (+1.5) |
| 32507 | zSIR | CD19 | RAJI (+/−) |
| 32508 | zSIR | CD19 | RAJI (+/−) |
| 32509 | zSIR | CD19 | RAJI (+/−) |
| 32510 | zSIR | CD19 | RAJI (+2) |
| 32511 | zSIR | CD19 | RAJI (+/−) |
| 32512 | zSIR | CD19 | RAJI (+/−) |
| 32513 | zSIR | CD19 | RAJI (+1) |
| 32514 | zSIR | CD19 | RAJI (+/−) |
| 32515 | zSIR | CD19 | RAJI (+/−) |
| 32516 | zSIR | CD19 | RAJI 2+ |
| 32517 | zSIR | CD19 | RAJI (+/−) |
| 32518 | zSIR | CD19 | RAJI (+3) |
| 32519 | zSIR | CD19 | RAJI (+/−) |
| 32520 | zSIR | CD19 | RAJI (+/−) |
| 32521 | zSIR | CD19 | RAJI (+1) |
| 32522 | zSIR | CD19 | RAJI (+/−) |
| 32523 | zSIR | CD19 | RAJI (+/−) |
| 32524 | zSIR | CD19 | RAJI (+/−) |
| 32525 | zSIR | CD19 | RAJI (+1) |
| 32526 | zSIR | CD19 | RAJI (+1.5) |
| 32527 | zSIR | CD19 | RAJI (+/−) |
| 32528 | zSIR | CD19 | RAJI (+1) |
| 32529 | zSIR | CD19 | RAJI (+2) |
| 32530 | zSIR | CD19 | RAJI (+1) |
| 32531 | zSIR | CD19 | RAJI (+/−) |
| 32532 | zSIR | CD19 | RAJI (+5) |
| 32533 | zSIR | CD19 | RAJI (+2.5) |
| 32534 | zSIR | CD19 | RPMI-8226 (−), RAJI (+/−) |
| 32535 | zSIR | CD19 | RAJI (+4.5) |
| 32536 | zSIR | CD19 | RAJI (+2.5) |
| 32537 | zSIR | CD19 | RAJI (+2.5) |
| 32540 | z16SIR | CD19 | RAJI (+5) |
| 32541 | z16SIR | CD19 | RAJI (+1.5) |
| 32542 | z16SIR | CD19 | RAJI (+2) |
| 32543 | z16SIR | CD19 | RAJI (+2) |
| 32544 | z16SIR | CD19 | RAJI (+1.5) |
| 32545 | z16SIR | CD19 | RAJI (+2) |
| 32546 | z16SIR | CD19 | RAJI (+1.5) |
| 32547 | z16SIR | CD19 | RAJI (+3) |
| 32548 | z16SIR | CD19 | RAJI (+1.5) |
| 32549 | z16SIR | CD19 | RAJI (+3) |
| 32550 | z16SIR | CD19 | RAJI (+1) |
| 32551 | HC-SAR | CD19 | RAJI (+1.5) |
| 32552 | HC-SAR | CD19 | RAJI (+1) |
| 32553 | HC-SAR | CD19 | RAJI (+1.5) |
| 32554 | HC-SAR | CD19 | RAJI (+2.5) |
| 32555 | HC-SAR | CD19 | RAJI (+2) |
| 32556 | HC-SAR | CD19 | RAJI (+2.5) |
| 32557 | HC-SAR | CD19 | RAJI (+/−) |
| 32558 | HC-SAR | CD19 | RAJI (+/−) |
| 32559 | HC-SAR | CD19 | RAJI (+1) |
| 32560 | HC-SAR | CD19 | RAJI (+1) |
| 32561 | HC-SAR | CD19 | RAJI (+1) |
| 32562 | HC-SAR | CD19 | RAJI (+/−) |
| 32563 | HC-SAR | CD19 | RAJI (+/−) |
| 32564 | HC-SAR | CD19 | RAJI (+2) |
| 32565 | HC-SAR | CD19 | RAJI (+/−) |
| 32566 | HC-SAR | CD19 | RAJI (+1) |
| 32567 | HC-SAR | CD19 | RAJI (+/−) |
| 32568 | HC-SAR | CD19 | RAJI (+/−) |
| 32569 | HC-SAR | CD19 | RAJI (+/−) |
| 32570 | HC-SAR | CD19 | RAJI (+/−) |
| 32571 | HC-SAR | CD19 | RAJI (+/−) |
| 32572 | HC-SAR | CD19 | RAJI (+2) |
| 32573 | HC-SAR | CD19 | RAJI (+2) |
| 32574 | HC-SAR | CD19 | RAJI (+/−) |
| 32575 | HC-SAR | CD19 | RAJI (+/−) |
| 32576 | HC-SAR | CD19 | RAJI (+1.5) |
| 32577 | HC-SAR | CD19 | RAJI (+/−) |
| 32578 | HC-SAR | CD19 | RAJI (+1.5) |
| 32579 | HC-SAR | CD19 | RAJI (+1) |
| 32580 | HC-SAR | CD19 | RAJI (+/−) |
| 32581 | HC-SAR | CD19 | RAJI (+2.5) |
| 32582 | HC-SAR | CD19 | RAJI (+2.5) |
| 32583 | HC-SAR | CD19 | RAJI (+/−) |
| 32584 | HC-SAR | CD19 | RAJI (+4) |
| 32585 | HC-SAR | CD19 | RAJI (+1.5) |
| 32586 | HC-SAR | CD19 | RAJI (+3) |
| 32587 | HC-SAR | CD19 | RAJI (+4) |
| 32588 | HC-SAR | CD19 | RAJI (+1.5) |
| 32589 | HC-SAR | CD19 | RAJI (+2) |
| 32590 | HC-SAR | CD19 | RAJI (+2.5) |
| 32591 | HC-SAR | CD19 | RAJI (+1.5) |
| 32592 | HC-SAR | CD19 | RAJI (+3.5) |
| 32593 | HC-SAR | CD19 | RAJI (+1) |
| 32594 | HC-SAR | CD19 | RAJI (+2) |
| 32595 | HC-SAR | CD19 | RAJI (+/−) |
| 32596 | HC-SAR | CD19 | RAJI (+1.5) |
| 32597 | HC-SAR | CD19 | RAJI (+/−) |
| 32598 | HC-SAR | CD19 | RAJI (+1) |
| 32599 | HC-SAR | CD19 | RAJI (+2) |
| 32600 | Link CAR | CD19 | RAJI (+2) |
| 32601 | Link CAR | CD19 | RAJI (+1.5) |
| 32602 | Link CAR | CD19 | RAJI (+1) |
| 32603 | Link CAR | CD19 | RAJI (+/−) |
| 32604 | Link CAR | CD19 | RAJI (+1.5) |
| 32605 | Link CAR | CD19 | RAJI (+3) |
| 32606 | Link CAR | CD19 | RAJI (+2) |
| 32607 | Link CAR | CD19 | RAJI (+2), Jeko1 (+1.5) |
| 32608 | Link CAR | CD19 | RAJI (+/−) |
| 32609 | Link CAR | CD19 | RAJI (+/−) |
| 32610 | | CD19 | RAJI (+/−) |
| 32613 | SIR | CD22 | SKOV3 (+/−), OVCAR3 (+1.5) |
| 32618 | z16SIR | CD70 | A-704 (+/−), ACHN (−), 786-O (+/−) |
| 32619 | SIR | CD79b | RAJI (+1.5), Jeko1 (+2.5) |
| 32620 | SIR | CD79b | RAJI (+/−), Jeko1 (−) |
| 32621 | SIR | CD79b | RAJI (+/−), Jeko1 (+/−) |
| 32622 | zSIR | CD79b | RAJI (+/−), Jeko1 (+/−) |
| 32623 | HC-SAR | CD79b | RAJI (+/−), Jeko1 (+/−) |
| 32624 | | CD123 | L428 (+/−) |
| 32625 | SIR | CD123 | MOLM-13 (+/−), L428 (+/−) |
| 32626 | SIR | CLD18A2 | KATOIII (+/−), NUGC4 (+1) |
| 32627 | SIR | CLD18A2 | KATOIII (+/−), NUGC4 (+1) |
| 32628 | SIR | CLD18A2 | KATOIII (+/−), NUGC4 (+1) |
| 32629 | SIR | CLD18A2 | KATOIII (+/−), NUGC4 (+/−) |
| 32630 | SIR | CLD18A2 | KATOIII (+1), NUGC4 (+/−) |
| 32631 | SIR | CLD18A2 | KATOIII (+/−), NUGC4 (+/−) |
| 32632 | SIR | CLD18A2 | KATOIII (+/−), NUGC4 (+/−) |
| 32633 | SIR | CLD18A2 | KATOIII (+/−), NUGC4 (+/−) |
| 32634 | SIR | CLD18A2 | KATOIII (+/−), NUGC4 (+1) |
| 32635 | SIR | CLD18A2 | KATOIII (+1), NUGC4 (+1) |
| 32636 | SIR | CLD18A2 | KATOIII (+/−), NUGC4 (+1) |
| 32637 | SIR | CLD18A2 | KATOIII (+/−), NUGC4 (+/−) |
| 32638 | CAR | CLD18A2 | KATOIII (+/−), NUGC4 (+1.5) |
| 32639 | SIR | CLDN6 | K OVCAR3 (+/−), OV90 (+/−), HepG2 (+/−), |
| 32640 | SIR | CLDN6 | OVCAR3 (+3), OV90 (+1) |
| 32641 | SIR | CLDN6 | OVCAR3 (+2), OV90 (+2) |
| 32644 | SIR | CLDN6 | OVCAR3 (−), OV90 (+/−) |
| 32645 | SIR | CLDN6 | OVCAR3 (+1.5), OV90 (+1) |
| 32646 | SIR | CLDN6 | OVCAR3 (+1), OV90 (+1) |
| 32647 | SIR | CLDN6 | OVCAR3 (+1.5), OV90 (+1) |
| 32650 | SIR | CLDN6 | OVCAR3 (+1.5), OV90 (+/−), |
| 32652 | SIR | CLDN6 | OVCAR3 (+2.5), OV90 (+2.5) |
| 32653 | SIR | CLDN6 | SKOV3 (+/−), |
| 32654 | SIR | CLDN6 | SKOV3 (−), OVCAR3 (+/−), |
| 32655 | SIR | CLDN6 | SKOV3 (−), OVCAR3 (+/−), OV90 (+/−), |
| 32656 | SIR | CLDN6 | SKOV3 (−), OVCAR3 (+/−), OV90 (+/−) |
| 32657 | SIR | CLDN6 | OVCAR3 (+2.5), OV90 (+1) |
| 32658 | SIR | CLDN6 | OVCAR3 (+3), OV90 (+1.5) |
| 32660 | SIR | CLDN6 | OVCAR3 (+3), OV90 (+/−) |
| 32661 | SIR | CLDN6 | OVCAR3 (+1.5), OV90 (+1) |
| 32662 | SIR | CLDN6 | OVCAR3 (+4.5), OV90 (+2.5) |
| 32663 | SIR | CLDN6 | OVCAR3 (+1), OV90 (+1) |
| 32664 | SIR | CLDN6 | OVCAR3 (+4), OV90 (+2) |
| 32665 | CAR | CLDN6 | SKOV3 (−), OVCAR3 (+/−), OV90 (+/−), |
| 32666 | CAR | CLDN6 | OVCAR3 (+1.5), OV90 (+1.5) |
| 32667 | CAR | CLDN6 | OVCAR3 (+/−), OV90 (+/−), SKOV3 (−) |
| 32669 | CAR | CLDN6 | OVCAR3 (+1), OV90 (+/−), SKOV3 (−) |
| 32670 | CAR | CLDN6 | OVCAR3 (+/−), OV90 (+/−), SKOV3 (−) |
| 32671 | SIR | EGFRviii | A431 (+/−), HeLa (−), Hela-EGFR (−) |
| 32672 | SIR | EGFRviii | A431 (+/−), HeLa (−), Hela-EGFR (−) |
| 32673 | CAR | EGFRviii | A431 (+1.5), HeLa (+1), Hela-EGFR (+1) |
| 32674 | HC-SAR | FR1 | SKOV3 (+3.5), OVCAR3 (+6) |
| 32675 | SIR | GD2 | A375 (−), SH-SY5Y (+/−) SK-MEL-5 (+/−) |
| 32676 | SIR | GD2 | A375 (+/−), SH-SY5Y (+1) SK-MEL-5 (+1.5) |
| 32678 | SIR | GD2 | A375 (+/−), SH-SY5Y (−) SK-MEL-5 (+/−) |
| 32679 | SIR | GD2 | A375 (+/−), SH-SY5Y (+/−) SK-MEL-5 (+/−) |
| 32680 | SIR | GD2 | A375 (+/−), SH-SY5Y (+/−) SK-MEL-5 (+/−) |
| 32681 | SIR | GD2 | A375 (+/−), SH-SY5Y (+/−) SK-MEL-5 (+/−) |
| 32682 | SIR | GD2 | A375 (+/−), SH-SY5Y (+1) SK-MEL-5 (−) |
| 32683 | SIR | GD2 | A375 (+/−), SH-SY5Y (+/−) SK-MEL-5 (+/−) |
| 32684 | SIR | GD2 | A375 (+/−), SH-SY5Y (+1) SK-MEL-5 (+1) |
| 32685 | SIR | GD2 | A375 (+/−), SH-SY5Y (+1.5) SK-MEL-5 (+1) |
| 32686 | SIR | GD2 | A375 (+1), SH-SY5Y (+2) SK-MEL-5 (+1.5) |
| 32687 | zSIR | GD2 | A375 (+/−), SH-SY5Y (−) SK-MEL-5 (+1) |
| 32688 | z16SIR | GD2 | A375 (+/−), SH-SY5Y (+1) SK-MEL-5 (+/−) |
| 32689 | z16SIR | GD2 | A375 (−), SH-SY5Y (+/−) SK-MEL-5 (+/−) |
| 32690 | z16SIR | GD2 | A375 (−), SH-SY5Y (+/−) SK-MEL-5 (+/−) |
| 32691 | z16SIR | GD2 | A375 (+/−), SH-SY5Y (+1) SK-MEL-5 (−) |
| 32692 | z16SIR | GD2 | A375 (−), SH-SY5Y (+/−) SK-MEL-5 (−) |
| 32693 | HC-SAR | GD2 | A375 (−), SH-SY5Y (+/−) SK-MEL-5 (−) |
| 32694 | z16SIR | GPC3 | HepG2 (−), Hutu-1080 (+/−), Huh7 (−) |
| 32696 | z16SIR | GPC3 | HepG2 (−), Hutu-1080 (+1), Huh7 (+/−) |
| 32697 | z16SIR | GPC3 | HepG2 (+/−), Hutu-1080 (−), Huh7 (−) |
| 32698 | z16SIR | GPC3 | HepG2 (−), Hutu-1080 (−), Huh7 (+1) |
| 32699 | z16SIR | GPC3 | HepG2 (+/−), Hutu-1080 (−), Huh7 (−) |
| 32700 | SIR | GPRC5D | L363 (+/−), MM1S (+1), OPM2 (+/−) |
| 32701 | SIR | GPRC5D | L363 (−), MM1S (+/−), OPM2 (−), Daudi (−), RPMI-8266 (+/−), |
| 32702 | zSIR | GPRC5D | OPM2 (+/−), Daudi (+/−), RPMI-8266 (+/−) |
| 32703 | SIR | HER2 | SKOV3 (+/−), OVCAR3 (+1) |
| 32704 | SIR | HER2 | SKOV3 (+/−), OVCAR3 (−) |
| 32705 | SIR | HLA-A2-SANG | U266 (+2), Bv173 (+2) |
| 32706 | SIR | HLA-A2-SANG | U266 (+/−), Bv173 (+/−) |
| 32707 | CAR | HLA-A2-SANG | U266 (+1.5), Bv173 (+2) |
| 32708 | CAR | HLA-A2-SANG | U266 (+1.5), Bv173 (+2) |
| 32709 | SIR | IL13Ra2 | SKOV3 (+/−), U87MG (+/−) |
| 32710 | SIR | IL13Ra2 | SKOV3 (−), U87MG (+/−) |
| 32711 | SIR | IL13Ra2 | SKOV3 (−), U87MG (+1) |
| 32712 | CAR | IL23R | DLD-1 (+2), HT29 (+2), L363 (−), U266 (+/−) |
| 32714 | SIR | Muc16 | OVCAR3 (+/−), OV90 (+/−), SKOV3 (−) |
| 32715 | SIR | MUC16 | SKOV3 (−), OVCAR3 (+/−) |
| 32716 | SIR | MSLN | SKOV3 (+1.5), OVCAR3 (+3.5), OV90 (−) |
| 32717 | zSIR | MSLN | SKOV3 (+/−) |
| 32718 | HC-SAR | MSLN | SKOV3 (+2), OVCAR3 (+3) |
| 32719 | HC-SAR | MSLN | SKOV3 (+3.5), OVCAR3 (+5.5) |
| 32720 | HC-SAR | MSLN | SKOV3 (+3), OVCAR3 (+4) |
| 32722 | zSIR | p53 | T2 (+/−), T2 + p53-WT (+/−), T2+ p53 R175 (+3) |
| 32723 | zSIR | p53 | T2 (+/−), T2+ p53-R175 (+2), T2 + p53-WT (+/−) |
| 32724 | z16SIR | p53 | T2 (+/−), T2 + p53-R175 (+4), T2 + p53-WT (+/−) |
| 32725 | z16SIR | p53 | T2 (+/−), T2 + p53-R248Q (+/−) |
| 32726 | z16SIR | p53 | T2 (+/−), T2 + p53-R175 (+4), T2 + p53-WT (+/−) |
| 32727 | z16SIR | p53 | T2 (+/−), T2 + p53-R248Q (+/−) |
| 32728 | z16SIR | p53 | T2 (+/−), T2 + p53-R175 (+2), T2 + p53-WT (+/−) |
| 32729 | z16SIR | p53 | T2 (+/−), T2 + p53-R248Q (+/−) |
| 32730 | SIR | PSCA | NCI-H1993 (+/−) |
| 32731 | SIR | PSCA | NCI-H1993 (+/−), NCI-H2126 (+/−) |
| 32732 | SIR | PSCA | NCI-H1993 (+1.5), NCI-H2126 (+1), NUGC-4 (−) |
| 32733 | SIR | PSCA | NCI-H1993 (+/−), NCI-H2126 (+1) |
| 32734 | SIR | PSCA | NCI-H1993 (+1) |
| 32735 | zSIR | PSCA | NCI-H1993 (+/−), NCI-H2126 (−) |
| 32736 | z16SIR | PSCA | NCI-H1993 (+1), NCI-H2126 (+1), NUGC-4 (−) |
| 32737 | z16SIR | PSCA | NCI-H1993 (+/−) |
| 32738 | HC-SAR | PSCA | NCI-H1993 (+/−), NCI-H2126 (+/−) |
| 32739 | HC-SAR | PSCA | NCI-H1993 (+/−) |
| 32740 | HC-SAR | PSCA | NCI-H1993 (+/−) |
| 32741 | HC-SAR | PSCA | NCI-H1993 (+/−), NCI-H2126 (−) |
| 32742 | HC-SAR | PSCA | NCI-H1993 (+/−), NCI-H2126 (−) |
| 32743 | HC-SAR | PSCA | NCI-H1993 (+/−), NCI-H2126 (+/−) |
| 32744 | HC-SAR | PSCA | NCI-H1993 (+/−), NCI-H2126 (+/−) |
| 32745 | HC-SAR | PSCA | NCI-H1993 (+/−), NCI-H2126 (+/−) |
| 32746 | HC-SAR | PSCA | NCI-H1993 (+/−), NCI-H2126 (+/−) |
| 32747 | HC-SAR | PSCA | NCI-H1993 (+/−), NCI-H2126 (+/−) |
| 32748 | HC-SAR | PSCA | NCI-H1993 (+/−), NCI-H2126 (+/−) |
| 32749 | HC-SAR | PSCA | NCI-H1993 (+/−), NCI-H2126 (+/−) |
| 32750 | HC-SAR | PSCA | NCI-H1993 (+/−), NCI-H2126 (+/−) |
| 32752 | zSIR | PSMA | LNCaP (+2) |
| 32753 | zSIR | PSMA | LNCaP (+1) |
| 32754 | zSIR | PSMA | LNCaP (+1.5) |
| 32755 | zSIR | PSMA | LNCaP (+1) |
| 32756 | zSIR | PSMA | LNCaP (+3) |
| 32757 | zSIR | PSMA | LNCaP (+1.5) |
| 32758 | zSIR | PSMA | LNCaP (+3.5) |
| 32759 | zSIR | PSMA | LNCaP (+2) |
| 32760 | zSIR | PSMA | LNCaP (+1.7) |
| 32761 | zSIR | PSMA | LNCaP (+2.1) |
| 32762 | zSIR | PSMA | LNCaP (+1.5) |
| 32763 | zSIR | PSMA | LNCaP (+3.5) |
| 32764 | z16SIR | PSMA | LNCaP (+4) |
| 32765 | z16SIR | PSMA | LNCaP (+2.5) |
| 32766 | z16SIR | PSMA | LNCaP (+2.2) |
| 32767 | z16SIR | PSMA | LNCaP (+1.7) |
| 32768 | z16SIR | PSMA | LNCaP (+3) |
| 32769 | z16SIR | PSMA | LNCaP (+5) |
| 32770 | z16SIR | PSMA | LNCaP (+2.5) |
| 32771 | z16SIR | PSMA | LNCaP (+1.5) |
| 32772 | z16SIR | PSMA | LNCaP (+2.5) |
| 32773 | zSIR | ROR1 | Jeko1 (+/−) |
| 32774 | z16SIR | ROR1 | Jeko1 (+/−) |
| 32775 | z16SIR | RORI | Jeko1 (+/−) |
| 32776 | z16SIR | ROR1 | Jeko1 (+/−) |
| 32777 | HC-SAR | ROR1 | Jeko-1 (+/−) |
| 32778 | HC-SAR | ROR1 | Jeko1 (+/−) |
| 32779 | SIR | STEAP2 | LNCaP (+1) |
| 32780 | SIR | STEAP2 | LNCaP (+3) |
| 32781 | SIR | STEAP2 | LNCaP (+/−) |
| 32782 | z16SIR | STEAP2 | LNCaP (+/−) |
| 32783 | z16SIR | STEAP2 | LNCaP (+/−) |
| 32784 | z16SIR | STEAP2 | LNCaP (+/−) |
| 32785 | HC-SAR | STEAP2 | LNCaP (+/−) |
| 32786 | HC-SAR | STEAP2 | LNCaP (+/−) |
| 32787 | SIR | GPRC5D-BCMA | L363 (+/−), MM1S (+/−) RPMI-8266 (+2.5), |
| 32788 | SIR | GPRC5D-BCMA | L363 (+1.5), MM1S (+2), Daudi (+1.5), RPMI-8266 |
| | | | (+2.5), NCI-H929 (+2) |
| 32790 | SIR | BCMA-CD20 | L363 (+/−), MM1S (+/−), OPM2 (+/−), RPMI-8266 (+1), |
| 32791 | SIR | CLD18A2-CD20 | KATOIII (+/−), NUGC4 (+/−) |
| 32792 | SIR | CLD18A2-CD20 | KATOIII (+/−), NUGC4 (+/−) |
| 32793 | SIR | BCMA-CLD18A2 | NCI-H929 (−), MM1S (+/−), RPMI-8226 (−), KATOIII (−) |
| 32794 | SIR | BCMA-CLD18A2 | MM1S (+1), RPMI-8226 (−), |
| 32795 | SIR | BCMA-CLD18A2 | U266 (+/−), NCI-H929 (−), MM1S (+1.5), NUGC4 (+/−) |
| 32796 | SIR | BCMA-CD20 | MM1S (+/−), RPMI-8226 (−) |
| 32798 | SIR | CD20-CD19 | U266 (+1), NCI-H929 (+1), MM1S (+1.5), RAJI (+1) |
| 32799 | SIR | CD20-CD19 | RAJI (+1) |
| 32800 | SIR | CD20-CD19 | RAJI (+1.5) |
| 32801 | SIR | MSLN-MUC16 | SKOV3 (+1.5), OVCAR3 (+3), OV90 (−) |
| 32802 | SIR | CD19-MUC16 | SKOV3 (+/−), OVCAR3 (−), OV90 (−), RAJI(+/−) |
| 32803 | SIR | CD19-MUC16 | SKOV3 (−), OVCAR3 (−), OV90 (−), RAJI(+/−) |
| 32804 | SIR | MUC16-MSLN | SKOV3 (+/−), OVCAR3 (+2.5), OV90 (−) |
| 32805 | SIR | CD20-CD19 | RAJI (+5) |
| 32806 | SIR | IL13Ra2-CD19 | RAJI (+5.5), U87MG (+1.5) |
| 32807 | SIR | IL13Ra2-CD19 | RAJI (+6.5), U87MG (+2) |
| 32808 | SIR | CD19-BCMA | RAJI (+5), MM1S (+1), RPMI-8226 (+2) |
| 32809 | SIR | CD19-BCMA | RAJI (+4), NCI-H929 (+1), MM1S (+1), RPMI-8226 (+2) |
| 32810 | SIR | GPRC5D-BCMA | RAJI (+1), NCI-H929 (+1), L363 (+1), MM1S (+1), |
| | | | RPMI-8226 (+1) |
| 32811 | SIR | GPRC5D-BCMA | RAJI (+5), NCI-H929 (+1), L363 (+3), MM1S (+4), |
| | | | RPMI-8226 (+1) |
| 32812 | SIR | GPRC5D-BCMA | RAJI (+5), NCI-H929 (+1), L363 (+2), MM1S (+4), |
| | | | RPMI-8226 (+4.5) |
| 32813 | SIR | MUC16-MSLN | SKOV3 (+1.5), OVCAR3 (−) |
| 32814 | SIR | BCMA-CD19 | RAJI (+/−), L363 (−), U266 (−) |
| 32815 | SIR | CD20-CD19 | Jeko1 (+/−), RAJI (+2) |
| 32816 | SIR | CD20-CD19 | Jeko1 (+/−), RAJI (+2) |
| 32817 | SIR | CD20-CD19 | Jeko1 (+/−), RAJI (+3) |
| 32818 | SIR | BCMA-CD19 | RAJI (+1.5), L363 (+/−), NCI-H929 (+/−), MM1S (+/−) |
| 32819 | SIR | BCMA-CD19 | RAJI (+/−), NCI-H929 (+/−), MM1S (+/−) |
| 32820 | SIR | BCMA-CD19 | RAJI (+/−), L363 (+/−), U266 (−), NCI-H929 (+/−) |
| 32821 | SIR | BCMA-CD19 | RAJI (+/−), L363 (+/−), U266 (−), NCI-H929 (+/−), |
| 32822 | SIR | CD20-CD19 | RAJI (+1.5), Jeko1 (+/−) |
| 32823 | SIR | CD20-CD19 | RAJI (+1), Jeko1 (+1) |
| 32824 | SIR | CD79b-CD20 | RAJI (+1), Jeko1 (+/−) |
| 32825 | SIR | CD79b-CD20 | RAJI (+2), Jeko1 (+2) |
| 32826 | SIR | CD79b-BCMA | RAJI (+2.5), Jeko1 (+2.5). L363 (+2.5), NCI-H929 (+3), |
| | | | MM1S (+3), RPMI-8226 (+3.5) |
| 32827 | SIR | CD22-CD20 | RAJI (+3.5), Jeko1 (+1.5) |
| 32828 | SIR | CD19-BCMA | RAJI (+1.5) |
| 32830 | zSIR | CD19-CD20 | RAJI (+1.5) |
| 32831 | zSIR | GPRC5D-BCMA | L363 (+/−), MM1S (+1), OPM2 (+1) |
| 32832 | zSIR | GPRC5D-BCMA | L363 (+1), MM1S (+2), OPM2 (+1.5) |
| 32833 | zSIR | GPRC5D-BCMA | L363 (+/−), MM1S (+1.5), OPM2 (+1) |
| 32835 | zSIR | GPRC5D-BCMA | MM1S (+1.5), U266 (+/−), RPMI-8266 (+1.5), |
| 32836 | zSIR | GPRC5D-BCMA | L363 (+/−), MM1S (+1), U266 (+/−) |
| 32837 | zSIR | GPRC5D-BCMA | L363 (+1), NCI-H929 (+1), MM1S (+2) |
| 32838 | zSIR | GPRC5D-BCMA | L363 (+/−), U266 (+1), NCI-H929 (+1), MM1S (+1.5) |
| 32839 | zSIR | GPRC5D-BCMA | L363 (+1), U266 (+2), NCI-H929 (+1.5), MM1S (+3), |
| | | | RPMI-8226 (+1.5) |
| 32840 | zSIR | CD20-CD19 | Jeko1 (+1), RAJI (+3) |
| 32841 | zSIR | BCMA-CD19 | RAJI (+1), L363 (+/−), U266 (−), RPMI-8226 (+/−) |
| 32842 | zSIR | CD19-CD20 | RAJI (+1.5), Jeko1 (+1) |
| 32845 | zSIR | CD19-CD20 | RAJI (+1.5), Jeko1 (+1) |
| 32846 | zSIR | CD19-CD22 | RAJI (+1.5), Jeko1 (+/−) |
| 32847 | zSIR | CD19-BCMA | RAJI (+1), Jcko1 (+/−). L363 (+/−), U266 (+1), MM1S |
| | | | (+1) |
| 32848 | zSIR | CD19-BCMA | Jeko1 (+/−). L363 (+/−), U266 (+/−), NCI-H929 (+/−) |
| 32849 | z16SIR | CD19-CD20 | RAJI (+2), Jeko1 (+/−). NCI-H929 (−) |
| 32850 | z16SIR | CD19-CD20 | RAJI (+2), NCI-H929 (−) |
| 32851 | HC-SAR | CD20-CD19 | Jeko1 (+1), RAJI (+2) |
| 32852 | HC-SAR | CD20-ROR1 | Jeko1 (+/−), RAJI (+2) |
| 32853 | HC-SAR | CD20-CD19 | Jeko1 (+1), RAJI (+2) |
| 32854 | HC-SAR | CD19-BCMA | RAJI (+1), Jeko1 (+/−), L363 (−), U266 (−) |
| 32855 | HC-SAR | CD19-CD22 | RAJI (+/−), Jeko1 (+/−) |
| 32856 | HC-SAR | CD19-CD22 | RAJI (+/−), Jeko1 (+/−) |
| 32857 | HC-SAR | CD19-CD22 | RAJI (+1), Jeko1 (+/−) |
| 32858 | HC-SAR | CD19-CD22 | RAJI (+1), Jeko1 (+1) |
| 32859 | HC-SAR | CD19-CD22 | RAJI (+/−), Jeko1 (+/−) |
| 32860 | HC-SAR | CD19-CD22 | RAJI (+1), Jeko1 (+/−) |
| 32861 | HC-SAR | CD19-BCMA | RAJI (+1), MM1S (+1.5), RPMI-8226 (+1.5) |
| 32863 | HC-SAR | CD19-BCMA | RAJI (+/−), NCI-H929 (+/−), MM1S (+1) |
| 32864 | HC-SAR | CD19-BCMA | RAJI (+1), L363 (+1), U266 (+1), NCI-H929 (+1), |
| | | | MM1S (+1) |
| 32865 | HC-SAR | CD19-BCMA | RAJI (+/−), Jcko1 (+/−). L363 (+/−), MM1S (+1), |
| 32866 | HC-SAR | CD19-BCMA | RAJI (+/−), NCI-H929 (+/−), MM1S (+/−) |
| 32867 | HC-SAR | CD19-BCMA | RAJI (+1), NCI-H929 (+1), MM1S (+1), RPMI-8226 (+1) |
| 32868 | HC-SAR | BCMA-CD19 | MM1S (+/−), NALM6 (−), |
| 32869 | HC-SAR | BCMA-CD19 | MM1S (+/−), NALM6 (+/−), |
| 32870 | HC-SAR | CD19-PSMA | RAJI (+1) |
| 32871 | HC-SAR | CD19-BCMA | RAJI (+1) |
| 32872 | HC-SAR | BCMA-CD19 | MM1S (+/−), NALM6 (+/−), U87MG (−) |
| 32873 | HC-SAR | CD19-PSMA | RAJI (+2) |
| 32874 | HC-SAR | CD19-BCMA | RAJI (+1.5) |
| 32875 | HC-SAR | CD20-CD19 | RAJI (+2), Jeko1 (+/−) |
| 32876 | HC-SAR | HER2-CD19 | RAJI (+1) |
| 32877 | Link CAR | CD19-CD20 | RAJI (+3), Jeko1 (+3) |
| 32878 | SIR | CD22-CD19-BCMA | L363 (+/−), MM1S (+1), U266 (+1) |
| 32879 | SIR | CD20-CD19-BCMA | L363 (+/−), NCI-H929 (+1), MM1S (+1),RAJI (+/−) |
| 32880 | SIR | BCMA-CD19-CD20 | MM1S (+1), RPMI-8226 (−), RAJI (−) |
| 32881 | SIR | CD20-CD19-BCMA | U266 (+1.5), NCI-H929 (+1.5), MM1S (+1), RPMI-8226 |
| | | | (+1), RAJI (+3.5) |
| 32882 | SIR | BCMA-CD20-CD19 | NCI-H929 (+/−), RAJI (+1) |
| 32883 | SIR | BCMA-CD20-CD19 | Jeko1 (+/−), RAJI (+/−) |
| 32884 | SIR | BCMA-CD19-CD20 | RAJI (+2), Jeko1 (+1.5), NCI-H929 (+1.5), MM1S |
| | | | (+1.5) |
| 32885 | SIR | BCMA-CD19-CD20 | NCI-H929 (+2), L363 (+2), MM1S (+3), RPMI-8226 |
| | | | (+2.5), U266 (+1.5), RAJI (+2), Jeko1 (+1) |
| 32886 | SIR | BCMA-CD20-CD19 | RAJI (+/−), Jeko1 (+/−). MM1S (+/−), RPMI-8226 (+/−) |
| 32887 | SIR | BCMA-CD20-CD19 | RAJI (+/−), Jeko1 (+/−). NCI-H929 (+/−) |
| 32888 | SIR | BCMA-CD20-CD19 | RAJI (+6), NCI-H929 (+1.5), MM1S (+1), RPMI-8226 |
| | | | (+1) |
| 32889 | SIR | CD20-BCMA-CD19 | RAJI (+6.5) |
| 32890 | SIR | CD20-BCMA-CD19 | RAJI (+5.5) |
| 32891 | SIR | BCMA-CD20-CD19 | RAJI (+2.5), NCI-H929 (+2), L363 (+1.5), MM1S (+2), |
| | | | RPMI-8226 (+3) |
| 32892 | SIR | BCMA-CD20-CD19 | RAJI (+1.5), NCI-H929 (+/−), MM1S (+/−) |
| 32893 | SIR | CD20-CD19-MSLN | SKOV3 (−), OVCAR3 (−), Jeko1 (+/−), RAJI (+1.5) |
| 32894 | SIR | HER3-HER2-CD19 | SKOV3 (−), OVCAR3 (+/−), HeLa (−), RAJI (−) |
| 32895 | HC-SAR | BCMA-CD20-CD19 | MM1S (+/−), NALM6 (+/−) |
| 32896 | HC-SAR | BCMA-CD20-CD19 | RAJI (+2.5), Jeko1 (+/−) |
| 40431 | HC-SAR | CD19 | RAJI-WT (+4) |
| 40432 | HC-SAR | CD22-CD19 | RAJI-WT (+2), RAJI-CD 19-KO (+2), RAJI-CD22-KO (+/−), |
| 40433 | HC-SAR | CD22-CD19 | RAJI-WT (+3), RAJI-CD 19-KO (+3), RAJI-CD22-KO (+/−), |
| 40434 | HC-SAR | CD22-CD19 | RAJI-WT (+2.5), RAJI-CD19-KO (+2), RAJI-CD22-KO (+/−), |
| 40435 | SIR | BCMA-CD22-CD19 | RAJI-WT (+1), RAJI-CD19-KO (+1), RAJI-CD22-KO (+1) |
| 40436 | SIR | CLD18A2 | NUGC4 (+1.5) |
| 40437 | SIR | CLDN6 | OVCAR3 (+3), OV90 (+3.5) |
| 40438 | z16SAR | CD19 | RAJI (+/−) |
| 40439 | CAR | CD19 | RAJI (+/−), HL60 (−) |
| 40440 | CAR | CD19 | RAJI (+/−), HL60 (−) |
| 40441 | CAR | BCMA | RAJI (+2), Nalm6 (−), L363 (+/−), NCI-H929 (+/−) |
| 40442 | CAR | CD33 | RAJI (−), HL60 (+/−) |
| 40443 | CAR | CD33 | RAJI (+/−), HL60 (+3) |
| 40444 | CAR | CD33 | RAJI (−), HL60 (+2) |
| 40445 | CAR | CD33 | RAJI (+/−), HL60 (+2) |
| 40446 | CAR | MSLN | SKOV3 (+3.5), OVCAR3 (+3) |
| 40447 | CAR | MSLN | SKOV3 (+3), OVCAR3 (+4) |
| 40448 | CAR | CD20 | RAJI (+/−), Jeko1 (+/−) |
| 40449 | z16SIR | PSMA | LNCaP (+/−) |
| 40450 | z16SIR | CD79b | RAJI (+/−), Jeko1 (+1) |
| 40451 | CAR | CD79b | RAJI (+/−), Jeko1 (+1) |
| 40452 | HC-SAR | CD19 | RAJI (+4) |
| 40453 | HC-SAR | CD19 | RAJI (+1) |
| 40454 | HC-SAR | CD19 | RAJI (+1.5) |
| 40455 | HC-SAR | CD19 | RAJI (+1) |
| 40456 | HC-SAR | CD19 | RAJI (+/−) |
| 40457 | Link | BCMA-CD19 | RAJI (+2.5), RAJI + Dasatinib (+3.5) |
| | z16SIR | | |
| 40458 | SIR | CD79b | RAJI (+5.5) |
| 40461 | HC-SAR | BCMA | MM1S (+3.5) |
| 40463 | HC-SAR | CD79b | Jeko1 (+2) |
| 40464 | SIR | CD19-CD20 | RAJI (+3.5) |
| 40480 | SIR | CD79b-CD20 | Jeko1 (+1.5) |
| 40481 | Link CAR | CD19 | RAJI (+3), RAJI + Dasatinib (+5) |
| 40482 | Link CAR | CD19 | RAJI (+4), RAJI + Dasatinib (+4) |
| 40483 | Link CAR | CD19 | RAJI (+4), RAJI + Dasatinib (+4) |
| 40484 | HC-SAR | CD19 | RAJI (+6) |
| 40485 | HC-SAR | CD19 | RAJI (+5.5) |
| 40486 | HC-SAR | CD19 | RAJI (+3.5) |
| 40487 | HC-SAR | CD19 | RAJI (+2.5) |
| 40488 | HC-SAR | CD19 | RAJI (+3) |
| 40489 | SIR | CD19 | RAJI (+4) |
| 40490 | HC-SAR | CD19 | RAJI (+/−) |
| 40491 | Link CAR | CD19 | RAJI (+3), RAJI + Dasatinib (+3) |
| 40492 | Link CAR | BCMA | MM1S (+1.5), MM1S + Dasatinib (+1.5) |
| 40493 | HC-SAR | CD19 | RAJI (+/−) |
| 40494 | HC-SAR | CD19 | RAJI (+5) |
| 40495 | HC-SAR | CD19 | RAJI (+3.5) |
| 40496 | HC-SAR | CD19 | RAJI (+4.5) |
| 40497 | HC-SAR | CD19 | RAJI (+/−) |
| 40498 | HC-SAR | CD19 | RAJI (+4) |
| 40499 | HC-SAR | CD19 | RAJI (+4.5) |
| 40500 | HC-SAR | CD19 | RAJI (+5) |
| 40511 | CAR | CD79b | Jeko1 (+1.5) |
| 40509 | z16SIR | CD79b | Jeko1 (+1.5) |

The above results also demonstrate that functional uni-specific, bispecific and trispecific SAR constructs (including HC-SAR, zSIR, zCD16 SAR or z16 SAR, etc.) can be constructed using the novel antigen binding domains (e.g., vL, vH, scFv and vHH) described in this disclosure. These novel antigen binding domains target antigens such as CD16, CD20, CD33, CD22, CD70, CD79b, MSLN, STEAP2, CLDN6, CLDN18.2, BCMA, PSCA, GD2, GPRC5D, ROR1, Folate receptor 1, mutant p53, NY-ESO-1, TAJ, DLL3, KLK2, IL13Ra2, MUC15, HLA-A2, etc.

The results also demonstrate the ability of zSAR constructs comprising dQ101 mutations in CD3z to show effective signaling (e.g., SEQ ID NO: 32540-50). The construct with SEQ ID NO: 32550 demonstrates that functional zSAR constructs can be constructed in which the vL and vH domains of an antibody are linked to CD3z chain via linkers comprising the Ig-like linker (or constant) domains of TCRγ and TCRδ chains.

The results also demonstrate that single and double chain CD16 SAR constructs which partially or completely lack the CD16 cytosolic domain are functionally active (e.g., SEQ ID NO; 32550). The results further demonstrate that the cytosolic domain of CD16 in a single or double chain CD16 based SAR can be replaced by the cytosolic domain of a costimulatory molecule (e.g., 41BB, CD28, etc.) (SEQ ID NO: 32553, 32545). Alternatively, the costimulatory domain (e.g., costimulatory domain of 41BB, CD28, etc.) can be attached to the C-terminal or the N-terminal of CD16 cytosolic domain.

The results also demonstrated signaling activity of SAR comprising linkers derived from TCRγ and TCRδ constant domains (e.g., SEQ ID NO: 32545) and long Ig linkers derived from TCR constant chains (e.g., SEQ ID ON: 32544)

The results also demonstrate several novel SIR/SAR designs. The construct with SEQ ID NO: 32532 comprises a construct in which the vH domain of an antibody is operationally linked via a linker (e.g., IgCL) to the hinge, TM, and CP domain of CD8 and the complementary vH fragment is attached via a linker (e.g., IgG1-CH1) to the hinge, TM of CD4 and CP domain of CD3z.

The results also demonstrated that LINK CAR (e.g., SEQ ID NO: 40481, 40457) comprising an antigen binding domain attached to the cytosolic domain of Lck can be regulated using an Lck inhibitor. Example LCK inhibitor includes Dasatinib and Ponatinib. The results demonstrated that treatment with Dasatinib can be used to upregulate the activity of a LINK CAR comprising the LCK cytosolic domain as a signaling domain. These results demonstrate that Lck inhibitors (e.g., Dasatinib, Ponatinib, etc.) can be used for the control of Lck based CAR activity in vitro and/or in vivo. The results further demonstrated that LINK CAR based on a Lck mutant (T3161) (e.g., SEQ ID NO:40491, 40492) are not responsive to Dasatinib. Dasatinib can be used at a dose of 40-200 mg/day orally (e.g., 40, 80, 100, 140, 180, 210 mg/day PO) to control the activity of Lck based SARs. Dasatinib can be given before, concurrently with or after administration of Lck based SARs.

The results also demonstrate signaling activity of double chain LINK SARs comprising the vL fragment of an antibody attached via a linker (e.g., Ig linker) to the hinge and TM domains of CD3z fused to Lck and the vH fragment of an antibody attached via a linker (e.g., Ig linker) to CD16 hinge and transmembrane domain and cytosolic domain (SEQ ID NO: 32602). In an embodiment, the vL and vH fragments are switched. In an embodiment, the hinge and TM domains of the second chain can be also derived from CD3z so that Lck domain is fused to the C-terminal of the TM of CD3z (e.g. SEQ ID NO: 32601). Alternatively, the hinge and TM domains of CD3z can be substituted by hinge and TM domains of another protein, such as CD16a, FcRy, etc. In an embodiment, one or both chains of the LINK CAR may further comprise a costimulatory domain (e.g., CD28 or 4-1BB costimulatory domain) that is optionally located C-terminal to the transmembrane domain.

When the LINK CAR is expressed in an immune cell, the vL and vH fragments attached to the two signaling chain can form an antigen binding module that can specifically bind to a cognate antigen and trigger immune cell signaling. The LINK CAR may further comprise one or more AABD that are attached N-terminal to the vL and/or vH fragments via optional linkers.

The results also demonstrated signaling activity of novel double chain LINK SARs comprising the vL fragment of an antibody attached via a linker (e.g., Ig linker) to the hinge and TM and CP domains of CD3z and the vH fragment of an antibody attached via a linker (e.g., Ig linker) to a hinge and transmembrane domain that is fused to LAT or a mutant thereof (LAT-200-262del) (SEQ ID NO: 32605). In an embodiment, the vL and vH fragments are switched. In an embodiment, the hinge and TM domains of the second chain can be also derived from CD3z so that LAT or a mutant thereof (LAT-200-262del) is fused to the C-terminal of the TM of CD3z. Alternatively, the hinge and TM domains of CD3z can be substituted by hinge and TM domains of another protein, such as CD16a, FcRy, etc. In an embodiment, one or both chains of the LAT based CAR may further comprise a costimulatory domain (e.g., CD28 or 4-1BB costimulatory domain) that is optionally located C-terminal to the transmembrane domain. When the LAT based SAR is expressed in an immune cell, the vL and vH fragments attached to the two signaling chain can form an antigen binding module that can specifically bind to a cognate antigen and trigger immune cell signaling. The LAT-LINK SAR may further comprise one or more AABD that are attached N-terminal to the vL and/or vH fragments via optional linkers. In an embodiment, the CD3z cytosolic domain comprises the dQ101 mutation. In an embodiment, the LAT domain is replaced by SLP-70 domain or mutant thereof (e.g., SEQ ID NO: 32604).

The results further demonstrate that accessory modules comprising co-receptors such as CD8a, CD8b, CD4 or combination thereof can be expressed with any of the SAR of the disclosure (e.g., uTCR-SAR, ZSAR, zCD16 SAR, HC-SAR, SIR, etc.) (see SEQ ID NO: 1045-1059). The results further demonstrate that accessory modules comprising co-stimulatory ligands (e.g., 4-1BBL, CD48, CD80, CD86, etc.) or combination thereof can be expressed with any of the SAR of the disclosure (e.g., uTCR-SAR, zSAR, zCD16 SAR, HC-SAR, SIR, etc.) to increase their activity (SEQ ID NO: 32320).

The results also demonstrated novel double chain zSARs comprising the vL fragment of an antibody attached via a linker (e.g., Ig linker) to the hinge and TM and CP domains of CD3z and the vH fragment of an antibody attached via a linker (e.g., Ig linker) to a hinge and transmembrane domain of a different signaling chain (SEQ ID NO: 32481-32484). In an embodiment, the vL and vH fragments are switched. In an embodiment, the second signaling chain may comprise the hinge, TM, and CP domain of CD3γ, CD3δ or CD3ε. In an embodiment, one or both chains of the SAR may further comprise a costimulatory domain (e.g., CD28 or 4-1BB costimulatory domain) that is optionally located C-terminal to the transmembrane domains. When the SAR is expressed in an immune cell, the vL and vH fragments attached to the two signaling chain can form an antigen binding module that can specifically bind to a cognate antigen and trigger immune cell signaling. The SAR may further comprise one or more AABD that are attached N-terminal to the vL and/or vH fragments via optional linkers. In an embodiment, the CD3z cytosolic domain comprises the dQ101 mutation.

SAR Constructs with Hybrid TCR Chains

The SAR constructs in the above Table are expressed in primary T cells using lentiviral mediated gene transfer and tested for cytotoxicity against the target cell lines expressing Gluc or LucPPE using Matador or Matador-Glo assays, respectively. The SAR constructs are shown to induce specific lysis of the target cells. The SAR constructs are also shown to induce TNFα and IFNγ production upon co-culture with their target cell lines. Finally, the SAR constructs are tested for in vivo activity using xenograft models in NSG mice as described in PCT/US2019/035096 and shown to be effective.

### Hybrid Chain SIR

Several hybrid chain SIR constructs targeting CD19 comprising vL and vH fragments derived from a humanized mouse monoclonal antibody (hu-mROO5) are constructed and are represented by SEQ ID NO: 1063-1066. The constructs are expressed in JNG cells and shown to induce GFP expression upon co-culture with CD19+ RAJI cells. The constructs are also expressed in primary T cells and shown to induce specific lysis of CD19+ target cell lines (e.g., RAJI, NALM6) using Matador assay. A number of additional one and a half chain and double chain hybrid chain SIR constructs selected from SEQ ID NO: 3931-8074, 8076-8103, 8106-8393, 23237-23264 and targeting different antigens and comprising different antigen binding domains are constructed. Similarly, SAR constructs comprising TCR chains with C-terminal CD3z activation and 41BB/CD28 costimulatory domains selected from SEQ ID NO: 27922-27 are constructed. These constructs are expressed in JNG cells and shown to induce GFP upon exposure to their target cell lines. They are also expressed in primary T cells and shown to induce cytotoxicity and cytokine production when exposed to their target cell lines.

### Comparative Analysis of Hybrid Chain SAR in JNG Cells

The JNG T cells expressing different uni-specific and bispecific CD19 SAR constructs (HC-SAR, zSIR, zCD16 SAR, etc.) were co-cultured with Raji target cells that express high level of CD19. The cells were plated in a 24-well plate at Effector: Target (E:T) ratio of 1:1 for overnight. Next day, these cells were collected and assayed for GFP induction using flow cytometry. The cell supernatants were collected from these co-cultured cells to measure hIL2 secretion using hIL2 ELISA duo kit (R&D Systems). The results are shown in the following Table 14. The construct with SEQ ID NO: 32450 is a conventional SIR construct comprising vL and vH fragments of hu-mROO5-1 attached to human TCRα and TCRβ chains that carry T48C and S57C mutations. The other constructs were hybrid chain SIR constructs, zSIR or zCD16 SAR. The Table 14 shows that JNG cells expressing the HC-SAR have varying level of NFAT-induced GFP induction and IL2 secretion with some constructs (e.g., SEQ ID NO: 32584, 32587 showing higher GFP induction as compared to SIR with SEQ ID NO: 32450. Both these constructs have the same hu-mROO5-1 derived vL and vH fragments as the binding domains. In the construct with SEQ ID NO: 32584 the human TCRβ chain of constructs with SEQ ID NO: 32450 is replaced by a TCRβγ hybrid chain comprising the constant domain and ConnP of human TCRβ and TM and CP domains of human TCRγ. The ability of this construct to induce GFP and IL2 was surprising as it has only one hybrid chain. In the construct with SEQ ID NO: 32587, the human TCRβ chain is replaced by a TCRγβ hybrid chain comprising the constant domain and ConnP of human TCRγ and TM and CP domains of human TCRβ.

Furthermore, the human TCRα chain is replaced by a TCRδα hybrid chain comprising the constant domain and ConnP of human TCRδ and TM and CP domains of human TCRα. The construct with SEQ ID NO: 32589 was also very effective. This construct is similar in design to the construct with SEQ ID NO: 32587 but has a different junction in the TCRδα hybrid chain. Several additional HC-SAR constructs showed significant GFP and IL2 induction. Another surprising result was that constructs with different hybrid chains showed different levels of IL2 induction although they showed similar levels of NFAT-driven GFP induction (e.g., SEQ ID NO: 32589 vs 32582, 32816, 32817, 32840 and 32851, etc.). Thus, the use of hybrid chains allows one to generate SIR constructs with differential activation of different effector functions. The JNG cells expressing different zSIR constructs also showed a wide range of GFP and IL2 induction abilities. Collectively, these surprising results provide a rapid and simple method to generate a panel of constructs with different signaling strengths and different abilities to induce cytokine production.

In an independent experiment, JNG cells expressing the HC-SAR constructs with SEQ ID NO: 40484, 40485 and 40486 showed very strong NFAT-induced GFP induction when exposed to RAJI cells. All these constructs comprise antibody based constant domains (e.g., IgCL and IgG1-CH1). The construct with SEQ ID NO: 40484 comprises one hybrid TCR chain comprising an IgCL constant domain linked to a hybrid human TCRγβ chain. This TCRγβ chain comprises the connP of human TCRγ fused to the TM and CP of human TCRβ. The second chain of this HC-SAR comprises an IgG1-CH1 domain linked to a hybrid human TCRδα chain. This chain comprises the connP of human TCRδ fused to the TM and CP of human TCRα. The construct with SEQ ID NO:40485 is similar to SEQ ID NO: 40484 but has a different junction in the hybrid human TCRδα chain. The construct with SEQ ID NO: 40486 resembles the constructs with SEQ ID NOs: 40484 and 40485 in one chain. However, in this construct, the hybrid TCRδα of constructs with SEQ ID NO: 40484 and 40485 is replaced by a fragment comprising the connP, TM and CP of human TCRα.

TABLE 14

| | HC-SAR | IL2 | | zSIR | IL2 |
|---|---|---|---|---|---|
| SEQ ID | % GFP | (pg/ml) | SEQ ID | % GFP | (pg/ml) |
| 32450 | 36.28% | 28900 | 32841 | 9.60% | 5461 |
| 32572 | 19.25% | 7867 | 32547 | 29.00% | 22167 |
| 32573 | 20.38% | 21067 | 32877 | 33.30% | 34333 |
| 32574 | 5.91% | | 32844 | 2.70% | |
| 32463 | 25.71% | | 32843 | 3.90% | 11250 |
| 1062 | 21.04% | 3944 | 32605 | 28.60% | 50383 |
| 1063 | 6.62% | | 32606 | 19.40% | 18517 |
| 1064 | 18.96% | 1450 | 32607 | 18.50% | 12717 |
| 1065 | 5.70% | | 32608 | 0.50% | |
| 1066 | 17.10% | 3011 | 32541 | 15.20% | |
| 32584 | 38.10% | 43583 | 32542 | 21.90% | 100 |
| 32585 | 14.70% | 16500 | 32543 | 19.60% | 2489 |
| 32587 | 41.60% | 45533 | 32544 | 15.90% | 2022 |
| 32588 | 17.70% | 27200 | 32545 | 28.06% | |
| 32589 | 22.20% | 34900 | 32546 | 17.50% | 3033 |
| 32591 | 14.32% | 18467 | 32547 | 16.60% | 72 |
| 32549 | 27.30% | 1878 | 32548 | 29.70% | 8539 |
| 32582 | 24.60% | 2706 | | | |
| 32815 | 20.90% | | | | |
| 32816 | 21.90% | 1000 | | | |
| 32817 | 31.60% | 2644 | | | |
| 32840 | 29.70% | 9172 | | | |
| 32851 | 23.60% | 4450 | | | |
| 32853 | 23.80% | 2589 | | | |
| 32818 | 17.10% | 4606 | | | |
| 32819 | 4.90% | | | | |
| 32820 | 3.60% | | | | |
| 32821 | 7.70% | | | | |

In an independent experiment, JNG cells expressing the HC-SAR constructs with SEQ ID NO: 40484, 40485 and 40486 showed very strong NFAT-induced GFP induction when exposed to RAJI cells with 59%, 55%, and 39% of cells, respectively, showing GFP induction after co-culture overnight. All these constructs comprise antibody based constant domains (e.g., IgCL and IgG1-CH1). The construct with SEQ ID NO: 40484 comprises one hybrid TCR chain comprising an IgCL constant domain linked to a hybrid human TCRγβ chain. This TCRγβ chain comprises the connP of human TCRγ fused to the TM and CP of human TCRβ. The second chain of this HC-SAR comprises an IgG1-CH1 domain linked to a hybrid human TCRδα chain. This chain comprises the connP of human TCRδ fused to the TM and CP of human TCRα. The construct with SEQ ID NO:40485 is similar to SEQ ID NO: 40484 but has a different junction in the hybrid human TCRδα chain. The construct with SEQ ID NO: 40486 resembles the constructs with SEQ ID NOs: 40484 and 40485 in one chain. However, in this construct, the hybrid TCRδα of constructs with SEQ ID NO: 40484 and 40485 is replaced by a fragment comprising the connP, TM and CP of human TCRα. These results demonstrate that it is possible to generate HC-SAR comprising the constant domains of antibodies and hybrid chains comprising connP, TM and CP derived from different TCR chains. These results demonstrate that it is possible to generate functional SIR constructs in which one of the two chains is a hybrid chain comprising fusion of at least three polypeptides (e.g., antibody constant domain, connP of one TCR chain and TM and/or CP of a different TCR chain). The second TCR chain in such a SIR can be a fusion of two proteins (e.g., antibody constant domain fused to connP, TM and CP of a TCR chain) or a fusion of three proteins (e.g., constant domains of antibodies and hybrid chains comprising connP, TM and CP derived from different TCR chains).

A number of additional uni-specific and bispecific HC-SAR comprising different antigen binding domains are constructed. These constructs comprise one or two hybrid chains where each chain is a fusion between two or three proteins. These constructs are tested as above and are shown to be active in NFAT-induced GFP induction. Representative constructs are shown by SEQ ID NO: 40140-40434.

### Comparative Analysis of Hybrid Chain SAR in Primary T Cells

T Cell were isolated using CD3 microbeads (Miltenyi) were infected with a panel of SAR (e.g., CAR, SIR and HC-SAR) constructs targeting CD19. All the SAR constructs comprised the hu-mROO5-1 vL, vH or scFv fragments as the antigen binding domain. The T cells were plated in 6-well plates in XVIVO15 medium supplemented with 50 IU/ml IL2 and 5% hAB serum for 4 days for expansion. For Matador assay, Raji cells expressing Luc-PPE were plated at 5,000/well/30 μL in triplicate in a 384-well plate in XVIVO15 medium without any supplements. Control T cells and SAR expressing T cells were added at Effector: Target (E:T=1:1) ratio in 30 μL/well in triplicate. Plates were incubated at 37° C. for 24 hours. Post-incubation, 15 μL of D-Luciferin luciferase assay buffer was added to each well and luminescence was read for 10 seconds. For ELISA assay, Raji cells expressing Luc-PPE were plated at 50,000/well/100 μL in triplicate in a U-bottom 96-well plate in XVIVO15 medium without any supplements. T cells added at E:T=1:1 ratio in 100 μL/well in triplicate. Plates were incubated at 37° C. for 48 hours. Post-incubation, samples were collected and hIFNg, hTNFa, and hIL2 secretion in the cell supernatants were measured by ELISA duo kits (R&D Systems). The results are shown in the following Table 15. The results demonstrate that several HC-SAR constructs (e.g., SEQ ID NO: 32587-32589 and 32591) show superior expression, cytotoxicity and cytokines production as compared to the SIR constructs (e.g., SEQ ID NO: 32450 and 32488). Several HC-SAR constructs (e.g., 32463 and 32591) showed equivalent cytotoxicity to CAR (SEQ ID NO: 32315) and SIR (SEQ ID NO: 32450 and 32488) constructs but with much lower cytokines (e.g., IFNγ, TNFα) production. A difference was also observed among the different constructs in their ability to induce production of IL2. Taken together, these results demonstrated that HC-SAR approach provides a rapid, economical, and efficient approach to generate a large panel of SIR constructs of different signaling strengths, cytotoxicity, cytokine production and expression based on a single antigen binding module (e.g., vL/vH fragments). Therefore, depending on the target indication, a SIR of desired affinity, cytotoxicity, cytokine production and strength can be rapidly selected from a panel of HC-SAR comprising the same antigen binding domain but different types and combinations of hybrid and non-hybrid chains. Thus, the HC-SAR approach obviates the need for generating and testing a large panel of antibodies to select a suitable antibody for generation of a SIR of desired affinity and strength.

TABLE 15

| | | Relative Luc | Protein | IFNg- | TNFa- | IL2- |
|---|---|---|---|---|---|---|
| | SEQ | (Matador | L | conc | conc | conc |
| SAR | ID | Cytotoxicity | staining | (pg/ | (pg/ | (pg/ |
| Architecture | NO: | Assay) | (%) | ml) | ml) | ml) |
| | Media | 4713 | | ND | ND | ND |
| | T-UI | 6519 | 10.00% | ND | ND | ND |
| CAR | 32315 | 10206 | 81.43% | 22688 | 11881 | 38150 |
| SIR | 32450 | 10513 | 15.15% | 23129 | 9074 | 19117 |
| SIR | 32448 | 10757 | 17.42% | 24238 | 10801 | 27750 |
| HC-SAR | 32573 | 7915 | 18.60% | 3854 | 327 | 1483 |
| HC-SAR | 32463 | 10092 | 40.12% | 9813 | 1234 | 2417 |
| HC-SAR | 1066 | 9041 | 12.37% | 9579 | 1081 | 3250 |
| HC-SAR | 40430 | 7598 | 20.68% | 1429 | ND | 317 |
| HC-SAR | 32563 | 9086 | 23.39% | 10913 | 1507 | 2150 |
| HC-SAR | 32597 | 7122 | 22.44% | ND | ND | ND |
| HC-SAR | 32598 | 8165 | 16.76% | 8629 | 601 | 550 |
| HC-SAR | 40431 | 9212 | 12.92% | 12338 | 3434 | 15617 |
| HC-SAR | 32587 | 11379 | 64.14% | 19054 | 5847 | 20417 |
| HC-SAR | 32588 | 11960 | 79.22% | 23979 | 8014 | 23717 |
| HC-SAR | 32589 | 10555 | 75.74% | 21571 | 7014 | 24817 |
| HC-SAR | 32591 | 10323 | 60.07% | 13429 | 4621 | 12650 |
| HC-SAR | 32581 | 11074 | 47.41% | 23888 | 11401 | 40683 |
| HC-SAR | 32582 | 10339 | 28.88% | 19563 | 9434 | 36383 |
| HC-SAR | 32551 | 9816 | 27.11% | 13646 | 4261 | 10183 |
| HC-SAR | 32552 | 8097 | 20.18% | 5288 | ND | 517 |

The above analysis also yielded several surprising and unanticipated results. First, several SAR constructs with one hybrid and one native chain shows strong signaling activity. Second, several types of hybrid chains with different locations of the junction between the two chains showed strong signaling activity indicating that there is no single site for joining the two chains. In fact, hybrid chains comprising the same two chains but different sites of junction showed slightly different signaling activity, suggesting that it is possible to generate additional diversity in SARs by simply using the different sites of junction. Third, several hybrid chains showed signaling activity when paired with two different wild type chains. Thus, a hybrid chain comprising the TCRβ constant domain and TCRγ connP, TM and CP showed signaling when paired with either TCRα or TCRδ chains. Finally, several hybrid chain SAR constructs showed expression and activity that was higher than constructs comprising wild-type TCR chains.

SAR and uTCR-SAR with CD3z Chain Comprising dQ101 Mutation

SAR (e.g., zSIR) and uTCR-SAR constructs comprising one or two CD3z chains comprising dQ101 mutation and targeting PSMA (SEQ ID NO: 1108-1110) are constructed and expressed in JNG cells. The constructs with the dQ101 mutation(s) induce greater GFP expression when exposed to LNCaP target cells as compared to similar constructs that lack the dQ101 mutation in one or both CD3z chains (e.g., SEQ ID NO: 1088-1089, 1017-1020). Similarly, uni-specific and bispecific SAR (e.g., zSIR constructs) targeting CD19 and/or CD20 comprising dQ101 mutations (e.g., SEQ ID NO:1105-1107) are expressed in JNG cells and found to induce strong GFP induction upon exposure to RAJI and RAJI-CD19-KO target cells. The results further show that constructs comprising one CD3z chain and one CD16 chain show strong GFP induction, which, in general, is stronger than constructs comprising two CD3z chains or one CD3z chain and one FcRy chain. Similarly, z-CD16 based double chain constructs with dQ101 mutation in the CD3z chain show effective in vitro and in vivo activity.

CD16-SAR with Mutant CD16 Chains

A number of CD16-based SAR comprising hu-mROO5-1 scFv attached to the extracellular and transmembrane domain of CD16 but lacking CD16 cytosolic domain are constructed. These constructs are represented by SEQ ID NO: 22717-18, 1214, 1495, 1776, 2057. The constructs are expressed in JNG cells and shown to induced GFP upon co-culture with RAJI cells. A number of double chain SAR constructs (SEQ ID NO:2231-2234) comprising CD3z chain and a mutant CD16 chain comprising the hinge and transmembrane domain of CD16 but lacking CD16 cytosolic domain. The constructs are expressed in JNG, primary T and NK cells and shown to be active based on JNG-NFAT-GFP and Matador assays.

### Tunable SAR Constructs

A CD19 targeted SAR construct (SEQ ID NO: 1040) comprising hu-mRO05-1 vL and vH fragments attached to CD3z chains via Ig-like linkers from TCRa and TCRb chains and also comprising a FKBP12-F36V domain attached to one of the CD3z chain is constructed and expressed in JNG cells. The JNG cells expressing the construct are treated with dTAG (100 nM) or left untreated and then co-cultured with RAJI cells. Treatment with dTAG is shown to reduce GFP induction following co-culture with RAJI cells.

A CD19 targeted SAR constructs (SEQ ID NO: 1041) comprising a IKZF1-ZF2-ZF3-141-243 domain attached to one of the CD3z chain is constructed and expressed in JNG cells. The JNG cells expressing the construct are treated with lenalidomide (LEN) or pomalidomide (POM) or left untreated and then co-cultured with RAJI cells. Treatment with lenalidomide and pomalidomide is shown to reduce GFP induction following co-culture with RAJI cells. Similar results are obtained with another CD19 SAR construct (SEQ ID NO: 1096) based on hu-mROO5-1 vL and vH domains and which carries a IKZF3-136-180-236-249-K0 module attached to one CD3z chain and a second IKZF3-136-180-236-249-KO module attached to a second FcRy chain.

T cells expressing CD79b SARs induce cytotoxicity in CD79b-expressing cells. A large panel of SAR on different architectures (e.g., SIR, zSIR, zCD16 SAR, CAR, hybrid chain SIR, etc.) and comprising the vL, vH and scFv fragments represented by SEQ ID NOs: 32028-32042, 32091-32105 and 32154-32168 are constructed and shown to possess varying level of activity when expressed in JNG cells and exposed to CD79b expressing JEKO-1 cell line. Human peripheral blood T cells isolated using CD3 magnetic beads were infected with lentiviruses expressing the SAR constructs (e.g., SEQ ID NO: 32770-32278) targeting CD79b. The SAR constructs with SEQ ID NO: 32770, 32774 and 32277 have the architecture of a SIR and differ in their vL and vH fragments. These constructs comprise the vL fragment of a CD79b antibody attached to a human codon optimized TCRβ constant chain comprising S57C mutation (SEQ ID NO: 470). The complementary vH fragment is attached to a human codon optimized human TCRα constant chain comprising T48C mutation (SEQ ID NO: 458). The constructs with SEQ ID NO: 32771 and 32773 are hybrid chain SAR constructs in which the vL fragment of a humanized CD79b antibody is linked to a hybrid TCR chain (SEQ ID NO: 23229) comprising the Ig-like linker domain and connecting peptide of human TCRβ chain fused in frame to the transmembrane and cytosolic domain of human TCRγ chain. The vH fragment of the antibody is operationally linked to the human TCRα constant chain comprising T48C mutation (SEQ ID NO: 458). In the SAR constructs 32271, 32273 and 32278 the vL fragment is attached to a fragment (SEQ ID NO: 946) encoding a TCRβ derived Ig-like linker fused to the hinge, transmembrane and cytosolic domain of CD3z (dQ101 variant). The complementary vH fragment is attached to a fragment (SEQ ID NO: 31997) encoding a TCRα derived Ig-like linker fused to the hinge, transmembrane and cytosolic domain of CD16a. The SAR construct with SEQ ID NO: 32276 is a second-generation CAR construct comprising a humanized CD79b scFv linked via human CD8 hinge and transmembrane domains to a 41BB costimulatory domain and a CD3z activation domain. Expression of SAR on the surface of T cells was examined by staining with Protein L followed by Flow Cytometry. The T cells expressing the different SAR constructs show variable Protein L staining. The T cells expressing the constructs 120323-EZwA1 (SEQ ID NO: 32270), 120323-EZwF1 (SEQ ID NO: 32271), 113023-EZtP1 (SEQ ID NO: 32272), 120323-EZw01 (SEQ ID NO: 32273), 112523-EZsD1 (SEQ ID NO: 32274), 112923-EZuM1 (SEQ ID NO:32276), 100123-EZxD1 (SEQ ID NO: 32277), and 112923-EZtG1 (SEQ ID NO: 32278) show 22%, 87%, 22%, 63%, 15%, 65%, 11% and 13% cells that bind to Protein L, respectively. To confirm the results and obtain a measure of affinity, T (0.5×10⁶) cells expressing the different SAR constructs were stained with 1 μg/mL human CD79-Fc Acro Biosystems (Cat. No. CDB-H5259) for 1 h at 4° C. followed by a wash with cold FACS-buffer (PBS+2% BSA). Cells are stained with Stain with Goat-Anti-Human-IgG-PE (Southern Biotech, Cat. No. 2040-09), incubated for 1 h at 4° C. and analyzed by Flow cytometry. The different SAR constructs again show variable staining with CD79b-Fc. The T cells expressing the SAR constructs 120323-EZwA1 (SEQ ID NO: 32270), 120323-EZwF1 (SEQ ID NO: 32271), 113023-EZtP1 (SEQ ID NO: 32272), 120323-EZwO1 (SEQ ID NO: 32273), 112523-EZsD1 (SEQ ID NO: 32274), 112923-EZuM1 (SEQ ID NO:32276), 100123-EZxD1 (SEQ ID NO: 32277), and 112923-EZtG1 (SEQ ID NO: 32278) show 43%, 70%, 36%, 36%, 21%, 53%, 3% and 1% CD79-Fc staining, respectively. The cytotoxicity of T cells expressing the different SAR constructs is examined by Matador Glo assay using JEKO-1 and RAJI cells expressing LucPPe-146-1H2 at different E:T ratios and co-culture times of 4 to 48 hours. T cells expressing all constructs show significant cytotoxicity. The percent specific lysis of the T cells expressing the constructs 120323-EZwA1 (SEQ ID NO: 32270), 120323-EZwF1 (SEQ ID NO: 32271), 113023-EZtP1 (SEQ ID NO: 32272), 120323-EZw01 (SEQ ID NO: 32273), 112523-EZsD1 (SEQ ID NO: 32274), 112923-EZuM1 (SEQ ID NO:32276) upon 24 h coculture with JEKO-1 cells at E:T ration of 5:1 was 44%, 46%, 43%, 35%, 42% and 36% respectively. The SAR-T cells were expanded in culture for over 10 days and cytokine production by different SAR-T cells was measured by ELISA in the supernatant after 48 h co-culture Daudi cells. Highest TNFα and IFNα production was seen with SAR-T cells expressing the second-generation CAR construct 112923-EZuM1 (SEQ ID NO:32276). The hybrid chain SAR construct 113023-EZtP1 (SEQ ID NO: 32272) on the double chain SIR architecture showed the next highest TNFα and IFNα secretion followed by the SIR constructs 120323-EZwA1 (SEQ ID NO: 32270) and 112523-EZsD1 (SEQ ID NO: 32274), respectively. The SAR constructs 120323-EZwF1 showed modest production of TNFα and IFNγ while the SAR construct 120323-EZwO1 showed low production of these cytokines. In contrast, T cells expressing the 112923-EZuM1 construct failed to produce IL2 upon co-culture with Daudi cells. T cells expressing the SAR constructs 120323-EZwA1 and 113023-EZtP1 showed highest IL2 secretion. T cells expressing the SAR constructs and 120323-EZwF1, 120323-EZwO1, 112523-EZsD1 showed moderate IL2 secretion.

The in vivo activity of the different SAR-T cells was tested using a JEKO-1 xenograft model in NSG mice. Jeko1-Luc-PPE cells were injected via tail vein injection (0.5 million cells/mouse) into mice (n=5 or 4). T cells (5 million cells/mouse) expressing the different SAR constructs were injected via tail vein injection 7 days later. Animals were examined using bioluminescence imaging. All animals given SAR-T cells showed effective tumor control as compared to animals given control T cells. The best tumor control was seen in animals given T cells expressing constructs EZwA1, 120323-EZwF1, 113023-EZtP1, respectively.

In an independent experiment, SAR constructs targeting CD79b with SEQ ID NO: 32277, 32279-32283 were expressed in T cells using the protocol described above. The construct with SEQ ID NO: 32279 is a uni-specific second-generation CAR construct that targets CD79b. The SAR constructs with SEQ ID NO: 32280-81 are uni-specific double chain SIR constructs that target CD79b. They differ in their antigen binding domains but are similar in design to the SIR construct with SEQ ID NO: 32277 described above. The SAR constructs with SEQ ID NO: 32282 and 32283 are bispecific SIR constructs that in addition to targeting CD79b also target CD20 and BCMA, respectively. The cytotoxicity of the T cells expressing the different SAR constructs against JEKO-1 (CD79b+) and RAJI (CD79b+, CD20+, BCMA+) cells was demonstrated using Matador-Glo cytotoxicity assay. All constructs demonstrated effective cytotoxicity against JEKO-1 and RAJI cells. The bispecific SAR constructs showed greater cytotoxicity against RAJI cells. The bispecific SIR construct with SEQ ID NO: 32283 also showed highest cytokine (TNFα, IFNγ, and IL2) production when co-cultured with RAJI cells. The uni-specific SIR construct (SEQ ID NO: 32277) showed effective cytotoxicity and TNFα and IFNγ production as compared to other uni-specific constructs when co-cultured with either JEKO-1 or RAJI cells. The in vivo activity of the different SAR-T cells is confirmed using JEKO-1 xenograft model in immunodeficient (NSG) mice (n=5). Mice given all SAR-T constructs showed effective tumor control as compared to mice given no T cells or mice given control T cells. The SAR-T cells expressing the SIR construct with SEQ ID NO: 32277 showed the best tumor control.

Next, a CD79b SAR construct with SEQ ID NO: 32426 was constructed in which the vL fragment of the construct SEQ ID NO: 32270 is replaced by a vL fragment represented by SEQ ID NO: 32034. The vH fragment of this construct is represented by SEQ ID NO: 32092. This SAR construct has the architecture of a double chain SIR and comprises the vL fragment joined in frame to human TCRβ chain (SEQ ID NO: 470) and the vH fragment joined in frame to human TCRα chain (SEQ ID Nos: 458). This SAR construct was expressed in JNG cells by lentiviral mediated gene transfer. JNG cells expressing this SAR construct were shown to result in strong induction of NFAT driven GFP expression when co-cultured with JEKO-1 cells. A construct represented by SEQ ID NO: 32427 in which the vH fragment of the construct with SEQ ID NO: 32426 is replaced by a vH fragment represented by SEQ ID NO: 32091 is also shown to be highly active when expressed in JNG cells. The SAR constructs with SEQ ID NO: 32426 and 32427 are expressed in primary T cells and tested for in vitro and in vivo activity using assays described above.

Representative uni-specific, bispecific and multi-specific SAR constructs targeting CD79b on different architectures and comprising different antigen binding domains (e.g., vL, vH and scFv fragments) are presented in SEQ ID NO: 37785-38569, 40187-40200, 40230-40243, 40275-40286, 50054-50084, 50177-50558, 50598-50609, 50631-50642 etc.). The SAR constructs with SEQ ID NO: 50054-50058 are transfected into 293T cells. These constructs belong to zCD16 architecture and comprise one CD3z chain with dQ101 mutation and a second chain comprising CD16 hinge, transmembrane and cytosolic domains. Next day, staining with Protein L and CD79b-Fc shows expression of the SAR on approximately 29-50% of transfected cells. In particular, 293 FT cells transfected with SAR constructs with SEQ ID NO: 50055 and 50056 show 41% and 51% staining with Protein L, respectively and 41% and 41% staining with CD79b-Fc reagent, respectively. The representative constructs are also expressed in T cells and their cytotoxicity, cytokine production and in vivo activity is demonstrated using assays described above. These constructs can be used to generate a polyfunctional and diverse immune response. The CD79b SAR constructs are also expressed in umbilical cord derived T cells and shown to demonstrate strong expression and in vitro and in vivo killing activity. Cord T cells expressing CD79b SAR construct are used as an off the shelf approach for the treatment of B cell leukemias, lymphomas and autoimmune disorders (e.g., lupus).

To detect the expression and expansion of CD79b SAR construct, a polynucleotide encoding a fusion protein comprising the extracellular domain of CD79b (SEQ ID NO: 32005) in fusion with a marine luciferase is constructed. The DNA fragment also carries a 5″ CD8 signal peptide (SEQ ID NO: 32004). The resulting DNA fragment is cloned in frame with a 3′ NLuc encoding polynucleotide cassette. The cassette also carries tandem epitope tags to allow protein purification and detection by FACS. The resulting fusion construct is represented by SEQ ID NO: 40530. The Nluc moiety in this construct can be replaced by any other marine luciferase, such as Gluc, Turbo-Luc, etc. The fusion protein is expressed in 293 FT cells and used to detect cells expressing CD79b targeted SAR constructs.

T cells expressing STEAP2 SARs induce cytotoxicity in STEAP2-expressing cells. Human peripheral blood T cells isolated using CD3 magnetic beads are infected with lentiviruses expressing the SAR constructs with SEQ ID NO: 32292-32295. These SAR constructs comprise vL, vH and scFv represented by SEQ ID NO: 19776, 19795, and 19814, respectively. The SAR with SEQ ID NO: 32294 is a second-generation CAR construct comprising a STEAP2 scFv (SEQ ID NO: 19814) operationally linked to a CD8 hinge and transmembrane domains, a 41-BB cytosolic domain and a CD3z cytosolic domain. The SAR constructs with SEQ ID NO: 32293-94 have the architecture of double chain SIR constructs. In the construct with SEQ ID NO: 32293, the vH fragment (SEQ ID NO: 19795) is operationally linked in frame to a human codon optimized TCRβ chain with S57C mutation (SEQ ID NO: 470) and vL fragment (SEQ ID NO: 19776) is operationally linked to a human codon optimized human TCRα chain with T48C mutation (SEQ ID NO: 458). In the construct with SEQ ID NO: 32294, the vH fragment is attached to a human codon optimized and murinized human TCRα chain with T48C mutation (SEQ ID NO: 455) and vL fragment (SEQ ID NO: 19776) is operationally linked in frame to a human codon optimized and murinized human TCRβ chain with S57C mutation (SEQ ID NO: 471). The SAR construct with SEQ ID NO: 32295 has the architecture of a CD3z-CD16 SAR. In this construct, the vH fragment (SEQ ID NO: 19795) is attached to a fragment (SEQ ID NO: 946) encoding a TCRβ derived Ig-like linker fused to the hinge, transmembrane and cytosolic domain of CD3z (dQ101 variant). The complementary vH fragment is attached to a fragment (SEQ ID NO: 31997) encoding a TCRα derived Ig-like linker fused to the hinge, transmembrane and cytosolic domain of CD16a. Expression of SAR on the surface of T cells was examined by staining with Protein L followed by Flow Cytometry. The T cells expressing the constructs with SEQ ID NO: 32292-32295 showed 91%, 63%, 77% and 76% surface staining with Protein L. All SAR-T cells showed effective and near equivalent cytotoxicity against LNCaP cells (Prostate cancer cell line) expressing Luc-PPE. Cytotoxicity was measured by Matador Glo assay after co-culture period of 24 h and 48 h and at E:T (Effector: Target) ratios of 3:1, 1:1 and 0.3:1, respectively. The T cells expressing the SAR construct with SEQ ID NO: 32292, which has the architecture of a second-generation CAR, showed very high basal TNFα and IFNγ production even when not exposed to target cells, as measured by ELISA. This was attributed to tonic signaling. There was further increase in TNFα and IFNγ secretion upon co-culture with LNCaP target cells. In contrast, T cells expressing the SAR with SEQ ID NO: 32293-95 showed very low basal TNFα and IFNγ production and showed minimal or no increase upon co-culture with the LNCaP target cells. The in vivo activity of the SARs was tested using a LNCaP xenograft model in NSG mice. Mice (n=5) were injected subcutaneously with 1 million LNCaP cells expressing Luc-PPE in Matrigel (50:50). Five days later, animals were injected with 10 million/mice control T cells or SAR-T cells. Animals were followed using bioluminescence imaging. Two of the five mice given T cells expressing the CAR (SEQ ID NO: 32292) died on Day 20 (i.e., 15 days after injection of SAR-T cells). In contrast, no early death was seen in animals given control T cells or T cells expressing the SAR constructs with SEQ ID NO: 32293-95. Mice administered SAR constructs with SEQ ID NO: 32293-95 also showed effective and near complete tumor control as compared to mice given control T cells or those given T cells expressing the 2^{nd} generation CAR construct with SEQ ID NO: 32294.

An independent experiment was conducted using T cells expressing the constructs with SEQ ID NO: 32292-94. The T cells expressing the SAR with SEQ ID NO: 32292 again showed very high basal TNFα and IFNγ production reflecting tonic signaling. The in vivo activity and safety were tested using LNCaP xenograft model in immunodeficient mice (NCG; Charles River). In this experiment, all immunodeficient mice given T cells expressing the SAR with SEQ ID NO: 32292 showed early mortality within approximately 20 days after injection of T cells. The mice given T cells expressing the SAR with SEQ ID NO: 32293 showed effective tumor control as compared to animals given control T cells and showed the best survival with four of the five animals survived up to day 62 after injection of LNCaP cells. Mice given SAR with SEQ ID NO: 32294 also showed effective tumor control but three of the five animals died by day 62 after tumor injection.

A large panel of additional SAR constructs targeting STEAP2 and comprising the same vL (SEQ ID NO: 19776) and vH (SEQ ID NO: 19795) fragment as the antigen binding domain are constructed and tested using in vitro and in vivo assays. Example SAR constructs targeting STEAP2 are represented by SEQ ID NO: 21893-21947. T cells expressing these SAR constructs show lower TNFα and IFNγ production as compared to the T cells expressing the 2^{nd} generation CAR construct represented by SEQ ID NO: 32294. A large panel of hybrid chain SAR constructs are constructed in which either one or both TCR chain is a hybrid chain. Furthermore, SAR constructs comprising the same vL (SEQ ID NO: 19776) and vH (SEQ ID NO: 19795) fragment but comprising N-termina deletions of one or more TCR chains (hybrid and non-hybrid chains) are constructed. T cells expressing these hybrid chain SAR targeting STEAP2 will show lower TNFα and IFNγ production as compared to the T cells expressing the 2^{nd} generation CAR construct represented by SEQ ID NO: 32294. Additionally, T cells expressing these hybrid chain SAR constructs will show superior safety in vivo when administered to immunodeficient NSG mice. Collectively, these results would demonstrate that it is possible to use hybrid and/or mutant TCR chains to quickly and economically generate a large panel of affinity attuned SAR constructs with varying level of affinity for the target antigen and comprising the same antigen binding domain (e.g., vL and vH fragment). The approach described herein is not limited to SAR comprising vL/vH fragments can be also used to generate affinity tuned SAR constructs comprising any antigen binding domain (e.g., vHH, FHVH, DARPIN, sd-TCR, adaptor, Vα, Vβ, Vγ and Vδ, etc.). This approach has inherent advantage over the conventional approach of CDR mutagenesis of the antigen binding domain (e.g., vL and/or vH fragment) to generate affinity tuned CAR constructs as CDR mutagenesis approach is expensive, time consuming and, in addition, carries the risk of acquiring new antigen specificity.

In vitro and in vivo activity of T cells expressing CD70 SARs. A large panel of SAR on different architectures comprising the vL, vH and scFv fragments represented by SEQ ID NOs: 19768-19772, 19787-19791 and 19806-19809 are constructed and tested in JNG cells. Human peripheral blood T cells isolated using CD3 magnetic beads are infected with lentiviruses expressing the SAR constructs with SEQ ID NO: 32387-32389. These constructs comprise vL, vH, and scFv fragments represented by SEQ ID NO: 19772, 19891 and 19809, respectively. The SAR construct with SEQ ID NO: 32387 is a second-generation CAR. The construct with SEQ ID NO: 32388 is a double chain SIR that is similar in architecture to SEQ ID NO: 32770. The SAR construct with SEQ ID NO:32389 is a zSAR in which the vL and vH fragments are operationally linked to CD3z chains via Ig-like linkers derived from TCRβ and TCRα chains. The expression of the SAR on T cells is demonstrated by staining with Protein L. The cytotoxicity of T cells expressing the different SAR constructs is demonstrated using Matador Glo assay following 18-hour co-culture with Luc-PPE expressing 786-O and ACHN cell lines. The cytokine production is demonstrated using ELISA. The T cells expressing the construct with SEQ ID NO: 32388 is found to be most potent in terms of cytotoxicity and cytokine production. The in vivo activity of the different SAR-T cells is demonstrated by xenograft of LucPPE expressing 786-O (CD70^{high}) and ACHN (CD70^{intermediate}) kidney cancer cell lines in NSG mice. For this purpose, 1 million 786-O-Luc PPE cells are injected subcutaneously on left flank in Matrigel and 1 million ACHN-Luc PPE are injected subcutaneously on right side of the same mouse in Matrigel. One week later, the animals receive 5 million control T cells or SAR-T cells by tail vein injection. Bioluminescence imaging (BLI) demonstrates superior tumor control in mice given all three SAR-T cells as compared to mice given control T cells. Mice given T cells expressing the SAR with SEQ ID NO: 32388 shows the best tumor control and survival.

A large panel of additional SAR constructs targeting CD70 and comprising the same vL and vH fragments as those in SEQ ID NO: 32388 as the antigen binding domain are constructed. The SAR-T cells are tested using in vitro and in vivo assays. These constructs can be used to generate a polyfunctional and diverse immune response. In addition to kidney cancer derived cell lines, these constructs will show activity against CD70-expressing AML cell lines.

In vitro and in vivo activity of T cells expressing CLDN6 (Claudin 6) SARs. A large panel of SAR on different architectures and comprising the vL, vH and scFv fragments represented by SEQ ID NOs: 32020-32023, 32083-32086 and 32146-32149, respectively are constructed and found to show NFAT activity against CLDN6 expressing target cells when expressed in JNG cells. Human peripheral blood T cells isolated using CD3 magnetic beads are infected with lentiviruses expressing the SAR constructs with SEQ ID NO: 32287-32291. These constructs comprise vL fragments represented by SEQ ID NO: 32021 and 32023, vH fragments represented by SEQ ID NO: 32083 and 32086 and scFv represented by SEQ ID NO: 32146 and 32149, respectively. The SAR constructs with SEQ ID NO: 32287 and 32290 are second-generation CAR. The other SAR constructs are double chain SIR in architecture. In the construct with SEQ ID NO: 32289 and 32291, the vH fragment is operationally linked in frame to a human codon optimized TCRβ chain with S57C mutation (SEQ ID NO: 470) and vL fragment is operationally linked to a human codon optimized human TCRα chain with T48C mutation (SEQ ID NO: 458). In the construct with SEQ ID NO: 32288, the vL fragment is attached to a human codon optimized and murinized human TCRα chain with T48C mutation (SEQ ID NO: 455) and vH fragment is operationally linked in frame to a human codon optimized and murinized human TCRβ chain with S57C mutation (SEQ ID NO: 471). The expression of the SAR on T cells is demonstrated by staining with Protein L. The CAR constructs show the highest expression. The cytotoxicity of T cells expressing the different SAR constructs is demonstrated using Matador Glo assay following 24 hours co-culture with Luc-PPE expressing OVCAR3 and OV90 ovarian cancer cell lines, which are known to express CLDN6. The T cells expressing all SAR constructs show increased cytotoxicity at E:T ratios of 3:1 and 1:1. The cytokine production is demonstrated using ELISA. The T cells expressing the CAR constructs with SEQ ID NO: 32287 and 32290 show high IFNγ and TNFα production when co-cultured with target cell lines for 48 h. The SIR constructs (SEQ ID NO: 32288, 32289, 32291) show low to negligible cytokine production. The in vivo activity of the different SAR-T cells is demonstrated by xenograft of LucPPE-expressing OV90 ovarian cancer cell line in NSG mice. 5 million OV90-Luc PPE cells are injected subcutaneously in Matrigel in the flank of male mice. Two weeks later, the animals receive 5 million control T cells or SAR-T cells by tail vein injection. Bioluminescence imaging (BLI) demonstrates superior tumor control and survival in mice given all SAR-T cells as compared to mice given control T cells.

A large panel of additional SAR constructs targeting CLDN6 and comprising the same vL and vH fragments are constructed. The SAR-T cells are tested using in vitro and in vivo assays. These constructs can be used to generate a polyfunctional and diverse immune response when administered to a subject in need thereof.

Expression and activity of T cells expressing CLDN18.2 SARs. A large panel of SAR on different architectures and comprising the vL, vH and scFv fragments represented by SEQ ID NOs: 32018-32019, 32081-32082 and 32144-32145, respectively are constructed and found to show NFAT activity against CLDN18.2 expressing target cells when expressed in JNG cells. Human peripheral blood T cells isolated using CD3 magnetic beads are infected with lentiviruses expressing the SAR constructs with SEQ ID NO: 32304-32307. The SAR constructs with SEQ ID NO: 32304 is a second-generation CAR. The other SAR constructs are double chain SIR in architecture. The expression of the SAR on T cells is demonstrated by staining with Protein L. The CAR constructs show the highest expression. The cytotoxicity of T cells expressing the different SAR constructs is demonstrated using Matador Glo assay following 24 hours co-culture with Luc-PPE expressing NUGC4 stomach cancer cell line. The T cells expressing all SAR constructs show increased cytotoxicity at E:T ratios of 3:1 and 1:1. The T cells expressing the CAR constructs with SEQ ID NO: 32304 shows high IFNγ and TNFα production when co-cultured with target cell lines for 48 h as measured by ELISA. The SIR constructs (SEQ ID NO:32305-32307) show low cytokine production. The in vivo activity of the different SAR-T cells is examined by xenograft of LucPPE-expressing NUGC4 cancer cell line in NSG mice. Approximately 0.1 million NUGC4-Luc PPE cells are injected subcutaneously in Matrigel in the flank of male mice. Next day, the animals receive 5 million control T cells or SAR-T cells by tail vein injection. Bioluminescence imaging (BLI) is used to monitor tumor control. The experiment is repeated a different panel of CLDN18.2 SAR constructs (SEQ ID NO: 32349-32352) based on the SIR architecture. T cells expressing these SAR constructs also show effective cytotoxicity against NUGC4 cells as measured by Matador-Glo assay. A large panel of additional SAR constructs targeting CLDN18.2 and comprising the same vL and vH fragments are constructed. The SAR-T cells are tested using in vitro and in vivo assays. These constructs can be used to generate a polyfunctional and diverse immune response when administered to a subject in need thereof.

Expression and activity of T cells expressing TAJ/TNFRSF19 SARs. A panel of scFvs are generated against TAJ/TNFRSF19 and screened using Malibu-Glo assay. The scFv with SEQ ID NO: 32169 is shown to specifically bind to 293 FT cells transfected with a TAJ expression vector. A large panel of SAR on different architectures and comprising the vL, vH and scFv fragments represented by SEQ ID NOs: 32043, 32106, 32169 respectively are constructed and found to show NFAT activity against TAJ/TNFRSF19 expressing target cells when expressed in JNG cells. Human peripheral blood T cells are infected with lentiviruses expressing the SAR constructs with SEQ ID NO: 32347, 32348 and 32354. The SAR construct with SEQ ID NO: 32347 is a second-generation CAR. The other two SAR constructs have the architecture of double chain SIR. The expression of the SAR on T cells is demonstrated by staining with Protein L. The CAR construct shows highest expression. The cytotoxicity of T cells expressing the different SAR constructs is demonstrated using Matador Glo assay following 18 hours co-culture with Luc-PPE expressing HepG2 (liver cancer), HuTu-80 and Huh7 cell lines. The T cells expressing all SAR constructs show increased cytotoxicity at E:T ratios of 3:1 and 1:1. The T cells expressing the CAR constructs with SEQ ID NO: 32347 shows higher basal IFNγ and TNFα secretion and further increase in secretion when co-cultured with target cell lines for 48 h. The SIR constructs with SEQ ID NO: 32349 and 32354 show low basal cytokine production and negligible increase upon culture with HepG2 and mild increase upon coculture with Huh7 cell lines. Both SIR constructs show increased IFNγ and TNFα production upon coculture with HuTu-80 cells. A large panel of additional SAR constructs targeting TAJ/TNFRSF19 and comprising the same vL and vH fragments are constructed. The SAR-T cells are tested using in vitro and in vivo assays. These constructs can be used to generate a polyfunctional and diverse immune response when administered to a subject in need thereof.

Expression and activity of T cells expressing GD2 SARs. A panel of scFv are generated against GD2 and screened using Malibu-Glo assay. A large panel of SAR on different architectures and comprising the vL, vH and scFv fragments represented by SEQ ID NOs: 32014-32017, 32077-32080, 32140-32143 respectively are constructed and found to show NFAT activity against GD2 expressing target cells when expressed in JNG cells. Human peripheral blood T cells are infected with lentiviruses expressing the SAR constructs with SEQ ID NO: 32296-32299. The SAR constructs with SEQ ID NO: 32296 and 32298 are second-generation CAR. The other SAR constructs have the architecture of double chain SIR. The expression of the SAR on T cells is examined by staining with Protein L. The CAR construct shows highest expression. The cytotoxicity of T cells expressing the different SAR constructs is demonstrated using Matador Glo assay following 48 hours co-culture with Luc-PPE expressing SK-MEL (melanoma), A375 (melanoma) and SH-SY5Y (neuroblastoma) cell lines. The T cells expressing all SAR constructs show increased cytotoxicity at E:T ratio of 1:1. The T cells expressing the CAR constructs with SEQ ID NO: 32296 and 32298 show higher IFNγ and TNFα secretion when co-cultured with target cell lines for 48 h. The SIR constructs with SEQ ID NO: 32297 and 32299 show low basal cytokine production and negligible increase upon culture with target cells. A large panel of additional SAR constructs targeting GD2 and comprising the same vL and vH fragments are constructed. The SAR-T cells are tested using in vitro and in vivo assays. These constructs can be used to generate a polyfunctional and diverse immune response when administered to a subject in need thereof.

### NK Cells Expressing CD19 SAR Show Effective Activity Against NALM6 Xenograft Model in Mice.

A number of SAR targeting CD19 were constructed in lentiviral vectors (SEQ ID NO: 4-6, 24) comprising vL and vH fragments of a humanized CD19 monoclonal hu-mRO05-1 represented by SEQ ID NOs 339 and 363 respectively. These constructs are represented by SEQ ID NO: 32315-32321. The construct with SEQ ID NO: 32315 is a second-generation CAR. The SAR with SEQ ID NO: 32316 is a single chain CD16-SAR in which the CD19 scFv is joined in frame to the N-terminus of a human CD16a variant. The SAR constructs with SEQ ID NO: 32317-32321 are double chain SARs in which the vL fragment is joined to a fragment (SEQ ID NO: 946) encoding a TCRβ-derived Ig-like linker that is joined in frame to the CD3z (dQ101 variant). The complementary vH fragment is attached to a fragment (SEQ ID NO: 31997) encoding a TCRα derived Ig-like linker that is joined in frame to the hinge, transmembrane and cytosolic domain of human CD16a. The SAR constructs with SEQ ID NO: 32318-32321 also comprise accessory modules that encode for soluble IL15 (sIL15) (SEQ ID NO: 31996), a membrane anchored IL15 variant (SEQ ID NO: 908), 41BBL and CD48 (SEQ ID NO: 32001), respectively. The constructs were pseudotyped with modified baboon envelope (072622-SyCD2; SEQ ID NO: 95). Blood was obtained from healthy male donor. Primary NK cells infected with different CD19 SAR constructs and pseudotyped with modified baboon envelope (072622-SyCD2; SEQ ID NO: 95). Cells were expanded in GRex plates for 5-7 days. Protein L staining showed that 52%, 55%, 62%, 45%, 51%, 49% and 31% of T cells showed expression of SAR constructs represented by SEQ ID NO: 32315-32321. Matador Glo cytotoxicity assay showed effective killing of RS4; 11 and NALM6 target cells by NK cells expressing all SAR constructs at E:T ratios of 3:1 and 1:1. In general, the SAR constructs with SEQ ID NO: 32317-32320 showed higher cytotoxicity in the Matador Glo assay and higher TNFα and IFNα secretion upon co-culture with the target cells as compared to the 2^{nd} generation CAR construct represented by SEQ ID NO: 32315.

9 weeks old NSG mice expressing human IL15 (NSG-hIL15) were obtained from Jackson Laboratory. Mice were injected with 0.1 million NALM6-LucPPE cells via tail vein. One day later, mice (n=4) were injected via tail vein with 10 million NK cells expressing the different SAR constructs. Remaining NK-cells were frozen. The mice received a second and a third injection of NK-SAR cells 7 and 16 days after the first injection. Tumor growth was monitored by BLI. Mice given NK cells expressing all SAR constructs showed better tumor control and survival as compared to mice given control NK cells that lack SAR. Mice given NK cells expressing the SAR construct represented by SEQ ID NO: 32319 showed complete tumor control in all 4 animals and best survival up to day 50. Mice given NK cells expressing the SAR construct represented by SEQ ID NO: 32316 showed the next best survival and 3 out of the 4 animals survived up to day 50.

T cells expressing CSF1R SARs induce cytotoxicity in CSF1R-expressing cells. Human peripheral blood T cells isolated using CD3 magnetic beads are infected with lentiviruses expressing the SAR constructs (e.g., SEQ ID NO: 20036-20353) targeting CSF1R. SAR-T cells are expanded in vitro for 10-14 days. K562 and HepG2 cells stably expressing GLuc are cocultured with T cells expressing the different SARs at an E:T ratio of 10:1 for 48 hours. SAR-T cells mediated induction of lysis of target cells is assayed using the Matador assay by measurement of GLuc activity. The in vivo activity of the SARs is demonstrated using a xenograft model in NSG mice. The CSF1R SARs are used for the treatment of patients with fibrotic diseases, including chronic GVHD.

T cells expressing HLA-A2 SARs induce cytotoxicity in HLA-S2-expressing cells. Human peripheral blood T cells are infected with lentiviruses expressing the SAR constructs (e.g., SEQ ID NO: 32705-06) targeting HLA-A2. SAR-T cells are expanded in vitro for 10-14 days. U266 and L363 cells stably expressing GLuc are cocultured with T cells expressing the different SARs at an E:T ratio of 10:1 for 48 hours. SAR-T cells mediated induction of lysis of target cells is assayed using the Matador assay by measurement of GLuc activity.

T cells expressing mutant p53 (R248Q) and (R175H) SARs induce cytotoxicity in mutant p53 (R248Q) expressing HLA-A2 cells. Human peripheral blood T cells isolated using CD3 magnetic beads are infected with lentiviruses expressing the SAR constructs (e.g., SEQ ID NO: 3816-3872 and 3768, 3905) targeting mutant p53 (R248Q) or R175H. SAR-T cells are expanded in vitro for 10-14 days. T2 cells stably expressing GLuc and loaded with the mutant p53 peptides are cocultured with T cells expressing the different SARs at an E:T ratio of 10:1 for 48 hours. SAR-T cells mediated induction of lysis of target cells is assayed using the Matador assay by measurement of GLuc activity. The in vivo activity of the SARs is demonstrated using a xenograft model in NSG mice.

T cells expressing KLK2 SARs induce cytotoxicity in KLK2-expressing cells. Human peripheral blood T cells isolated using CD3 magnetic beads are infected with lentiviruses expressing the SAR constructs (e.g., SEQ ID NO: 1015-1016) targeting KLK2. SAR-T cells are expanded in vitro for 10-14 days. LNCaP cells stably expressing GLuc are cocultured with T cells expressing the different SARs at an E:T ratio of 10:1 for 48 hours. SAR-T cells mediated induction of lysis of target cells is assayed using the Matador assay by measurement of GLuc activity.

T cells expressing GPRC5D SARs induce cytotoxicity in GPRC5D-expressing cells. Human peripheral blood T are infected with lentiviruses expressing the unispecific and bispecific SAR constructs (e.g., SEQ ID NO: 35904-36000) targeting GPRC5D. SAR-T cells are expanded in vitro for 10-14 days. L363 and MM1s cells stably expressing GLuc are cocultured with T cells expressing the different SARs at an E:T ratio of 10:1 for 48 hours. SAR-T cells mediated induction of lysis of target cells is assayed using the Matador assay by measurement of GLuc activity.

### JNG Cells Expressing MHC-SAR Respond to T Cells Expressing a TCR Targeting RQ13

MHC-SARs (SEQ ID NO: 23780-23794) targeting the TCRs that bind to RQ13/HLA-DR complex are expressed in JNG cells. The JNG cells expressing the SAR can recognize T cells expressing a TCR (SEQ ID NO (DNA): 23236 that binds to RQ13/HLA-DR complex and induce GFP expression. Experiment is repeated by expressing the MHC-SARs in primary T cells which recognize T cells expressing the TCR (SEQ ID NO (DNA): 23236. In another example, primary NK cells expressing the SAR (SEQ ID NO: 23780-23787) recognize T cells expressing the TCR SEQ ID NO (DNA): 23236.

T cells expressing hybrid chain TCR targeting NY-ESO-1/HLA-A2 complex induce cytotoxicity against NY-ESO-1 peptide/HLA-A2 expressing target cells. Human peripheral blood T cells isolated using CD3 magnetic beads are infected with lentiviruses expressing the SAR constructs (e.g., SEQ ID NO: 19760-61) targeting NY-ESO-1/HLA-A2. SAR-T cells are expanded in vitro for 10-14 days. T2 and L363 cells stably expressing GLuc are loaded with the NY-ESO-1 peptide and then cocultured with T cells expressing the different SARs at an E:T ratio of 10:1 for 48 hours. SAR-T cells mediated induction of lysis of target cells is assayed using the Matador assay by measurement of GLuc activity.

SAR with αβ TCR Chains Expressed in γδ T Cells

A CD19 targeted SAR construct CD8SP-CD19-hu-mRO05-1-vL-[hTCRb-opt2]-F-P2A-SP-CD19-hu-mRO05-1-vH-[hTCRa-opt2]-F-F2A-PAC (SEQ ID NO: 3258) comprising vL and vH of hu-mROO5-1 antibody attached to wild-type amino acid sequence of TCRb and TCRa constant chains, respectively, is expressed in αβ and γδ T cells. The TCRb and TCRa chains in this construct are comprised of their human codon-optimized sequence. The construct shows good expression and activity when expressed in γδ T cells.

### Use of Mitomycin Treated Cells for Matador Assay

NK-92 and primary NK cells are co-cultured with K562-LucPPE (UT, treated with Mitomycin C at 30 μg/ml for 1 h or irradiated at 100Gy) at E:T ratio 0.3:1 and E:T 1:1 for 3 h. An increase in the Luc activity is measured by addition of D-luciferin containing buffer as described for Matador-Glo assay. The results show that mitomycin or irradiated cells can be used in the Matador assay.

### Embodiments

1. A SAR comprising at least two chains wherein
a) a first polypeptide chain comprises a first antigen-binding domain comprising a vL, a Vα or a Vγ domain and a first Membrane associated module (MAM); and
b) a second polypeptide chain comprises a second antigen-binding domain comprising a vH, a Vβ or a Vδ domain and a second Membrane associated module (MAM);
wherein the vL, Vα or Vγ domain of the first antigen-binding domain and the complementary vH, Vβ or Vδ domain of the second antigen-binding domain form a Fv- or TCR-Fv like antigen-binding module that specifically binds to the target antigen; and
wherein the first MAM and the second MAM form a non-T cell receptor module (NTCRM) that is capable of activating at least one signaling pathway and/or recruiting at least one signaling adaptor; and
wherein one or both MAM comprise a sequence with SEQ ID NO:8925-8948, 9310-9321, 9323-9346 or a sequence with at least 70% homology thereto or a functional variant of any of the foregoing and fragments thereof.
2. The SAR of embodiment 1, where the first polypeptide chain further comprises a first peptide linker between the first antigen-binding domain and the first MAM, and the second polypeptide chain further comprises a second peptide linker between the second antigen-binding domain and the second MAM.
3. The SAR of embodiment 2, wherein the first and/or second peptide linkers comprise, individually, a constant domain or fragment thereof from an immunoglobulin or T cell receptor subunit.
4. The SAR of embodiment 3, wherein the first and/or second peptide linkers comprise, individually, a CH1, CH2, CH3, CH4 or CL antibody domain, or a fragment thereof.
5. The SAR of embodiment 4, wherein the first and/or second peptide linkers comprise, individually, a Cα, Cβ, Cγ, or Cδ TCR domain, or a fragment thereof.
6. The SAR of embodiments 3 or 4, wherein the first polypeptide chain and the second polypeptide chain are linked via one or more disulfide bonds.
7. The SAR of embodiment 4, wherein first and/or second peptide linkers comprise mutations that increase the expression, affinity and/or pairing of the two polypeptide chains.
8. The SAR of embodiment 4, wherein the first and/or second peptide linkers comprise a sequence as set forth in any one of SEQ ID NO: 8961-8994 and 21466-21471 or a sequence with at least 70% identity thereto.
9. The SAR of embodiment 3, wherein the first polypeptide further comprises a first hinge domain or fragment thereof N-terminal to the first MAM; and/or wherein the second polypeptide further comprises a second hinge domain or fragment thereof N-terminal to the second MAM.
10. A SAR of embodiment 1, comprising a disulfide bond between a residue in the first MAM and the second MAM and/or a residue in the first hinge domain and a residue in the second hinge domain.
11. A SAR of embodiment 1, wherein the first polypeptide further comprises a first homologous antigen binding domain or fragment thereof N-terminal to the first hinge domain and/or the second polypeptide further comprises a second homologous antigen binding domain or fragment thereof N-terminal to the second hinge domain, wherein the two homologous antigen binding domains are derived from the same naturally occurring non-T cell receptor as the corresponding hinge domains.
12. A SAR of embodiment 1, wherein the first polypeptide further comprises a first cytosolic domain containing an optional activation domain C-terminal to the first transmembrane/membrane-anchoring domain comprising the first MAM; and/or wherein the second polypeptide further comprises a second cytosolic containing an optional activation domain C-terminal to the second transmembrane/membrane anchoring domain comprising the second MAM.
13. The SAR of embodiment 1, wherein the first polypeptide chain further comprises a first accessory intracellular domain comprising a co-stimulatory or a co-receptor domain sequence C-terminal to the first transmembrane/membrane anchoring domain of the first MAM; and/or wherein the second polypeptide chain further comprises a second accessory intracellular domain comprising a co-stimulatory, a coreceptor domain sequence or a signaling molecule C-terminal to the second transmembrane/membrane anchoring domain comprising the second MAM.
14. A SAR of embodiment 13, wherein the a) co-stimulatory domain is selected from the cytosolic domain of CD28, 4-1BB, OX40, 2B4, CD27, CD81, CD2, CD5, BAFF-R, CD30, CD40, HVEM or ICOS, or a variant or a fragment thereof; and b) co-receptor domain is selected from the cytosolic domain of CD8a, CD8b or CD4, or a variant or a fragment thereof and c) signaling molecule is a kinase optionally selected from the group of Lck, FYN, ZAP-70, PLCγ1, SLP-76 and LAT or a functional variant or a fragment thereof.
15 A SAR of embodiment 1, wherein the first and/or the second MAM and the NTCRM are comprised of the transmembrane/membrane anchored domain, optional cytosolic domain, optional hinge domain and/or optional extracellular domain of a non-T cell receptor and/or a signaling adaptor.
16. A SAR of embodiment 15, wherein the first and/or the second MAM and the NTCRM are comprised of the transmembrane/membrane anchored domain, optional cytosolic domain, optional hinge domain and/or optional extracellular domain that are all derived from a single non-T cell receptor and/or a signaling adaptor or variants thereof.
17. A SAR of embodiment 15, wherein the first and/or the second MAM and the NTCRM are comprised of the transmembrane/membrane anchored domain, optional cytosolic domain, optional hinge domain and/or optional extracellular domain that are derived from different non-T cell receptor and/or a signaling adaptor or variants thereof.
18. A SAR of embodiment 15, wherein the two transmembrane/membrane anchored domains, optional cytosolic domains, optional co-stimulatory domain, optional hinge domains and/or optional extracellular domains are identical in sequence and are derived from the same protein.
19. A SAR of embodiment 15, wherein the two transmembrane/membrane anchored domains, optional cytosolic domains, optional co-stimulatory domain, optional hinge domains and/or optional extracellular domains are different in sequence and/or are derived from different proteins.
20. A SAR of embodiment 15, wherein
a. the non T cell receptor is a naturally occurring receptor and is selected from the group consisting of: CD16A, CD16B, CD64, CD32, NKp30, NKp44, NKp46, KIR2DL1, KIR2DL2, KIR2DL3, KIR2DL5A, KIR2DL5B, KIR3DL1, KIR3DL2, KIR3DL4, KIR2DL4, KIR2DS1, KIR2DS2, KIR2DS3, KIR2DS4, KIR2DS5, KIR3DS1, NKG2D, NKG2C, NKG2A, NKG2E, NKG2F, DNAM-1, 2B4, OX40, CD28, 4-1BB, CD27, CD81, CD2, CD5, TNFR-I, TNFR-II, Fas, CD30, CD40, CRTAM, TIGIT, CD96, SLAMF6, SLAMF7, CD100, CD160, CEACAM, ILT2, KLRG1, LAIR1, CD161, a variant of any of the foregoing, and fragments thereof; and
b. the signaling adaptor is selected from the group consisting of: CD3ζ, FcRγ, DAP10, a variant of any of the foregoing and fragments thereof.
21. A SAR of embodiment 20, where the a) CD16 lacks a partial or complete cytosolic domain and/or comprise a mutation in the transmembrane domain; and/or b) one or both CD3ζ cytosolic domains comprises a deletion of residue Q101; c) both a) and b).
22. A SAR of embodiment 1, wherein one or more autonomous antigen binding domains (AABD) or fragments thereof are operationally linked to the N-terminus or near the N-terminus of the vL, vH, Vα, Vβ, Vγ and/or Vδ domain via one or more optional linkers.

What is claimed is:

1. A recombinant polynucleotide encoding at least one hybrid chain synthetic antigen receptor (HC-SAR) or a functional variant thereof, the at least one SAR or the functional variant comprising a heterodimer of two T-cell receptor (TCR) constant chains, the heterodimer having one or more non-TCR antigen binding domains that are operably linked to the TCR constant chains via optional linkers, wherein at least one TCR constant chain of the HC-SAR is a hybrid TCR chain, wherein the hybrid TCR chain comprises at least one domain selected from the group of domains consisting of a TCR chain constant domain (C), a TCR chain connecting peptide (ConnP), a TCR chain transmembrane (TM) domain, and a TCR chain cytoplasmic domain (CP) that is heterologous.

2. The HC-SAR of claim 1, where the at least one domain that is heterologous to the TD of the hybrid TCR chain is derived from the group of domains heterologous to the TD of the hybrid TCR chain consisting of
a. a TCRα, TCRβ, TCRγ, TCRδ or pre-TCRα chain that lacks the TCR chain transmembrane domain present in the hybrid TCR chain;
b. a TCR chain with a TM domain with less than 50% sequence identity to the TM of the hybrid TCR chain;
c. a TCR constant chain or a functional variant from a different species; and
d. any combination thereof.

3. The HC-SAR of claim 1, wherein more than one domain selected from the group of domains consisting of the TCR chain constant domain (C), the TCR chain connecting peptide (ConnP), the transmembrane (TM) domain, and the cytoplasmic domain (CP) is heterologous.

4. The HC-SAR of claim 1, wherein the ConnP is heterologous to a) TM domain; b) constant domain (C); or c) both a) and b).

5. The HC-SAR of claim 1, wherein the TM domain encodes
a. a peptide with a sequence selected from SEQ ID NO: 31985-88 and 31992, and/or
b. a peptide with a sequence selected from the group of sequences consisting of
i) a transmembrane domain with SEQ ID NO: 40606 to 40670 and 40737-40758 or a functional variant or a homolog thereof with 1, 2, 3, 4, 5, or 6 amino acid substitutions except at residues 7, 12, 17, and 21; and/or
ii) b) transmembrane domain with SEQ ID NO: 40671-40708 or a functional variant or a homolog thereof with 1, 2, 3, 4, 5, or 6 amino acid substitutions except at residues 6, 12, and 16.

6. The HC-SAR of claim 5, wherein
the first hybrid TCR chain further comprises a second TCR chain connecting peptide or a functional variant or fragment thereof, but does not comprise a first TCR chain connecting peptide; and
wherein the second TCR hybrid chain further comprises a first TCR chain connecting peptide or a functional variant or fragment thereof, but does not comprise a second TCR chain connecting peptide,
wherein the first TCR chain connecting peptide comprises a TCR alpha or a TCR gamma chain connecting peptide or a functional variant or fragment thereof and the second TCR chain connecting peptide comprises a TCR beta chain or a TCR delta connecting peptide or a functional variant or fragment thereof, or
wherein the first TCR chain connecting peptide comprises a TCR alpha or a TCR delta chain connecting peptide or a functional variant or fragment thereof and the second TCR chain connecting peptide comprises a TCR beta chain or a TCR gamma connecting peptide or a functional variant or fragment thereof.

7. The HC-SAR of claim 6, wherein
a first hybrid chain comprises a first TCR chain transmembrane domain, a non-TCR antigen binding domain and a second TCR chain constant domain, or a functional variant or a fragment thereof, but does not comprise a first TCR chain constant domain; and
a second hybrid TCR chain comprises a second TCR chain transmembrane domain, an optional non-TCR antigen-binding domain, and a first TCR chain constant domain or a functional variant or a fragment thereof, but does not comprise a second TCR chain constant domain;
wherein the first TCR chain constant domain comprises a TCR alpha or a TCR gamma chain constant domain or a functional variant or fragment thereof, the second TCR chain constant domain comprises a TCR beta or a TCR delta chain constant domain or a functional variant or fragment thereof, and the second TCR chain transmembrane domain comprises a TCR beta or a TCR delta chain transmembrane domain or a variant thereof; or
wherein, the first TCR chain constant domain comprises a TCR alpha or a TCR delta chain constant domain or a functional variant or fragment thereof, the first TCR chain transmembrane domain comprises a TCR alpha or a TCR delta chain transmembrane domain or a functional variant thereof, the second TCR chain constant domain comprises a TCR beta or a TCR gamma chain constant domain or a functional variant thereof, and the second TCR chain transmembrane domain comprises a TCR beta or a TCR gamma chain transmembrane domain or a variant thereof.

8. The HC-SAR of claim 1, wherein both of the TCR constant chains are hybrid chains.

9. The HC-SAR of claim 1, wherein the constant domain of at least one hybrid chain is derived from an immunoglobulin, or wherein both hybrid chains are derived from an immunoglobulin.

10. The HC-SAR of claim 1, comprising:
a first polypeptide chain comprising a first antigen-binding domain having a vH antibody domain, a TCR constant domain or a functional variant or fragment thereof, and a first T cell receptor domain (TCRD) having a first transmembrane domain of a first TCR subunit; and
a second polypeptide chain comprising a second antigen-binding domain having a vL antibody domain, a TCR constant domain or a functional variant or fragment thereof, and a second TCRD comprising a second transmembrane domain of a second TCR subunit,
wherein the vH antibody domain of the first antigen-binding domain and the vL antibody domain of the second antigen-binding domain form an antigen-binding module that specifically binds to a target antigen; or
a first polypeptide chain comprising a first antigen-binding domain having a vL antibody domain, a TCR constant domain or a functional variant or fragment thereof of a first TCR subunit, and a T cell receptor domain (TCRD) comprising a first transmembrane domain of a second TCR subunit; and
a second polypeptide chain comprising a second antigen-binding domain having a vH antibody domain, a TCR constant domain or a functional variant or fragment thereof of the second TCR subunit, and a second TCRD comprising a second transmembrane domain of the first TCR subunit,
wherein the vH antibody domain of the first antigen-binding domain and the vL antibody domain of the second antigen-binding domain form an antigen-binding module that specifically binds to a target antigen;
wherein (i) the first TCR subunit is a TCR γ chain and the second TCR subunit is a TCR δ or TCR α chain; or (ii) the first TCR subunit is a TCR δ chain and the second TCR subunit is a TCR γ or TCR β chain; or (iii) the first TCR subunit is a TCR α chain and the second TCR subunit is a TCR β or TCRγ chain; or (iv) the first TCR subunit is a TCR β chain, and the second TCR subunit is a TCR α or TCR δ chain; and
wherein optionally the first polypeptide chain further comprises a TCR connecting peptide or a functional variant or fragment thereof, and the second polypeptide chain further comprise a TCR connecting peptide or a functional variant or fragment thereof, wherein the TCR connecting peptides or functional variants or fragments are located N-terminal to the transmembrane domain; and
wherein optionally one or both connecting peptides are heterologous to a) transmembrane domain; b) constant domain; c) both a) and b).

11. The HC-SAR of claim 1, comprising:
a first polypeptide chain comprising a first antigen-binding domain having a vH antibody domain, a constant antibody domain, or a functional variant or fragment thereof, and a first T cell receptor domain (TCRD) comprising a first transmembrane domain of a first TCR subunit; and
a second polypeptide chain comprising a second antigen-binding domain having a vL antibody domain, a TCR constant domain or a functional variant or fragment thereof, and a second TCRD comprising a second transmembrane domain of a second TCR subunit,
wherein the vH antibody domain of the first antigen-binding domain and the vL antibody domain of the second antigen-binding domain form an antigen-binding module that specifically binds to a target antigen; or
a first polypeptide chain comprising a first antigen-binding domain having a vL antibody domain, a constant antibody domain or a functional variant or fragment thereof, and a T cell receptor domain (TCRD) comprising a first transmembrane domain of a first TCR subunit; and
a second polypeptide chain comprising a second antigen-binding domain having a vH antibody domain, a TCR constant domain or a functional variant or fragment thereof, and a second TCRD comprising a second transmembrane domain of a second TCR subunit,
wherein the vH antibody domain of the first antigen-binding domain and the vL antibody domain of the second antigen-binding domain form an antigen-binding module that specifically binds to a target antigen;
wherein (i) the first TCR subunit is a TCR γ chain and the second TCR subunit is a TCR δ or TCR α chain, or (ii) the first TCR subunit is a TCR δ chain and the second TCR subunit is a TCR γ or TCR β chain, or (iii) the first TCR subunit is a TCRα chain and the second TCR subunit is a TCRβ or TCR γ chain, or (iv) the first TCR subunit is a TCRβ chain and the second TCR subunit is a TCRα or TCR δ chain; and
wherein optionally the first polypeptide chain further comprises a TCR connecting peptide or a functional variant or fragment thereof and the second polypeptide chain further comprise a TCR connecting peptide or a functional variant or fragment thereof and wherein the TCR connecting peptides or functional variants or fragments are located N-terminal to the transmembrane domain; and
wherein optionally one or both connecting peptides are heterologous to a) the transmembrane domain, b) the constant domain; c) both a) and b).

12. The HC-SAR of claim 1, comprising:
a first polypeptide chain comprising a first antigen-binding domain having a vH antibody domain, a first constant antibody domain or a functional variant or fragment thereof, and a first T cell receptor domain (TCRD) comprising a first ConnP, a TM and a CP domain of a first TCR subunit; and
a second polypeptide chain comprising a second antigen-binding domain having a vL antibody domain, a second constant antibody domain or a functional variant or fragment thereof and a second TCRD comprising a second ConnP, TM and CP domains of a second TCR subunit, wherein the vH antibody domain of the first antigen-binding domain and the vL antibody domain of the second antigen-binding domain form an antigen-binding module that specifically binds to a target antigen; or
a first polypeptide chain comprising a first antigen-binding domain having a vL antibody domain, a first constant antibody domain or a functional variant or fragment thereof, and a first T cell receptor domain (TCRD) comprising a first ConnP, a TM and a CP domain of a first TCR subunit; and
a second polypeptide chain comprising a second antigen-binding domain comprising a vH antibody domain, a second constant antibody domain or a functional variant or fragment thereof and a second TCRD comprising a second ConnP, TM and CP domains of a second TCR subunit, wherein the vH antibody domain of the first antigen-binding domain and the vL antibody domain of the second antigen-binding domain form an antigen-binding module that specifically binds to a target antigen; and
wherein optionally i) the ConnP, the TM and the CP domains of the at least one TCR subunits are heterologous; b) the ConnP and the TM of at least one of the TCR subunits are heterologous; c) the ConnP, the TM, and the CP of both TCR subunits are heterologous; d) the ConnP and the TM domains of both of the TCR subunits are heterologous.

13. The HC-SAR of claim 1, 10, 11, or 12, wherein
the first TCR subunit comprises the ConnP of TCRβ and TM of TCRγ and the second TCR subunit is TCRα or TCRδ; or
the first TCR subunit comprises the ConnP of TCRγ and TM of TCRβ and the second TCR subunit is TCRα or TCRδ; or
the first TCR subunit comprises the ConnP of TCRα and TM of TCRδ and the second TCR subunit is TCRβ or TCRγ; or
the first TCR subunit comprises the ConnP of TCRδ and TM of TCRα and the second TCR subunit is TCRβ or TCRγ; or
the first TCR subunit comprises the ConnP of TCRβ and TM of TCRγ and the second TCR subunit comprises the ConnP of TCRα and TM of TCRδ or the ConnP of TCRδ and TM of TCRα; or
the first TCR subunit comprises the ConnP of TCRγ and TM of TCRβ and the second TCR subunit comprises ConnP of TCRα and TM of TCRδ or the ConnP of TCRδ and TM of TCRα; or
the first TCR subunit comprises the ConnP of TCRα and TM of TCRδ and the second TCR subunit comprises ConnP of TCRβ and TM of TCRγ or ConnP of TCRγ and TM of TCRβ; or
the first TCR subunit comprises the ConnP of TCRδ and TM of TCRα and the second TCR subunit comprises ConnP of TCRβ and TM of TCRγ or ConnP of TCRγ and TM of TCRβ.

14. The HC-SAR of claim 1, wherein the TCR constant chain comprises one or more of the following features:
mutations that enhance the dimerization of the constant chains and reduce pairing with the endogenous T cell receptor chains;
one or both of the TCR constant chains are human codon optimized; and/or
are of human, mouse, or dog origin.

15. The HC-SAR of claim 1, wherein the non-TCR antigen binding domain is selected from the group of binding domains consisting of
(a) an antibody;
(b) an antibody fragment selected from a Fv, a Fab, and a (Fab′)2;
(c) a heavy chain variable region of an antibody (vH domain);
(d) a light chain variable region of an antibody (vL domain);
(e) a single chain variable fragment (scFv);
(f) a single domain antibody (SDAB);
(g) a camelid VHH domain;
(h) a monomeric variable region of an antibody;
(i) a non-immunoglobulin antigen binding scaffold optionally selected from a DARPIN, D domain (DD), an affibody, an affilin, an adnectin, an affitin, an obodies, a repebody, a fynomer, an alphabody, an avimer, an atrimer, a centyrin, a pronectin, an anticalin, a kunitz domain, an Armadillo repeat protein or a binding fragment thereof;
(j) the extracellular domain of receptor or a binding fragment thereof;
(k) a ligand or a binding fragment thereof;
(l) a bispecific antibody;
(m) an autoantigen;
(n) an HLA molecule or a fragment thereof;
(o) a β2M molecule or a fragment thereof; and
(p) an HLA/peptide complex.

16. The HC-SAR of claim 1, wherein one or more autonomous antigen binding domains (AABD) or fragments thereof are operationally linked to the N-terminus or proximate to the N-terminus of one or more non-TCR antigen binding domains via an optional linker.

17. A HC-SAR of claim 16, wherein the one or more autonomous antigen binding domains (AABDs) or fragments thereof are selected from the group of antigen binding domains consisting of
(a) a single vH domain (SVH) or a fragment thereof;
(b) a single vL domain (SVL) or a fragment thereof;
(c) a vHH domain or a fragment thereof;
(d) a single domain antibody or a fragment thereof;
(e) a single variable domain of a TCR (svd-TCR) or a fragment thereof;
(f) a non-immunoglobulin antigen binding scaffold or a fragment thereof;
(g) a ligand-binding domain of a receptor or a fragment thereof;
(h) a receptor-binding domain of a ligand;
(i) an autoantigen or a fragment thereof;
(j) an adaptor binding domain or a fragment thereof;
(k) an adaptor or a fragment thereof;
(l) an epitope or a fragment thereof; and
(m) an Fc binding domain or a fragment thereof.

18. The HC-SAR of claim 17, wherein a non-immunoglobulin antigen binding scaffold is selected from the group of binding scaffolds consisting of a DARPIN, a D domain (DD), an affibody, an affilin, an adnectin, an affitin, an obodies, a repebody, a fynomer, an alphabody, an avimer, an atrimer, a centyrin, a pronectin, an anticalin, a kunitz domain, and an Armadillo repeat protein.

19. The HC-SAR of claim 18, wherein the one or more non-TCR antigen binding domain(s) and/or AABD bind to one or more of a disease-associated antigen selected from the group of disease-associated antigens consisting of CD19, CD5, CD123, CD22, CD30, CD171, a CS-1 (CRACC, SLAMF7, CD319, and 19A24), CD45, a C-type lectin-like molecule-1 (CLL-1 or CLECL1), CD33, epidermal growth factor receptor variant III (EGFRviii), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), FmsLike Tyrosine Kinase 3 (FLT3), Tumor-associated glycoprotein 72 (TAG72), CD38, CD44v6, a glycosylated CD43 epitope expressed on acute leukemia or lymphoma but not on hematopoietic progenitors, a glycosylated CD43 epitope expressed on non-hematopoietic cancers, Carcinoembryonic antigen (CEA), Epithelial cell adhesion molecule (EPCAM), B7H3 (CD276), KIT (CD117), Interleukin-13 receptor subunit alpha-2 (IL-13Ra2 or CD213A2), Mesothelin, Interleukin 11 receptor alpha (IL-11Ra), prostate stem cell antigen (PSCA), vascular endothelial growth factor receptor 2 (VEGFR2), Lewis (Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), carbonic anhydrase IX (CAIX), tyrosinase, Fucosyl GM1, sialyl Lewis adhesion molecule (sLe), ganglioside GM3, transglutaminase 5 (TGS5), high molecular weight-melanoma associated antigen (HMWMAA), claudin 6 (CLDN6), thyroid stimulating hormone receptor (TSHR), G protein coupled receptor class C group 5 member D (GPRC5D), chromosome X open reading frame 61 (CXORF61), CD97; CD179a, anaplastic lymphoma kinase (ALK), mammary gland differentiation antigen (NY-BR-1), Wilms tumor protein (WT1), Cancer/testis antigen 1 (NY-ESO-1), Melanoma-associated antigen 1 (MAGE-A1), melanoma antigen recognized by T cells 1 (MelanA or MARTI), Rat sarcoma (Ras) mutant, human Telomerase reverse transcriptase (hTERT), human papilloma virus E6 (HPV E6), human papilloma virus E7 (HPV E7), CD79a, CD79b, CD72, Leukocyte-associated immunoglobulin-like receptor 1 (LAIR1), C-type lectin domain family 12 member A (CLEC12A), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLLI), Biotin, c-MYC epitope Tag, CD34, LAMP1 TROP2, GFRalpha4, CDH17, CDH6, CDH19, CD200R, Slea (CA19.9, Sialyl Lewis Antigen) Fucosyl-GM1, PTK7, CDH1-CD324, DLL3, CD276/B7H3, IL11Ra, IL13Ra2, CD179b-IGL11, ALK, TCR-gamma-delta, NKG2D, CD32 (FCGR2A), CSPG4-HMW-MAA, Tim1-/HVCR1, CSF2RA (GM-CSFR-alpha), TGFbetaR2, VEGFR2/KDR, Lewis Ag, TCR-alpha chain, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, auto antibody to desmoglein 3 (Dsg3), autoantibody to desmoglein 1 (Dsg1), HLA-A2, HLA-A2:01, HLA-B; HLA-C; HLA-DP, HLA-DM, HLA-DOA, HLA-DOB, HLA-DQ, HLA-DR; HLA-G, IGE, CD99, Lym1, Lym2, RAS G12V, Tissue Factor 1 (TF1), AFP, GPRC5D, claudin18.2 (CLD18A2 or CLDN18A.2), STEAP1, STEAP2, LIV1, NECTIN-4, CRIPTO, GPA33, BST1/CD157, low conductance chloride channel, TAJ/TNFRSF19, MPL (TPO-R), KIR3DL2, CD32b, CD229, Toso, BAFF-R, OR2H1, p95-Her2, huTAG2, immunoglobulin kappa light chain, immunoglobulin gamma light chain, SARS-cov2 spike glycoprotein, SARS-cov2 Receptor binding domain, CSF1R, mutant p53, p53-R175H mutant, p53-R248Q mutant, NPM1c, PRAME1, Melanoma-associated antigen 4 (MAGE-A4), gp100, IL23R, MYCN, and Myelin Oligodendrocyte Glycoprotein (MOG).

20. The HC-SAR of claim 17, wherein the one or more non-TCR antigen binding domain(s) and/or AABD is selected from the group of antigen binding domains consisting of:
a light chain variable region (vL) encoded by a polynucleotide having a sequence of any one of SEQ ID NO 339-354, 19766-19776, 32912-33120, and 32006-32068, or sequences with at least 75% identity thereto in the framework regions and contains the complementarity determining regions (CDRs) of any of the forgoing polypeptides and which encodes a polypeptide that binds to its antigen and a complementary heavy chain variable region (vH) encoded by a polynucleotide having a sequence of any one of SEQ ID NO: 363-378, 19785-19795, 33121-33329, and 32069-32131 or sequences with at least 75% identity thereto in the framework regions and contains the CDRs of any of the forgoing polypeptides and which encodes a polypeptide that binds to its antigen;
a single chain variable fragment (scFv) encoded by a polynucleotide having a sequence of any one of SEQ ID NO 387-402, 19804-19814, 33330-33538 and 32132-32194, or sequences with at least 75% identity thereto in the framework regions and contains the CDRs of any of the forgoing polypeptides and which encodes a polypeptide that binds to its antigen;
a camelid VHH domain encoded by a polynucleotide having a sequence of any one of SEQ ID NO 412-426, 32195-32213, or sequences with at least 75% identity thereto in the framework regions and contains the CDRs of any of the forgoing polypeptides and which encodes a polypeptide that binds to its antigen;
a non-immunoglobulin scaffold encoded by a polynucleotide having a sequence of any one of SEQ ID NO 435-450, or sequences with at least 75% identity thereto and which encodes a polypeptide that binds to its antigen;
a receptor encoded by a polynucleotide having a sequence of any one of SEQ ID NO 437, 438, 445-448, or sequences with at least 75% identity thereto and which encodes a polypeptide that binds to its cognate;
a ligand encoded by a polynucleotide having a sequence of any one of SEQ ID NO 439 or sequences with at least 75% identity thereto and which encodes a polypeptide that binds to its cognate; and
a polynucleotide encoding a light chain variable region (vL) that comprises one or more light chain complementary determining regions 1-3 (LC-CDR1-3) for a selected target antigen as set forth in any of SEQ ID Nos: 20989-21015, 41591-41861; 21024-21050, 41862-42132, and 21059-21085, 42133-42403 and a polynucleotide encoding a complementary heavy chain variable region (vH) that comprises one or more of heavy chain complementary determining regions 1-3 (HC-CDR1-3) for a selected target antigen as set forth in any of SEQ ID Nos: 21094-21120 and 42404-42674; 21129-21155, 42675-42945, 21164-21190, and 42946-43216.

21. The HC-SAR of claim 17, wherein the one or more non-TCR antigen binding domain(s) and/or AABD are selected from the group consisting of
a variable light (vL) domain comprising a sequence of any one of SEQ ID Nos: 8719-8734, 20386-20396, 40759-41029, a sequence with at least 85% identity thereto in the framework region, or a sequence having up to 10 conservative amino acid substitutions in the framework region and containing the CDRs of any of the forgoing polypeptides and a complementary variable heavy (vH) domain comprising a sequence of any one of SEQ ID Nos: 8743-8758, 20405-20415, 41030-41300 or a sequence with at least 85% identity thereto in the framework region or a sequence having up to 10 conservative amino acid substitutions in the framework region and containing the CDRs of any of the forgoing polypeptides wherein the non-natural TCR antigen binding domain binds to its antigen;
a single domain antibody, a vHH domain, a SVH, and/or FHVH domain comprising a sequence as set forth in any one of SEQ ID NOs 8792-8806, 41572-41590, 43217-43318, a sequence with at least 85% identity thereto in the framework region, or a sequence having up to 10 conservative amino acid substitutions in the framework regions and containing the CDRs of any of the forgoing polypeptides and which binds to its antigen;
a non-immunoglobulin antigen binding domains having a sequence as set forth in any of SEQ ID Nos 43364-43375, 43406-43410, a sequence with at least 85% identity thereto, or a sequence having up to 10 conservative amino acid substitutions and which bind to its antigen;
an scFv domains comprising light chain complementary determining regions 1-3 (LC-CDR1-3) for a selected target antigen as set forth in any of SEQ ID Nos. 20989-21015, 41591-41861; 21024-21050, 41862-42132, 21059-21085, 42133-42403, and complementary heavy chain variable region (vH) that comprises one or more of heavy chain complementary determining regions 1-3 (HC-CDR1-3) for a selected target antigen as set forth in any of SEQ ID Nos. 21094-21120 and 42404-42674; 21129-21155, 42675-42945, and 21164-21190, 42946-43216;
an scFv fragment having a sequence selected from the group consisting of SEQ ID Nos. 8767-8782, 20424-20434, 41301-41571, a sequence with at least 85% identity thereto in the framework region, or a sequence each having up to 10 conservative amino acid substitutions in the framework regions and containing the CDRs of any of the forgoing polypeptides and which bind to its antigen;
one or more receptors comprising of amino acid sequences of any of SEQ ID Nos. 43377-43392 a sequence with at least 85% identity thereto, or sequence having up to 10 conservative amino acid substitutions;
one or more ligands comprising a sequence of any of SEQ ID Nos. 43394-43404, a sequence with at least 85% identity thereto, or sequence having up to 10 conservative amino acid substitutions;
an extracellular domain of CD16A, NKG2D, CD4, PD1, or desmoglein 3 (Dsg3);
an extracellular domain of one or more of hTPO, mTPO, CGHα chain, CGHβ chain, FHβ chain, LHβ chain, TSHβ chain, APRIL, or any combination thereof;
a light chain variable region (vL) comprising one or more of light chain complementary determining regions 1-3 (LC-CDR1-3) for a selected target antigen as set forth in any of SEQ ID Nos. 20989-21015, 41591-41861, 21024-21050, 41862-42132, 21059-21085, 42133-42403, and a complementary heavy chain variable region (vH) comprising one or more of heavy chain complementary determining regions 1-3 (HC-CDR1-3) for a selected target antigen as set forth in any of SEQ ID Nos. 21094-21120, 42404-42674, 21129-21155, 42675-42945, 21164-21190, 42946-43216; and
any combination thereof.

22. The HC-SAR of claim 1, wherein both of the TCR constant chains are hybrid chains.

23. The HC-SAR of claim 1, wherein the constant domain of at least one hybrid chain is derived from an immunoglobulin, or wherein both hybrid chains are derived from an immunoglobulin.

24. The HC-SAR of claim 1, wherein the one or more non-TCR antigen binding domains is selected from the group of antigen binding domains consisting of
a variable region of a heavy and a light chain of an antibody or fragments thereof specific for a predefined target antigen such that when expressed, one of the heavy and the light chains of the antibody or fragments thereof is attached to one of the two chains of the T-cell constant chains and the other of the heavy and the light chains of the antibody or fragments thereof is attached to the other of the two chains of the T-cell constant chains;
two single chain variable fragments (scFv) specific for one or more predefined target antigens, such that, when expressed, one of said scFv is attached to one of said two chains of said T-cell constant chains and the other of said scFv is attached to the other of said two chains of said T-cell constant chains;
two antibody fragment specific for one or more predefined target antigens, such that, when expressed, one of said antibody fragments is attached to one of said two chains of said T-cell constant chains and the other of said antibody fragments is attached to the other of said two chains of said T-cell constant chains;
two single domain antibody (SDAB) fragments specific for one or more predefined target antigens, such that, when expressed, one of said SDAB fragments is attached to one of said two chains of said T-cell constant chains and the other of SDAB fragments is attached to the other of said two chains of said T-cell constant chains;—two camelid vHH domains specific for one or more predefined target antigens, such that, when expressed, one of said vHH domains is attached to one of said two chains of said T-cell constant chains and the other of vHH domains is attached to the other of said two chains of said T-cell constant chains;
two non-immunoglobulin antigen binding scaffolds specific for one or more predefined target antigens, such that, when expressed, one of said non-immunoglobulin antigen binding scaffolds is attached to one of said two chains of said T-cell constant chains and the other of said non-immunoglobulin antigen binding scaffolds domains is attached to the other of said two chains of said T-cell constant chains;
two receptors or a fragment thereof specific for one or more predefined target antigens, such that, when expressed, one of said receptors or a fragment thereof is attached to one of said two chains of said T-cell constant chains and the other of said receptors or a fragment thereof is attached to the other of said two chains of said T-cell constant chains;
two ligands or a fragment thereof specific for one or more predefined target antigens, such that, when expressed, one of said ligands or a fragment thereof is attached to one of said two chains of said T-cell constant chains and the other of said ligands or a fragment thereof is attached to the other of said two chains of said T-cell constant chains;
two structurally distinct antigen binding fragments specific for one or more predefined target antigens, such that, when expressed, one of said antigen binding fragments is attached to one of said two chains of said T-cell constant chains and the other of said antigen binding fragments is attached to the other of said two chains of said T-cell constant chains;
two binding fragments one or both of which are bispecific or multi-specific such that, when expressed, one of said antigen binding fragments is attached to one of said two chains of said T-cell constant chains and the other of said antigen binding fragments is attached to the other of said two chains of said T-cell constant chains;
two autoantigens or fragment thereof, such that, when expressed, one of said autoantigens or fragments thereof is attached to one of said two chains of said T-cell constant chains and the other of said autoantigens or fragments thereof is attached to the other of said two chains of said T-cell constant chains;
two vL or fragment thereof, such that, when expressed, one of said vL or fragments thereof is attached to one of said two chains of said T-cell constant chains and the other of said vL or fragments thereof is attached to the other of said two chains of said T-cell constant chains; and
two vH or fragment thereof, such that, when expressed, one of said vH or fragments thereof is attached to one of said two chains of said T-cell constant chains and the other of said vH or fragments thereof is attached to the other of said two chains of said T-cell constant chains.
